SEQUENCE LISTING (32 sequences)

SEQ ID NO: 1 - protein, 477 aa, Homo sapiens
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPFRYQSLLTRARARGLVCTVWAISALVSFLPILMHWWRAESDEARRCYNDPKCCDFVTNRAYAIASSVVSFYVPLCIMAFVYLRVFREAQKQVKKIDSCERRFLGGPARPPSPSPSPVPAPAPPPGPPRPAAAAATAPLANGRAGKRRPSRLVALREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 2 - DNA, 9862 bp, Homo sapiens
  source 1..9862: mol_type = genomic DNA
tatagcttgatttcaaatagcttcaggctattatcagcctcaaggcacattttgctaacagatttcgtacacttcttggggttttattaagaaattcccccacatatcccttggcgtatcttccagcagttgtccagttaggctactcacttttatttttaactcattttctagtaaagtcaactcaagaatgtaaatgtctttgccaggctcatggcttgcaaccttggatgggttagatgcctattcatcactgctctgcagactaatttgatatacaacttgaaaccacattcaagtcatcagcaactctcctggggcagaaatggctggatatatagattcggtatcataaataatggctaaagaaaaagactcttcacctgggtcatttatgaagaataggtagtacttttttcttttcttttcttttcttttcttttcttttttttttttttttgagttagggtctcactctgtcacccaggctggagtgcagtggcacaaccacatttcactgcagcctcaactcctgggctcaagtgatcctcctgactcagcctcccaaagtgttgggattacaggcaggagccactgagcctggccaggtagtattttttatggacgtggacttgcatgtctcctttggaaaatctcttacctatgtctcgttttatttactacgaataggtatgatatacatttatctcatgagcctaccttgcttttgtaaagtattttgaaatgtttatgtgaaaatgttgaataggtggtaagaatatggattattgctgtaaattataagcaagagtgaagattgtaactactaaaacactcaattatgtctcaagtgtataaaaatgagaatattttaataaattaaatatcatatatttatgaatcaaagctaaatgttcattaatatcaagtaaatatcagtaaacatgggagatataaaaatgtatgagacaattctggctctcagtgagcttacaatctataagagaagaaaaggggaaaatataaaaattaaataacaacataagaagtatcaaaaggcaacctattgtaaattgccacctgaaatgaaattggcagagtcactactataggtcagaggaaggagagatcattttcagaagcagtgggcagagaagataaagagtccagccacatctccaaggctgaattcgctgggaagcaggaggaggttttggtggagggaacccagtgagaaagtatagatgtacaaaaggaaggagtatggatccccaaaaggaaaaagaatacagaaatgcaagttatggtgataaggagagttctaatatgaaatagggctggaaaggccatttagaattggaccggggagaactgtgaatgttaaattgaagaggtaaggttgctgaacagaaaagcatcttggtcaaagcagcattttggaaatactcctttggttatgatatgccagattaaatggagcagggaggaccagagacagggagatcaaatagaacccatggcaagtcccggtgtggggtgaaggtggggggatgggaaaggaagaagttgagataggaactctgtggaagaagtgcacgacctctcaagtgtgctgatcagccctcattgttggagctttctgtcattttccaaagcattgtgaaatatatgggtttttagaactgactctctttaacagttgccaagtgaagaatgaagaaatgaaggagttttatttaacttacaatatccgatacctgtaaaagttgatacgttgaaccttttcttcttgatcttgtatccttacctaagaataaaaagtgttaggctaaaaaaaaagttaaaaaaaaaaaacaccaatcataaaatgtaggaattaaaggagttctgagaacattttatttttattagacaaccaactctagttgaacatgtactattccaaactcttctgtcagtaaaattgaaggaaatcccccagaattcctgagatccctgtactggtactcctgctagaataaccttctggtttcgagggtttataatttaatgtattatacagaaattcggagaaacttgatgtatctctgaatgagcagagaaaaaaagtttcaggaaagttttccaacaattccaatgtcacgatgtgtttatatatataatgatgatacggcaaagttactggcaatctggcttctgccctgaacctctacgaccaagttctggccttatcgcacacagaaaagcaatgccttccacccttcgggggcatttaaggttgacacactctattccggtaacaccacttgacagaaagatggcaaggaaaagcctcttccccacattccctatgcctcaccctgcagctcagcactcagaactcctttcctgggcagcgtcaatttcaacttcttccaatcccttggctattttgagactgccccttgtgggtccagaaggaagccaggaggccggggacagtggctcatgcctctaatcccagcactttgggaggccaaggtgggtggatcacctgaggtcaggagttcgagaccagcctgaccaacatggtaaaacgccatctccactaaaaatacaaaattagccgtgtgtggcggcacacgcctgtaatcccagctacttgacaggctgaggcaggagaatcacttgaacccgggaggcagaggttgcagtgggccaagatcacgccactgaactccagcctgggcaacaaaagtgaaactctgtctcaaaaaaataaaaagaaagaagccaggagtctcaggctttcagtgacttggataacttggggtgtagtctccttcctcccctttctaacgcctcttgtttcctggccactcctcctcaacctccccaagttggaaaatcagctgataaatcatgggcagggggtgaataaacaaatcactccccagttttaacatactgatgctgaggtttgggcctaaagcaaattcatatgtttgcatttatgtaagagaaagtggttaaaactgcttgattttatataaacttgaaaaacattactttttgcttttgacctggacagctgtggaagtcggtctctccttgcaaggtaaggatatgagcttggtcatattgatgaccatcaacgaacatgatgttcgttacgggcgctgttcttattccaatgctagaaggacctgtgagcattatataacattttcatgcacccttacataaacaattagaagctgtttactcctcaaggactcaattagagatttttttaatttttttatttttttttttatttcaggcctgagctgaggacactacaggagccaaattagagaaggggcttgcagtcctgccatattgcatattcatggctttcttgtcccctcacccccagtataatcctttttgccaggttaacaactcactaatttcaaagcaattcatttgccaactcaaaaaaaaaaaaaaaaaaaaccacagatacaactttaaatgcttttggattttaatcctctggagtatttatatgttttcttgtctcatcccttcaaaactaatctcagagttttgagaatctgggaacttgggcaaaggagaaaacaagcacgcagaccaaggaatctgaaatcgcagttcattacgtcagcataaactgacagtacctttttgtttgtattgacccagctcaaattataaaatcacatgagtaataaaacacaaaatacaggtgttatcattgaaaagtctaaaatgtaattaaacgcgttttttccccctcgcggtggtatcgtttactctgtatctcaacgtttctgtctccctaagcctctcctttcatatcatgagcatacatttttgcatcatgctcacacgttcattagctaggactgaggagtgtgtacgattccaaagggccctcagcgttagctgttagatgcacaaaccttcgcttcctttccacatctactgcactcgaggttcaacagaggatccttgcaagaacagcgcccagagagcattcttgacagatgcgcgcttggctccagcaacccgctctgctgggaagtttcttctaaccactaacacccacctccaatcccccaagctgtcacgacgcatgctggctgggtcccgtcttgacgggggaagggttttacacacaccctgctaggctgccccacatcacaaccaagctcgcagggcaaactcctccaagcctggcggacaagctgtcccaggcgctctggcgcttcctgaacaccaaggtcccctccccgctcaagggagctagcgctctgttccgcagagaaccccggaactgcaggtcgaggggatgcggggggagcgggggcgcaggagggagccgagtgctggaggcaaacggggcgcaggagcgggtgcgggaggcaaacggggcgcaggagcgggtgcgggaggcaaacggggcgcaggagtgggtgcgggagcgagtgggggctgagggaggggacggggccccggggggagccaggcgcggaagggggcgcgggggaacagggaccaggaaccagcgggcgcaggaaggggtgcgtccgcaggaacccgcgggcgcacgggaggcactagctacgcgatcagctcgggactctcaggagccgctcaattgccaacgggaggggggtgcggggagttggaggtgggggtgcagaccagacgggggcgtgcctttgcccggattggctgcaggagcctgacgcgaggccccgggggttggcttggggagtgggagcggggtggggtgggtgctgggtgccggagctgcgggcccggcgcgctcagaaacatgctgaagtcccggcggctcttccagcagcggcagcggctccagcagcagcggcggcggcggcggcggcggcagcggcagcgacagcgctcggctcctgcgggaaaggcgcccggcgcccatgcctccggccccgcgccgcggctgccctgacccggccgcgacctccctctgcgcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaagaccgatagcaggtgaactcgaagcccacaatcctcgtctgaatcatccgaggcaaagagaaaagccacggaccgttgcacaaaaaggaaagtttgggaagggatgggagagtggcttgctgatgttccttgttgttttttttttcttttcttttctttcttcttcttttttttttttttttttttttctgtttgtggtccggccttcttttgtgtgtgcgtgtgatgcatctttagatttttttcccccaccaggtggtttttgacactctctgagaggaccggagtggaagatgggtgggttaggggaagggagaagcattaggaggggattaaaatcgatcatcgtggctcccatccctttcccgggaacaggaacacactaccagccagagagaggagaatgacagtttgtcaagacatatttccttttgctttccagagaaatttcattttaatttctaagtaatgatttctgctgttatgaaagcaaagagaaaggatggaggcaaaataaaaaaaaatcacgtttcaagaaatgttaagctcttcttggaacaagccccaccttgctttccttgtgtagggcaaacccgctgtcccccgcgcgcctgggtggtcaggctgagggatttctacctcacactgtgcatttgcacagcagatagaaagacttgtttatattaaacagcttatttatgtatcaatattagttggaaggaccaggcgcagagcctctctctgtgacatgtgactctgtcaattgaagacaggacattaaaagagagcgagagagagaaacagttcagattactgcacatgtggataaaaacaaaaacaaaaaaaaggagtggttcaaaatgccatttttgcacagtgttaggaattacaaaatccacagaagatgttacttgcacaaaaagaaattaaatattttttaaagggagaggggctgggcagatcttaaataaaattcaaactctacttctgttgtctagtatgttattgagctaatgattcattgggaaaatacctttttatactcctttatcatggtactgtaactgtatccatattataaatataattatcttaaggattttttatttttttttatgtccaagtgcccacgtgaatttgctggtgaaagttagcacttgtgtgtaaattctacttcctcttgtgtgttttaccaagtatttatactctggtgcaactaactactgtgtgaggaattggtccatgtgcaataaataccaatgaagcacaatcaagattatgtactgtgtgtctgtaaagggtcagtgacaatgaaaaagacagcttgttttgttcaaaatatagactggatttcccatagagctcttttaataggtttccatgactcaataacatagcaaaatgcctccagacctaaataaggtgtttacctactgagagctacagatttaccctacattttcacagccggattcaaggtgttctagactacttgtaggcactttcaagatcccatctgctgcacttgactgaagaagtgacctttgtgattgcgtagctcctaaaaaaaaaaaaaaaaaaaaaaaagtgacgcggtcatttaactcagctgcaacttttcacggaaatgcaggaaagactaactcattgaatgatcagttgcctacttggaatgcaataagtggcttccacagcttatttttgttttccaatagaaaatcacagcctgcggatgatcagtgtgtgcagatttctccagaggctgtttagaatgaaaaatgctttcatggttaagcaaaaatgtataaaagggcttctgaagtaaatttttttctcttatgtagttaaatagatcattttgccagaagttctgggtggacagatcataggtaaaagcaagtgtgaaaggatttaagtttccaacaaaattacagcacccaaaggatggggtatttttcataaagttatttcctaaaggaagggatagtgcaattctgtcctgagattaattttacattctccaaaatacatttaaagttcttactatatttttaaataagctttttatttcatgaccatacagtaaagaaaccaaagtaaagaatattaacttctggcttatgctgcactttaaatgagcggagtattctttaacagggaaaagttgagactgaatattcttttttgcaatggcatattctaaagacaaggcaaccatcttctgagaaaggttaatgacgtagatgttttagagaccagcagaaggttgcatgaacagtaacagcctcaacaaaagctgctatgaagtttaacccctttccagcaaaaaaaaaaaaaaaaaaaggaaaaggaaaaaggagagcttctttaagaatacaaagataaatacttaaacatattttgaagaacataaaatcagtctcaggatgaaatgtcactataatttccctaggaaccaaatatggttaatatccatccacccccgaccatgagtatgtaagatgcattttcttaggggttttgtccttctttgtttgaaattactagacactgaagtaccaggtaaatgcttcactttcttggtttcctgtttatttacttattttgtctagataagtcttcacatttaaataaaaagaaatcttcccagcccaaaacatatacgcaggcccttcaaatctcaggtcttaaaatcactcaggtcttaaaatcatgaaagtcctttaagctttaagaacaaatgtgtataatttaactacttagtacacaggtttagtcactttagtgagttagaggaaaccactgtaatcttggggccggtttctatgtattggcaggaaatagaaaagatttttgtgtgatctcccaagatctccaactgaaaattacatggattcagaaacagaccatgtcagtgattccaaatccccatgaagactactttttgtcgcttttgaagaaggcagccattgatttataataggaaatgtggggagctgggctgtcagtcccagtagcgatggcagtgggaggagagagcagccagtcctgcttaatctggtgagattgtgcactgcagaaataggaagaagaagacattgagtgtgcaaaaaggcagccttacccatgggcttgtgtgtatcaaagtaccaaaagcataaaccttaaaatcaaagcaggaaaatgtgatcaagatccatagatttgctacattggacgttacattcaggttaaacactaacaataactcaatggagattttccaggatttata

SEQ ID NO: 3 - DNA, 3039 bp, synthetic construct
  source 1..3039: mol_type = other DNA
agaaacatgctgaagtcccggcggctcttccagcagcggcagcggctccagcagcagcggcggcggcggcggcggcggcagcggcagcgacagcgctcggctcctgcgggaaaggcgcccggcgcccatgcctccggccccgcgccgcggctgccctgacccggccgcgacctccctctgcgcaccacgccgcccgggcttctggggtgttccccaaccacggcccagccctgccacaccccccgcccccggcctccgcagctcggcatgggcgcgggggtgctcgtcctgggcgcctccgagcccggtaacctgtcgtcggccgcaccgctccccgacggcgcggccaccgcggcgcggctgctggtgcccgcgtcgccgcccgcctcgttgctgcctcccgccagcgaaagccccgagccgctgtctcagcagtggacagcgggcatgggtctgctgatggcgctcatcgtgctgctcatcgtggcgggcaatgtgctggtgatcgtggccatcgccaagacgccgcggctgcagacgctcaccaacctcttcatcatgtccctggccagcgccgacctggtcatggggctgctggtggtgccgttcggggccaccatcgtggtgtggggccgctgggagtacggctccttcttctgcgagctgtggacctcagtggacgtgctgtgcgtgacggccagcatcgagaccctgtgtgtcattgccctggaccgctacctcgccatcacctcgcccttccgctaccagagcctgctgacgcgcgcgcgggcgcggggcctcgtgtgcaccgtgtgggccatctcggccctggtgtccttcctgcccatcctcatgcactggtggcgggcggagagcgacgaggcgcgccgctgctacaacgaccccaagtgctgcgacttcgtcaccaaccgggcctacgccatcgcctcgtccgtagtctccttctacgtgcccctgtgcatcatggccttcgtgtacctgcgggtgttccgcgaggcccagaagcaggtgaagaagatcgacagctgcgagcgccgtttcctcggcggcccagcgcggccgccctcgccctcgccctcgcccgtccccgcgcccgcgccgccgcccggacccccgcgccccgccgccgccgccgccaccgccccgctggccaacgggcgtgcgggtaagcggcggccctcgcgcctcgtggccctgcgcgagcagaaggcgctcaagacgctgggcatcatcatgggcgtcttcacgctctgctggctgcccttcttcctggccaacgtggtgaaggccttccaccgcgagctggtgcccgaccgcctcttcgtcttcttcaactggctgggctacgccaactcggccttcaaccccatcatctactgccgcagccccgacttccgcaaggccttccagggactgctctgctgcgcgcgcagggctgcccgccggcgccacgcgacccacggagaccggccgcgcgcctcgggctgtctggcccggcccggacccccgccatcgcccggggccgcctcggacgacgacgacgacgatgtcgtcggggccacgccgcccgcgcgcctgctggagccctgggccggctgcaacggcggggcggcggcggacagcgactcgagcctggacgagccgtgccgccccggcttcgcctcggaatccaaggtgtagggcccggcgcggggcgcggactccgggcacggcttcccaggggaacgaggagatctgtgtttacttaagaccgatagcaggtgaactcgaagcccacaatcctcgtctgaatcatccgaggcaaagagaaaagccacggaccgttgcacaaaaaggaaagtttgggaagggatgggagagtggcttgctgatgttccttgttgttttttttttcttttcttttctttcttcttcttttttttttttttttttttttctgtttgtggtccggccttcttttgtgtgtgcgtgtgatgcatctttagatttttttcccccaccaggtggtttttgacactctctgagaggaccggagtggaagatgggtgggttaggggaagggagaagcattaggaggggattaaaatcgatcatcgtggctcccatccctttcccgggaacaggaacacactaccagccagagagaggagaatgacagtttgtcaagacatatttccttttgctttccagagaaatttcattttaatttctaagtaatgatttctgctgttatgaaagcaaagagaaaggatggaggcaaaataaaaaaaaatcacgtttcaagaaatgttaagctcttcttggaacaagccccaccttgctttccttgtgtagggcaaacccgctgtcccccgcgcgcctgggtggtcaggctgagggatttctacctcacactgtgcatttgcacagcagatagaaagacttgtttatattaaacagcttatttatgtatcaatattagttggaaggaccaggcgcagagcctctctctgtgacatgtgactctgtcaattgaagacaggacattaaaagagagcgagagagagaaacagttcagattactgcacatgtggataaaaacaaaaacaaaaaaaaggagtggttcaaaatgccatttttgcacagtgttaggaattacaaaatccacagaagatgttacttgcacaaaaagaaattaaatattttttaaagggagaggggctgggcagatcttaaataaaattcaaactctacttctgttgtctagtatgttattgagctaatgattcattgggaaaatacctttttatactcctttatcatggtactgtaactgtatccatattataaatataattatcttaaggattttttatttttttttatgtccaagtgcccacgtgaatttgctggtgaaagttagcacttgtgtgtaaattctacttcctcttgtgtgttttaccaagtatttatactctggtgcaactaactactgtgtgaggaattggtccatgtgcaataaataccaatgaagcacaa

SEQ ID NO: 4 - protein, 413 aa, Homo sapiens [sequence omitted]

SEQ ID NO: 5 - DNA, 9042 bp, Homo sapiens
  source 1..9042: mol_type = genomic DNA
cacaagaaaggactcttgtctaagccaagacccagtataagattttcctaagcatccagctattggtgtgagggcatgctctggcaccatggaagcttactcctattcagacattcattaggtaaatccagatatgaggttttattcctgtttgacaggctgggaacgacaaatcagtgtcttaagggattgacaaaatatgtaattagatgctatcaatcaccataaaagagcctgctttttcttacatcagataaaggtgcgtttagtctactaagaactttccagttcaaatgaagcattaactctctaaggtcatgtgaacagtaagcagtgctactcgaactcctctgctgggaaataaaataacagcattaggcagggaaaacttgtgaggccatgagcagacctggactcctaattttccttaacactttggcctggacacttcttgttttttcacttcagtaacctcatttgcaaacttgggggaaataaattaactgatatattaagctctttcccagtctaactttctctaaacctaaaatcacaaaaggaggtcaggagaaaagactcttagttagctgactacatcttgacatttctaccagcttggtgaagcagcagggaaagagcgtgcatccattcgtcaaggctggagggcaaaggcccagaacttcctaattgatataagcagattctcctttttttattattattatgctttaagttctaggatacatgtgcagaacgtgcaggtctgttacataggtatacatgtgccatggtggtttgctgcacccatcaacctgtcatctactttaagtatttctcctaatgctatccctcccctaggcccccacccccaaacaggccctggtgtatgatgttcctctccctgtgtccatgtgttctcattgttcaactcccacttatgagtgagaacatgaggtgtttggttttctgttcctgtgttagtttgctgagaatgatggtttccagcttcatccatgcccctgcaaaagacatgaagtcatccttttttatggctgcacagtattccatggtgtatatgtgccacattttctttatcaagtctattcttgatgggcatttgggttggttccaagtctttgctattgtgaacagtgctgcaataaacatacatgtccatgtgtctttatagtagaatgatttataatccttagggtatataccaggtaatgggattgctgggtcaaatggtatttcttgttctagatccttgagggattgccacactgtcttccacaatgtttgaactaatttacattcctaccaacagtgtaaaagtgttcctatttctccacatcctttccagcatctgttgtttcctgacttcctgttttttttttgagacggagtctcactgtgtcaccccggatggagtgcagtggcacaatctcggctcactgcaacctccacctcccaggttcaagcgattctcctgtttcagcccccagagtaggtcagactacaggcatgccacaacttctggctaatttttgtatttttattagagacgaagtttcaccatgttggtcaggctgctctcgaagtcctgacctcaggtgatccacccacctcagcctcccaaagtgctaggattacaggcatgagccactgcgcccggtctgtttcctgatgtgttaatgatcgccattgtatctggtgtgagatggtatttcattgtggttttgatttgcatttcactaataaccagtgctgatgatcttttcttcatatgtttgttggctgcattaatgtcttcttttgagaagtgtctgttcatatcctttgcccactttttcatggggctgtttgttttttcttgtaaatttgtttaagttctttgtagattctggatattagccctttgtcagatggatagattgcaaaatttttccccctttctgtaggttgcctgttcaccctgatgatagtttattttgctgtgcagaaactctttagtttaattagatcccatttgtcaattttggcttttgttgccattgcttttgatgttttagtcatgaagtctttgcccatgcctatgtcctggatgttattgcctagattttcttctagggtttttatggttttaggtcttacgtttaagtctttaattcatcttgagttaattcttgtataaggtgtaaggaaggggtccagtttcagttttctgcatatggctagccagtccttcttgatttagtatttgtgggttttaaaaaaggagtttcccaaaatattcagttaaacttttaagtgacttacgtgtatatctaaatacatgatcagttaatatttgtcttaaaggggttttctttgttcttttcttattataggaaggttaaacaatatgcttatttatgccatagcttcacaaacaggaaggaggttttaaatggtttagttccacaatttgagtagatgcatatttaaagaaacgttgttgcataataaatactgcctcttcctaaaatgcatcatgccacagccaattttggaaaacacaaatatgaggtgagtgtattttgaaaactatgtgaatataatagatctttaattcatatttgtggattttatgggaaatacttgttttctaaggcatctgtcttgcaaaaagtcagtttctgctatgaaggatgttaaaggggatatgtaggttaaattctgtttctgagctttgcttccagagtaaacacccaacttacttttgccctaaagtattttattgttctagtagagaagactaacaacatattctaaaccactaagtaatttatgtaaacttcgcttacaaactacacttgtgtgacacttatatgagcaaaagcattttcatatttcttactatatcattcaattcttgcttaccccaatggaagtgactttatgcccctttagagacaatggaaatcaggtacttcgtgatttctcttaaaaaaaaaaaaaatgaactagaaagctccaagtttggtgaatctggaacctgggtattccagttccagttgtagcccttcctccctatccatcactcctgtctgcatgtaattatgcaatacattgaaaagattaaaagatgggtcttggactcaggcagacctgggtcaaatccagattctggcactgcccagccattgcccctgggcaagccattttcctctttgaacctcatttgtgaattaagctaaaaatagtccccacccccatgggactgtgggaaggattaaatagaataatgcatgaaaagcaaatagcagaatggtccataaatgttaaccattgttatgttattatgtaatctacaaagtacgtttagttacacttcatgaaatactttcagtttttcaaagacaccactaatacatgggaaatcaaaccctgaaaattaatttcactttagcagtaaagtcacatgccagatggaaaggatagtatttcatgaacaaagatcttacttttgagatttggtcttacttttttctttttcttaagggagaattatcttgtgttttttgttttgttttgttttgagatggagtcttgttctgtcacccaggctggagcgcagtgacgtgatctcggctcactgcaaccttcacctcccgggttcaagagattctcctgtctcagcctcccgagtagctgggactacaggtacgtgccaccacacctggctaatttttgtatttttagtagagacaagagttacaccatattggccaggatcttttgctttctatagcttcaaaatgttcttaatgttaagacattcttaatactctgaaccatatgaatttgccattttggtaagtcacagacgccagatggtggcaatttcacatggcacaacccgaaagattaacaaactatccagcagatgaaaggattttttttagtttcattgggtttactgaagaaattgtttgaattctcattgcatctccagttcaacagataatgagtgagtgatgccacactctcaagagttaaaaacaaaacaacaaaaaaattaaaacaaaagcacacaactttctctctctgtcccaaaatacatacttgcatacccccgctccagataaaatccaaagggtaaaactgtcttcatgcctgcaaattcctaaggagggcacctaaagtacttgacagcgagtgtgctgaggaaatcagcagctgttgaagtcacctcctgtgctcttgccaaatgtttgaaagggaatacactgggttaccgggtgtatgttgggaggggagcattatcagtgctcgggtgaggcaagttcggagtacccagatggagacatccgtgtctgtgtcgctctggatgcctccaagccagcgtgtgtttactttctgtgtgtgtcaccatgtctttgtgcttctgggtgcttctgtgtttgtttctggccgcgtttctgtgttggacaggggtgactttgtgccggatggcttctgtgtgagagcgcgcgcgagtgtgcatgtcggtgagctgggagggtgtgtctcagtgtctatggctgtggttcggtataagtctgagcatgtctgccagggtgtatttgtgcctgtatgtgcgtgcctcggtgggcactctcgtttccttccgaatgtggggcagtgccggtgtgctgccctctgccttgagacctcaagccgcgcaggcgcccagggcaggcaggtagcggccacagaagagccaaaagctcccgggttggctggtaaggacaccacctccagctttagccctctggggccagccagggtagccgggaagcagtggtggcccgccctccagggagcagttgggccccgcccgggccagccccaggagaaggagggcgaggggaggggagggaaaggggaggagtgcctcgccccttcgcggctgccggcgtgccattggccgaaagttcccgtacgtcacggcgagggcagttcccctaaagtcctgtgcacataacgggcagaacgcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatggaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatgccttcattgcacctgtttctccaaaaccccttgactggagtgctgttgcctcccccactggaaaccgcaggtaactacttgtaattactgcccatgacttaatgtagaatgatacaagaatgacatgcacagattgcttaaccctttcatttgcctttgagtctgctgctgcaaagctgcatctctcctgacacttgtgccccaaatcagttctgcctgctcttagtatagctcaactctccctatggttattgttctgtgttgttacctcagaaacactgactcacagaagcggagttaaggggatatgtttttttctctccacgtgcacccaccacccaccttccagttctacttgtttcaaaactgtttatatttctgtcttggccatgtgttacagtggagctctttgtactgcatcagggcttggcattttagggataaggaagatgttcttatgaggaagctactcagacatggccccgtaattctgagggaaaattcaaaaggcattggtcatggggagaaaagctggagaacacataactgatggatacctcatgaactagaaacagaattttaaccccttttccttctttcctttggtccctgttttcttctcccactgactctcctcgattcagtgtaaaccaaggttctgagtcttagcactgttagcattttggaccagataactctttgttatgggggctgcatcattgtatgtgtagcacctctggcctctgttcattagatgccaatagcaccccctgcttataacaagcaaaaatgtttccagacattgcaaaagagcccctgaggtgcgaattagcccctggttgagagtcactggtagaaactgtaaaaatctcagcagatacatacattctttctaatgcaagcgcttgattgtgcagagccttagagagggattttcacagttcacctaggcagtaacagaccctcaccagcactctttccattccatcatgctgccttctaaacttgttttctagctgcccaaatagtgatcatgaaatgttaagaaggctttaagtctgtacatgaattgtttgagagggtttatcaatggaggtgaggcctgtgggccatgactcctgtttgtgaagagattataatactgtcaagaggcacgttaggggaaatcacaaaagtaaacacatttcttctcccagcccctttctatttttgcctgtgtgtctgagccagagcttggcccaggtttgatgaagtggatcgtcctccttggcaacgccaggctagagcagatcagcctgcagggttcattgccattccactggctcatgaagctgactccactcccctcttcctttctgttgcagccaaggtccccaaccagaaaagcattggccttctctgcttcctgtcaactcaatgatgggatgtttgggtgagcaccgagctatcaggagaaggttaggcgcctgtgattttggaacatgccatggcaaattggagaatgtgtgtcattcagtgcttttacttttttccaagggttttcatacctattgaaaacccttattacacattatcaccttctctttctactgctattatcacattctctttctactgctctggtctccacactcagagatttgggcagcttctttggctaatattcatgctccctgtagcctcataagatctcagacatggaagagcccatagaaagtatttaacatctgatgagactgaaacgctgtgaggtgaagggcttgcccaaggtaagcagccaaggatgtcagagtgggactcaagccagggaacccaactgctattccaggaactgctgcattctctccaccacattagcattgcgttctttcctcaccctcaactggggctataacataacacattcatttcagccaatatatttttcttttgtccttaacacaaaatttagagcataaacaaataatctgcaatagagacaaaagaaataattgttcatttaactcaacaa

SEQ ID NO: 6 - DNA, 2013 bp, synthetic construct
  source 1..2013: mol_type = other DNA
gcactgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgctcacctgccagactgcgcgccatggggcaacccgggaacggcagcgccttcttgctggcacccaatggaagccatgcgccggaccacgacgtcacgcaggaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccaacagaacactaaacagactatttaacttgagggtaataaacttagaataaaattgtaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaagtctaaagtttacagtaaataaattgtttgaccatg

SEQ ID NO: 7 - RNA, 23 nt, synthetic construct
  source 1..23: mol_type = other RNA
gtagaaggagactacggacgagg

SEQ ID NO: 8 - RNA, 23 nt, synthetic construct
  source 1..23: mol_type = other RNA
tacggacgaggcgatggcgtagg

SEQ ID NO: 9 - RNA, 23 nt, synthetic construct
  source 1..23: mol_type = other RNA
tggattccgaggcgaagccgggg

SEQ ID NO: 10 - RNA, 23 nt, synthetic construct
  source 1..23: mol_type = other RNA
cgacgacaggttaccgggctcgg

SEQ ID NO: 11 - RNA, 23 nt, synthetic construct
  source 1..23: mol_type = other RNA
tgcggccgacgacaggttaccgg

SEQ ID NO: 12 - RNA, 23 nt, synthetic construct
  source 1..23: mol_type = other RNA
ccaacgggcgtgcgggtaagcgg

SEQ ID NO: 13 - RNA, 23 nt, synthetic construct
  source 1..23: mol_type = other RNA
ttggattccgaggcgaagccggg

SEQ ID NO: 14 - RNA, 23 nt, synthetic construct
  source 1..23: mol_type = other RNA
gtacctgcgggtgttccgcgagg

SEQ ID NO: 15 - RNA, 23 nt, synthetic construct
  source 1..23: mol_type = other RNA
cgagcccggtaacctgtcgtcgg

SEQ ID NO: 16 - RNA, 23 nt, synthetic construct
  source 1..23: mol_type = other RNA
gccgttcggggccaccatcgtgg

SEQ ID NO: 17 - RNA, 23 nt, synthetic construct
  source 1..23: mol_type = other RNA
cagacgctcgaacttggcaatgg

SEQ ID NO: 18 - RNA, 23 nt, synthetic construct
  source 1..23: mol_type = other RNA
cgtctgcagacgctcgaacttgg

SEQ ID NO: 19 - RNA, 23 nt, synthetic construct
  source 1..23: mol_type = other RNA
gccggaccacgacgtcacgcagg

SEQ ID NO: 20 - RNA, 23 nt, synthetic construct
  source 1..23: mol_type = other RNA
ccacgacgtcacgcaggaaaggg

SEQ ID NO: 21 - RNA, 23 nt, synthetic construct
  source 1..23: mol_type = other RNA
aagaatatgggcggccccaaagg

SEQ ID NO: 22 - RNA, 23 nt, synthetic construct
  source 1..23: mol_type = other RNA
tcctgcgtgacgtcgtggtccgg

SEQ ID NO: 23 - RNA, 23 nt, synthetic construct
  source 1..23: mol_type = other RNA
cgctaggcacagtaccttgatgg

SEQ ID NO: 24 - RNA, 23 nt, synthetic construct
  source 1..23: mol_type = other RNA
cggtaccagtgcatctgaatggg

SEQ ID NO: 25 - RNA, 23 nt, synthetic construct
  source 1..23: mol_type = other RNA
ttgccccatggcgcgcagtctgg

SEQ ID NO: 26 - RNA, 23 nt, synthetic construct
  source 1..23: mol_type = other RNA
cctgccagactgcgcgccatggg

SEQ ID NO: 27 - DNA, 97 bp, synthetic construct
  source 1..97: mol_type = other DNA
tgctgttgacagtgagcgacagactcagaagtaattgatatagtgaagccacagatgtatatcaattacttctgagtctgctgcctactgcctcgga

SEQ ID NO: 28 - RNA, 20 nt, synthetic construct
  source 1..20: mol_type = other RNA
tggccatcgccaagaccccg

SEQ ID NO: 29 - DNA, 21 bp, synthetic construct
  source 1..21: mol_type = other DNA
ctcatcgtggtgggtaacgtg

SEQ ID NO: 30 - DNA, 21 bp, synthetic construct
  source 1..21: mol_type = other DNA
acacacagcacatctaccgaa

SEQ ID NO: 31 - DNA, 21 bp, synthetic construct
  source 1..21: mol_type = other DNA
gcaaatgtggcaggaaaaagt

SEQ ID NO: 32 - DNA, 22 bp, synthetic construct
  source 1..22: mol_type = other DNA
tgatccagctacagaaacctga

(19) United States
(12) Patent Application Publication
(10) Pub. No.: US 2026/0207668 A1
(43) Pub. Date: Jul. 23, 2026

(54) MANIPULATION OF ADRENERGIC RECEPTORS TO INFLUENCE IMMUNE CELL DIFFERENTIATION AND FUNCTION

(71) Applicant: Salk Institute for Biological Studies, La Jolla, CA (US)

(72) Inventors: Anna-Maria Globig, San Diego, CA (US); Susan M. Kaech, Del Mar, CA (US); Steven Zhao, San Diego, CA (US)

(73) Assignee: Salk Institute for Biological Studies, La Jolla, CA (US)

(21) Appl. No.: 19/144,360

(22) PCT Filed: Dec. 28, 2023
(86) PCT No.: PCT/US2023/086231

§ 371 Date: Jun. 27, 2025

Related U.S. Application Data
(60) Provisional application No. 63/436,401, filed on Dec. 30, 2022.

(51) Int. Cl. A61K 35/17; A61K 31/165; A61K 35/15; A61K 40/11; A61K 40/13; A61K 40/15; A61K 40/17; A61K 40/19; A61K 40/42; A61K 40/46; A61P 35/00; C12N 5/0781; C12N 5/0783; C12N 5/0784; C12N 5/0786; C12N 9/22; C12N 15/113; C12N 15/85

(52) U.S. Cl. CPC A61K 35/17; A61K 31/165; A61K 35/15; A61K 40/11; A61K 40/13; A61K 40/15; A61K 40/17; A61K 40/19; A61K 40/42; A61K 40/46; A61P 35/00; C12N 5/0635; C12N 5/0636; C12N 5/0639; C12N 5/0645; C12N 5/0646; C12N 9/222; C12N 15/113; C12N 15/85; C12N 2310/11; C12N 2310/14; C12N 2310/20; C12N 2310/531; C12N 2501/15; C12N 2501/2302; C12N 2501/2307; C12N 2501/2315; C12N 2501/385; C12N 2510/00

(57) ABSTRACT

Exposure of ADRB1-expressing T cells to catecholamines suppresses cytokine production and impairs T cell proliferation. Genetic or pharmacological ablation of β-adrenergic signaling via ADRB1 reduces development of T cell exhaustion in a chronic infection model and improves cytotoxic functions in tumor-infiltrating lymphocytes (TILs) when combined with immune checkpoint blockade (ICB) therapy in a melanoma model. In an ICB-resistant tumor model of murine pancreatic cancer, beta-blockers and ICB synergize to enhance and reprogram T cell responses. Disclosed are modified PBMCs with decreased ADRB1 and/or ADRB2 function, and their use to treat cancer and viral infection. Also disclosed are modified PBMCs with increased ADRB1 and/or ADRB2 function, and their use to treat autoimmune diseases.

### CROSS REFERENCE TO RELATED APPLICATIONS

This application claims priority to U.S. Provisional Application No. 63/436,401, filed Dec. 30, 2022, which is herein incorporated by reference in its entirety.

### ACKNOWLEDGMENT OF GOVERNMENT SUPPORT

This invention was made with government support under Grant Nos. R01 CA240909. RO1 CA216101, and K00CA222741 awarded by the National Institutes of Health. The government has certain rights in the invention.

### FIELD

This relates to immunotherapies, compositions and methods of preventing or reducing immune cell exhaustion, methods of inducing immune cell tolerance, and uses thereof for treating cancer, viral infections, and autoimmunity.

### INCORPORATION OF ELECTRONIC SEQUENCE LISTING

The Sequence Listing is submitted as an XML file in the form of the file named “7158-109406-02_ST26.xml” (~53,884 bytes), which was created on Nov. 28, 2023 which is incorporated by reference herein.

### BACKGROUND

T cell ‘exhaustion’ is a specific CD8⁺ T cell differentiation state that is induced by chronic exposure to antigen as found in chronic viral infections or cancer, and is characterized by a progressive loss of effector functions as well as expression of multiple inhibitory receptors such as PD-1 and TIM3 and the transcription factor TOX. In recent years, several subsets of TOX⁺ exhausted CD8⁺ T cells (TEX) have been defined, leading to the establishment of a progenitor-progeny lineage relationship with progressive development of exhaustion. In this paradigm, TOX⁺ progenitor exhausted cells (TEX_{prog}, PD-1⁺ SLAMF6⁺ TCF1⁺), which exhibit stem cell-like properties, sustain the T cell response by proliferating to self-renew as well as producing more terminally differentiated effectors (TEX_{eff}, PD-1⁺ CX3CR1⁺ TIM3⁺) that display some effector functions and terminally exhausted cells (TEXₜₑᵣₘ, PD-1⁺ CD101⁺ TIM3+) that have impaired effector functions. Progenitor cells provide the proliferative burst following ICB targeting inhibitory receptors like PD-1 expressed on exhausted T cells. The advent of checkpoint therapy to reinvigorate T cell responses has led to major improvements in clinical outcomes of cancer patients. However there remains a need to further decipher the physiological processes that govern T cell exhaustion.

The autonomic nervous system consists of the sympathetic and the parasympathetic nervous systems that respectively control the “fight or flight” or “rest and digest” responses. Acute or chronic stress triggers the activation of sympathetic nerves, leading to release of the catecholamines noradrenaline and adrenaline (also called norepinephrine and epinephrine) into tissues and circulation to achieve a broad range of effects on host physiology. Activation of the sympathetic nervous system also influences the immune system, including T helper cell differentiation and CD8′ T cell responses to infection and tumors by altering priming and migration. Catecholamines achieve these effects by signaling through the α-adrenergic receptors and the β-adrenergic receptors ADRB1, ADRB2 and ADRB3. Despite expression of adrenergic receptors on T cells, the regulation of T cell responses by the sympathetic nervous system has frequently been ascribed to indirect mechanisms, e.g., via induction of noradrenaline-mediated hypoxia or the modulation of other immune cell populations.

### SUMMARY

It is demonstrated herein that catecholamines contribute to the progression of CD8⁺ T cell exhaustion in chronic viral infection and cancer through the β1-adrenergic receptor, ADRB1, and that ablation of beta-adrenergic signaling synergizes with ICB to improve T cell effector functions, form tissue-resident memory like cells and suppress tumor growth.

Based on these observations, disclosed herein are modified PBMCs which can include one or both of (a) an agent that reduces Adrb1 expression or a non-naturally occurring genetic modification that reduces an amount of functional ADRB1; and/or (b) an agent that reduces Adrb2 expression or a non-naturally occurring genetic modification that reduces an amount of functional ADRB2. Also disclosed are methods of treating cancer and viral infections using the modified PBMCs with reduced Adrb1 and/or Adrb2 expression/activity. Further disclosed herein are modified PBMCs including (a) an agent that increases Adrb1 expression or a non-naturally occurring genetic modification that increases an amount of functional ADRB1; and/or (b) an agent that increases Adrb2 expression or a non-naturally occurring genetic modification that increases an amount of functional ADRB2. Also disclosed are methods of treating autoimmune disease the modified PBMCs with increased Adrb1 and/or Adrb2 expression/activity. Further disclosed is a method for preventing or treating cancer in a subject, including administering an agent that reduces or inhibits ADRB1 signaling and/or an agent that reduces or inhibits ADRB2 signaling; and a therapeutically effective amount of immune checkpoint blockade (ICB) agent.

The foregoing and other features of this disclosure will become more apparent from the following detailed description of several aspects which proceeds with reference to the accompanying figures.

### BRIEF DESCRIPTION OF THE FIGURES

FIGS. 1A-1H show that exhausted CD8⁺ T cells express ADRB1 and are associated with sympathetic nerves in the spleen and in tumors. (A) Volcano plot depicting genes upregulated in terminal exhausted cells vs in progenitor exhausted cells respectively. Data from PRJNA497086, TEX_{prog}=CD101⁻ TTM3⁻, TEX_{eff}=CD101⁻ TTM3⁺, TEXₜₑᵣₘ=CD101⁺ TIM3⁺. (B) Frequency of ADRB1 expressing cells within exhausted subsets depicted at d30 p.i, with LCMV-clone 13. PD-1⁻=PD-1⁻ P14⁺, TEX_{prog}=TCF1⁺ PD-1⁺ P14⁺. TEX_{eff}=CX3CR1⁺ TIM3⁺ PD-1⁺ P14⁺. TEXₜₑᵣₘ=CD101⁺ TIM3⁺ PD-1⁺ P14⁺. N=7 per subset pooled from 2 independent experiments, Friedman test with Dunn's multiple comparisons test. (C) Representative flow plots depicting ADRB1 expression on naïve CD8⁺ T cells (CD44⁻) and gp33⁺ CD8⁺ T cells from mice infected with LCMV-Armstrong (d8) and LCMV-clone 13 (d30), representative of 3 independent experiments. (D) Representative overview image of WT P14⁺ cells, CD101 expression, and tyrosine hydroxylase at d14 p.i. in the spleen of a recipient mouse infected with LCMV-clone 13. Four different regions such as this were imaged across the spleens of two different mice from 2 independent experiments and the shortest distance to TH signal for CD101⁺ P14⁺ cells vs CD101⁻ P14⁺ cells was calculated. Image collected using 20× objective tiled across a 3×3 region. (E+F) Representative images of nerves within PDAC tumors with T cell clusters (representative of 5 individual tumors from 2 independent experiments). Scale bar: 50 μm. Image was collected using a 40× objective tiled across the entire PDAC tumor within the pancreas, with 1.5 μm z step sizes to acquire a 30 μm imaging depth. (G) Density of TH⁺ stromal cells across 164 primary human NSCLCs. (H) Density of CD8⁺ TILs in NSCLCs with low (bottom 80%, n=130) and high TH (top 20%, n=34) expression. Mann-Whitney test. Unless otherwise specified, mean±SEM is indicated in scatter plots. Two-sided statistical tests were used. **** indicates a p value<0.0001, ***<0.001, **<0.01, *<0.05.

FIGS. 2A-20 show that exhausted CD8⁺ T cells express the adrenergic receptor ADRB1 and are associated with sympathetic nerves in the chronically infected spleen and in human and murine tumors. (A) Expression of Adrb1, Adrb2, Adrb3 and Crem in TEX. Data from PRJNA497086, TEX_{prog}=CD101⁻ TIM3⁻, TEX_{eff}=CD101⁻ TIM3⁺, TEXₜₑᵣₘ=CD101⁺ TIM3⁺. (B) Annotation of TEX subsets and expression of Adrb1 in scRNA-Seq (GSE122713, d28 clone 13). (C) ADRB1 expression on gp33⁺ CD8⁺ T cells in LCMV Armstrong (d8, n=6), clone 13 (d30, n=7) and naïve CD8⁺ T cells (n=6), pooled from 3 independent experiments. One-way ANOVA with Dunnett's multiple comparisons test. (D) GSEA using GO adrenergic receptor signaling pathway and d45 LCMV-clone 13 data from PRJNA497086. (E) Serum noradrenaline levels of mice infected with LCMV-clone 13 at d7 and d21 p.i., and of naïve mice (n=5 mice per group, pooled from 2 independent experiments). Mann-Whitney test. (F) Expression of ADRB1 and other canonical TEX genes in CD8⁺ T cells isolated from HIV infected and uninfected donors. Data from GSE157829. (G) ADRB1 expression on PD-1⁻ and PD-1⁺ CD8⁺ T cells in the blood of HIV infected donors (triangles, n=4) and uninfected donors (circles, n=2). Paired t test. (H) Flow plots showing CD8⁺ T cells in the blood of a HIV infected donor. Frame indicates ADRB1⁺ cells. (I) Expression of exhaustion markers on ADRB1⁻ vs ADRB1⁺ CD8⁺ T cells in the blood of HIV infected donors (triangles, n=4) and uninfected donors (circles, n=2). Paired t test. (J) Representative image of WT P14⁺ cells and CD101 expression in the spleen at d14 p.i, with LCMV-clone 13. Tyrosine hydroxylase (TH) as marker for sympathetic nerves. (K) Quantification of shortest distance to TH signal for CD101⁺ P14⁺ cells vs CD101⁻ P14⁺ cells calculated from 4 different splenic regions of 2 different mice. 20× objective. Dashed line indicates the median distance of CD101⁻ P14⁺ cells to TH signal. Linear mixed model. Boxplots show median. The lower and upper hinges correspond to the first and third quartiles. The upper whisker extends from the hinge to the largest value no further than 1.5*IQR from the hinge. (L) Representative images of nerves within PDAC tumors with T cell clusters (representative of 5 individual tumors). Scale bar: 50 μm, 40× objective. (M) Distances of CD8⁺ T cell subsets to TH⁺ nerves in PDAC tumors, pooled from 5 individual tumors. Kruskal-Wallis test with Dunn's multiple comparisons test. Dotted line indicates median. (N) Representative images of human NSCLC samples with low (left) and high local TH expression (right). (O) Density of exhausted CD8⁺ TILs in human NSCLCs with low (bottom 80%, n=130) and high TH expression (top 20%, n=34). Mann-Whitney test. Unless otherwise specified, mean±SEM is indicated in scatter plots. Two-sided statistical tests were used. **** indicates ap value<0.0001, ***<0.001, **<0.01, *<0.05.

FIGS. 3A-3E illustrate that catecholamine signaling through ADRB1 impairs CD8⁺ T cell cytokine production and proliferation. (A) Verification of Adrb1 overexpression. qPCRs were performed in duplicates. (B) Flow plots of cytokine production by Adrb1 OE P14⁺ cells and control empty vector (EV) P14⁺ cells. Cells were stimulated with gp33 in the presence or absence of 10 μM adrenaline (A) or noradrenaline (NA). Plots are gated on GFP⁺ CD8⁺ T cells and indicative of one of 3 independent experiments. (C) Cytokine production by control empty vector (EV) retroviral (RV)-transduced P14⁺ cells (n=3 individual experiments). Cells were stimulated with gp33 in the presence or absence of 10 μM A or NA. (D) Representative flow plots depicting frequency of Adrb1 overexpressing (OE) P14⁺ cells and control (EV) P14⁺ cells at d0 and d6 of culture. Cells were mixed at a 1:1 ratio and cultured for 6 days total with and without the addition of 10 μM A or NA. Representative of 4 independent experiments. (E) Ratio of Adrb1 OE P14⁺ cells to control (EV) P14⁺ cells over 6 days of culture. Cells were mixed at a 1:1 ratio and cultured for 6 days total with and without the addition of 10 μM A or NA. Quantification from 4 independent experiments. Unless otherwise specified, mean±SEM is indicated in scatter plots.

FIGS. 4A-4K show that Adrb1 overexpression promotes CD8⁺ T cell exhaustion through elevated cAMP levels. (A) Cytokine production by Adrb1 overexpressing (OE) P14⁺ cells and control empty vector (EV) retroviral (RV)-transduced P14⁺ cells (n=3 individual experiments) stimulated with gp33 in the presence or absence of 10 μM adrenaline (A) or noradrenaline (NA). Ordinary one-way ANOVA with Dunnett's multiple comparisons test. (B) CTV staining of Adrb1 OE P14⁺ cells and control EV RV transduced P14⁺ cells cultured for 3 days with or without 10 μM A or NA. One of three experiments is shown. Plots gated on congenic⁺ GFP⁺ CD8⁺ T cells. Experimental setup (C) and representative Calcium flux plots (D). Adrb1 OE P14⁺ cells and EV control P14⁺ cells were cultured in the presence or absence of catecholamines and calcium flux was measured after TCR stimulation with CD3 crosslink or gp33 stimulation using Indo-1 staining. One of four experiments is shown. Plots gated on congenic⁺ GFP⁺ CD8⁺ T cells. (E) Western Blot showing phosphorylation of PLCγ1 in Adrb1 OE and EV control P14⁺ cells after TCR stimulation in the presence or absence of NA. Western Blot is representative of one of 7 independent experiments quantified in bottom figure panel. Wilcoxon test. For source data, see FIG. 1. (F+G) Frequency and phenotype of Adrb1 OE P14⁺ cells on day 7 p.i, with LCMV-clone 13. 10 k Adrb1 OE P14⁺ cells and EV P14⁺ cells each were transferred into recipient mice at a 1:1 ratio (n=11, pooled from 3 independent experiments). Wilcoxon test. (H) cAMP levels in Adrb1 OE P14⁺ cells and EV P14⁺ cells on day 7 p.i, with LCMV-clone 13 (n=11, pooled from 3 independent experiments). Wilcoxon test. (I) cAMP levels in gp33⁺ CD8⁺ T cells from mice infected with LCMV Arm (d8 p.i.), LCMV-clone 13 (d30 p.i.) or from naïve cells (CD44⁻ CD8⁺ T cells). N=5 mice per group, representative of one of 2 independent experiments. cAMP levels in ADRB1+ and ADRB1⁻ gp33⁺ CD8⁺ T cells on d30 p.i, with LCMV-clone 13 (n=13 per group, pooled from 2 independent experiments). CREM expression in gp33⁺ CD8⁺ T cells from mice infected with LCMV Arm (d8 p.i., n=4) or LCMV-clone 13 (d30 p.i., n=5) or from naïve cells (CD44⁻ CD8⁺ T cells, n=4, representative of one of 3 independent experiments). CREM expression in ADRB1+ and ADRB1⁻ gp33+CD8⁺ T cells on d30 p.i, with LCMV-clone 13 (n=10 per group, pooled from 2 independent experiments). Kruskal-Wallis test with Dunn's multiple comparisons test or Wilcoxon test. (J) Left, cAMP levels in subsets of exhausted gp33⁺ CD8⁺ T cells. PD-1⁻ cells, TEX_{prog}=TIM3⁻ PD-1⁺, TEX_{eff}=CX3CR1⁺ TIM3+PD-1⁺, TEXₜₑᵣₘ=CD101⁺ TIM3⁺ PD-1⁺, n=13 per group, pooled from 2 independent experiments. Right, CREM expression in subsets of exhausted gp33⁺ CD8⁺ T cells. PD-1⁻=PD-1⁻, TEX_{prog}=TCF1⁺ PD-1⁺, TEX_{eff}=CX3CR1⁺ TIM3⁺ PD-1⁺, TEXₜₑᵣₘ=CD101⁺ TIM3⁺ PD-1⁺, n=8 per group, pooled from 2 independent experiments. Friedman test with Dunn's multiple comparisons test. (K) Representative overlay histogram of CREM expression in Adrb1 KO and control cells. Representative of 2 independent experiments. Unless otherwise specified, mean±SEM is indicated in scatter plots. Two-sided statistical tests were used. **** indicates a p value<0.0001, ***<0.001, **<0.01, *<0.05.

FIGS. 5A-5G show that Adrb1 knockout prevents terminal differentiation of antigen-specific CD8⁺ T cells. (A) Verification of knockout in Adrb1^{fl/fl} Granzyme B^{Cre+} mice (Adrb1 cKO). Splenocytes were isolated and stimulated with anti-CD3/CD28. Cells were cultured in vitro for 4 days with IL-2 and subsequently CD8⁺ T cells were sorted. Adrb1 expression was assessed with qPCR performed in triplicates. (B) Expression of PD-1 on Adrb1 cKO P14⁺ cells and WT P14⁺ cells at d7 p.i. (n=9) and d40 p.i. (n=16). Paired t test. Flow data for both d7 and d40 are each pooled from 3 independent experiments. (C) Cytokine production of Adrb1 cKO P14⁺ cells and wild type P14⁺ cells at d40 p.i. after 6 h stimulation with gp33. N=16, paired t-test, pooled from 3 independent experiments. (D) Viral titers in spleen from Adrb1 cKO and WT P14⁺ recipients at d36 p.i, with LCMV-clone13 and treated with anti-PD-L1 or IgG2B from d23-d36 p.i. Ordinary one-way ANOVA with Holm-Šidák's multiple comparisons test with a single pooled variance. WT IgG2B n=7. WT anti-PD-L1 n=8, cKO IgG2B n=8, cKO anti-PD-L1 n=8, pooled from 2 independent experiments. (E) Verification of Crem knockdown using shCrem and qPCR performed in triplicates to determine Crem expression. Splenocytes were transduced with shCrem or shCd19 as control and cultured for 3 days before sorting on Ametrine⁺ CD8⁺ T cells. Unpaired t test. (F) Frequency and phenotype of Crem knockdown P14+ and control knockdown P14⁺ at d7 p.i, with LCMV-clone 13. 15 k Crem knockdown P14⁺ cells and control knockdown P14⁺ cells each were mixed at a 1:1 ratio and transferred into recipient mice that were infected with LCMV-clone 13 on the same day (n=15, pooled from 3 independent experiments). Wilcoxon test. (G) Representative image of Adrb1 cKO P14+ and WT P14⁺ cells at d31 p.i. in the spleen of a recipient mouse infected with LCMV-clone 13. Image is representative of 3 independent regions and 2 independent experiments. B220 and F4/80 also stained. Image was collected using a 20× objective tiled across a 3×3 region. Unless otherwise specified, mean±SEM is indicated in scatter plots. Two-sided statistical tests were used. **** indicates ap value<0.0001, ***<0.001, **<0.01, *<0.05.

FIGS. 6A-6M show that Adrb1 knockout prevents terminal differentiation of antigen-specific CD8⁺ T cells in chronic viral infection. (A) Experimental setup. Adrb1^{fl/fl} Granzyme B^{Cre+} or ^{Cre-} P14⁺ cells with different congenic markers were mixed at a 1:1 ratio with 7.5 k P14⁺ cells of each genotype co-adoptively transferred into B6 recipients that were infected with LCMV-clone 13 one day later. Flow data for d7 and d40 are each pooled from 3 independent experiments. (B) Frequency of Adrb1 conditional knockout P14⁺ (Adrb1 cKO) and wild type P14⁺ cells (WT) at d7 and d40 p.i, with LCMV-clone 13. n=9 for d7, n=16 for d40, paired t test. (C) Expression of TOX on Adrb1 cKO P14⁺ and WT P14⁺ cells at d7 p.i. (n=9) and d40 p.i. (n=16). Paired t test. (D+E) Representative flow plots gated on P14⁺ CD8⁺ T cells showing KLRG1 and CD127 expression of Adrb1 cKO P14⁺ and WT P14⁺ cells at d7 p.i, with LCMV-clone 13 (D). Quantification of CD127⁻ KLRG1⁺ Adrb1 cKO P14⁺ and WT P14⁺ cells at d7 p.i. (E). N=9, paired t test. (F-H) Expression of TIM3, CD101, CD39, CXCR6, CX3CR1 and TCF1 on Adrb1 cKO P14⁺ and WT P14⁺ cells (F) and quantification of exhaustion subsets (G+H) at d40 p.i. N=16, paired t test. (I) GRANZYME B production of Adrb1 cKO P14⁺ and WT P14⁺ cells at d40 p.i. N=16, paired t-test. (J) Viral titers from spleen of Adrb1 cKO (n=10) and WT P14⁺ recipients (n=9) at d30 p.i, with LCMV-clone 13, pooled from 2 independent experiments. Unpaired t test. (K) Frequency of Adrb1 cKO P14⁺ and WT P14⁺ cells after treatment with α-PD-L1 or IgG2B from d23-d36. Frequency assessed at d36 p.i, with LCMV-clone 13. Values shown as ratio to mean WT IgG2B, n=5 per group pooled from 2 independent experiments. Mixed effects-analysis with Dunnett's multiple comparisons test. (L) cAMP levels and CREM expression in Adrb1 cKO P14⁺ and WT P14⁺ cells at d40 p.i., n=16, paired t test. (M) Representative image of Adrb1 cKO P14+ and WT P14⁺ cells in the spleen at d31 p.i. Tyrosine hydroxylase staining as marker for sympathetic nerves shown. Quantification of shortest distance to nerve staining for Adrb1 cKO P14⁺ and WT P14⁺ cells calculated from 6 different splenic regions of 2 different mice. Image collected using a 10× objective, scale bar: 50 μm. Linear mixed model. Boxplots show median. The lower and upper hinges correspond to the first and third quartiles. The upper whisker extends from the hinge to the largest value no further than 1.5*IQR from the hinge. Unless otherwise specified. mean±SEM is indicated in scatter plots. Two-sided statistical tests were used. **** indicates a p value<0.0001. ***<0.001, **<0.01, *<0.05.

FIGS. 7A-7C show that pharmacological blockade of ADRB1 prevents advanced exhaustion differentiation of antigen-specific CD8⁺ T cells. (A+B) Absolute cell counts of gp33⁺ CD8⁺ T cells (A), and of different exhausted subsets of gp33⁺ CD8⁺ T cells (B) isolated from the spleens of mice treated with atenolol or control water during chronic infection with LCMV-clone 13, assessed at d37 p.i. (n=7 per group, pooled from 3 independent experiments). Mann-Whitney test. (C) Absolute cell counts of cytokine producing cells after antigen-specific stimulation with gp33. Cells were isolated from the spleens of mice treated with atenolol or control water during chronic infection with LCMV-clone 13, assessed at d37 p.i. (n=8 per group, pooled from 3 independent experiments). Mann-Whitney test. Unless otherwise specified, mean±SEM is indicated in scatter plots. Two-sided statistical tests were used. **** indicates a p value<0.0001, ***<0.001, **<0.01. *<0.05.

FIGS. 8A-8L depict that pharmacological blockade of ADRB1 prevents advanced exhaustion differentiation of antigen-specific CD8⁺ T cells and combines with checkpoint therapy to increase T cell function. (A) LCMV-clone 13 infected mice were left untreated or treated with atenolol throughout the infection and analyzed at d37 p.i. Data are pooled from 3 independent experiments, n=7/group. Mann-Whitney test. Flow plot gated on CD8⁺ T cells. (B) Graphs show frequency of TEX_{prog}, TEX_{eff} and TEXₜₑᵣₘ, subtypes of gp33⁺ CD8⁺ T cells in mice treated with atenolol (n=7) or without treatment (n=7), pooled from 3 independent experiments. Mann-Whitney test. (C) Flow plot gated on CD8⁺ T cells, depicting cytokine production of CD8⁺ T cells in mice treated with atenolol or without treatment at d37 p.i. after antigen-specific stimulation with gp33 for 6 h. (D) Cytokine production of CD8⁺ T cells in mice treated with atenolol (n=8) or without treatment (n=8) at d37 p.i. after stimulation with gp33 for 6 h, pooled from 3 independent experiments. Mann-Whitney test. (E+F) cAMP (E) and CREM (F) levels in gp33⁺ CD8⁺ T cells at d37 p.i. in untreated mice and mice treated with atenolol. Histograms show representative cAMP staining (left, E) and bar graphs show cumulative data from 3 independent experiments (e, n=10 or 14 mice/group) or CREM staining (F) from 2 independent experiments (n=4 mice/group). Mann-Whitney test. (G) Viral titers in the serum of LCMV-clone 13-infected mice treated with atenolol (n=10) or without treatment (n=8), pooled from 3 independent experiments. Mann-Whitney test. (H) Flow plots (left) and quantification (right, n=17 each, pooled from 3 independent experiments) of ADRB1 expression by TEX isolated from YUMMER tumors implanted into WT B6 mice, plots gated on the indicated cell populations. (Friedman test with Dunn's multiple comparisons test. PD-1⁻=PD-1-negative CD8⁺. (I) Experimental setup for YUMMER tumor experiments. (J) Normalized tumor mass of YUMMER tumors relative to IgG control. (IgG n=14, atenolol n=9, ICB n=12, atenolol+ICB n=13, pooled from 3 independent experiments). Plot shows median with IQR. Kruskal-Wallis Test with Dunn's multiple comparisons test. (K) Flow plots depicting IFNg and TNF production by CD8⁺ T cells isolated from YUMMER tumors following stimulation with PMA/ionomycin for 5 h. Plots gated on CD8⁺ T cells. (L) Cytokine production by CD8⁺ T cells after stimulation with PMA/ionomycin (IgG n=10, atenolol n=9. ICB n=7, atenolol+ICB n=8, pooled from 2 independent experiments). Kruskal-Wallis Test with Dunn's multiple comparisons test. Unless otherwise specified, mean±SEM is indicated in scatter plots. Two-sided statistical tests were used. **** indicates a p value<0.0001, ***<0.001, **<0.01, *<0.05.

FIGS. 9A-9H show that pharmacological blockade of ADRB1 synergizes with immune checkpoint blockade to increase T cell function. (A) Flow plots depicting ADRB1 expression by different subsets of exhausted T cells isolated from MC38 tumors, plots are gated on the indicated cell populations (left panel). Quantification of ADRB1 expression on different subsets of exhausted CD8⁺ T cells isolated from MC38 tumors implanted into wild type B6 mice (right panel, n=16 each, pooled from 3 independent experiments). Friedman test with Dunn's multiple comparisons test. (B) cAMP levels in ADRB1⁻ vs ADRB1⁺ CD8⁺ T cells isolated from MC38 tumors (left panel, n=8), Wilcoxon test was used to determine statistical significance. cAMP levels in different subsets of exhausted CD8⁺ T cells isolated from MC38 tumors (right panel. n=8 each). Friedman test with Dunn's multiple comparisons test. Data pooled from 2 independent experiments. PD-1⁻=PD-1⁻ CD8⁺, TEX_{prog}=TIM3⁻ PD-1⁺ CD8⁺. TEX_{eff}=CX3CR1⁺ TIM3⁺ PD-1⁺ CD8⁺, TEXₜₑᵣₘ=CD101⁺ TIM3⁺ PD-1⁺ CD8⁺. (C) Expression analysis of the indicated exhaustion markers in ADRB1^{high} and ADRB1^{low} CD8⁺ T cells in RNA Seq data generated from 16 human colorectal cancer samples. Data from GSE200997. Statistics were calculated using a linear mixed model. Boxplots show median. The lower and upper hinges correspond to the first and third quartiles. The upper whisker extends from the hinge to the largest value no further than 1.5*IQR from the hinge. (D) cAMP levels in ADRB1⁻ vs ADRB1⁺ CD8⁺ T cells isolated from YUMMER tumors (n=4), Wilcoxon test was used to determine statistical significance. cAMP levels in different subsets of exhausted CD8⁺ T cells isolated from YUMMER tumors (n=4 each). Friedman test with Dunn's multiple comparisons test. Data representative of one of 2 independent experiments. PD-1⁻=PD-1⁻ CD8⁺, TEX_{prog}=TIM3⁻ PD-1⁺ CD8⁺, TEX_{eff}=CX3CR1⁺ TIM3+PD-1⁺ CD8⁺, TEXₜₑᵣₘ=CD101⁺ TIM3⁺ PD-1⁺ CD8⁺. (E) Normalized tumor mass of YUMMER tumors from mice under the indicated treatment conditions relative to IgG control (IgG n=9, atenolol+ICB n=8, atenolol+ICB+CD8⁺ depletion n=9), pooled from 2 independent experiments. Kruskal-Wallis Test with Dunn's multiple comparisons test. (F) ADRB1/ADRB2 selectivity of atenolol and CGP 20712A. (G) Schematic of the experimental setup used in the subsequent figure panels showing YUMMER tumor experiments (left panel). Normalized tumor mass of YUMMER tumors relative to IgG control (IgG n=5, CGP 20712A n=7, ICB n=8, CGP 20712A+ICB n=8, pooled from 2 independent experiments). Kruskal-Wallis Test with Dunn's multiple comparisons test (right panel). (H) Flow cytometric assessment of production of cytokines by CD8⁺ T cells after stimulation with PMA/ionomycin (IgG n=5, CGP 20712A n=7, ICB n=8, CGP 20712A+1CB n=7, pooled from 2 independent experiments). Kruskal-Wallis Test with Dunn's multiple comparisons test. Unless otherwise specified, mean±SEM is indicated in scatter plots. Two-sided statistical tests were used. **** indicates a p value<0.0001, ***<0.001, **<0.01, *<0.05.

FIGS. 10A-10B show that exhausted CD8⁺ T cells in PDAC tumors express ADRB1. (A) Representative flow cytometry plots depicting ADRB1 expression of different CD8⁺ T cell subsets in PDAC tumors implanted into wild type B6 mice. Plots are gated on the indicated cell populations and representative of 2 independent experiments. (B) Expression analysis of the indicated exhaustion markers in ADRB1^{high} and ADRB1^{low} CD8⁺ T cells in RNA Seq data generated from 16 human pancreatic cancer samples. Data from GSE155698. Statistics were calculated using a linear mixed model. Boxplots show median. The lower and upper hinges correspond to the first and third quartiles. The upper whisker extends from the hinge to the largest value no further than 1.5*IQR from the hinge. Two-sided statistical tests were used. **** indicates ap value<0.0001, ***<0.001, **<0.01, *<0.05.

FIGS. 11A-11C show that pharmacological blockade of ADRB1 or ADRB2 alone is ineffective in PDAC. (A) cAMP levels in ADRB1⁻ vs ADRB1⁺ CD8⁺ T cells isolated from PDAC tumors (left panel, n=6 pooled from 2 independent experiments), Wilcoxon test. cAMP levels in different subsets of exhausted CD8⁺ T cells isolated from PDAC tumors (second to left panel, n=6 each pooled from 2 independent experiments). Friedman test with Dunn's multiple comparisons test. PD-1⁻=PD-1⁻ CD8⁺, TEX_{prog}=TIM3⁻ PD-1⁺ CD8⁺, TEX_{eff}=CX3CR1⁺ TIM3⁺ PD-1⁺ CD8⁺, TEXₜₑᵣₘ=CD101⁺ TIM3⁺ PD-1⁺ CD8⁺. CREM expression in ADRB1⁺ vs ADRB1⁻ CD8⁺ T cells isolated from PDAC tumors (second to right panel, n=6 pooled from 2 independent experiments), Wilcoxon test. CREM expression in different subsets of exhausted CD8⁺ T cells isolated from PDAC tumors (right panel, n=10 each, pooled from 3 independent experiments). Friedman test with Dunn's multiple comparisons test. PD-1⁻=PD-1⁻ CD8⁺. TEX_{prog}=SLAMF6⁺ PD-1+CD8⁺, TEX_{eff}=CX3CR1⁺ TIM3⁺ PD-1⁺ CD8⁺, TEXₜₑᵣₘ=CD101⁺ TIM3⁺ PD-1⁺ CD8⁺. (B) Normalized tumor mass of PDAC tumors relative to IgG control (IgG n=5, atenolol n=5, ICB n=5, atenolol+ICB n=5), representative of 2 independent experiments. Kruskal-Wallis Test with Dunn's multiple comparisons test. (C) ADRB1/ADRB2 selectivity of atenolol and ICI 118551 (left panel). Normalized tumor mass of PDAC tumors relative to IgG control (IgG n=9, ICI 118551 n=9, ICB n=8, ICI 118551+ICB n=10), pooled from 2 independent experiments. ICI 118551 hydrochloride was administered at 0.2 μg/g i.p. daily. Kruskal-Wallis Test with Dunn's multiple comparisons test. Unless otherwise specified, mean±SEM is indicated in scatter plots. Two-sided statistical tests were used. **** indicates a p value<0.0001, ***<0.001, **<0.01, *<0.05.

FIGS. 12A-12L depict that beta-blocker treatment enables effective checkpoint therapy in pancreatic cancer. (A) ADRB1 expression on TEX subsets (n=9, pooled from 2 independent experiments). One-Way ANOVA with Friedman test. PD-1⁻=PD-1-negative CD8⁺, TEX_{prog}=SLAMF6⁺ PD-1⁺ CD8⁺, TEX_{eff}=CX3CR1⁺ TIM3⁺ PD-1⁺ CD8⁺, TEXₜₑᵣₘ=CD101⁺ TIM3⁺ PD-1⁺ CD8⁺. (B) Experimental setup. (C) Tumor volume of PDAC tumors determined by ultrasound (IgG n=9, Propranolol n=9, ICB n=10, Propranolol+ICB n=9). Ordinary two-way ANOVA with Tukey's multiple comparisons test, pooled from 2 independent experiments. (D) Normalized tumor mass of PDAC tumors relative to IgG control (IgG n=21, Propranolol n=18, ICB n=19, Propranolol+ICB n=24). Ordinary one-way ANOVA with Holm-Sídák's multiple comparisons test. Pooled from 5 independent experiments. (E) Tumor volume determined by ultrasound in mice treated with isotype control (n=5), Propranolol+ICB (n=5) or Propranolol+ICB with CD8⁺ depletion (n=7). Ordinary two-way ANOVA with Tukey's multiple comparisons test, pooled from 2 independent experiments. (F) Frequency of CD8⁺ T cells, expression of exhaustion markers and production of cytokines after stimulation with PMA/ionomycin (IgG n=17, Propranolol n=13, ICB n=14, Propranolol+ICB n=18 for CD8, PD-1, TIM3: IgG n=7. Propranolol n=8, ICB n=10. Propranolol+ICB n=9 for SLAMF6; IgG n=12, Propranolol n=10, ICB n=9, Propranolol+ICB n=14 for IFNγ and GRANZYME B, pooled from 4 independent experiments). Kruskal-Wallis Test with Dunn's multiple comparisons test. (G) UMAP dimensional reduction of scRNA Seq from PDAC tumors colored by cell types. (H) UMAP depicting 8 T cell clusters within the T cell cluster identified in (G). Stacked bar graphs depicting frequency of T cell clusters in different treatment conditions. (I) Heatmap of manually selected marker genes used for cell type annotation as depicted in h. (J) Gene signature analysis in CD8⁺ T cells using AUCell. Boxplots show median. The lower and upper hinges correspond to the first and third quartiles. The upper whisker extends from the hinge to the largest value no further than 1.5*IQR from the hinge. (K) Heatmap of manually selected cytokines and chemokines in different treatment conditions. (L) Venn diagram depicting upregulated genes in CD8⁺ T cells in the indicated treatment conditions vs IgG control. Numbers in the Venn diagram indicate number of upregulated genes. Unless otherwise specified, mean±SEM is indicated in scatter plots. **** indicates ap value<0.0001. ***<0.001, **<0.01, *<0.05.

FIGS. 13A-13C depict single cell RNA Seq analysis of tumor infiltrating T cells from PDAC tumors. (A-C) UMAP and violin plots depicting the MAGIC imputed expression of the indicated marker genes for the T cell clusters within the PDAC scRNA Seq dataset.

FIGS. 14A-14D depict that pharmacological blockade of adrenergic receptors reprograms tumor infiltrating T cells in PDAC. (A) Heatmap showing the top 10 marker genes for each T cell cluster. Expression is normalized across cells. (B) Heatmap showing differentially regulated pathways in CD8⁺ T cells extracted from the PDAC scRNA Seq dataset. T cells were grouped by condition and z score of median pathway activity is shown. (C) UMAP visualization of CD8⁺ T cells extracted from the PDAC scRNA Seq dataset. Cells were annotated using SingleR with the published exhaustion subsets from PRJNA497086. Stacked bar graphs show frequency of exhaustion subsets per treatment condition as annotated per PRJNA497086. (D) Expression of the beta-blocker signature identified in FIG. 12L in human T cells isolated from PDAC patients under beta-blocker therapy (n=2) vs PDAC patients without beta-blocker therapy (n=15). Data from GSE155698. Tukey's test. Boxplots show median. The lower and upper hinges correspond to the first and third quartiles. The upper whisker extends from the hinge to the largest value no further than 1.5*IQR from the hinge. Two-sided statistical tests were used. **** indicates a p value<0.0001, ***<0.001, **<0.01, *<0.05.

FIGS. 15A-15C show that pharmacological blockade of adrenergic receptors does not directly affect tumor cell proliferation. (A) Tumor expression of Ki67 relative to IgG determined by IHC in YUMMER tumors from mice under the indicated treatment conditions (IgG n=14, atenolol n=6, pooled from 4 independent experiments). Mann Whitney test. (B) Tumor expression of Ki67 relative to IgG determined by IHC in YUMMER tumors from mice under the indicated treatment conditions (IgG n=7, CGP 20712A n=7, pooled from 2 independent experiments). Mann Whitney test. (C) Tumor expression of Ki67 relative to IgG determined by IHC in PDAC tumors from mice under the indicated treatment conditions (IgG n=8, propranolol n=8, pooled from 2 independent experiments). Mann Whitney test. Unless otherwise specified, mean±SEM is indicated in scatter plots. Two-sided statistical tests were used.

### SEQUENCES

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand.

In the accompanying sequence listing:

SEQ ID NO: 1 is an exemplary amino acid sequence
of human ADRB1, from NCBI reference
sequence NP_000675.1.
mgagvlvlgasepgnlssaaplpdgaataarllvpasppasllppasespeplsqqwtag
mgllmalivllivagnvlvivaiaktprlqtltnlfimslasadlvmgllvvpfgativv
wgrweygsffcelwtsvdvlcvtasietlcvialdrylaitspfryqslltrararglvc
tvwaisalvsflpilmhwwraesdearrcyndpkccdfvtnrayaiassvvsfyvplcim
afvylrvfreaqkqvkkidscerrflggparppspspspvpapapppgpprpaaaaatap
langragkrrpsrlvalreqkalktlgiimgvftlcwlpfflanvvkafhrelvpdrlfv
ffnwlgyansafnpiiycrspdfrkafqgllccarraarrrhathgdrprasgclarpgp
ppspgaasddddddvvgatpparllepwagonggaaadsdssldepcrpgfaseskv
SEQ ID NO: 2 is an exemplary nucleotide sequence
of human Adrb1, from NCBI reference
sequence NG_012187.1.
tatagcttgatttcaaatagcttcaggctattatcagcctcaaggcacattttgctaaca
gatttcgtacacttcttggggttttattaagaaattcccccacatatcccttggcgtatc
ttccagcagttgtccagttaggctactcacttttatttttaactcattttctagtaaagt
caactcaagaatgtaaatgtctttgccaggctcatggcttgcaaccttggatgggttaga
tgcctattcatcactgctctgcagactaatttgatatacaacttgaaaccacattcaagt
catcagcaactctcctggggcagaaatggctggatatatagattcggtatcataaataat
ggctaaagaaaaagactcttcacctgggtcatttatgaagaataggtagtacttttttct
tttcttttcttttcttttcttttcttttttttttttttttgagttagggtctcactctgt
cacccaggctggagtgcagtggcacaaccacatttcactgcagcctcaactcctgggctc
aagtgatcctcctgactcagcctcccaaagtgttgggattacaggcaggagccactgagc
ctggccaggtagtattttttatggacgtggacttgcatgtctcctttggaaaatctctta
cctatgtctcgttttatttactacgaataggtatgatatacatttatctcatgagcctac
cttgcttttgtaaagtattttgaaatgtttatgtgaaaatgttgaataggtggtaagaat
atggattattgctgtaaattataagcaagagtgaagattgtaactactaaaacactcaat
tatgtctcaagtgtataaaaatgagaatattttaataaattaaatatcatatatttatga
atcaaagctaaatgttcattaatatcaagtaaatatcagtaaacatgggagatataaaaa
tgtatgagacaattctggctctcagtgagcttacaatctataagagaagaaaaggggaaa
atataaaaattaaataacaacataagaagtatcaaaaggcaacctattgtaaattgccac
ctgaaatgaaattggcagagtcactactataggtcagaggaaggagagatcattttcaga
agcagtgggcagagaagataaagagtccagccacatctccaaggctgaattcgctgggaa
gcaggaggaggttttggtggagggaacccagtgagaaagtatagatgtacaaaaggaagg
agtatggatccccaaaaggaaaaagaatacagaaatgcaagttatggtgataaggagagt
tctaatatgaaatagggctggaaaggccatttagaattggaccggggagaactgtgaatg
ttaaattgaagaggtaaggttgctgaacagaaaagcatcttggtcaaagcagcattttgg
aaatactcctttggttatgatatgccagattaaatggagcagggaggaccagagacaggg
agatcaaatagaacccatggcaagtcccggtgtggggtgaaggtggggggatgggaaagg
aagaagttgagataggaactctgtggaagaagtgcacgacctctcaagtgtgctgatcag
ccctcattgttggagctttctgtcattttccaaagcattgtgaaatatatgggtttttag
aactgactctctttaacagttgccaagtgaagaatgaagaaatgaaggagttttatttaa
cttacaatatccgatacctgtaaaagttgatacgttgaaccttttcttcttgatcttgta
tccttacctaagaataaaaagtgttaggctaaaaaaaaagttaaaaaaaaaaaacaccaa
tcataaaatgtaggaattaaaggagttctgagaacattttatttttattagacaaccaac
tctagttgaacatgtactattccaaactcttctgtcagtaaaattgaaggaaatccccca
gaattcctgagatccctgtactggtactcctgctagaataaccttctggtttcgagggtt
tataatttaatgtattatacagaaattcggagaaacttgatgtatctctgaatgagcaga
gaaaaaaagtttcaggaaagttttccaacaattccaatgtcacgatgtgtttatatatat
aatgatgatacggcaaagttactggcaatctggcttctgccctgaacctctacgaccaag
ttctggccttatcgcacacagaaaagcaatgccttccacccttcgggggcatttaaggtt
gacacactctattccggtaacaccacttgacagaaagatggcaaggaaaagcctcttccc
cacattccctatgcctcaccctgcagctcagcactcagaactcctttcctgggcagcgtc
aatttcaacttcttccaatcccttggctattttgagactgccccttgtgggtccagaagg
aagccaggaggccggggacagtggctcatgcctctaatcccagcactttgggaggccaag
gtgggtggatcacctgaggtcaggagttcgagaccagcctgaccaacatggtaaaacgcc
atctccactaaaaatacaaaattagccgtgtgtggcggcacacgcctgtaatcccagcta
cttgacaggctgaggcaggagaatcacttgaacccgggaggcagaggttgcagtgggcca
agatcacgccactgaactccagcctgggcaacaaaagtgaaactctgtctcaaaaaaata
aaaagaaagaagccaggagtctcaggctttcagtgacttggataacttggggtgtagtct
ccttcctcccctttctaacgcctcttgtttcctggccactcctcctcaacctccccaagt
tggaaaatcagctgataaatcatgggcagggggtgaataaacaaatcactccccagtttt
aacatactgatgctgaggtttgggcctaaagcaaattcatatgtttgcatttatgtaaga
gaaagtggttaaaactgcttgattttatataaacttgaaaaacattactttttgcttttg
acctggacagctgtggaagtcggtctctccttgcaaggtaaggatatgagcttggtcata
ttgatgaccatcaacgaacatgatgttcgttacgggcgctgttcttattccaatgctaga
aggacctgtgagcattatataacattttcatgcaccettacataaacaattagaagctgt
ttactcctcaaggactcaattagagatttttttaatttttttatttttttttttatttca
ggcctgagctgaggacactacaggagccaaattagagaaggggcttgcagtcctgccata
ttgcatattcatggctttcttgtcccctcacccccagtataatcctttttgccaggttaa
caactcactaatttcaaagcaattcatttgccaactcaaaaaaaaaaaaaaaaaaaacca
cagatacaactttaaatgcttttggattttaatcctctggagtatttatatgttttcttg
tctcatcccttcaaaactaatctcagagttttgagaatctgggaacttgggcaaaggaga
aaacaagcacgcagaccaaggaatctgaaatcgcagttcattacgtcagcataaactgac
agtacctttttgtttgtattgacccagctcaaattataaaatcacatgagtaataaaaca
caaaatacaggtgttatcattgaaaagtctaaaatgtaattaaacgcgttttttccccct
cgcggtggtatcgtttactctgtatctcaacgtttctgtctccctaagcctctcctttca
tatcatgagcatacatttttgcatcatgctcacacgttcattagctaggactgaggagtg
tgtacgattccaaagggccctcagcgttagctgttagatgcacaaaccttcgcttccttt
ccacatctactgcactcgaggttcaacagaggatccttgcaagaacagcgcccagagagc
attcttgacagatgcgcgcttggctccagcaacccgctctgctgggaagtttcttctaac
cactaacacccacctccaatcccccaagctgtcacgacgcatgctggctgggtcccgtct
tgacgggggaagggttttacacacaccctgctaggctgccccacatcacaaccaagctcg
cagggcaaactcctccaagcctggcggacaagctgtcccaggcgctctggcgcttcctga
acaccaaggtcccctccccgctcaagggagctagcgctctgttccgcagagaaccccgga
actgcaggtcgaggggatgcggggggagcgggggcgcaggagggagccgagtgctggagg
caaacggggcgcaggagcgggtgcgggaggcaaacggggcgcaggagcgggtgcgggagg
caaacggggcgcaggagtgggtgcgggagcgagtgggggctgagggaggggacggggccc
cggggggagccaggcgcggaagggggcgcgggggaacagggaccaggaaccagcgggcgc
aggaaggggtgcgtccgcaggaacccgcgggcgcacgggaggcactagctacgcgatcag
ctcgggactctcaggagccgctcaattgccaacgggaggggggtgcggggagttggaggt
gggggtgcagaccagacgggggcgtgcctttgcccggattggctgcaggagcctgacgcg
aggccccgggggttggcttggggagtgggagcggggtggggtgggtgctgggtgccggag
ctgcgggcccggcgcgctcagaaacatgctgaagtcccggcggctcttccagcagcggca
gcggctccagcagcagcggcggcggcggcggcggcggcagcggcagcgacagcgctcggc
tcctgcgggaaaggcgcccggcgcccatgcctccggccccgcgccgcggctgccctgacc
cggccgcgacctccctctgcgcaccacgccgcccgggcttctggggtgttccccaaccac
ggcccagccctgccacaccccccgcccccggcctccgcagctcggcatgggcgcgggggt
gctcgtcctgggcgcctccgagcccggtaacctgtcgtcggccgcaccgctccccgacgg
cgcggccaccgcggcgcggctgctggtgcccgcgtcgccgcccgcctcgttgctgcctcc
cgccagcgaaagccccgagccgctgtctcagcagtggacagcgggcatgggtctgctgat
ggcgctcatcgtgctgctcatcgtggcgggcaatgtgctggtgatcgtggccatcgccaa
gacgccgcggctgcagacgctcaccaacctcttcatcatgtccctggccagcgccgacct
ggtcatggggctgctggtggtgccgttcggggccaccatcgtggtgtggggccgctggga
gtacggctccttcttctgcgagctgtggacctcagtggacgtgctgtgcgtgacggccag
catcgagaccctgtgtgtcattgccctggaccgctacctcgccatcacctcgcccttccg
ctaccagagcctgctgacgcgcgcgcgggcgcggggcctcgtgtgcaccgtgtgggccat
ctcggccctggtgtccttcctgcccatcctcatgcactggtggcgggcggagagcgacga
ggcgcgccgctgctacaacgaccccaagtgctgcgacttcgtcaccaaccgggcctacgc
catcgcctcgtccgtagtctccttctacgtgcccctgtgcatcatggccttcgtgtacct
gcgggtgttccgcgaggcccagaagcaggtgaagaagatcgacagctgcgagcgccgttt
cctcggcggcccagcgcggccgccctcgccctcgccctcgcccgtccccgcgcccgcgcc
gccgcccggacccccgcgccccgccgccgccgccgccaccgccccgctggccaacgggcg
tgcgggtaagcggcggccctcgcgcctcgtggccctgcgcgagcagaaggcgctcaagac
gctgggcatcatcatgggcgtcttcacgctctgctggctgcccttcttcctggccaacgt
ggtgaaggccttccaccgcgagctggtgcccgaccgcctcttcgtcttcttcaactggct
gggctacgccaactcggccttcaaccccatcatctactgccgcagccccgacttccgcaa
ggccttccagggactgctctgctgcgcgcgcagggctgcccgccggcgccacgcgaccca
cggagaccggccgcgcgcctcgggctgtctggcccggcccggacccccgccatcgcccgg
ggccgcctcggacgacgacgacgacgatgtcgtcggggccacgccgcccgcgcgcctgct
ggagccctgggccggctgcaacggcggggcggcggcggacagcgactcgagcctggacga
gccgtgccgccccggcttcgcctcggaatccaaggtgtagggcccggcgcggggcgcgga
ctccgggcacggcttcccaggggaacgaggagatctgtgtttacttaagaccgatagcag
gtgaactcgaagcccacaatcctcgtctgaatcatccgaggcaaagagaaaagccacgga
ccgttgcacaaaaaggaaagtttgggaagggatgggagagtggcttgctgatgttccttg
ttgttttttttttcttttcttttctttcttcttcttttttttttttttttttttttctgt
ttgtggtccggccttcttttgtgtgtgcgtgtgatgcatctttagatttttttcccccac
caggtggtttttgacactctctgagaggaccggagtggaagatgggtgggttaggggaag
ggagaagcattaggaggggattaaaatcgatcatcgtggctcccatccctttcccgggaa
caggaacacactaccagccagagagaggagaatgacagtttgtcaagacatatttccttt
tgctttccagagaaatttcattttaatttctaagtaatgatttctgctgttatgaaagca
aagagaaaggatggaggcaaaataaaaaaaaatcacgtttcaagaaatgttaagctcttc
ttggaacaagccccaccttgctttccttgtgtagggcaaacccgctgtcccccgcgcgcc
tgggtggtcaggctgagggatttctacctcacactgtgcatttgcacagcagatagaaag
acttgtttatattaaacagcttatttatgtatcaatattagttggaaggaccaggcgcag
agcctctctctgtgacatgtgactctgtcaattgaagacaggacattaaaagagagcgag
agagagaaacagttcagattactgcacatgtggataaaaacaaaaacaaaaaaaaggagt
ggttcaaaatgccatttttgcacagtgttaggaattacaaaatccacagaagatgttact
tgcacaaaaagaaattaaatattttttaaagggagaggggctgggcagatcttaaataaa
attcaaactctacttctgttgtctagtatgttattgagctaatgattcattgggaaaata
cctttttatactcctttatcatggtactgtaactgtatccatattataaatataattatc
ttaaggattttttatttttttttatgtccaagtgcccacgtgaatttgctggtgaaagtt
agcacttgtgtgtaaattctacttcctcttgtgtgttttaccaagtatttatactctggt
gcaactaactactgtgtgaggaattggtccatgtgcaataaataccaatgaagcacaatc
aagattatgtactgtgtgtctgtaaagggtcagtgacaatgaaaaagacagcttgttttg
ttcaaaatatagactggatttcccatagagctcttttaataggtttccatgactcaataa
catagcaaaatgcctccagacctaaataaggtgtttacctactgagagctacagatttac
cctacattttcacagccggattcaaggtgttctagactacttgtaggcactttcaagatc
ccatctgctgcacttgactgaagaagtgacctttgtgattgcgtagctcctaaaaaaaaa
aaaaaaaaaaaaaaagtgacgcggtcatttaactcagctgcaacttttcacggaaatgca
ggaaagactaactcattgaatgatcagttgcctacttggaatgcaataagtggcttccac
agcttatttttgttttccaatagaaaatcacagcctgcggatgatcagtgtgtgcagatt
tctccagaggctgtttagaatgaaaaatgctttcatggttaagcaaaaatgtataaaagg
gcttctgaagtaaatttttttctcttatgtagttaaatagatcattttgccagaagttct
gggtggacagatcataggtaaaagcaagtgtgaaaggatttaagtttccaacaaaattac
agcacccaaaggatggggtatttttcataaagttatttcctaaaggaagggatagtgcaa
ttctgtcctgagattaattttacattctccaaaatacatttaaagttcttactatatttt
taaataagctttttatttcatgaccatacagtaaagaaaccaaagtaaagaatattaact
tctggcttatgctgcactttaaatgagcggagtattctttaacagggaaaagttgagact
gaatattcttttttgcaatggcatattctaaagacaaggcaaccatcttctgagaaaggt
taatgacgtagatgttttagagaccagcagaaggttgcatgaacagtaacagcctcaaca
aaagctgctatgaagtttaacccctttccagcaaaaaaaaaaaaaaaaaaaggaaaagga
aaaaggagagcttctttaagaatacaaagataaatacttaaacatattttgaagaacata
aaatcagtctcaggatgaaatgtcactataatttccctaggaaccaaatatggttaatat
ccatccacccccgaccatgagtatgtaagatgcattttcttaggggttttgtccttcttt
gtttgaaattactagacactgaagtaccaggtaaatgcttcactttcttggtttcctgtt
tatttacttattttgtctagataagtcttcacatttaaataaaaagaaatcttcccagcc
caaaacatatacgcaggcccttcaaatctcaggtcttaaaatcactcaggtcttaaaatc
atgaaagtcctttaagctttaagaacaaatgtgtataatttaactacttagtacacaggt
ttagtcactttagtgagttagaggaaaccactgtaatcttggggccggtttctatgtatt
ggcaggaaatagaaaagatttttgtgtgatctcccaagatctccaactgaaaattacatg
gattcagaaacagaccatgtcagtgattccaaatccccatgaagactactttttgtcgct
tttgaagaaggcagccattgatttataataggaaatgtggggagctgggctgtcagtccc
agtagcgatggcagtgggaggagagagcagccagtcctgcttaatctggtgagattgtgc
actgcagaaataggaagaagaagacattgagtgtgcaaaaaggcagccttacccatgggc
ttgtgtgtatcaaagtaccaaaagcataaaccttaaaatcaaagcaggaaaatgtgatca
agatccatagatttgctacattggacgttacattcaggttaaacactaacaataactcaa
tggagattttccaggatttata
SEQ ID NO: 3 is an exemplary nucleotide sequence of
human Adrb1 transcript cDNA from NCBI
reference sequence NM_000684.3.
agaaacatgctgaagtcccggcggctcttccagcagcggcagcggctccagcagcagcgg
cggcggcggcggcggcggcagcggcagcgacagcgctcggctcctgcgggaaaggcgccc
ggcgcccatgcctccggccccgcgccgcggctgccctgacceggcegcgacctccctctg
cgcaccacgccgcccgggcttctggggtgttccccaaccacggcccagccctgccacacc
ccccgcccccggcctccgcagctcggcatgggcgcgggggtgctcgtcctgggcgcctcc
gagcccggtaacctgtcgtcggccgcaccgctccccgacggcgcggccaccgcggcgcgg
ctgctggtgcccgcgtcgccgcccgcctcgttgctgcctcccgccagcgaaagccccgag
ccgctgtctcagcagtggacagcgggcatgggtctgctgatggcgctcatcgtgctgctc
atcgtggcgggcaatgtgctggtgatcgtggccatcgccaagacgccgcggctgcagacg
ctcaccaacctcttcatcatgtccctggccagcgccgacctggtcatggggctgctggtg
gtgccgttcggggccaccatcgtggtgtggggccgctgggagtacggctccttcttctgc
gagctgtggacctcagtggacgtgctgtgcgtgacggccagcatcgagaccctgtgtgtc
attgccctggaccgctacctcgccatcacctcgcccttccgctaccagagcctgctgacg
cgcgcgcgggcgcggggcctcgtgtgcaccgtgtgggccatctcggccctggtgtccttc
ctgcccatcctcatgcactggtggcgggcggagagcgacgaggcgcgccgctgctacaac
gaccccaagtgctgcgacttcgtcaccaaccgggcctacgccatcgcctcgtccgtagtc
tccttctacgtgcccctgtgcatcatggccttcgtgtacctgcgggtgttccgcgaggcc
cagaagcaggtgaagaagatcgacagctgcgagcgccgtttcctcggcggcccagcgcgg
ccgccctcgccctcgccctcgcccgtccccgcgcccgcgccgccgcccggacccccgcgc
cccgccgccgccgccgccaccgccccgctggccaacgggcgtgcgggtaagcggcggccc
tcgcgcctcgtggccctgcgcgagcagaaggcgctcaagacgctgggcatcatcatgggc
gtcttcacgctctgctggctgcccttcttcctggccaacgtggtgaaggccttccaccgc
gagctggtgcccgaccgcctcttcgtcttcttcaactggctgggctacgccaactcggcc
ttcaaccccatcatctactgccgcagccccgacttccgcaaggccttccagggactgctc
tgctgcgcgcgcagggctgcccgccggcgccacgcgacccacggagaccggccgcgcgcc
tcgggctgtctggcccggcccggacccccgccatcgcccggggccgcctcggacgacgac
gacgacgatgtcgtcggggccacgccgcccgcgcgcctgctggagccctgggccggctgc
aacggcggggcggcggcggacagcgactcgagcctggacgagccgtgccgccccggcttc
gcctcggaatccaaggtgtagggcccggcgcggggcgcggactccgggcacggcttccca
ggggaacgaggagatctgtgtttacttaagaccgatagcaggtgaactcgaagcccacaa
tcctcgtctgaatcatccgaggcaaagagaaaagccacggaccgttgcacaaaaaggaaa
gtttgggaagggatgggagagtggcttgctgatgttccttgttgttttttttttcttttc
ttttctttcttcttcttttttttttttttttttttttctgtttgtggtccggccttcttt
tgtgtgtgcgtgtgatgcatctttagatttttttcccccaccaggtggtttttgacactc
tctgagaggaccggagtggaagatgggtgggttaggggaagggagaagcattaggagggg
attaaaatcgatcatcgtggctcccatccctttcccgggaacaggaacacactaccagcc
agagagaggagaatgacagtttgtcaagacatatttccttttgctttccagagaaatttc
attttaatttctaagtaatgatttctgctgttatgaaagcaaagagaaaggatggaggca
aaataaaaaaaaatcacgtttcaagaaatgttaagctcttcttggaacaagccccacctt
gctttccttgtgtagggcaaacccgctgtcccccgcgcgcctgggtggtcaggctgaggg
atttctacctcacactgtgcatttgcacagcagatagaaagacttgtttatattaaacag
cttatttatgtatcaatattagttggaaggaccaggcgcagagcctctctctgtgacatg
tgactctgtcaattgaagacaggacattaaaagagagcgagagagagaaacagttcagat
tactgcacatgtggataaaaacaaaaacaaaaaaaaggagtggttcaaaatgccattttt
gcacagtgttaggaattacaaaatccacagaagatgttacttgcacaaaaagaaattaaa
tattttttaaagggagaggggctgggcagatcttaaataaaattcaaactctacttctgt
tgtctagtatgttattgagctaatgattcattgggaaaatacctttttatactcctttat
catggtactgtaactgtatccatattataaatataattatottaaggattttttattttt
ttttatgtccaagtgcccacgtgaatttgctggtgaaagttagcacttgtgtgtaaattc
tacttcctcttgtgtgttttaccaagtatttatactctggtgcaactaactactgtgtga
ggaattggtccatgtgcaataaataccaatgaagcacaa
SEQ ID NO: 4 is an exemplary amino acid sequence
of human ADRB2, from NCBI reference
sequence NP_000015.2.
mgqpgngsafllapngshapdhdvtqerdevwvvgmgivmslivlaivfgnvlvitaiak
ferlqtvtnyfitslacadlvmglavvpfgaahilmkmwtfgnfwcefwtsidvlcvtas
ietlcviavdryfaitspfkyqslltknkarviilmvwivsgltsflpiqmhwyrathqe
aincyanetccdfftnqayaiassivsfyvplvimvfvysrvfqeakrqlqkidksegrf
hvqnlsqveqdgrtghglrrsskfclkehkalktlgiimgtftlcwlpffivnivhviqd
nlirkevyillnwigyvnsgfnpliyorspdfriafqellclrrsslkaygngyssngnt
geqsgyhveqekenkllcedlpgtedfvghqgtvpsdnidsqgrncstndsll
SEQ ID NO: 5 is an exemplary nucleotide sequence
of human Adrb2, from NCBI reference
sequence NG_016421.2.
cacaagaaaggactcttgtctaagccaagacccagtataagattttcctaagcatccagc
tattggtgtgagggcatgctctggcaccatggaagcttactcctattcagacattcatta
ggtaaatccagatatgaggttttattcctgtttgacaggctgggaacgacaaatcagtgt
cttaagggattgacaaaatatgtaattagatgctatcaatcaccataaaagagcctgctt
tttcttacatcagataaaggtgcgtttagtctactaagaactttccagttcaaatgaagc
attaactctctaaggtcatgtgaacagtaagcagtgctactcgaactcctctgctgggaa
ataaaataacagcattaggcagggaaaacttgtgaggccatgagcagacctggactccta
attttccttaacactttggcctggacacttcttgttttttcacttcagtaacctcatttg
caaacttgggggaaataaattaactgatatattaagctctttcccagtctaactttctct
aaacctaaaatcacaaaaggaggtcaggagaaaagactcttagttagctgactacatctt
gacatttctaccagcttggtgaagcagcagggaaagagcgtgcatccattcgtcaaggct
ggagggcaaaggcccagaacttcctaattgatataagcagattctcctttttttattatt
attatgctttaagttctaggatacatgtgcagaacgtgcaggtctgttacataggtatac
atgtgccatggtggtttgctgcacccatcaacctgtcatctactttaagtatttctccta
atgctatccctcccctaggcccccacccccaaacaggccctggtgtatgatgttcctctc
cctgtgtccatgtgttctcattgttcaactcccacttatgagtgagaacatgaggtgttt
ggttttctgttcctgtgttagtttgctgagaatgatggtttccagcttcatccatgcccc
tgcaaaagacatgaagtcatccttttttatggctgcacagtattccatggtgtatatgtg
ccacattttctttatcaagtctattcttgatgggcatttgggttggttccaagtctttgc
tattgtgaacagtgctgcaataaacatacatgtccatgtgtctttatagtagaatgattt
ataatccttagggtatataccaggtaatgggattgctgggtcaaatggtatttcttgttc
tagatccttgagggattgccacactgtcttccacaatgtttgaactaatttacattccta
ccaacagtgtaaaagtgttcctatttctccacatcctttccagcatctgttgtttcctga
cttcctgttttttttttgagacggagtctcactgtgtcaccccggatggagtgcagtggc
acaatctcggctcactgcaacctccacctcccaggttcaagcgattctcctgtttcagcc
cccagagtaggtcagactacaggcatgccacaacttctggctaatttttgtatttttatt
agagacgaagtttcaccatgttggtcaggctgctctcgaagtcctgacctcaggtgatcc
acccacctcagcctcccaaagtgctaggattacaggcatgagccactgcgcccggtctgt
ttcctgatgtgttaatgatcgccattgtatctggtgtgagatggtatttcattgtggttt
tgatttgcatttcactaataaccagtgctgatgatcttttcttcatatgtttgttggctg
cattaatgtcttcttttgagaagtgtctgttcatatcctttgcccactttttcatggggc
tgtttgttttttcttgtaaatttgtttaagttctttgtagattctggatattagcccttt
gtcagatggatagattgcaaaatttttccccctttctgtaggttgcctgttcaccctgat
gatagtttattttgctgtgcagaaactctttagtttaattagatcccatttgtcaatttt
ggcttttgttgccattgcttttgatgttttagtcatgaagtctttgcccatgcctatgtc
ctggatgttattgcctagattttcttctagggtttttatggttttaggtcttacgtttaa
gtctttaattcatcttgagttaattcttgtataaggtgtaaggaaggggtccagtttcag
ttttctgcatatggctagccagtccttcttgatttagtatttgtgggttttaaaaaagga
gtttcccaaaatattcagttaaacttttaagtgacttacgtgtatatctaaatacatgat
cagttaatatttgtcttaaaggggttttctttgttcttttcttattataggaaggttaaa
caatatgcttatttatgccatagcttcacaaacaggaaggaggttttaaatggtttagtt
ccacaatttgagtagatgcatatttaaagaaacgttgttgcataataaatactgcctctt
cctaaaatgcatcatgccacagccaattttggaaaacacaaatatgaggtgagtgtattt
tgaaaactatgtgaatataatagatctttaattcatatttgtggattttatgggaaatac
ttgttttctaaggcatctgtcttgcaaaaagtcagtttctgctatgaaggatgttaaagg
ggatatgtaggttaaattctgtttctgagctttgcttccagagtaaacacccaacttact
tttgccctaaagtattttattgttctagtagagaagactaacaacatattctaaaccact
aagtaatttatgtaaacttcgcttacaaactacacttgtgtgacacttatatgagcaaaa
gcattttcatatttcttactatatcattcaattcttgcttaccccaatggaagtgacttt
atgcccctttagagacaatggaaatcaggtacttcgtgatttctcttaaaaaaaaaaaaa
atgaactagaaagctccaagtttggtgaatctggaacctgggtattccagttccagttgt
agcccttcctccctatccatcactcctgtctgcatgtaattatgcaatacattgaaaaga
ttaaaagatgggtcttggactcaggcagacctgggtcaaatccagattctggcactgccc
agccattgcccctgggcaagccattttcctctttgaacctcatttgtgaattaagctaaa
aatagtccccacccccatgggactgtgggaaggattaaatagaataatgcatgaaaagca
aatagcagaatggtccataaatgttaaccattgttatgttattatgtaatctacaaagta
cgtttagttacacttcatgaaatactttcagtttttcaaagacaccactaatacatggga
aatcaaaccctgaaaattaatttcactttagcagtaaagtcacatgccagatggaaagga
tagtatttcatgaacaaagatcttacttttgagatttggtcttacttttttctttttctt
aagggagaattatcttgtgttttttgttttgttttgttttgagatggagtcttgttctgt
cacccaggctggagcgcagtgacgtgatctcggctcactgcaaccttcacctcccgggtt
caagagattctcctgtctcagcctcccgagtagctgggactacaggtacgtgccaccaca
cctggctaatttttgtatttttagtagagacaagagttacaccatattggccaggatott
ttgctttctatagcttcaaaatgttcttaatgttaagacattcttaatactctgaaccat
atgaatttgccattttggtaagtcacagacgccagatggtggcaatttcacatggcacaa
cccgaaagattaacaaactatccagcagatgaaaggattttttttagtttcattgggttt
actgaagaaattgtttgaattctcattgcatctccagttcaacagataatgagtgagtga
tgccacactctcaagagttaaaaacaaaacaacaaaaaaattaaaacaaaagcacacaac
tttctctctctgtcccaaaatacatacttgcatacccccgctccagataaaatccaaagg
gtaaaactgtcttcatgcctgcaaattcctaaggagggcacctaaagtacttgacagcga
gtgtgctgaggaaatcagcagctgttgaagtcacctcctgtgctcttgccaaatgtttga
aagggaatacactgggttaccgggtgtatgttgggaggggagcattatcagtgctcgggt
gaggcaagttcggagtacccagatggagacatccgtgtctgtgtcgctctggatgcctcc
aagccagcgtgtgtttactttctgtgtgtgtcaccatgtctttgtgcttctgggtgcttc
tgtgtttgtttctggccgcgtttctgtgttggacaggggtgactttgtgccggatggctt
ctgtgtgagagcgcgcgcgagtgtgcatgtcggtgagctgggagggtgtgtctcagtgtc
tatggctgtggttcggtataagtctgagcatgtctgccagggtgtatttgtgcctgtatg
tgcgtgcctcggtgggcactctcgtttccttccgaatgtggggcagtgccggtgtgctgc
cctctgccttgagacctcaagccgcgcaggcgcccagggcaggcaggtagcggccacaga
agagccaaaagctcccgggttggctggtaaggacaccacctccagctttagccctctggg
gccagccagggtagccgggaagcagtggtggcccgccctccagggagcagttgggccccg
cccgggccagccccaggagaaggagggcgaggggaggggagggaaaggggaggagtgcct
cgccccttcgcggctgccggcgtgccattggccgaaagttcccgtacgtcacggcgaggg
cagttcccctaaagtcctgtgcacataacgggcagaacgcactgcgaagcggcttcttca
gagcacgggctggaactggcaggcaccgcgagcccctagcacccgacaagctgagtgtgc
aggacgagtccccaccacacccacaccacagccgctgaatgaggcttccaggcgtccgct
cgcggcccgcagagccccgccgtgggtccgcccgctgaggcgcccccagccagtgegctc
acctgccagactgcgcgccatggggcaacccgggaacggcagcgccttcttgctggcacc
caatggaagccatgcgccggaccacgacgtcacgcaggaaagggacgaggtgtgggtggt
gggcatgggcatcgtcatgtctctcatcgtcctggccatcgtgtttggcaatgtgctggt
catcacagccattgccaagttcgagcgtctgcagacggtcaccaactacttcatcacttc
actggcctgtgctgatctggtcatgggcctggcagtggtgccctttggggccgcccatat
tcttatgaaaatgtggacttttggcaacttctggtgcgagttttggacttccattgatgt
gctgtgcgtcacggccagcattgagaccctgtgcgtgatcgcagtggatcgctactttgc
cattacttcacctttcaagtaccagagcctgctgaccaagaataaggcccgggtgatcat
tctgatggtgtggattgtgtcaggccttacctccttcttgcccattcagatgcactggta
ccgggccacccaccaggaagccatcaactgctatgccaatgagacctgctgtgacttctt
cacgaaccaagcctatgccattgcctcttccatcgtgtccttctacgttcccctggtgat
catggtcttcgtctactccagggtctttcaggaggccaaaaggcagctccagaagattga
caaatctgagggccgcttccatgtccagaaccttagccaggtggagcaggatgggcggac
ggggcatggactccgcagatcttccaagttctgcttgaaggagcacaaagccctcaagac
gttaggcatcatcatgggcactttcaccctctgctggctgcccttcttcatcgttaacat
tgtgcatgtgatccaggataacctcatccgtaaggaagtttacatcctcctaaattggat
aggctatgtcaattctggtttcaatccccttatctactgccggagcccagatttcaggat
tgccttccaggagcttctgtgcctgcgcaggtcttctttgaaggcctatgggaatggcta
ctccagcaacggcaacacaggggagcagagtggatatcacgtggaacaggagaaagaaaa
taaactgctgtgtgaagacctcccaggcacggaagactttgtgggccatcaaggtactgt
gcctagcgataacattgattcacaagggaggaattgtagtacaaatgactcactgctgta
aagcagtttttctacttttaaagacccccccccccaacagaacactaaacagactattta
acttgagggtaataaacttagaataaaattgtaaaattgtatagagatatgcagaaggaa
gggcatccttctgccttttttatttttttaagctgtaaaaagagagaaaacttatttgag
tgattatttgttatttgtacagttcagttcctctttgcatggaatttgtaagtttatgtc
taaagagctttagtcctagaggacctgagtctgctatattttcatgacttttccatgtat
ctacctcactattcaagtattaggggtaatatattgctgctggtaatttgtatctgaagg
agattttccttcctacacccttggacttgaggattttgagtatctcggacctttcagctg
tgaacatggactcttcccccactcctcttatttgctcacacggggtattttaggcaggga
tttgaggagcagcttcagttgttttcccgagcaaagtctaaagtttacagtaaataaatt
gtttgaccatgccttcattgcacctgtttctccaaaaccccttgactggagtgctgttgc
ctcccccactggaaaccgcaggtaactacttgtaattactgcccatgacttaatgtagaa
tgatacaagaatgacatgcacagattgcttaaccctttcatttgcctttgagtctgctgc
tgcaaagctgcatctctcctgacacttgtgccccaaatcagttctgcctgctcttagtat
agctcaactctccctatggttattgttctgtgttgttacctcagaaacactgactcacag
aagcggagttaaggggatatgtttttttctctccacgtgcacccaccacccaccttccag
ttctacttgtttcaaaactgtttatatttctgtcttggccatgtgttacagtggagctct
ttgtactgcatcagggcttggcattttagggataaggaagatgttcttatgaggaagcta
ctcagacatggccccgtaattctgagggaaaattcaaaaggcattggtcatggggagaaa
agctggagaacacataactgatggatacctcatgaactagaaacagaattttaacccctt
ttccttctttcctttggtccctgttttcttctcccactgactctcctcgattcagtgtaa
accaaggttctgagtcttagcactgttagcattttggaccagataactctttgttatggg
ggctgcatcattgtatgtgtagcacctctggcctctgttcattagatgccaatagcaccc
cctgcttataacaagcaaaaatgtttccagacattgcaaaagagcccctgaggtgcgaat
tagcccctggttgagagtcactggtagaaactgtaaaaatctcagcagatacatacattc
tttctaatgcaagcgcttgattgtgcagagccttagagagggattttcacagttcaccta
ggcagtaacagaccctcaccagcactctttccattccatcatgctgccttctaaacttgt
tttctagctgcccaaatagtgatcatgaaatgttaagaaggctttaagtctgtacatgaa
ttgtttgagagggtttatcaatggaggtgaggcctgtgggccatgactcctgtttgtgaa
gagattataatactgtcaagaggcacgttaggggaaatcacaaaagtaaacacatttett
ctcccagcccctttctatttttgcctgtgtgtctgagccagagcttggcccaggtttgat
gaagtggatcgtcctccttggcaacgccaggctagagcagatcagcctgcagggttcatt
gccattccactggctcatgaagctgactccactcccctcttcctttctgttgcagccaag
gtccccaaccagaaaagcattggccttctctgcttcctgtcaactcaatgatgggatgtt
tgggtgagcaccgagctatcaggagaaggttaggcgcctgtgattttggaacatgccatg
gcaaattggagaatgtgtgtcattcagtgcttttacttttttccaagggttttcatacct
attgaaaacccttattacacattatcaccttctctttctactgctattatcacattctct
ttctactgctctggtctccacactcagagatttgggcagcttctttggctaatattcatg
ctccctgtagcctcataagatctcagacatggaagagcccatagaaagtatttaacatct
gatgagactgaaacgctgtgaggtgaagggcttgcccaaggtaagcagccaaggatgtca
gagtgggactcaagccagggaacccaactgctattccaggaactgctgcattctctccac
cacattagcattgcgttctttcctcaccctcaactggggctataacataacacattcatt
tcagccaatatatttttcttttgtccttaacacaaaatttagagcataaacaaataatct
gcaatagagacaaaagaaataattgttcatttaactcaacaa
SEQ ID NO: 6 is an exemplary nucleotide sequence of
human Adrb2 transcript cDNA, from NCBI
reference sequence NM_000024.6.
gcactgcgaagcggcttcttcagagcacgggctggaactggcaggcaccgcgagccccta
gcacccgacaagctgagtgtgcaggacgagtccccaccacacccacaccacagccgctga
atgaggcttccaggcgtccgctcgcggcccgcagagccccgccgtgggtccgcccgctga
ggcgcccccagccagtgcgctcacctgccagactgcgcgccatggggcaacccgggaacg
gcagcgccttcttgctggcacccaatggaagccatgcgccggaccacgacgtcacgcagg
aaagggacgaggtgtgggtggtgggcatgggcatcgtcatgtctctcatcgtcctggcca
tcgtgtttggcaatgtgctggtcatcacagccattgccaagttcgagcgtctgcagacgg
tcaccaactacttcatcacttcactggcctgtgctgatctggtcatgggcctggcagtgg
tgccctttggggccgcccatattcttatgaaaatgtggacttttggcaacttctggtgcg
agttttggacttccattgatgtgctgtgcgtcacggccagcattgagaccctgtgcgtga
tcgcagtggatcgctactttgccattacttcacctttcaagtaccagagoctgctgacca
agaataaggcccgggtgatcattctgatggtgtggattgtgtcaggccttacctccttct
tgcccattcagatgcactggtaccgggccacccaccaggaagccatcaactgctatgcca
atgagacctgctgtgacttcttcacgaaccaagcctatgccattgcctcttccatcgtgt
ccttctacgttcccctggtgatcatggtcttcgtctactccagggtctttcaggaggcca
aaaggcagctccagaagattgacaaatctgagggccgcttccatgtccagaaccttagcc
aggtggagcaggatgggcggacggggcatggactccgcagatcttccaagttctgcttga
aggagcacaaagccctcaagacgttaggcatcatcatgggcactttcaccctctgctggc
tgcccttcttcatcgttaacattgtgcatgtgatccaggataacctcatccgtaaggaag
tttacatcctcctaaattggataggctatgtcaattctggtttcaatccccttatctact
gccggagcccagatttcaggattgccttccaggagcttctgtgcctgcgcaggtcttctt
tgaaggcctatgggaatggctactccagcaacggcaacacaggggagcagagtggatatc
acgtggaacaggagaaagaaaataaactgctgtgtgaagacctcccaggcacggaagact
ttgtgggccatcaaggtactgtgcctagcgataacattgattcacaagggaggaattgta
gtacaaatgactcactgctgtaaagcagtttttctacttttaaagacccccccccccaac
agaacactaaacagactatttaacttgagggtaataaacttagaataaaattgtaaaatt
gtatagagatatgcagaaggaagggcatccttctgccttttttatttttttaagctgtaa
aaagagagaaaacttatttgagtgattatttgttatttgtacagttcagttcctctttgc
atggaatttgtaagtttatgtctaaagagctttagtcctagaggacctgagtctgctata
ttttcatgacttttccatgtatctacctcactattcaagtattaggggtaatatattgct
gctggtaatttgtatctgaaggagattttccttcctacacccttggacttgaggattttg
agtatctcggacctttcagctgtgaacatggactcttcccccactcctcttatttgctca
cacggggtattttaggcagggatttgaggagcagcttcagttgttttcccgagcaaagtc
taaagtttacagtaaataaattgtttgaccatg
SEQ ID NO: 7 is an exemplary Adrb1-targeting gRNA that
targets human chr10: 114044812.
GTAGAAGGAGACTACGGACGAGG
SEQ ID NO: 8 is an exemplary Adrb1-targeting gRNA that
targets human chr10: 114044800.
TACGGACGAGGCGATGGCGTAGG
SEQ ID NO: 9 is an exemplary Adrb1-targeting gRNA that
targets human chr10: 114045536.
TGGATTCCGAGGCGAAGCCGGGG
SEQ ID NO: 10 is an exemplary Adrbl-targeting gRNA that
targets human chr10: 114044164.
CGACGACAGGTTACCGGGCTCGG
SEQ ID NO: 11 is an exemplary Adrb1-targeting gRNA that
targets human chr10: 114044170.
TGCGGCCGACGACAGGTTACCGG
SEQ ID NO: 12 is an exemplary Adrb1-targeting gRNA that
targets human chr10: 114045037.
CCAACGGGCGTGCGGGTAAGCGG
SEQ ID NO: 13 is an exemplary Adrb1-targeting gRNA that
targets human chr10: 114045537.
TTGGATTCCGAGGCGAAGCCGGG
SEQ ID NO: 14 is an exemplary Adrb1-targeting gRNA that
targets human chr10: 114044861.
GTACCTGCGGGTGTTCCGCGAGG
SEQ ID NO: 15 is an exemplary Adrb1-targeting gRNA that
targets human chr10: 114044165.
CGAGCCCGGTAACCTGTCGTCGG
SEQ ID NO: 16 is an exemplary Adrb1-targeting gRNA that
targets human chr10: 114044468.
GCCGTTCGGGGCCACCATCGTGG
SEQ ID NO: 17 is an exemplary Adrb2-targeting gRNA that
targets human chr5: 148827001.
CAGACGCTCGAACTTGGCAATGG
SEQ ID NO: 18 is an exemplary Adrb2-targeting gRNA that
targets human chr5: 148827007.
CGTCTGCAGACGCTCGAACTTGG
SEQ ID NO: 19 is an exemplary Adrb2-targeting gRNA that
targets human chr5: 148826888.
GCCGGACCACGACGTCACGCAGG
SEQ ID NO: 20 is an exemplary Adrb2-targeting gRNA that
targets human chr5: 148826894.
CCACGACGTCACGCAGGAAAGGG
SEQ ID NO: 21 is an exemplary Adrb2-targeting gRNA that
targets human chr5: 148827094.
AAGAATATGGGCGGCCCCAAAGG
SEQ ID NO: 22 is an exemplary Adrb2-targeting gRNA that
targets human chr5: 148826889.
TCCTGCGTGACGTCGTGGTCCGG
SEQ ID NO: 23 is an exemplary Adrb2-targeting gRNA that
targets human chr5: 148827998.
CGCTAGGCACAGTACCTTGATGG
SEQ ID NO: 24 is an exemplary Adrb2-targeting gRNA that
targets human chr5: 148827333.
CGGTACCAGTGCATCTGAATGGG
SEQ ID NO: 25 is an exemplary Adrb2-targeting gRNA that
targets human chr5: 148826818.
TTGCCCCATGGCGCGCAGTCTGG
SEQ ID NO: 26 is an exemplary Adrb2-targeting gRNA that
targets human chr5: 148826814.
CCTGCCAGACTGCGCGCCATGGG
SEQ ID NO: 27 is an exemplary hairpin sequence for
knockdown of CREM in mouse.
TGCTGTTGACAGTGAGCGACAGACTCAGAAGTAATTGATATAGTGAAGCCACAGATGTAT
ATCAATTACTTCTGAGTCTGCTGCCTACTGCCTCGGA
SEQ ID NO: 28 is an exemplary murine Adrb1-targeting gRNA.
TGGCCATCGCCAAGACCCCG
SEQ ID NO: 29 is an exemplary Adrbl forward primer for mouse.
CTCATCGTGGTGGGTAACGTG
SEQ ID NO: 30 is an exemplary Adrbl reverse primer for mouse.
ACACACAGCACATCTACCGAA
SEQ ID NO: 31 is an exemplary Crem forward primer for mouse.
GCAAATGTGGCAGGAAAAAGT
SEQ ID NO: 32 is an exemplary Crem reverse primer for mouse.
TGATCCAGCTACAGAAACCTGA

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017; The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

In order to facilitate review of the various aspects of the disclosure, the following explanations of specific terms are provided:

About: Unless context indicated otherwise, “about” refers to plus or minus 5% of a reference value. For example, “about” 100 refers to 95 to 105.

Administration: To provide or give a subject an agent, such as a modified PBMC described herein, by any effective route. Administration can be local or systemic. Exemplary routes of administration include, but are not limited to, injection (such as subcutaneous, intramuscular, intradermal, intraperitoneal, intratumoral, intraprostatic, intrathecal, intraosseous, and intravenous), oral, sublingual, rectal, transdermal, intranasal, vaginal and inhalation routes. In some examples, modified PBMCs provided herein (such as T cells) are administered by intravenous injection.

Adoptive Cell Transfer (ACT) Therapy: A type of immunotherapy in which a T cell that has been modified (e.g., modified to recognize a tumor-specific antigen or viral antigen) and/or expanded in vitro (or ex vivo) is administered to a patient in need thereof. T cells for ACT therapy can be a patient's own T cells or T cells from a donor. ACT therapies include, for example, Chimeric Antigen Receptor T cell (CAR-T). Engineered T Cell Receptor (TCR), or Tumor-Infiltrating Lymphocyte (TIL) therapies. ACT therapy is also sometimes referred to as adoptive cell therapy, cellular adoptive immunotherapy, or T-cell transfer therapy.

Agent: Any substance or any combination of substances (small molecules, proteins, peptides, nucleic acid molecules, antisense molecules etc.) that is useful for achieving an end or result; for example, a substance or combination of substances useful for reducing or inhibiting ADRB1 and/or ADRB2 activity.

Autoimmune disease (or autoimmune disorder): Autoimmune disorders include a broad range of related diseases in which a person's immune system produces an inappropriate response (e.g., a B cell or a T cell response) against an endogenous antigen, with consequent injury to its own cells, tissues, and/or organs, resulting in inflammation and damage. There are over 80 different autoimmune diseases. The injury may be localized to certain organs or tissue, such as Sjogren's disease, or may be systemic, such as psoriasis. Although symptoms can vary with type of autoimmune disease, common symptoms include fatigue, achy muscles, swelling and redness, low-grade fever, trouble concentrating, numbness and tingling in the hands and feet, hair loss, and skin rashes. Tests for autoimmune disorders similarly vary with types, but typical tests include an antinuclear antibody test (ANA) and tests for specific autoantibodies produced in certain disorder types as well as an examination for inflammation in the body.

In some examples, autoimmune diseases that can be treated with the disclosed methods (e.g., with PBMCs having increased ADRB1 and/or ADRB2 activity) include rheumatoid arthritis, systemic lupus erythematosus, type 1 and type 2 diabetes, multiple sclerosis, acute disseminated encephalomyelitis, Sjögren's syndrome, Graves' disease, myasthenia gravis, ulcerative colitis, Hashimoto's thyroiditis, celiac disease, Crohn's disease, arthritis, inflammatory bowel disease, psoriasis, autoimmune hepatitis, autoimmune pancreatitis, autoimmune encephalitis, or scleroderma, autoimmune hemolytic anemia, autoimmune thrombocytopenia, autoimmune neutropenia, atopic dermatitis, alopecia, ankylosing spondylitis, Addison's disease, alopecia areata, anti-phospholipid antibody syndrome, Gooodpastue's Syndrome, Grave's disease, Guillain-Barre syndrome, IgA Nephropathy, pemphigoid, pemphigus, polyglandular autoimmune syndrome type 2, psoriatic arthritis, Takayasu's arteriosus, or undifferentiated connective tissue disease (UCTD).

In some examples, the disclosed methods further include administration of one or more anti-inflammatories and/or immunosuppressing agents.

Beta-1-adrenergic receptor (ADRB1): (e.g., OMIM 109630) ADRB1 mediates catecholamine signaling via activation of adenylate cyclase thereby increasing intracellular cAMP levels. Adrb1 sequences are publicly available, and exemplary sequences include Amino Acid NCBI Reference Sequences: NP_000675.1. (human), NP_031445.2 (Mus musculus), NP_001116546.1 (Sus scrofa); Nucleotide NCBI Reference Sequences: NG_012187.1. (human), NM_000684.3. (human), NM_007419.3 (Mus musculus), NM_001123074.1 (Sus scrofa), each of which is herein incorporated by reference in their entirety. NCBI Gene ID: 153. In this disclosure reference to Adrb1 or ADRB1 includes the corresponding gene or protein in any species: human, mouse, or otherwise, such as any mammalian Adrb1. Also known as ADRB1R and B1AR.

Beta-2-adrenergic receptor (c): (e.g., OMIM 109690) ADRB2 mediates catecholamine signaling. Adrb2 sequences are publicly available, and exemplary sequences include Amino Acid NCBI Reference Sequences: NP_000015.2 (human) NP 031446.2 (Mus musculus), NP 001121908.1 (Sus scrofa); Nucleotide NCBI Reference Sequences: NG_016421.2 (human), NM_000024.6 (human), NM_007420.3 (Mus musculus). NM_001128436.1 (Sus scrofa), each of which is herein incorporated by reference in their entirety. NCBI Gene ID: 154. In this disclosure reference to Adrh2 or ADRB2 includes the corresponding gene or protein in any species: human, mouse, or otherwise, such as any mammalian Adrb2. Also known as ADRBR, BAR, B2AR, and ADRB2R.

//Inventors: please review the definitions and sequences for ADRB1/2 for accuracy, including checking to see that the cited sequences are correct.

Beta-Blocker: Agents which block the receptor sites for endogenous catecholamines, including epinephrine and norepinephrine. Some beta-blockers block all beta-adrenergic receptors, while others are more selective.

In some examples beta-blockers block signaling mediated by ADRB1 and/or ADRB2. Exemplary beta-blockers that can be used with the disclosed methods include atenolol, bisoprolol, metoprolol, propranolol, bucindolol, oxprenolol, carteolol, pindolol, oxprenolol, penbutolol, betaxolol, celiprolol, acebutolol, labetalol, carvedilol, pronethalol, sotalol, nebivolol, esmolol, butaxamine, alprenolol, bupranolol, nadolol, timolol, CGP 20712A (specific ADRB1 blocker), ICI 118551 (specific ADRB2 blocker). For additional information on the selectivity of beta receptors, see Baker, The selectivity of β-adrenoceptor antagonists at the human β1, β2 and β3 adrenoceptors, B. J. Pharm. (2005) 144:317-22.

Inversely, the stimulation of beta-receptors is possible with beta-agonists, such as salbutamol, terbutaline, and salmeterol.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Cas9: An RNA-guided DNA endonuclease enzyme that that participates in the CRISPR-Cas immune defense against prokaryotic viruses. Cas9 has two active cutting sites (HNH and RuvC), one for each strand of the double helix. Thus, Cas9 proteins can be used to edit DNA in combination with an appropriate guide RNA. Cas9 sequences are publicly available. For example, GenBank® Accession Nos. nucleotides 796693 . . . 800799 of CP012045.1 and nucleotides 1100046 . . . 1104152 of CP014139.1 disclose exemplary Cas9 nucleic acids, and GenBank® Accession Nos. NP_269215.1, AMA70685.1, and AKP81606.1 disclose exemplary Cas9 proteins.

Catalytically inactive (deactivated or dead) Cas9 (dCas9) proteins, which have reduced or abolished endonuclease activity but still binds to dsDNA, are also encompassed by this disclosure. In some examples, a dCas9 includes one or more mutations in the RuvC and HNH nuclease domains, such as one or more of the following point mutations: D10A, E762A, D839A, H840A, N854A, N863A, and D986A. An exemplary dCas9 sequence includes both a D10A and H840A substitutions. In one example, the dCas9 protein has mutations D10A, H840A, D839A, and N863A (see, e.g., Esvelt et al., Nat. Meth. 10:1116-21, 2013). Exemplary dCas9 sequences are provided in GenBank® Accession Nos. AKA60242.1 and KR011748.1.

Chimeric antigen receptor (CAR): Artificial, engineered T cell receptors, which graft an arbitrary specificity onto an immune effector cell. These receptors can be used to graft the specificity of a monoclonal antibody onto a T cell: with transfer of their coding sequence facilitated by vectors. Thus, a CAR that “specifically binds” or is “specific” for an antigen is a CAR that binds the antigen with high affinity and does not significantly bind other unrelated antigens. Using adoptive cell transfer, CARs can be useful to treat cancer. For example, T cells (obtained from the patient or from a donor) are modified such that they express receptors specific to the patient's particular cancer. The modified T cells, which can then recognize and kill the cancer cells, are introduced into the patient. In some examples, the modified PBMC disclosed herein express a CAR.

First generation CARs typically included the intracellular domain from the CD3 ζ-chain, which is the primary transmitter of signals from endogenous TCRs. Second generation CARs added intracellular signaling domains from various costimulatory protein receptors (e.g., CD28, 41BB, ICOS) to the cytoplasmic tail of the CAR to provide additional signals to the T cell. Third generation CARs combine multiple signaling domains, such as CD3z-CD28-41BB or CD3z-CD28-OX40, to augment potency. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells expressing a bispecific or bicistronic CAR can bind cells that express both of the antigens to which the binding moieties are directed (see, for example, Qin et al., Blood 130:810, 2017; and WO/2018/213337). Any of these CARs can be used with the methods described herein.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, and 10 out of 10 being 50%, 60%, 70%, 80%, 90%, and 100% complementary, respectively).

In some aspects, a disclosed nucleic acid molecule (such as a disclosed gRNA or RNAi) hybridizes to a target nucleic acid, thus the nucleic acid molecule is complementary to the target sequence. For example, in some examples, a RNAi or gRNA specific for Adrb1 gene or transcript is at least 80%, at least 85%, at least 90%, at least 91%, at least 92%, at least 93%, at least 94%, at least 95%, at least 96%, at least 97%, at least 98% or at least 99% complementary to a unique portion of a target gene, such as Adrb1. In some examples, the target sequence is at least 10 contiguous nucleotides, for example, at least 12, at least 15, at least 20, at least 25, at least 30, at least 35, or at least 40 contiguous nucleotides. In further examples, the target sequence is 10-50 contiguous nucleotides, for example, 12-40, 12-30, 12-20, 12-15, 15-30, 15-20, 20-30, or 20-40 contiguous nucleotides. In some examples, the targeting sequence is at least 80%, at least 85%, at least 90%, at least 91%, at least 92%, at least 93%, at least 94%, at least 95%, at least 96%, at least 97%, at least 98% or at least 99% complementary to a sequence about 20 nucleotides long in a Adrb1 gene or transcript.

Control: A reference standard. In some aspects, the control is a negative control. In other aspects, the control is a positive control. In some examples, a suitable control is a historical control or standard reference value or range of values (such as a previously tested control sample, such as a group of patients diagnosed with a disease or condition, for example cancer, autoimmunity, or viral infection, that have a known prognosis or outcome, or a group of samples that represent baseline or normal values). In some examples, the control may be a subject not receiving treatment with an agent (e.g., the disclosed modified PBMCs) or receiving an alternative treatment, or a baseline reading of the subject prior to treatment with an agent.

A difference between a test sample and a control can be an increase or conversely a decrease. The difference can be a qualitative difference or a quantitative difference, for example a statistically significant difference. In some examples, a difference is an increase relative to a control, for example, an increase of at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%. In other examples, a difference is a decrease relative to a control, for example, a decrease of at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, or at least about 100%.

CRISPR/Cas system: A prokaryotic immune system that confers resistance to foreign genetic elements, such as plasmids and phages, and provides a form of acquired immunity. In the endogenous system, a trans-activating crRNA (tracrRNA) hybridizes with the repeat sequence of another RNA molecule known as CRISPR RNA (crRNA) to form a unique dual-RNA hybrid structure that binds Cas endonuclease, forming a ribonucleoprotein (RNP) complex. The crRNA contains a targeting sequence complementary to a target gene, which guides the CRISPR/Cas RNP complex to the target.

In some examples, the Cas endonuclease is Cas9, which induces a double stranded DNA break in the target gene. The CRISPR/Cas9 system can be used to decrease gene expression, for example, by targeting and inducing double-stranded DNA breaks in a target gene, such as Adrb1. Similarly, a CRISPR/Cas13 system can be used to cut RNA. Cas endonucleases (or cas nucleases) include, but are not limited to, Cas3, dCas3, Cas9, dCas9, Cas12, dCas12, Cas13a, dCas13a, Cas13b, dCas13b, Cas13d, or dCas13d. Cas endonucleases can cleave RNA or DNA depending on the specific endonuclease chosen.

Down Regulation and Knock-Out: When used in reference to the expression of a molecule, such as a target, “down regulation” refers to any process which results in a decrease in production of an RNA of interest. In one example, downregulation decreases detectable RNA expression or RNA activity. A “knock-out” is the removal or inactivation of one or more genes (such as Adrb1 and/or Adrb2) from an organism, which can result in a decrease in detectable RNA expression or RNA activity.

Downregulation includes any detectable decrease in the RNA. In certain examples, detectable RNA in a cell or cell free system decreases by at least 10%, at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of Adrb1 and/or Adrb2 RNA detected in a corresponding non-treated cell or sample).

Effective amount: The amount of an agent (such as the modified PBMC, RNAi, gRNA, or other composition disclosed herein) that is sufficient to effect beneficial or desired results. An effective amount (also referred to as a therapeutically effective amount) may vary depending upon one or more of: the subject and disease condition being treated, the weight and age of the subject, the severity of the disease condition, the manner of administration and the like. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In one aspect, an “effective amount” of a therapeutic agent (e.g., a modified PBMC disclosed herein) is an amount sufficient to reduce the volume/size of a tumor, the weight of a tumor, the number of metastases, reduce the volume/size of a metastasis, the weight of a metastasis, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In one aspect, an “effective amount” of a therapeutic agent (e.g., a modified PBMC disclosed herein) is an amount sufficient to reduce signs or symptoms of the viral infection in a subject, reduce the viral load in a subject, reduce infectivity of a virus, reduce cytopathic effect in the subject's cells, or combinations thereof, for example by at least 5%, at least 10%, at least 15%, at least 20%, at least 25%, at least 30%, at least 40%, at least 50%, at least 60%, at least 70%, at least 75%, at least 80%, at least 90%, at least 95%, or at least 99% (as compared to a suitable control, such as no administration of the therapeutic agent). In some aspects an “effective amount” of a therapeutic agent (e.g., a gRNA or siRNA disclosed herein) is an amount sufficient to reduce signs or symptoms of autoimmunity, reduce markers of activation on immune cells in a subject with autoimmunity, increase markers of tolerance on immune cells in a subject with autoimmunity, and/or reduce autoantibodies in a subject with autoimmunity, for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 98%, or even 100% (as compared to a suitable control, such as no administration of the therapeutic agent). In one aspect, an “effective amount” of a therapeutic agent (e.g., a gRNA or siRNA disclosed herein) is an amount sufficient to reduce activity or expression of a target (e.g., ADRB1 and/or ADRB2), for example by at least 5%, at least 10%, at least 15%, at least 20%, at least 25%, at least 30%, at least 40%, at least 50%, at least 60%, at least 70%, at least 75%, at least 80%, at least 90%, at least 95%, at least 98%, or even 100% (as compared to a suitable control, such as expression or activity prior to administering the therapeutic agent). In some examples, combinations of these effects are achieved.

Guide RNA (gRNA): An RNA component of a CRISPR/Cas system that targets the CRISPR/Cas ribonucleoprotein (RNP) complex to a target nucleic acid sequence, such as a target DNA (e.g., genomic sequence) or target RNA sequence, gRNA molecules include (1) a portion with sequence complementarity to the target nucleic acid (such as at least 80%, at least 90%, at least 95%, or 100% sequence complementarity), and (2) a portion with secondary structure that binds to the Cas nuclease. Such portions can be part of the same molecule (e.g., sgRNA: a synthetic chimera that combines a crRNA and tracrRNA into a single RNA transcript), or divided over two or more separate molecules (e.g., 2 part gRNA wherein the crRNA and tracrRNA are separate RNA transcripts). For simplicity, both types of molecules are referred to herein as gRNA.

Many techniques for genome editing using the CRISPR/Cas system have been described. In brief, gRNA directs a Cas DNA nuclease (such Cas9) to a target gene (DNA). Cas9 then introduces a double stranded break at the target site. Disruptive mutations can be introduced through non-homologous end joining of the cut DNA. Cas9 can also be used to delete larger DNA fragments, for example, by using two gRNAs targeting separate sites, thus causing a deletion of the intervening sequence between the two cut sites. A DNA template with homology to the target site can also be added to introduce insertions using homology directed DNA repair mechanisms.

In RNA editing, the gRNA directs a Cas RNA nuclease (such Cas13d) to a target RNA. In one such example, the gRNA includes from 5′ to 3′ (1) a crRNA containing a direct repeat (DR) region and (2) a spacer, for example for Cas13a, Cas13c, and Cas 13d nucleases. In one example includes about 36 nt of DR followed by about 28-32 nt of spacer sequence. In another such example, the gRNA includes from 5′ to 3′ (1) a spacer and (2) a crRNA containing a DR region, for example for Cas13b nuclease. In some examples, the gRNA is processed (truncated/modified) by a Cas RNA nuclease or other RNases into the shorter “mature” form. The DR is the constant portion of the sgRNA, containing secondary structure which facilitates interaction between the Cas RNA nuclease protein and the gRNA. The spacer portion is the variable portion of the gRNA, and includes a sequence designed to hybridize to a target RNA sequence (and in some examples edit the target RNA sequence). In some examples, the full length spacer is about 28-32 nt (such as 30-32 nt) long while the mature (processed) spacer is about 14-30 nt.

The targeting portion of the gRNA can be modified to facilitate targeting of any DNA or RNA sequence of interest. (See CRISPR-Cas9 Structures and Mechanisms. Fuguo Jiang and Jennifer A. Doudna, Annual Review of Biophysics, 46:1, 505-529 (2017)). A gRNA that is “specific” for a target has sufficient complementarity to the target sequence that it binds the target and does not significantly hybridize with other unrelated sequences. The targeting sequence of the gRNA is typically about 20 nucleotides, for example, about 15, about 16, about 17, about 18, about 19, about 20, about 21, about 22, about 23, about 24, or about 25 nucleotides. The degree of complementarity between a targeting sequence and its corresponding target sequence, when optimally aligned using a suitable alignment algorithm, is about or more than about 50%, about 60%, about 75%, about 80%, about 85%, about 90%, about 95%, about 97.5%, about 98%, about 99%, or more. In some aspects, the degree of complementarity is 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X. BLAT, Novoalign (Novocraft Technologies, ELAND (Illumina, San Diego, Calif.), SOAP (available at soap.genomics.org.cn), and Maq (available at maq.sourceforge.net). In some examples, the targeting sequence is at least 80%, at least 85%, at least 90%, at least 91%, at least 92%, at least 93%, at least 94%, at least 95%, at least 96%, at least 97%, at least 98% or at least 99% complementary to a contiguous amino acid sequence about 20 nucleotides long in a Adrb1 and/or Adrb2 gene or transcript.

Heterologous: A heterologous protein or polypeptide refers to a protein or polypeptide derived from a different source or species. A heterologous nucleic acid molecule refers to a nucleic acid molecule derived from a different source or species. Thus, a heterologous protein, polypeptide, or nucleic acid molecule in a cell, refers to a protein, polypeptide, or nucleic acid molecule not naturally found in the cell in nature (e.g., an exogenous protein, polypeptide, or nucleic acid molecule). In some examples, a cell expressing a heterologous protein, polypeptide, or nucleic acid molecule is transgenic.

Immune Checkpoint Blockade (ICB) (or Checkpoint Inhibitor or Checkpoint Blockade): A therapeutic that targets a checkpoint protein. Checkpoint proteins help prevent over-active immune responses or autoimmunity, and in some examples reduce the ability of T cells to reduce/eliminate cancerous cells. When checkpoints are blocked (e.g., PD-1 blockade) T cells can better target and kill cancerous cells. Examples of checkpoint proteins found on T cells or cancerous cells include PD-1/PD-L1/PD-L2, and CTLA-4/B7-1/B7-2.

Exemplary ICB agents include ipilimumab (Yervoy®), nivolumab (Opdivo®), pembrolizumab (Keytruda®), atezolizumab (Tencentriq®), avelumab (Bavencio®), durvalumab (Imfinzi®), cemiplimab (Libtayo®), palbociclib (Ibrance®), ribociclib (Kisquali®), pidilizumab, avelumab, and abemaciclib (Verzenio®). Further examples are provided in Qiu et al., Journal of the European Society for Therapeutic Radiology and Oncology, 126(3):450-464, 2018; Visconti et al., J Exp Clin Cancer Res. 35(1): 153, 2016; and Mills et al. Cancer Res. 77(23): 6489-6498, 2017. Further exemplary ICB agents include anti-PD-1, anti-PD-L1, anti-CTLA-4, anti-LAG3 anti-GITR, anti-4-1BB, anti-CD40, and anti-OX40, anti-TIGIT, anti-VISTA, anti-CD73, anti-CD39, anti-HVEM, anti-BTLA, and anti-CD27.

Exemplary anti-PD-1 mAbs include nivolumab, pembrolizumab, pidilizumab, and cemiplimab. Exemplary anti-PD-L1 mAbs include atezolizumab, avelumab, durvalumab, cosibelimab, KN035 (envafolimab), BMS-936559, BMS935559, MEDI-4736, MPDL-3280A, and MEDI-4737. Exemplary anti-CTLA-4 mAbs include ipilimumab and tremelimumab.

Immunostimulatory antibody: Antibodies that enhance an immune response.

Immunosuppressive agent: A molecule, such as a chemical compound, small molecule, steroid, nucleic acid molecule, or other biological agent, that can decrease an immune response such as an inflammatory reaction. Specific, non-limiting examples of immunosuppressive agents that can be used with the disclosed methods are steroids, azathioprine, methotrexate, anti-TNF antibodies, anti-Il-12 antibodies, anti-Il-23 antibodies, corticosteroids, cyclosporine A, FK506, anti-CD52 antibodies, and anti-CD4 antibodies. In additional examples, the agent is a biological response modifier, such as KINERET® (anakinra), ENBREL® (etanercept), or REMICADE® (infliximab), HUMIRA® (adalimumab), CIMZIA® (certolizumab), a disease-modifying antirheumatic drug (DMARD), such as ARAVA® (leflunomide). Agents of use to treat inflammation include non-steroidal anti-inflammatory drugs (NSAIDs) such as aspirin, ibuprofen, and naproxen, a Cyclo-Oxygenase-2 (COX-2) inhibitor, such as CELEBREX® (celecoxib) and VIOXX® (rofecoxib), or another product, such as HYALGAN® (hyaluronan) and SYNVISC® (hylan G-F20). Agents of use to treat inflammation also include Janus kinase inhibitors (JAK inhibitors) such as ruxolitinib, tofacitinib, oclacitinib, baricitinib, peficitinib, upadacitinib, fedratinib, delgocitinib, filgotinib, abrocitinib, ruxolitinib, pacritinib, and deucravacitinib.

Immunotherapy: A therapy that uses an agent to stimulate or suppress the immune system to treat a disease, such as cancer or autoimmunity. Some examples of cancer immunotherapy include immune checkpoint inhibitors, adoptive cell transfer (ACT) immunotherapy, antibodies, vaccines, and immune system modulators. Specific, non-limiting examples include anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, anti-CD27, CD40 agonists, CSF1R inhibitory antibody, anti-CD47, STING agonists, and recombinant or reengineered cytokines such as Il-2, Il-10, Il-1β. Non-limiting examples of autoimmune immunotherapy include alemtuzumab, fingolimod, and natalizumab.

Increase or Decrease: A positive or negative change, respectively, in quantity from a control value (such as a value representing no therapeutic agent). An increase is a positive change, such as an increase at least 25%, at least 50%, at least 100%, at least 200%, at least 300%, at least 400% or at least 500%, as compared to the control value. A decrease is a negative change, such as a decrease of at least 10%, at least 20%, at least 30%, at least 40%, at least 50%, at least 60%, at least 70%, at least 75%, at least 80%, at least 85%, at least 90%, at least 95%, at least 98%, at least 99%, or at least 100% decrease as compared to a control value. In some examples, the increase or decrease is statistically significant relative to a suitable control.

An agent (e.g., the RNAi or a gRNA specific for Adrb1 disclosed herein) that decreases expression or activity of a gene (e.g., Adrb1) or gene product (e.g., ADRB1) is a compound that reduces the level of the mRNA or a functional product encoded by the gene in a cell or tissue (e.g., a PBMC), or reduces (including eliminates or inhibits) one or more activities of the gene product. In some aspects, expression of Adrb1 is reduced at least 10%, at least 25%, at least 50%, at least 75%, at least 90%, at least 95%, at least 99%, at least 99.9%, or even 100% relative to a control, such as an untreated subject or cells. Conversely, an agent that increases expression or activity of a gene or gene product is a compound that increases the level of the mRNA or protein product encoded by the gene in a cell or tissue, or increases one or more activities of the gene product.

In some aspects, an agent (e.g., the RNAi or a gRNA specific for Adrb1 disclosed herein) or non-naturally occurring genetic modification can increase or decrease an activity of a PBMC (e.g., a T cell) when it is present in the PBMC. For example, in some aspects the PBMC is a T cell and the agent (e.g., the RNAi or a gRNA specific for Adrb1 disclosed herein) or genetic modification (a point mutation, a partial deletion, full deletion, or insertion that reduces expression of Adrb1, as disclosed herein) reduces T cell exhaustion, for example, by at least 10%, at least 20%, at least 30%, at least 40%, at least 50%, at least 60%, at least 70%, at least 80%, at least 90%, at least 95%, or 100% relative to a suitable control (e.g., measurements prior to treatment or comparison to an untreated control group). A decrease in T cell exhaustion can be measured, for example, by a decrease in expression of TOX, PDCD-1, TIM3, TIGIT, ENTPD1 (CD39) or LAG3, CXCR6, or by an increase in cytokine production (e.g., IFNg, TNFa or IL-2), an increase in cytotoxic activity (e.g., increased tumor specific targeting or killing or Granzyme A, B, K expression), an increase in proliferative capacity (in vitro or in vivo expansion), or an increase in markers for effector-like cells (for example TBX21, EOMES, PRDM1, KLRG1, CX3CR1) or circulating memory or tissue-resident memory-like cells (for example, TCF7, FOXO1, SLAMF6, IL-7R, CD103, CD69, CXCR3) or measuring another indicator of T cell effector activity, relative to a suitable control. In some examples, combinations of these effects are achieved.

In some aspects the agent (e.g., the RNAi or a gRNA specific for Adrb1 disclosed herein) or genetic modification (a point mutation, a partial deletion, full deletion, or insertion that reduces expression of Adrb1, as disclosed herein) increases the activity or function of the PBMC. For example, in some examples the PBMC is a T cell and the agent can increase the activity or effector function of a T cell by at least 10%, at least 25%, at least 50%, at least 75%, at least 90%, at least 95%, at least 100%, at least 200%, at least 300%, at least 400%, or at least 500% relative to a suitable control (e.g., measurements prior to treatment or comparison to an untreated control group). An increase in effector function of a T cell can be measured, for example, by a decrease in expression of TOX, PDCD-1, TIM3, TIGIT, ENTPD1 or LAG3, CXCR6, or an increase in cytokine production (e.g., IFNg, TNFa or IL-2), an increase in cytotoxic activity (e.g., increased tumor specific targeting or killing or Granzyme expression, such as Granzymes A, B, and K), an increase in proliferative capacity (in vitro or in vivo expansion), or an increase in markers for effector-like cells (for example TBX21, EOMES, PRDM1, KLRG1, CX3CR1) or by measuring another indicator of T cell effector activity, relative to a suitable control. In some examples, combinations of these effects are achieved.

In some aspects the agent (e.g., the expression vector encoding Adrb1) can increase or decrease the activity of a PBMC (e.g., a T cell) when it is present in the PBMC. For example, in some aspects the PBMC is a T cell and the agent (e.g., the expression vector encoding Adrb1 and/or Adrb2 and/or the heterologous nucleic acid encoding Adrb1 and/or Adrb2) decreases T cell effector function. A decrease in effector function of a T cell can be measured, for example, by an increase in expression of TOX, PDCD-1, TIM3, TIGIT, ENTPD1 or LAG3, CXCR6, or a decrease in cytokine production (e.g., IFNg, TNFa or IL-2), a decrease in cytotoxic activity (e.g., decreased tumor specific targeting or killing or Granzyme A, B, K expression), a decrease in proliferative capacity (in vitro or in vivo expansion), or a decrease in markers for effector-like cells (for example TBX21, EOMES, PRDM1, KLRG1, CX3CR1) or measuring another indicator of T cell effector activity, relative to a suitable control. In some examples, combinations of these effects are achieved. In some aspects the PBMC is a T cell and the agent (e.g., the expression vector encoding Adrb1 and/or Adrb2 and/or the heterologous nucleic acid encoding Adrb1 and/or Adrb2) increases T cell tolerance. An increase in T cell tolerance can be measured, for example, by a decrease in genes encoding effector cytokines such as IFNG, PRF1, GZMM, GRN, changes in transcription factor expression (TBX21, EOMES; GATA3, EGR1, EGR2) and chemokine and cytokine receptors (CXCR3, CCR5, IL12RB1), an increase in expression of LAG3, or a decrease in cytokine production (e.g., IFNg, TNFa or IL-2), a decrease in cytotoxic activity (e.g., decreased tumor specific targeting or killing or Granzyme A, B, K expression), a decrease in proliferative capacity (in vitro or in vivo expansion), or a decrease in markers for effector-like cells (for example TBX21, EOMES, PRDM1, KLRG1, CX3CR1), an increase in genes not previously linked to unresponsive T cells (cell cycle genes, cell division genes, nucleosome and spindle assembly genes, DNA replication genes) or measuring another indicator of T cell effector activity, relative to a suitable control. In some examples, combinations of these effects are achieved.

Isolated: An “isolated” biological component (e.g., a cell, PBMC, nucleic acid, protein) has been substantially separated, produced apart from, or purified away from other biological components in the cell or tissue of an organism in which the component occurs, such as other cells (e.g., RBCs), chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. For example, PBMCs or TILs isolated from patient blood, tumor, or other sample, are at least 50% pure, such as at least 60%, such as at least 75%, at least 80%, at least 90%, at least 95%, at least 98%, at least 99%, or more, pure.

Modification: A change in the sequence of a nucleic acid (a “genetic modification”) or protein molecule. For example, amino acid or nucleic acid sequence modifications include mutations thereof, for example, substitutions, insertions, and deletions, or combinations thereof. Insertions include 3′ or 5′ end fusions or amino and/or carboxyl terminal fusions as well as intrasequence insertions of single or multiple amino acid residues or nucleotides. Deletions are characterized by the removal of one or more amino acid residues from a protein sequence or nucleotides from a nucleic acid sequence. In some aspects herein, the modification (such as a substitution, insertion, or deletion) results in a change in function, such as a reduction or enhancement of a particular activity of a protein. Substitutional modifications are those in which at least one residue or nucleotide has been removed and a different residue or nucleotide inserted in its place. Substitutions, deletions, insertions, or any combination thereof may be combined to arrive at a final mutant sequence. Amino acid modifications can be prepared by modification of nucleotides in the DNA encoding the protein, thereby producing DNA encoding the modification. In particular examples, the presence of one or more modifications in a gene can significantly inactivate that gene. A “modified” protein, nucleic acid, or organism is one that has one or more modifications as outlined above. Genetic modifications can include point mutations, partial deletions, full deletions, or insertions.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence (for example, a promoter that drives expression of the heterologous nucleic acid sequence encoding the siRNA or gRNA disclosed herein, in another example, a promoter that drives expression of a heterologous nucleic acid encoding Adrb1 and/or Adrb2). Generally, operably linked DNA sequences are contiguous and, where necessary to join two protein coding regions, are in the same reading frame.

Peripheral Blood Mononuclear Cell (PBMC): Cells that have one round nucleus. This term includes cells found in the blood. In this disclosure, this term is not limited to cells found in peripheral blood. In this disclosure PBMC is inclusive of tissue resident populations of cells, such as tissue resident memory T cells or tumor infiltrating lymphocytes, which are not commonly found in the peripheral blood. Examples include mast cells, macrophages, natural killer cells, monocytes, T cells, B cells, plasma cells, and dendritic cells. PBMCs do not include neutrophils, eosinophils or basophils. In one example, PBMCs are substantially isolated from other blood cells prior to use. In another example, PBMCs includes CD8+ TRM and TILs isolated from a solid tumor. In a further example, PBMCs includes immune cells with one round nucleus which are isolated from a solid tumor, or a non-peripheral blood tissue.

Pharmaceutically acceptable carriers: The pharmaceutically acceptable carriers useful in this disclosure are conventional. Remington's Pharmaceutical Sciences, 23rd Edition, Academic Press, Elsevier, (2020), describes compositions and formulations suitable for pharmaceutical delivery of a therapeutic agent, such as modified PBMCs disclosed herein.

In general, the nature of the carrier depends on the particular mode of administration being employed. For instance, parenteral formulations usually comprise injectable fluids that include pharmaceutically and physiologically acceptable fluids such as water, physiological saline, balanced salt solutions, aqueous dextrose, 5% human serum albumin, glycerol, or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. Supplementary active compounds can also be incorporated into the compositions.

Programmed cell death protein 1 (PD-1): A cell surface receptor that belongs to the immunoglobulin superfamily and is expressed on T cells and pro-B cells. PD-1 binds two ligands, PD-L1 and PD-L2. The human form is a 268 amino acid type 1 transmembrane protein. PD-1 is an inhibitory receptor that suppresses T cell activity and mediates T-cell exhaustion. PD-1 sequences are publicly available, for example from the GenBank® sequence database (e.g., Accession Nos. NP_005009.2 (mature peptide is aa 21-288), CAA48113.1, NP_001301026.1 (mature peptide is aa 25-288), and CAA48113.1 (mature peptide is aa 21-288) provide exemplary PD-1 protein sequences, while Accession Nos. L27440.1, NM_0050182, X67914.1, AB898677.1 and EU295528.2 provide exemplary PD-1 nucleic acid sequences).

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Examples of promoters include, but are not limited to the SV40 promoter, the CMV enhancer-promoter, the CMV enhancer/β-actin promoter, EF1a promoter, or PGK promoter. In one example, expression of a gRNA is driven by a polymerase III promoter, such as U6 or H1, such as human or mouse U6 or H1 promoter. Both constitutive and inducible promoters are included (see e.g., Bitter et al., Methods in Enzymology 153:516-544, 1987). Also included are those promoter elements that are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Prevent: Preventing a condition refers to reducing, delaying, or inhibiting the full development of a condition, for example preventing, reducing, or slowing the progression of a T cell to an exhausted T cell. In one example an agent that reduces Adrb1 and/or Adrb2 expression, or a non-naturally occurring genetic modification that reduces an amount of functional ADRB1 and/or ADRB2, when present in a PBMC, such a T cell, such as a CAR or TCR, prevents or reduces the likelihood that the cell will become exhausted (e.g., prevents or reduces the likelihood a T cell will overexpress or become positive for TOX, PD-1, CD39, TIM-3, LAG3, CXCR6 and/or CD101, or may slow the progression of the cell to an exhausted state. In some examples, the disclosed modified PBMCs, such as modified T cells, do not become exhausted. In some examples, the disclosed modified PBMCs, such as modified T cells, show a reduction in exhaustion, such as a reduction of least 10%, at least 25%, at least 50%, at least 75%, at least 90%, at least 95%, at least 99%, or at least 99.9%, relative to an unmodified PBMC or T cell. In some examples, the disclosed modified PBMCs, such as modified T cells, show a slower progression to exhaustion, such as an increase in the number of days to exhaustion of least 10%, at least 25%, at least 50%, at least 75%, at least 90%, at least 95%, at least 99%, slower relative to an unmodified PBMC or T cell.

In some examples the agent that increases expression of Adrb1 and/or Adrb2, when present in an antigen presenting cell, such as a dendritic cell, induces immune tolerance (e.g. reduced functional responsiveness in antigen-experienced cells) or may hasten the progression to a tolerant state. In some examples, the disclosed modified PBMCs become tolerant. In some examples the disclosed modified PBMCs show an increase in tolerance, such as an increase of at least 10%, at least 25%, at least 50%, at least 75%, at least 90%, at least 95%, at least 99%, or at least 99.9%, relative to an unmodified PBMC. In some examples, the disclosed modified PBMCs show a faster progression to exhaustion, such as a decrease in the number of days to tolerance by at least 10%, at least 25%, at least 50%, at least 75%, at least 90%, at least 95%, at least 99% faster, relative to an unmodified PBMC.

RNA interference or interfering RNA (RNAi): A cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. RNA molecules that inhibit gene expression through the RNAi pathway can include siRNAs, miRNAs, gRNAs, and shRNAs. In one example, an RNAi is specific for Adrb1, and can specifically hybridize to a Adrb1 nucleic acid molecule.

Sequence identity: The similarity between amino acid or nucleotide sequences is expressed in terms of the similarity between the sequences, otherwise referred to as sequence identity. Sequence identity is frequently measured in terms of percentage identity (or similarity or homology); the higher the percentage, the more similar the two sequences are. Homologs of a polypeptide (or nucleotide sequence) will possess a relatively high degree of sequence identity when aligned using standard methods.

Methods of alignment of sequences for comparison have been described. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Short hairpin RNA (shRNA): A sequence of RNA that makes a tight hairpin turn and can be used to silence gene expression via the RNAi pathway. The shRNA hairpin structure is cleaved by cellular machinery into siRNA. A shRNA that is “specific” for a target sequence (such as Adrb1) has sufficient complementarity to the target sequence that it binds the target and does not significantly hybridize with other unrelated sequences.

Small interfering RNA (siRNA): A double-stranded nucleic acid molecule that modulates gene expression through the RNAi pathway. siRNA molecules are generally 15 to 40 nucleotides in length, such as 20-30 or 20-25 nucleotides in length, with 0 to 5 (such as 2)-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.” A siRNA that is “specific” for a target sequence (such as Adrb1) has sufficient complementarity to the target sequence that it binds the target and does not significantly hybridize with other unrelated sequences.

Subject: A vertebrate, such as a mammal, for example a human. Mammals include, but are not limited to, murines, simians, humans, farm animals, sport animals, and pets. In one aspect, the subject is a non-human mammalian subject, such as a monkey or other non-human primate, mouse, rat, rabbit, pig, goat, sheep, dog, cat, horse, or cow. In some examples, the subject has cancer (or a tumor), that can be treated using the modified PBMCs disclosed herein or the agents that inhibit ADRB1 and/or ADRB2 disclosed herein. In some examples, the subject has a viral infection, that can be treated using the modified PBMCs disclosed herein or the agents that inhibit ADRB1 and/or ADRB2 disclosed herein. In some examples the subject has an autoimmune disease that can be treated using the modified PBMCs disclosed herein. In some examples, the subject is a laboratory animal/organism, such as a mouse, rabbit, or rat.

Suppressor cells: A subpopulation of immune cells which modulate the immune system, maintain tolerance to self-antigens, and abrogate autoimmune disease. These cells generally suppress or downregulate induction, proliferation, and function of other immune cells such as effector T cells, NK cells and dendritic cells. Suppressor cells come in many forms, including those that express CD4, CD25, and Foxp3 (CD4+CD25+Foxp3 regulatory T cells). Other examples of suppressor cells that can be targeted by the disclosed therapies include type II natural killer T (NK-T) cells, CD8+CD122+Treg, M2 and neuronal associated macrophages (NAMs), tumor infiltrating fibroblasts, and myeloid-derived suppressor cells A suppressor cell surface protein is a protein expressed at least in part on the surface of a suppressor cell, which that the protein can specially bind to an antibody or antibody fragment. Thus, the suppressor cell surface protein can be one that only found on the surface, or has a portion on the surface (such as a transmembrane protein having one or more extracellular domains that can specifically bind to an appropriate antibody). Examples of suppressor cell surface proteins include but are not limited to: CD25, CD4, C—X—C chemokine receptor type 4 (CXCR4), C—C chemokine receptor type 4 (CCR4), cytotoxic T-lymphocyte-associated protein 4 (CTLA4), glucocorticoid induced TNF receptor (GITR), OX40, folate receptor 4 (FR4), CD16, CD56, CD8, CD122, CD23, CD163, CD206, CD11 b, Gr-1, CD14, interleukin 4 receptor alpha chain (IL-4Ra), interleukin-1 receptor alpha (IL-1Ra), CD103, interleukin-1 decoy receptor, fibroblast activation protein (FAP), CXCR2, CD33, and CD66b).

T cells: A white blood cell (lymphocyte) that is an important mediator of the immune response. T cells include, but are not limited to, CD3+ T cells, CD4+ T cells and CD8+ T cells. T cells also include Tregs. A CD4+ T cell is an immune cell that carries a marker on its surface known as “cluster of differentiation 4” (CD4). These cells, also known as helper T cells, help orchestrate the immune response, including antibody responses as well as killer T cell responses. CD8+ T cells carry the “cluster of differentiation 8” (CD8) marker. In some examples, a CD8+ T cell is a cytotoxic T lymphocyte (CTL). CD3+ T cells carry the “cluster of differentiation 3” (CD3) marker, a multimeric protein complex historically known as the T3 complex. Regulatory T cells (Tregs) are often identified by expression of the markers CD4, CD25, and FoxP3, they can suppress immune activation, for example by secreting IL-10.

Activated T cells can be detected by an increase in cell proliferation and/or expression of or secretion of one or more cytokines (such as IL-2, IL-4, IL-6. IFN-γ, or TNFα). Activation of CD8+ T cells can also be detected by an increase in cytolytic activity in response to an antigen. “Exhausted T cells” are dysfunctional (hyporesponsive) T cells, commonly found in cancer environments. T cell exhaustion is characterized by a progressive loss of effector function (for example, loss of IL-2, TNF-α, and IFN-γ production) and sustained expression of inhibitory receptors such as PD-1, T cell immunoglobulin domain and mucin domain-containing protein 3 (TIM-3), CTLA-4, lymphocyte-activation gene 3 (LAG-3), CD101, TIGIT, and CD160. In some examples, the exhausted T cell is a CD3+ T cell, CD4+ T cell or CD8+ T cell. In some examples, the exhausted T cell is a terminally exhausted T cell (a terminally differentiated T cell that is exhausted). In some examples the exhausted cell is an “exhausted progenitor T cell” (TEX_{Prog}). TEX_{prog} may have high or persistent expression of any or all of TOX, PD-1, SLAMF6, ID3, CD127, CXCR5 and TCF1 relative to other T cells (PD-1+SLAMF6+ and/or TCF1+ T cells). In some examples the exhausted cell is an “exhausted effector-like T cell” (TEX_{Eff}). TEX_{Eff} may have high or persistent expression of any or all of PD-1 CX3CR1, TBX21 and TIM3 relative to other T cells (PD-1+CX3CR1+TIBX21+ and/or TIM3+). “Terminally Exhausted T cells” (TEX_{Term}) may have high or persistent expression of any or all of TIM3, PD-1, CXCR6, CD39, LAYN, EOMES and CD101 relative to other T cells (TIM3+ PD-1+ CD101+ T cells). Expression of the above proteins can be determined by FACs analysis, for example, by FACs analysis of a population of T cells.

In some examples, terminally exhausted T cells and other subsets of exhausted T cells such as TEX_{Prog} and TEX_{Eff} express ADRB1. A possible cause of T cell exhaustion is chronic activation or prolonged antigen stimulation. In some examples, the modified PBMC is an exhausted T cell (including a terminally exhausted T cell).

A “Therapeutic T Cell” is a T cell that is used for therapy, such as immunotherapy (e.g., cancer immunotherapy, and/or autoimmune immunotherapy). Therapeutic T cells are administered to a subject for treatment of a particular disease, for example, cancer, a viral infection, or an immune disease. In some examples, the therapeutic T cell recognizes and kills target cells, for example, cancerous cells, thereby treating a disease, such as cancer. In some examples, the therapeutic T cell recognizes and kills virally infected cells. In some examples the therapeutic T cell recognizes and kills a population of cells causing autoimmunity. Therapeutic T cells may be autologous or allogeneic to the subject. In some examples, the therapeutic T cell is a T cell to be used for Adoptive Cell Transfer (ACT) immunotherapy. In further examples, the therapeutic T cell expresses a Chimeric Antigen Receptor (CAR) or Engineered T Cell Receptor (TCR), and/or is a Tumor-Infiltrating Lymphocyte (TIL). In other examples the T cell is an exhausted T cell or a tissue resident memory (TRM) T cell.

T cell receptor (TCR): A receptor found on the surface of T lymphocytes (or T cells) responsible for recognizing fragments of antigen as peptides bound to major histocompatibility complex (MHC) molecules. The TCR is composed of two different protein chains. In humans, in 95% of T cells the TCR consists of an alpha (α) and beta (β) chain, whereas in 5% of T cells the TCR consists of gamma and delta (γ/δ) chains. This ratio changes during ontogeny and in diseased states as well as in different species. When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors. In one example, a TCR is a recombinant TCR, such as one used in TCR-engineered T cells for ACT therapy.

Tissue Resident Memory (TRM): Immune memory subset cells that reside in situ, typically in nonlymphoid tissues, rather than recirculating. In some examples, the TRM are CD8+ TRM cells. In some examples, the TRM are CD4+ TRM cells. Exemplary TRM genetic markers include one or more of Itgae, Itga1, Runx3, Cxcr3, Prdmn1, Notch2, Tcf7, Cxcr5, I17r, Id3, P2rx7, and/or Cd69. In some examples CD8+ or CD4+ TRM express CD69 and/or CD103 on the cell's surface in tissues, but not the blood. In some examples TRM upregulate CD49a, CRTAM, CD103, CD69, CXCR6, CD101, and/or PD-1. In some examples TRM downregulate CD62L, SIPR1, S1PR5, CX3CR1, KLF2, KLF3, and/or Ki67.

Transformed: A transformed cell is a cell (such as a PBMC, such as a T cell) into which a nucleic acid molecule has been introduced by molecular biology techniques. As used herein, the term transformed and the like (e.g., transformation, transfection, transduction, etc.) encompass all techniques by which a nucleic acid molecule might be introduced into such a cell, including viral vectors, plasmid vectors, nucleic acid-protein complexes (e.g., ribonucleoprotein), or naked nucleic acids (e.g., oligonucleotides).

Exemplary methods of transformation include chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, blood and other clinical tests, and the like. In some examples, treatment with the disclosed methods results in a decrease in the number, volume, and/or weight of a tumor and/or metastases. In some examples, treatment with the disclosed methods results in a decrease in signs or symptoms of the viral infection in a subject, and/or reduces viral load in a subject, and/or reduces infectivity of a virus, and/or reduce cytopathic effect in the subject's cells. In some examples treatment with the disclosed methods results in a decrease in signs or symptoms of autoimmunity in the subject, decreases immune cell activation in the subject, and/or reduces autoantibodies in the subject.

Tumor-Infiltrating Lymphocyte (TIL): lymphocytes that invade tumor tissue. For example, T cells found within a tumor sample. In ACT therapy, TIL therapy generally involves isolating TILs from a patient tumor, activating and expanding the TILs in culture, and then re-infusing into the patient. In some examples, the modified PBMC disclosed herein is a TIL. In some examples the TIL is identified by expression of: PD-1+, CD39+, LAG3+, TIGIT+, CD69+, TOX+, TIM3+, EOMES+, TBET+, CXCR6+, CD38+, CD101+, 2B4+, and/or CTLA-4+.

Tumor, neoplasia, or malignancy: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be treated using the disclosed modified PBMCs include solid tumors, such as breast carcinomas (e.g. lobular and duct carcinomas, such as a triple negative breast cancer), sarcomas, carcinomas of the lung (e.g., non small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors. Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is a melanoma.

The disclosed modified PBMCs can also be used to treat liquid tumors, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

Tumor-Specific Antigen: antigens unique to cancer cells or much more abundant on them, as compared to other cells, such as normal cells. Exemplary tumor-specific antigens include, but are not limited to, CD19, CD20, BCMA, MUC1 PSA, CEA, HER1, HER2, TRP-2, EpCAM, GPC3, mesothelin 1 (MSLN), and EGFR.

Up Regulation and Knock-In: When used in reference to the expression of a molecule, such as a target, “up regulation” refers to any process which results in an increase in production of an RNA of interest. In one example, upregulation increases detectable RNA expression or RNA activity. A “knock-in” is an increase in expression due to the introduction of a nucleic acid molecule encoding a protein of interest, such as ADRB1 and/or ADRB2.

Upregulation includes any detectable increase in the RNA. In certain examples, detectable RNA in a cell or cell free system increases by at least 10%, at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such an amount of Adrb1 and/or Adrb2 RNA detected in a corresponding non-treated cell or sample).

Vector: A nucleic acid molecule that can be introduced into a host cell (for example, by transfection or transformation), thereby producing a transformed host cell (such as a transformed PBMC). Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors (such as AAV and lentiviral vectors) are recombinant nucleic acid vectors having at least some nucleic acid sequences derived from one or more viruses. A replication deficient viral vector is a vector that requires complementation of one or more regions of the viral genome required for replication due to a deficiency in at least one replication-essential gene function.

Virus (or viral infection): Infective agents typically including a nucleic acid molecule surrounded by a protein coat. Viruses that can be treated with the disclosed methods include positive-strand RNA viruses and negative-strand RNA viruses. Exemplary positive-strand RNA viruses include, but are not limited to: Picornaviruses (such as Aphthoviridae [for example foot-and-mouth-disease virus (FMDV)]). Cardioviridae; Enteroviridae (such as Coxsackie viruses, Echoviruses, Enteroviruses, and Polioviruses); Rhinoviridae (Rhinoviruses)); Hepataviridae (Hepatitis A viruses): Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); hepaciviruses, (such as Hepatitis C virus), and Coronaviruses (examples of which include SARS coronaviruses, such as the Urbani strain, SARS-CoV and SARS-CoV-2). Exemplary negative-strand RNA viruses include, but are not limited to: Orthomyxyoviruses (such as the influenza virus). Rhabdoviruses (such as Rabies virus), vesiculoviruses (such as VSV) and Paramyxoviruses (examples of which include measles virus, respiratory syncytial virus, and parainfluenza viruses).

Viruses that can be treated with the disclosed methods also include DNA viruses. DNA viruses include, but are not limited to: Hepadnaviridae (Hepatitis B virus) Herpesviruses (such as Varicella-zoster virus (VZV), for example the Oka strain; cytomegalovirus (CMV); epstein-barr virus (EBV), and Herpes simplex virus (HSV) types 1 and 2), Adenoviruses (such as Adenovirus type 1 and Adenovirus type 41), Poxviruses (such as Vaccinia virus), papillomavaridae (such as human papillomavirus (HPV)), (and Parvoviruses (such as Parvovirus B19).

Another group of viruses that can be treated with the disclosed methods includes Retroviruses. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

Viral infections of long duration or which recur over a long period of time are sometimes referred to as chronic viral infections. Exemplary viruses which can establish a chronic infection include adenovirus (Ad), a herpes simplex virus (HSV), a hepatitis B virus (HBV), a hepatitis C virus (HCV), a vesicular stomatitis virus (VSV), a human immunodeficiency virus (HIV), an influenza virus, a varicella zoster virus (VZV), a human papillomavirus (HPV), an Epstein-Barr virus (EBV), a cytomegalovirus (CMV), an enterovirus, a togavirus, a SARS-CoV virus, a SARS-CoV-2 virus, or a flavivirus.

The disclosed methods can be used in combination with one or more antiviral agents. Antiviral agents can work by a variety of mechanisms, including inhibiting any or all of: attachment, entry, uncoating, protease activity, polymerase activity, nucleoside and/or nucleotide reverse transcriptase activity, nonnucleoside reverse transcriptase activity, and integrase activity. Antiviral agents might also physically disrupt a virion. Exemplary antiviral agents that can be used with the methods provided herein include Lopinavir (for HIV), remdesivir (for SARS-CoV-2), acyclovir (for Herpes viruses), ribavirin (for viral hemomoragic fevers), emtricitabine/tenofovir (for HIV), pegylated interferon alpha (for viral hepatitis), bamlanivimab/etesevimab (for SARS-CoV-2), and ZMapp (for ebolavirus). Antiviral agents can be given in combination, for example to prevent the target virus from developing resistance to the therapy. In some examples, the antiviral agents include aciclovir, ganciclovir, zidovudine, interferon alpha, and/or direct acting antiviral agents.

### II. Overview

The adrenergic receptor ADRB1 is shown herein to be a novel immune checkpoint with increased expression on exhausted T cells. ADRB1 is upregulated after the induction of the exhaustion process, during the transition of progenitors of exhausted T cells to more terminal differentiated exhausted cells, promotes several features of T cell exhaustion such as impaired proliferation and cytokine production, and determines the perineural localization of exhausted T cells. It is demonstrated herein that in settings of chronic antigen exposure, adrenergic signaling on T cells via ADRB1 is a significant signal contributing to terminal differentiation of T cells to the exhausted state. It is shown herein that genetic or pharmacological blockade of ADRB1 with existing clinically approved drugs (such as beta-blockers) prevents terminal exhaustion differentiation of T cells and increases T cell function in chronic viral infection and in a melanoma model. The emergence of CD8⁺ T cells with a T_{RM} signature in animals treated with a combination of beta-blockers and checkpoint blockade is associated with an effective T cell response in an otherwise non-checkpoint responsive orthotopic model of pancreatic cancer.

Disclosed herein are modified PBMCs which have one or both of (a) an agent that reduces Adrb1 expression or a non-naturally occurring genetic modification that reduces an amount of functional ADRB1; and/or (b) an agent that reduces Adrb2 expression or a non-naturally occurring genetic modification that reduces an amount of functional ADRB2. In some examples the modified PBMCs include both (a) the agent that reduces Adrb1 expression or the non-naturally occurring genetic modification that reduces an amount of functional ADRB1 and (b) the agent that reduces Adrb2 expression or the non-naturally occurring genetic modification that reduces an amount of functional ADRB2. In some examples the modified PBMCs include only one of (a) the agent that reduces Adrb1 expression or the non-naturally occurring genetic modification that reduces an amount of functional ADRB1 and (b) the agent that reduces Adrb2 expression or the non-naturally occurring genetic modification that reduces an amount of functional ADRB2.

In some aspects the agent that reduces Adrb1 expression includes an inhibitory RNA (RNAi) specific for Adrb1 or a guide RNA (gRNA) specific for Adrb1. In some aspects the agent that reduces Adrb2 expression includes an RNAi specific for Adrb2 or a gRNA specific for Adrb2. In some aspects the RNAi specific for Adrb1 is a short hairpin RNA (shRNA) molecule, short interfering RNA (siRNA) molecule, or antisense RNA molecule. In some aspects the RNAi specific for Adrb2 is a shRNA molecule, siRNA molecule, or antisense RNA molecule. In some aspects the agent that reduces Adrb1 expression is a heterologous nucleic acid molecule encoding at least one of: (i) the RNAi specific for Adrb1 gene or transcript, where the RNAi specific for Adrb1 comprises at least 90% complementarity to a portion of the Adrb1 gene or transcript. (ii) the gRNA specific for Adrb1 gene or transcript, where the gRNA specific for Adrb1 comprises at least 90% sequence identity to a portion of the Adrb1 gene or transcript, or (iii) the gRNA specific for Adrb1 gene or transcript, where the gRNA specific for Adrb1 comprises at least 90% sequence identity to a portion of the Adrb1 gene or transcript and a Cas nuclease. In some aspects the agent that reduces Adrb2 expression is a heterologous nucleic acid molecule encoding at least one of (i) the RNAi specific for Adrb2 gene or transcript, where the RNAi specific for Adrb2 comprises at least 90% complementarity to a portion of the Adrb2 gene or transcript, (ii) the gRNA specific for Adrb2 gene or transcript, wherein the gRNA specific for Adrb2 comprises at least 90% sequence identity to a portion of the Adrb2 gene or transcript, or (iii) the gRNA specific for Adrb2 gene or transcript, where the gRNA specific for Adrb2 comprises at least 90% sequence identity to a portion of the Adrb2 gene or transcript and a Cas nuclease. In some aspects the PBMC includes a heterologous nucleic acid molecule encoding a gRNA specific for Adrb1, including any or all of SEQ ID NOs: 7, 8, 9, 10, 11, 12, 13, 14, 15, or 16. In some aspects, the PBMC includes a heterologous nucleic acid molecule encoding a gRNA specific for Adrb2, including any or all of SEQ ID NOs: 17, 18, 19, 20, 21, 22, 23, 24, 25 or 26.

In some examples the modified PBMCs include an expression vector encoding the heterologous nucleic acid. In some examples the modified PBMC includes a gRNA specific for the Adrb1 or Adrb2 gene or transcript and a Cas nuclease, such as Cas3, dCas3, Cas9, dCas9, Cas12, dCas12, Cas13a, dCas13a, Cas13b, dCas13b. Cas13d, or dCas13d.

In some examples, the modified PBMCs include a genetic modification that reduces ADRB1 or ADRB2. In some aspects the genetic modification that reduces ADRB1 is a point mutation, a partial deletion, full deletion, or insertion of Adrb1 that reduces expression of Adrb1 and/or reduces activity of ADRB1. In some aspects the genetic modification that reduces ADRB2 is a point mutation, a partial deletion, full deletion, or insertion of Adrb2 that reduces expression of Adrb2 and/or reduces activity of ADRB2.

In some examples, the modified PBMC is a T cell. In some examples the T cell is a CD8+ T cell, a CD4+ T cell, a CD3+ T cell, a NKT cell, a gamma delta T cell, or a MAIT cell. In some examples the T cell is a therapeutic T cell. In some examples the T cell is an exhausted T cell, a tissue resident memory T cell (TRM), a chimeric antigen receptor (CAR) T cell, an engineered T cell receptor (TCR) T cell, or a tumor-infiltrating lymphocyte (TIL). In some examples the PBMC is a tissue resident memory cell, or a tissue infiltrating lymphocyte. In some examples the T cell is reactive to a tumor-specific antigen such as CD19, CD20, BCMA, MUC1, PSA, CEA, HER1, HER2, TRP-2, EpCAM, GPC3, mesothelin 1 (MSLN), and EGFR. In some examples the modified PBMC is reactive to a viral antigen. In some examples the T cell is reactive to a viral antigen. In some examples the modified PBMC is an antigen presenting cell, such as a monocyte/macrophage, dendritic cell, NK cell or B cell.

Further disclosed is a method of generating a modified PBMC including (a) introducing the agent that reduces Adrb1 expression or non-naturally occurring genetic modification that reduces functional ADRB1 into a PBMC, thereby generating the modified PBMC with reduced expression of Adrb1, reduced activity of ADRB1, or both; or (b) introducing the agent that reduces Adrb2 expression or non-naturally occurring genetic modification that reduces functional ADRB2 into a PBMC, thereby generating the modified PBMC with reduced expression of Adrb2, reduced activity of ADRB2, or both. In some examples the modified PBMC is a T cell. In some examples this method further includes incubating the modified PBMC with interleukin 2, (IL-2), interleukin 7 (IL-7), interleukin 15 (IL-15), TGF-beta, retinoic acid or a combination thereof. In some examples the modified PBMC is reactive to a tumor-specific antigen, such as CD19, CD20, BCMA, MUC1, PSA, CEA, HER1, HER2, TRP-2, EpCAM, GPC3, mesothelin 1 (MSLN), and EGFR. In some examples, reduced expression of Adrb1, reduced activity of ADRB1, reduced expression of Adrb2, or reduced activity of ADRB2, increases effector function of the T cell, reduces exhaustion of the T cell, causes the T cell to express Itgae, Itga1, Runx3, Cxcr3, Prdmn1, Notch2, Tcf7, Cxcr5, I17r, Id3, or Cd69 and/or causes reduced expression of S1pr1, Klf2, Klf3, Pdcd1, Tox, Entpd1, Cxcr6, Eomes, Tbx21, Tigit, Cd38, Lag3, Cx3cr1, Cd101, Havcr2 by the T cell.

In some examples the PBMC is an antigen presenting cell, such as a monocyte/macrophage, a dendritic cell, NK cell or a B cell. In some examples the method further includes (a) selecting the modified PBMC with reduced expression of Adrb1, reduced activity of ADRB1, or both; or (b) selecting the modified PBMC with reduced expression of Adrb2, reduced activity of ADRB2, or both. In some examples the method includes introducing the selected modified PBMC into a subject. In some examples the selecting includes the use of flow cytometry, panning, or magnetic separation. In some examples the subject has cancer. In some examples the method includes selecting the subject who has cancer. In some examples the subject has a viral infection. In some examples the method includes selecting a subject who has a viral infection.

Further disclosed is a pharmaceutical composition including the modified PBMC described above or the modified PBMC as generated by the methods described above, and a pharmaceutically acceptable carrier. In some examples the composition is in an intravenous formulation. In some examples the pharmaceutical composition includes one or more immune checkpoint blockade (ICB) agents. In some examples the pharmaceutical composition includes one or more antiviral agents. In some examples the pharmaceutical composition includes one or more additional anti-cancer agents, such as an anti-cancer monoclonal antibody or chemotherapeutic.

Further disclosed is a method for treating cancer or a tumor by administering a therapeutically effective amount of the modified PBMC as described above, a therapeutically effective amount of the modified PBMC generated as described above, or a therapeutically effective amount of the pharmaceutical composition described above to the subject having cancer or the tumor, thereby treating the cancer or the tumor. In some examples the modified PBMC is autologous to the subject. In some examples the modified PBMC is allogenic to the subject. In some examples the method of treating cancer or a tumor includes administering a therapeutically effective amount of Il-2, Il-7, and/or Il-15 to the subject. In some examples the method of treating cancer or a tumor further includes treating the subject with one or more of surgery, radiation, chemotherapy, biologic therapy (such as a monoclonal antibody), or immunotherapy. In some examples the method of treating cancer or a tumor includes administering to the subject a therapeutically effective amount of one or more of: a T cell agonist antibody, an oncolytic virus, or an adoptive cell transfer (ACT) immunotherapy. In some examples the method of treating cancer or a tumor includes administering to the subject a therapeutically effective amount of immune checkpoint blockade (ICB) agent or immunostimulatory antibody. In some aspects, the ICB agent comprises anti-PD-1, anti-PD-L1, anti-CTLA-4, anti-LAG3 anti-GITR, anti-4-IBB, anti-CD40, anti-CD40L, and anti-OX40, anti-TIGIT, anti-VISTA, anti-CD73, anti-CD39, anti-HVEM, anti-BTLA, anti-CD27, or a combination of two or more thereof. In some aspects, the anti-PD-1 is nivolumab, pembrolizumab, pidilizumab, or cemiplimab. In some aspects, the anti-PD-L1 atezolizumab, avelumab, durvalumab, cosibelimab, KN035 (envafolimab), BMS-936559, BMS935559, MEDI-4736, MPDL-3280A, or MEDI-4737. In some aspects, the anti-CTLA-4 is ipilimumab or tremelimumab. In some examples the modified PBMC is administered simultaneously with the ICB agent or the immunostimulatory antibody. In other examples the modified PBMC is administered before the ICB agent or the immunostimulatory antibody. In still other examples the modified PBMC is administered after the ICB agent or the immunostimulatory antibody. In some examples non-modified lymphocytes are depleted in the subject prior to administering the modified PBMC. In some examples the cancer or tumor is an acute or chronic leukemia, Hodgkin or Non-Hodgkin lymphoma, myeloma, gastric cancer, esophageal cancer, colorectal cancer, hepatocellular carcinoma or other liver cancer, cholangiocellular carcinoma, melanoma, cervical cancer, uterine cancer, lung cancer, ovarian cancer, bladder cancer, urothelial cancer, breast cancer, pancreatic cancer, renal cell carcinoma, prostate cancer, testicular cancer, glioblastoma, nephroblastoma, neuroblastoma, neuroendocrine cancer, pheochromocytoma, sarcoma, thyroid cancer, laryngeal cancer or head and neck cancer. In some examples the cancer is non-checkpoint responsive pancreatic cancer. In some examples the cancer is non-checkpoint responsive cancer.

Further disclosed is a method for treating a viral infection (such as a chronic viral infection), including administering a therapeutically effective amount of the modified PBMC as described above, a therapeutically effective amount of the modified PBMC generated by the methods described above, and/or a therapeutically effective amount of the pharmaceutical composition described above to a subject having a viral infection, thereby treating the viral infection. In some examples, the subject is also treated with an antiviral agent, such as aciclovir, ganciclovir, zidovudine, interferon alpha, and direct acting antiviral agents. In some examples the method of treating a viral infection further includes administering to the subject a therapeutically effective amount of immune checkpoint blockade (ICB) agent or immunostimulatory antibody. In some aspects, the ICB agent comprises anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, anti-CD27, or a combination of two or more thereof. In some examples the method of treating a viral infection includes administering to the subject interferon alpha or pegylated interferon alpha. In some aspects the viral infection is caused by an adenovirus (Ad), a herpes simplex virus (HSV, type 1 and 2), a hepatitis B virus (HBV), a hepatitis C virus (HCV), a hepatitis D virus (HDV), a hepatitis E virus (HEV), a vesicular stomatitis virus (VSV), a human immunodeficiency virus (HIV), an influenza virus, a varicella zoster virus (VZV), a human papillomavirus (HPV), an Epstein-Barr virus (EBV), a cytomegalovirus (CMV), a human herpesvirus (HHV-6, HHV-7), a human T-cell leukemia virus (HTLV-1, HTLV-2), JC virus. BK virus, an enterovirus, a parvovirus, a paramyxovirus (e.g. measles), a togavirus or a flavivirus.

Further disclosed are modified PBMCs with increased expression of Adrb1, Adrb2, or both, including (a) an agent that increases Adrb1 expression or a non-naturally occurring genetic modification that increases an amount of functional ADRB1; and/or (b) an agent that increases Adrb2 expression or a non-naturally occurring genetic modification that increases an amount of functional ADRB2. In some aspects, the agent that increases expression of Adrb1 includes an expression vector encoding Adrb1, optionally operably linked to a promoter. In some aspects, the agent that increases expression of Adrb2 includes an expression vector encoding Adrb2 optionally operably linked to a promoter. In some aspects, the agent that increases expression of Adrb1 includes a heterologous nucleic acid encoding Adrb1 optionally operably linked to a promoter. In some aspects, the agent that increases expression of Adrb2 includes a heterologous nucleic acid Adrb2 optionally operably linked to a promoter. In some examples the agent that increases expression of Adrb1 or Adrb1 includes a gRNA and a Cas nuclease. In some examples the expression vector or heterologous nucleic acid encoding Adrb1 includes a sequence having at least 90%, at least 95%, or 100% sequence identity to SEQ ID NO: 3. In some examples the expression vector or heterologous nucleic acid encoding Adrb1 encodes an ADRB1 protein having at least 90%, at least 95%, or 100% sequence identity to SEQ ID NO: 1. In some examples the expression vector or heterologous nucleic acid encoding Adrb2 includes a sequence having at least 90%, at least 95%, or 100% sequence identity to SEQ ID NO: 6. In some examples the expression vector or heterologous nucleic acid encoding Adrb1 encodes an ADRB2 protein having at least 90%, at least 95%, or 100% sequence identity to SEQ ID NO: 4. In some examples the modified PBMC is a T cell. In some examples the modified PBMC is a CD3+, a CD4+, a CD8+ and/or a CD25+ T cell. In some examples the modified PBMC is a NKT cell, a gamma delta T cell, or a MAIT cell. In some examples the modified PBMC is a Treg cell. In some examples the modified PBMC is an antigen presenting cell, such as a monocyte/macrophage, a dendritic cell, a NK cell or a B cell

Further disclosed is a method of generating a modified PBMC including (a) introducing the agent that increases Adrb1 expression or non-naturally occurring genetic modification that increases functional ADRB1 into a PBMC, thereby generating the modified PBMC with increased expression of Adrb1, increased activity of ADRB1, or both; or introducing the agent that increases Adrb2 expression or non-naturally occurring genetic modification that increases functional ADRB2 into a PBMC, thereby generating the modified PBMC with increased expression of Adrb2, increased activity of ADRB2, or both. In some examples the method also includes (a) selecting the modified PBMC with increased expression of Adrb1, increased activity of ADRB1, or both; or (b) selecting the modified PBMC with increased expression of Adrb2, increased activity of ADRB2, or both. In some examples the method includes introducing the selected modified PBMC into a subject. In some examples the selecting includes use of flow cytometry, panning, or magnetic separation. In some examples the subject has an autoimmune disease.

Further disclosed is a method for treating an autoimmune disease including administering a therapeutically effective amount of the modified PBMCs described above or the modified PBMCs generated by the methods described above to the subject having the autoimmune disease, thereby treating the autoimmune disease. In some aspects the autoimmune disease is rheumatoid arthritis, systemic lupus erythematosus, type 1 and type 2 diabetes, multiple sclerosis, acute disseminated encephalomyelitis, Sjögren's syndrome, Graves' disease, myasthenia gravis, ulcerative colitis, Hashimoto's thyroiditis, celiac disease, Crohn's disease, arthritis, inflammatory bowel disease, psoriasis, autoimmune hepatitis, autoimmune pancreatitis, autoimmune encephalitis, scleroderma, autoimmune hemolytic anemia, autoimmune thrombocytopenia, autoimmune neutropenia, atopic dermatitis, alopecia, or ankylosing spondylitis. In some examples the method also includes treating the subject with an immunosuppressive agent such as steroids, azathioprine, methotrexate, anti-TNF, anti-Il-12, and/or anti-Il-23.

Further disclosed is a method for preventing or treating cancer in a subject, including administering an agent that inhibits ADRB1 signaling administered in an amount effective to inhibit ADRB1 signaling on PBMCs and/or an agent that inhibits ADRB2 signaling administered in an amount effective to inhibit ADRB2 signaling on the PBMCs; and a therapeutically effective amount of immune checkpoint blockade (ICB) agent. Inhibition does not require 100% reduction in signaling, but can include a reduction in signally by at least 20%, at least 50%, at least 75%, at least 90%, at least 95%, at least 99%, or 100%. In some examples the method includes selecting the subject with cancer for treatment. In some examples, the cancer is a solid tumor. In some examples the amount effective to inhibit ADRB1 signaling is an amount effective to inhibit ADRB1 signaling on PBMCs localized within the solid tumor. In some examples the amount effective to inhibit ADRB2 signaling is an amount effective to inhibit ADRB2 signaling on PBMCs localized within the solid tumor. In some examples the cancer is a liquid cancer. In some examples the amount effective to inhibit ADRB1 signaling is an amount effective to inhibit ADRB1 signaling on PBMCs localized in the subject's bloodstream or lymphatic system. In some examples the amount effective to inhibit ADRB1 signaling, and the amount effective to inhibit ADRB2 signaling is an amount effective to inhibit ADRB2 signaling on PBMCs localized in the subject's bloodstream or lymphatic system. In some examples the method includes treating the subject with one or more of surgery, radiation, chemotherapy, biologic therapy, or immunotherapy. In some examples the method includes administering to the subject a therapeutically effective amount of one or more of: a T cell agonist antibody, an oncolytic virus, or an adoptive cell transfer (ACT) immunotherapy. In some examples the method includes administering to the subject a therapeutically effective amount of immune checkpoint blockade (ICB) agent or immunostimulatory antibody. In some aspects, the ICB agent comprises anti-PD-1, anti-PD-L1, anti-CTLA-4, anti-LAG3 anti-GITR, anti-4-IBB, anti-CD40, anti-CD40L, and anti-OX40, anti-TIGIT, anti-VISTA, anti-CD73, anti-CD39, anti-HVEM, anti-BTLA, anti-CD27, or a combination of two or more thereof. In some aspects, the anti-PD-1 is nivolumab, pembrolizumab, pidilizumab, or cemiplimab. In some aspects, the anti-PD-L1 atezolizumab, avelumab, durvalumab, cosibelimab, KN035 (envafolimab), BMS-936559, BMS935559, MEDI-4736, MPDL-3280A, or MEDI-4737. In some aspects, the anti-CTLA-4 is ipilimumab or tremelimumab. In some examples the cancer or tumor is an acute or chronic leukemia, Hodgkin or Non-Hodgkin lymphoma, myeloma, gastric cancer, esophageal cancer, colorectal cancer, hepatocellular carcinoma or other liver cancer, cholangiocellular carcinoma, melanoma, cervical cancer, uterine cancer, lung cancer, ovarian cancer, bladder cancer, urothelial cancer, breast cancer, pancreatic cancer, renal cell carcinoma, prostate cancer, testicular cancer, glioblastoma, nephroblastoma, neuroblastoma, neuroendocrine cancer, pheochromocytoma, sarcoma, thyroid cancer, laryngeal cancer or head and neck cancer. In some examples the cancer is non-checkpoint responsive pancreatic cancer. In some examples the cancer is non-checkpoint responsive cancer.

In some examples the agent that inhibits ADRB1 signaling and the agent that inhibits ADRB2 signaling are both a beta-blocker. In some examples the beta-blocker is atenolol, bisoprolol, metoprolol, propranolol, bucindolol, oxprenolol, carteolol, pindolol, oxprenolol, penbutolol, betaxolol, celiprolol, acebutolol, labetalol, carvedilol, pronethalol, sotalol, nebivolol, esmolol, butaxamine, alprenolol, bupranolol, nadolol, or timolol. In some examples, the agent that inhibits ADRB1 is CGP 20712A. In some examples the agent that inhibits ADRB2 signaling is ICI 118551. In some examples the agent that inhibits ADRB1 signaling or the agent that inhibits ADRB2 signaling is administered before the ICB agent. In other examples the agent that inhibits ADRB1 signaling or the agent that inhibits ADRB2 signaling is administered concurrently with the ICB agent. In still other examples the agent that inhibits ADRB1 signaling or the agent that inhibits ADRB2 signaling is administered after the ICB agent.

### III. RNAi and gRNA

Disclosed herein are interfering RNAs (RNAi) or guide nucleic acids (gRNA) specific for Adrb1 (ADRB1) and/or Adrb2 (ADRB2). The RNAi or gRNA targets an Adrb1 and/or Adrb2 nucleic acid molecule, such as a gene or transcript, to reduce expression of Adrb1 and/or Adrb2, such as a reduction in expression of at least 20%, at least 30%, at least 40%, at least 50%, at least 60%, at least 75%, at least 90%, at least 95%, at least 99%, at least 99.9% or even 100% (for example relative to an amount prior to introduction/expression of the RNAi or gRNA). In some examples, the RNAi or gRNA are introduced into a cell, for example, a PBMC, T cell, or exhausted T cell (including a terminally exhausted T cell). In other examples the RNAi or gRNA are introduced into a CD8+ T cell, a CD8+ TRM cell, a chimeric antigen receptor T cell, an engineered T cell receptor T cell, a tumor-infiltrating lymphocyte, or a CD8+ tumor-infiltrating tissue resident memory T cell, optionally, wherein the antigen receptor is reactive to a tumor-specific antigen. In further examples, the RNAi or gRNA target an antigen presenting cell, such as a dendritic cell, a natural killer (NK) cell, a monocyte/macrophage, or a B cell. In some examples, the RNAi or gRNA molecules are directly introduced into the cell, for example, as oligonucleotides. In some examples, RNAi of gRNA molecules are expressed from a vector that is introduced into the cell. In examples where a guide RNA is expressed (e.g., from an expression cassette or vector) the guide RNA may be encoded as DNA.

In some aspects, an RNAi specific for a Adrb1 and/or Adrb2 gene or transcript is used to reduce or inhibit expression of Adrb1 and/or Adrb2. The specificity of the RNAi for Adrb1 and/or Adrb2 allows hybridization of the RNAi molecule to Adrb1 and/or Adrb2 DNA or RNA, thereby reducing or inhibiting Adrb1 and/or Adrb2 expression. RNAi generically refers to a cellular process that inhibits expression of genes by inhibiting transcription and/or translation. Molecules that inhibit gene expression through the RNAi pathway include siRNAs, miRNAs, antisense RNAs, and shRNAs. In some examples, the RNAi specific for Adrb1 and/or Adrb2 includes a sequence at least 70%, at least 80%, at least 90%, at least 95%, at least 99%, or 100% complementary to a unique (e.g., not found elsewhere in the genome of the cell or organism into which the RNAi is introduced) contiguous portion of a Adrb1 and/or Adrb2 gene or transcript (such as a portion of SEQ ID NO: 2, 3, 5 or 6). In some examples, the RNAi specific for Adrb1 and/or Adrb2 consists of a sequence at least 90% complementary (e.g., at least 91%, 92%, 93%, 94%, 95%, 96%, 97%, 98% 99% or 100% complementary) to a unique contiguous portion of Adrb1 and/or Adrb2 gene or transcript (such as a portion of SEQ ID NO: 2, 3, 5 or 6).

In a specific, non-limiting example, the RNAi is a shRNA specific for a Adrb1 and/or Adrb2 gene or transcript. Methods of designing shRNA have been described, for example, see Moore et al. (2010) Short Hairpin RNA (shRNA): Design, Delivery, and Assessment of Gene Knockdown, Methods Mol. Biol., 629:141-158. In some examples, the shRNA is specific to a unique contiguous portion of a sequence with at least 70%, at least 80%, at least 90%, at least 95%, at least 98%, at least 99%, or 100% sequence identity to one of SEQ ID NO: 2, 3, 5 or 6. In some examples, the shRNA is specific to a unique contiguous portion of a sequence with at least 90% sequence identity to one of SEQ ID NO: 2, 3, 5 or 6.

In other examples, the RNAi is a siRNA specific for Adrb1 and/or Adrb2 gene or transcript, for example the siRNA is specific for a sequence with at least 70%, at least 80%, at least 90%, at least 95%, at least 98%, at least 99%, or 100% sequence identity to a unique contiguous portion (such as a contiguous portion of at least 8, at least 9, at least 10, at least 12, at least 15, or at least 20 contiguous nucleotides) of one of SEQ ID NOs: 2, 3, 5 or 6. For example, the siRNA can be specific to a sequence with at least 95% sequence identity to a unique contiguous portion of SEQ ID NO: 6.

In some aspects, a gRNA specific for a Adrb1 and/or Adrb2 gene or transcript is used to reduce or inhibit expression of Adrb1 and/or Adrb2. For example, CRISPR/Cas methods can be used with a gRNA specific for a Adrb1 gene or transcript to reduce or inhibit expression of Adrb1. In another example, CRISPR/Cas methods can be used with a gRNA specific for a Adrb2 gene or transcript to reduce or inhibit expression of Adrb2. In a further example, CRISPR/Cas methods can be used with a gRNAs specific for Adrb1 and Adrb2 to reduce or inhibit expression of Adrb1 and Adrb2.

The specificity of the gRNA for Adrb1 and/or Adrb2, in combination with a Cas nuclease or dead nuclease (such as Cas3, dCas3, Cas9, dCas9, Cas12, dCas12, Cas13a, dCas13a, Cas13b, dCas13b, Cas13d, or dCas13d) allows hybridization of the gRNA molecule to Adrb1 and/or Adrb2 DNA and/or RNA, thereby editing the Adrb1 and/or Adrb2 (for example mutating it, such as knocking it down or knocking it out) to reduce or inhibit its expression. In some examples, the Cas nuclease (or a dead Cas nuclease) sequence is codon optimized for expression in a host cell. In some examples, gRNA molecules and Cas nucleases are expressed from a vector introduced into a host cell (e.g., PBMC, antigen presenting cell. B cell, dendritic cell, monocyte/macrophage, NK cell, T cell, CD8+ TRM T cell, tumor infiltrating lymphocyte, CAR T cell, exhausted T cell, terminally exhausted T cell, or a cell with an antigen receptor reactive to a tumor-specific antigen). In some examples, an RNP complex containing gRNA molecules and Cas nucleases are introduced into a cell (e.g., PBMC, antigen presenting cell, B cell, dendritic cell, monocyte/macrophage, NK cell, T cell, CD8+ TRM T cell, tumor infiltrating lymphocyte, CAR T cell, exhausted T cell, terminally exhausted T cell, or a cell with an antigen receptor reactive to a tumor-specific antigen). In some examples, the gRNAs are introduced into a cell, for example, as oligonucleotides.

In some examples, the gRNA is specific for Adrb1 (ADRB1) and/or Adrb2 (ADRB2) gene or transcript. For example, the gRNA is specific for a sequence with at 70%, at least 80%, at least 90%, at least 95%, at least 98%, at least 99%, or 100% sequence identity to a unique contiguous portion (such as a contiguous portion of at least 8, at least 9, at least 10, at least 12, at least 15, at least 20, or at least 25 contiguous nucleotides) of one of SEQ ID NOs: 2, 4, or 6. In some examples, the gRNA is specific to a sequence with at least 90% sequence identity to a unique contiguous portion of one of SEQ ID NOs: 2, 4, or 6. In some examples, the gRNA comprises a targeting sequence (sometimes referred to as a spacer) specific to Adrb1 and/or Adrb2 genes or transcripts, for example, by having a targeting sequence that is at least 70%, at least 80%, at least 90%, at least 95%, at least 98%, at least 99%, or 100% complementary to a unique contiguous portion of one of SEQ ID NOs: 2, 4, or 6. The targeting sequence of the gRNA is typically about 20 nucleotides, for example, about 15, about 16, about 17, about 18, about 19, about 20, about 21, about 22, about 23, about 24, or about 25 nucleotides. In a specific non-limiting example, the targeting sequence is about 20 nucleotides. The degree of complementarity between a targeting sequence and its corresponding target sequence, when optimally aligned using a suitable alignment algorithm, is about or more than about 50%, about 60%, about 75%, about 80%, about 85%, about 90%, about 95%, about 97.5%, about 98%, about 99%, or more. In some aspects, the degree of complementarity is about 100%. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some examples, the targeting sequence is at least 80%, at least 85%, at least 90%, at least 91%, at least 92%, at least 93%, at least 94%, at least 95%, at least 96%, at least 97%, at least 98% or at least 99% complementary to a unique, contiguous amino acid sequence about 20 nucleotides long in a Adrb1 and/or Adrb2 gene or transcript.

In some examples, the gRNA specific for a Adrb1 and/or Adrb2 gene or transcript includes a contiguous sequence at least 70%, at least 80%, at least 90%, at least 95%, at least 98%, at least 99%, or 100% sequence identity to one of SEQ ID NOs: 7, 8, 9, 10, 11, 12, 13, 14, 15, 16, 17, 18, 19, 20, 21, 22, 23, 24, 25, 26, or 28. In some examples, the gRNA specific for Adrb1 and/or Adrb2 gene or transcript includes one of SEQ ID NOs: 7, 8, 9, 10, 11, 12, 13, 14, 15, 16, 17, 18, 19, 20, 21, 22, 23, 24, 25, 26, or 28. In some examples, the gRNA specific for Adrb1 and/or Adrb2 gene or transcript includes one having at least 90%, at least 95%, or at least 98% sequence identity to SEQ ID NOs: 7, 8, 9, 10, 11, 12, 13, 14, 15, 16, 17, 18, 19, 20, 21, 22, 23, 24, 25, 26, or 28 (such as substitution of 1, 2, 3, 4, 5, or 6 nt).

In some examples the gRNA is a sgRNA specific for a Adrb1 and/or Adrb2 gene or transcript. In some examples, the sgRNA includes a contiguous sequence having at least 70%, at least 80%, at least 90%, at least 95%, at least 98%, at least 99%, or 100% sequence identity to one of SEQ ID NOs: 7, 8, 9, 10, 11, 12, 13, 14, 15, 16, 17, 18, 19, 20, 21, 22, 23, 24, 25, 26, or 28. In some examples, the sgRNA includes one of SEQ ID NOs: 7, 8, 9, 10, 11, 12, 13, 14, 15, 16, 17, 18, 19, 20, 21, 22, 23, 24, 25, 26, or 28 (such as substitution of 1, 2, 3, 4, 5, or 6 nt).

Methods of designing gRNA and determining appropriate targeting sequences have been described (see e.g., Hanna and Doench (2020) Design and analysis of CRISPR-Cas experiments, Nature Biotechnology, 38:813-823(2020)). Software tools can be used to design and analyze of CRISPR-Cas experiments, including resources to design optimal gRNAs for various modes of manipulation and to analyze the results of such experiments. Online databases of validated gRNAs are also readily available (see addgene.org/crispr/reference/grna-sequence/and genscript.com/gRNA-database.html).

### IV. Nucleic Acids, Expression Vectors, Overexpression, and Knock-Ins

Nucleic acids (e.g., heterologous nucleic acids or isolated nucleic acid molecules, such as DNA, cDNA, RNA (e.g., mRNA)) encoding the RNAi, gRNAs, Cas proteins, agents that increase expression of Adrb1, and agents that increase expression of Adrb2 are also provided herein. Nucleic acids can readily be produced using the disclosed sequences provided herein, sequences available in the art, and the genetic code. In one example, nucleic acids are DNA. In one example, nucleic acids are RNA.

Degenerate variants of the disclosed nucleic acid sequences are also disclosed. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA.

Codon preferences and codon usage tables for a particular species can be used to engineer isolated nucleic acid molecules encoding protein products, such as Cas9, that take advantage of the codon usage preferences of that particular species. For example, the nucleic acid can be designed to have codons that are preferentially used by a particular organism of interest (e.g., the organism of origin for a PBMC to be modified, or an organism to be administered the nucleic acid). In some examples, the nucleic acids are codon optimized for expression in human. Thus, in some examples a Cas nuclease (or dead nuclease) sequence is codon optimized for expression in a human PBMC (PBMC, antigen presenting cell, B cell, dendritic cell, monocyte/macrophage, NK cell, T cell, CD8+ TRM T cell, tumor infiltrating lymphocyte, CAR T cell, exhausted T cell, or a cell with an antigen receptor reactive to a tumor-specific antigen).

The disclosed nucleic acids can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Nucleic acid sequences can be prepared using any suitable method, including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979: the diethylphosphoramidite method of Beaucage et al., Tetra. Lett. 22:1859-1862, 1981: the solid phase phosphoramidite triester method described by Beaucage & Caruthers, Tetra. Letts. 22(20):1859-1862, 1981, for example, using an automated synthesizer as described in, for example, Needham-VanDevanter et al., Nucl. Acids Res. 12:6159-6168, 1984; and, the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. While chemical synthesis of DNA is generally limited to sequences of about 100 bases, longer sequences may be obtained by the ligation of shorter sequences.

The disclosed nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements). The nucleic acids can also be prepared by amplification methods. Amplification methods include the polymerase chain reaction (PCR), the ligase chain reaction (LCR), the transcription-based amplification system (TAS), the self-sustained sequence replication system (3SR), and the Qβ replicase amplification system (QB). A wide variety of cloning and in vitro amplification methodologies exist.

In some aspects, the disclosed nucleic acids are included in an expression vector (e.g., viral vector, plasmid, or other vehicle) for expression in a host, or specifically in a target cell (e.g., PBMC, antigen presenting cell, B cell, dendritic cell, monocyte/macrophage, NK cell, T cell, CD8+ TRM T cell, tumor infiltrating lymphocyte, CAR T cell, exhausted T cell, terminally exhausted T cell, or a cell with an antigen receptor reactive to a tumor-specific antigen) In some examples, the expression vector includes a promoter operably linked to a disclosed nucleic acid molecule. For example, a promoter can be operably linked to an RNAi, gRNA, or Cas nuclease (or dead nuclease) to drive its expression. In some examples, a vector encodes both a Cas nuclease (or dead nuclease) and a gRNA. Additional expression control sequences, such as one or more enhancers, transcription and/or translation terminators, and initiation sequences can also be included in the expression vector. In some aspects, the disclosed nucleic acids are included in a viral vector. Exemplary viral vectors that can be used include, but are not limited to, polyoma, SV40, adenovirus, vaccinia virus, adeno-associated virus (AAV), herpes viruses including HSV and EBV, Sindbis viruses, alphaviruses and retroviruses of avian, murine, and human origin. Baculovirus (Autographa californica multinuclear polyhedrosis virus; AcMNPV) vectors can also be used. Other suitable vectors include orthopox vectors, avipox vectors, fowlpox vectors, capripox vectors, suipox vectors, lentiviral vectors, alpha virus vectors, and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio. Biologically functional viral and plasmid DNA vectors capable of expression and replication in a cell. In some examples, the vector includes a selectable marker (such as an antibiotic resistance gene (e.g., puromycin) or a reporter gene (e.g., green fluorescent protein (GFP)). In other examples, a selectable marker and/or reporter is not included in the vector.

The disclosed nucleic acids can be introduced into a host cell by DNA transfer (e.g., oligonucleotides), or introduced and expressed in a suitable host cell (e.g., expression cassette or vector). In some examples, the expressed product is an RNA (e.g., siRNA or gRNA), in other examples, the expressed product is a protein (e.g., Cas9). The cell may be prokaryotic or eukaryotic. In some aspects, the host cell is a PBMC (e.g., B cell, monocyte/macrophage, dendritic cell, T cell). Methods of transient or stable transfer can be used. Transient transfer indicates that the foreign nucleic acid is only present transiently (e.g., degraded after a period of time, cleared by the host cell, or otherwise not stably replicated). Stable transfer indicates that the foreign nucleic acids is continuously maintained in the host.

To obtain optimal expression of the disclosed nucleic acids, expression cassettes can contain, for example, a strong promoter to direct transcription, a ribosome binding site for translational initiation (e.g., internal ribosomal binding sequences), and a transcription/translation terminator can be used. For expression in E. coli, a promoter, such as the T7, trp, lac, or lamda promoters, a ribosome binding site, and preferably a transcription termination signal can be used. For eukaryotic cells, such as a PBMC, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). Additional operational elements include, but are not limited to, leader sequence, termination codons, polyadenylation signals and any other sequences necessary for the appropriate transcription and subsequent translation of the nucleic acid sequence.

The disclosed nucleic acids or vectors can be introduced into the host cell by any suitable method (e.g., transformation). Numerous methods of transformation can be used, such as: chemical methods (e.g., calcium-phosphate transfection), physical methods (e.g., electroporation, microinjection, particle bombardment), fusion (e.g., liposomes), lipofection, nucleofection, receptor-mediated endocytosis (e.g., DNA-protein complexes, viral envelope/capsid-DNA complexes), particle gun accelerator (gene gun), and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA. Successfully transformed cells can be selected by resistance to antibiotics conferred by genes contained in the vector, such as the amp, gpt, neo and hyg genes. In some examples, a disclosed nucleic acid (e.g., gRNA) is incorporated in a ribonucleoprotein (RNP) complex (e.g., a gRNA-Cas complex). RNPs can be introduced into a host cell by transformation, for example, by nucleofection.

Modifications can be made to the disclosed nucleic acids without diminishing biological activity of the encoded product. For example, modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

(a) Overexpression of Adrb1 and/or Adrb2

Adrb1 and/or Adrb2 can be overexpressed in PBMCs, such as a T cell, using appropriate coding sequences. DNA sequences encoding Adrb1 and/or Adrb2 can be expressed in PBMCs. For techniques for the propagation of mammalian cells in culture see, e.g., Helgason and Miller (Eds.), 2012, Basic Cell Culture Protocols (Methods in Molecular Biology), 4^{th} Ed., Humana Press. In some examples the host cell is derived from a subject.

Methods of transfection of DNA, such as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40), AAV, or bovine papilloma virus, to transiently infect or transform eukaryotic cells and express the protein (see for example, Viral Expression Vectors, Springer press, Muzyczka ed., 2011). Plasmids and vectors can also be used.

In some aspects, nucleic acid molecules encoding ADRB1 and/or ADRB2, a precursor thereof, variant thereof, or fragment thereof are also of use in the disclosed methods. By introducing a nucleic acid encoding ADRB1 and/or ADRB2, a precursor thereof, variant thereof, or fragment thereof, the amount ADRB1 and/or ADRB2 is increased, and thus the activity of ADRB1 and/or ADRB2 is also increased.

Nucleic acid molecules can be prepared by amplification methods. Amplification methods include polymerase chain reaction (PCR), the ligase chain reaction (LCR), the transcription-based amplification system (TAS), the self-sustained sequence replication system (3SR).

The polynucleotides encoding ADRB1 and/or ADRB2 (or a precursor, variant, or fragment thereof), can include a recombinant DNA which is incorporated into a vector (such as an expression vector) into an autonomously replicating plasmid or virus or into the genomic DNA of a PBMC, or which exists as a separate molecule (such as a cDNA) independent of other sequences. The nucleotides can be ribonucleotides, deoxyribonucleotides, or modified forms of either nucleotide. The term includes single and double forms of DNA. Polynucleotides encoding ADRB1 and/or ADRB2 (or a precursor, variant, or fragment thereof), are of use in the disclosed methods include DNA and cDNA sequences themselves, and an RNA sequences that encodes ADRB1 and/or ADRB2 (or a precursor, variant, or fragment thereof). Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC: aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W.H. 5 Freeman and Co., NY). Degenerate variants are also of use in the methods disclosed herein.

Additional nucleic acid molecules encoding ADRB1 and/or ADRB2 (or a precursor, variant, or fragment thereof), can readily be produced using the amino acid sequences provided herein and the genetic code. Nucleic acid sequences encoding ADRB1 and/or ADRB2 (or a precursor, variant, or fragment thereof), can be prepared by any suitable method including, for example, cloning of appropriate sequences or by direct chemical synthesis by methods such as the phosphotriester method of Narang et al., Meth. Enzymol. 68:90-99, 1979: the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22(20):1859-1862, 1981, for example, using an automated synthesizer as described in, for example, Needhan-VanDevanter et al., Nucl. Acids Res. 12:6159-6168, 1984 and the solid support method of U.S. Pat. No. 4,458,066. Chemical synthesis produces a single-strand (ss) oligonucleotide, which can be converted into double-strand (ds) DNA by hybridization with a complementary sequence or by polymerization with a DNA polymerase using the single strand as a template. Exemplary nucleic acids that include sequences encoding ADRB1 and/or ADRB2 (or a precursor, variant, or fragment thereof) can be prepared by cloning.

A nucleic acid molecule encoding ADRB1 and/or ADRB2 (or a precursor, variant, or fragment thereof) can be cloned or amplified by in vitro methods, such as the polymerase chain reaction (PCR), the ligase chain reaction (LCR), the transcription-based amplification system (TAS), the self-sustained sequence replication system (3SR), and the Qβ replicase amplification system (QB). For example, a polynucleotide encoding ADRB1 and/or ADRB2 (or a precursor, variant, or fragment thereof) can be isolated by a polymerase chain reaction of cDNA using primers based on the DNA sequence of the molecule. A wide variety of cloning and in vitro amplification methodologies can be used. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

Typically, a polynucleotide sequence encoding ADRB1 and/or ADRB2 (or a precursor, variant, or fragment thereof) is operably linked to transcriptional control sequences including, for example a promoter and a polyadenylation signal. Any promoter can be used that is a polynucleotide sequence recognized by the transcriptional machinery of the host cell (or introduced synthetic machinery) that is involved in the initiation of transcription. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma virus, hepatitis B virus, and human and simian immunodeficiency viruses. Other promoters include promoters isolated from mammalian genes, such as the immunoglobulin heavy chain, immunoglobulin light chain, T cell receptor, HLA DQ α and DQ β, β-interferon, interleukin-2, interleukin-2 receptor, MHC class II, HLA-DRα, β-actin, muscle creatine kinase, prealbumin (transthyretin), elastase I, metallothioncin, collagenase, albumin, fetoprotein, β-globin, c-fos, c-HA-ras, neural cell adhesion molecule (NCAM), α1-antitrypsin, H2B (TH2B) histone, type I collagen, glucose-regulated proteins (GRP94 and GRP78), rat growth hormone, human serum amyloid A (SAA), troponin I (TNI), platelet-derived growth factor, and dystrophin.

The promoter can be inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, α-2-macroglobulin, MHC class I gene h-2 kb, HSP70, proliferin, tetracycline inducible, tumor necrosis factor, or thyroid stimulating hormone gene promoter. One example of an inducible promoter is the interferon inducible ISG54 promoter. In some examples, the promoter is a constitutive promoter that results in high levels of transcription upon introduction into a host cell in the absence of additional factors. In more examples, the constitutive promoter is a human β-actin, human elongation factor-1α, chicken β-actin combined with cytomegalovirus early enhancer, cytomegalovirus (CMV), simian virus 40, or a herpes simplex virus thymidine kinase promoter (see Damdindorj et al., PLOS One 9(8): e106472, 2014). The promoter can be a human β-actin promoter, human elongation factor-1α promoter, β-actin promoter, simian virus 40 promoter, or a herpes simplex virus thymidine kinase promoter.

Optionally, transcription control sequences include one or more enhancer elements, which are binding recognition sites for one or more transcription factors that increase transcription above that observed for the minimal promoter alone, and also be operably linked to the polynucleotide encoding the nucleic acid molecule encoding ADRB1 and/or ADRB2 (or a precursor, variant, or fragment thereof). Introns can also be included that help stabilize mRNA and increase expression.

A polyadenylation signal can be included to effect proper termination and polyadenylation of the transcript. Exemplary polyadenylation signals have been isolated from beta globin, bovine growth hormone, SV40, and the herpes simplex virus thymidine kinase genes.

A nucleic acid molecule encoding ADRB1 and/or ADRB2 (or a precursor, variant, or fragment thereof) can be included in a viral vector, for example for expression of the protomer to produce the corresponding protein, variant or fragment thereof, or for administration to a subject as disclosed herein. Typically, such viral vectors include a nucleic acid molecule encoding ADRB1 and/or ADRB2 (or a precursor, variant, or fragment thereof). In some examples, the viral vector encoding ADRB1 and/or ADRB2 (or a precursor, variant, or fragment thereof) can be replication-competent. For example, the viral vector can have a mutation (e.g., insertion of nucleic acid encoding the protomer) in the viral genome that attenuates, but does not completely block viral replication in host cells.

Various viral vectors which can be utilized for nucleic acid based therapy as taught herein include adenovirus or adeno-associated virus (AAV), herpes virus, vaccinia, or an RNA virus such as a retrovirus (including HVJ, see Kotani et al., Curr. Gene Ther. 4:183-194, 2004). In one example, the retroviral vector is a derivative of a murine or avian retrovirus, or a human or primate lentivirus. Examples of retroviral vectors in which a foreign gene can be inserted include, but are not limited to: Moloney murine leukemia virus (MoMLV), Harvey murine sarcoma virus (HaMuSV), murine mammary tumor virus (MuMTV), and Rous Sarcoma Virus (RSV). In one example, when the subject is a human, a vector such as the gibbon ape leukemia virus (GaLV) can be utilized. A pseudotyped retroviral vector can be utilized that includes a heterologous envelope gene. In one example the viral vector is AAV.

Some retroviral vectors can incorporate multiple genes. These vectors can transfer or incorporate a gene for a selectable marker so that transduced cells can be identified and generated. By inserting a nucleic acid encoding ADRB1 and/or ADRB2 (or a precursor, variant, or fragment thereof) into the viral vector, along with another gene which can serve as viral envelope protein and also can encode the ligand for a receptor on a specific target cell, for example, the vector is now target specific. Retroviral vectors can be made target specific by modifications of the envelope protein by attaching, for example, a sugar, a glycolipid, or a protein. In one specific, non-limiting example, targeting is accomplished by using an antibody to target the retroviral vector.

Since recombinant retroviruses are non-replicating by design, they require assistance i to produce infectious vector particles. This assistance can be provided, for example, by using helper cell lines that contain plasmids encoding all of the structural genes of the retrovirus under the control of regulatory sequences within the long terminal repeat (LTR). These plasmids are missing a nucleotide sequence which enables the packaging mechanism to recognize an RNA transcript for encapsidation. Helper cell lines which have deletions of the packaging signal include, but are not limited to ψ2, PA317, and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Alternatively, NIH 3T3 or other tissue culture cells can be directly transfected with plasmids encoding the retroviral structural genes gag, pol and env, by conventional transfection methods. These cells are then transfected with the vector plasmid containing the genes of interest. The resulting cells release the retroviral vector into the culture medium.

The adenovirus vectors include replication competent, replication deficient, gutless forms thereof. Defective viruses, such as adenovirus vectors or adeno-associated virus (AAV) vectors, that entirely or almost entirely lack viral genes, can be used. Use of defective viral vectors allows for administration to specific cells without concern that the vector can infect other cells. The AAV vectors of use are replication deficient. In some non-limiting examples, a vector of use is an attenuated adenovirus vector, such as the vector described by Stratford-Perricaudet et al. (J. Clin. Invest., 90:626-630 1992: La Salle et al., Science 259:988-990, 1993); or a defective AAV vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Recombinant AAV vectors are capable of directing the expression and the production of the selected transgenic products in targeted cells. Thus, the recombinant vectors can include at least all of the sequences of AAV essential for encapsidation and the physical structures for infection of target cells.

In some examples, the AAV DNA includes a nucleic acid including a promoter operably linked to a nucleic acid molecule encoding ANG, a precursor, variant, or fragment thereof, or encoding a tRNA fragment. Further provided are recombinant vectors, such as recombinant adenovirus vectors and recombinant adeno-associated virus (rAAV) vectors comprising a nucleic acid molecule(s) disclosed herein. In some examples, the AAV is rAAV8, and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes. An exemplary AAV8 vector is disclosed, for example, in PCT Publication No. WO 2014/127196.

Gene therapy vectors that utilize AAV can infect both dividing and quiescent cells and persist in an extrachromosomal state without integrating into the genome of the host cell. The present disclosure contemplates the use of an rAAV for the methods disclosed herein. AAV can be used to transfect cells, see for example, U.S. Published Patent Application No. 2014/0037585, incorporated herein by reference. Methods for producing rAAV suitable for gene therapy are known (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

In some examples, the vector is a rAAV8 vector, a rAAV2 vector, a rAAV9 vector. In a specific non-limiting example, the vector is an AAV8 vector. AAV8 vectors are disclosed, for example, in U.S. Pat. No. 8,692,332, which is incorporated by reference herein. The location and sequence of the capsid, rep 68/78, rep 40/52, VP1, VP2 and VP3 are disclosed in this U.S. Pat. No. 8,692,332. The location and hypervariable regions of AAV8 are also provided. In some examples, the vector is an AAV2 variant vector, such as AAV7m8.

The vectors of use in the methods disclosed herein can contain nucleic acid sequences encoding an intact AAV capsid which may be from a single AAV serotype (e.g., AAV2, AAV6. AAV8 or AAV9). As disclosed in U.S. Pat. No. 8,692,332, vectors of use can also be recombinant, and thus can contain sequences encoding artificial capsids which contain one or more fragments of the AAV8 capsid fused to heterologous AAV or non-AAV capsid proteins (or fragments thereof). These artificial capsid proteins are selected from non-contiguous portions of the AAV2, AAV6, AAV8 or AAV9 capsid or from capsids of other AAV serotypes. For example, a AAV vector may have a capsid protein comprising one or more of the AAV8 capsid regions selected from the VP2 and/or VP3, or from VP1, or fragments thereof selected from amino acids 1 to 184, amino acids 199 to 259: amino acids 274 to 446; amino acids 603 to 659; amino acids 670 to 706; amino acids 724 to 738 of the AAV8 capsid, which is presented as SEQ ID NO: 2 in U.S. Pat. No. 8,692,332. In another example, it may be desirable to alter the start codon of the VP3 protein to GTG. Alternatively, the AAV may contain one or more of the AAV serotype 8 capsid protein hypervariable regions, for example aa 185-198; an 260-273; aa447-477; aa495-602; aa660-669; and aa707-723 of the AAV8 capsid which is presented as SEQ ID NO: 2 in U.S. Pat. No. 8,692,332.

Additional viral vectors that can be used for expression of ADRB1 and/or ADRB2 (or a precursor, variant, or fragment thereof), include polyoma, i.e., SV40 (Madzak et al., 1992, J. Gen. Virol., 73:15331536), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors.

Another targeted delivery system that can be used for a polynucleotide encoding ADRB1 and/or ADRB2 (or a precursor, variant, or fragment thereof) is a colloidal dispersion system. Colloidal dispersion systems include macromolecule complexes, nanocapsules, microspheres, beads, and lipid based systems including oil-in-water emulsions, micelles, mixed micelles, and liposomes. One colloidal dispersion system is a liposome. Liposomes are artificial membrane vesicles which are useful as delivery vehicles in vitro and in vivo. Large unilamellar vesicles (LUV), which range in size from about 0.2 4 microns, can encapsulate a substantial percentage of an aqueous buffer containing large macromolecules. RNA, DNA and intact virions can be encapsulated within the aqueous interior and be delivered to cells in a biologically active form (Fraley et al., Trends Biochem. Sci. 6:77, 1981).

The composition of the liposome is usually a combination of phospholipids, particularly high-phase-transition-temperature phospholipids, usually in combination with steroids, especially cholesterol. Other phospholipids or other lipids may also be used.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. One example are diacylphosphatidyl-glycerols, where the lipid moiety contains 14-18 carbon atoms, such as 16-18 carbon atoms, and is saturated. Illustrative phospholipids include, for example, phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

Biodegradable and biocompatible polymer scaffolds can also be used (see Jang et al., Expert Rev. Medical Devices 1:127-138, 2004) for use in the bone. These scaffolds usually contain a mixtures of one or more biodegradable polymers, for example and without limitation, saturated aliphatic polyesters, such as poly(lactic acid) (PLA), poly(glycolic acid), or poly(lactic-co-glycolide) (PLGA) copolymers, unsaturated linear polyesters, such as polypropylene fumarate (PPF), or microorganism produced aliphatic polyesters, such as polyhydroxyalkanoates (PHA), (see Rezwan et al., Biomaterials 27:3413-3431, 2006; Laurencin et al., Clin. Orthopaed. Rel. Res. 447:221-236). By varying the proportion of the various components, polymeric scaffolds of different mechanical properties are obtained. An exemplary scaffold contains a ratio of PLA to PGA is 75:25, but this ratio may change depending upon the specific application. Other exemplary scaffolds include surface bioeroding polymers, such as poly(anhydrides), such as trimellitylimidoglycine (TMA-gly) or pyromellitylimidoalanine (PMA-ala), or poly(phosphazenes), such as high molecular weight poly(organophasphazenes) (P[PHOS]), and bioactive ceramics. The gradual biodegradation of these scaffolds allows the gradual release of drugs or gene from the scaffold. Thus, these polymeric carriers represent not only a scaffold but also a drug or gene delivery system. This system is applicable to the delivery of plasmid DNA and also applicable to viral vectors, such as AAV or retroviral vectors, as well as transposon-based vectors.

The disclosed nucleic acid molecules can be included in a nanodispersion system, see, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006.

Dendrimers can also be used. Dendrimers include an initiator core, surrounded by a layer of a selected polymer that is grafted to the core, forming a branched macromolecular complex. Dendrimers are typically produced using polymers such as poly(amidoamine) or poly(L-lysine). A dendrimer can be synthesized from the repeated addition of building blocks to a multifunctional core (divergent approach to synthesis), or towards a multifunctional core (convergent approach to synthesis) and each addition of a three-dimensional shell of building blocks leads to the formation of a higher generation of the dendrimers. Polypropylenimine dendrimers contain 100% protonable nitrogens and up to 64 terminal amino groups. Protonable groups are usually amine groups which are able to accept protons at neutral pH. For nucleic acid molecules, dendrimers can be formed from polyamidoamine and phosphorous containing compounds with a mixture of amine/amide or N—P(O2)S as the conjugating units. Dendrimers of use for delivery of nucleic acid molecules is disclosed, for example, in PCT Publication No. 2003/033027, incorporated herein by reference.

The surface of the targeted delivery system may be modified in a variety of ways. In the case of a liposomal targeted delivery system, lipid groups can be incorporated into the lipid bilayer of the liposome to maintain the targeting ligand in stable association with the liposomal bilayer. Various linking groups can be used for joining the lipid chains to the targeting ligand.

In another example, an mRNA can be used to deliver a nucleic acid encoding ADRB1 and/or ADRB2 (or a precursor, variant, or fragment thereof), directly into cells. In some examples, nucleic acid-based vaccines based on mRNA may provide a potent alternative to the previously mentioned approaches. mRNA delivery precludes safety concerns about DNA integration into the host genome and can be directly translated in the host cell cytoplasm. Two exemplary forms of RNA that can be used to deliver a nucleic acid include conventional non-amplifying mRNA (see, e.g., Petsch et al., “Protective efficacy of in vitro synthesized, specific mRNA vaccines against influenza A virus infection,” Nature biotechnology, 30(12):1210-6, 2012) and self-amplifying mRNA (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015).

(b) Insertion of Adrb1 and/or Adrb2 Via CRISPR/Cas9

Included are in the present disclosure are methods for site-specific modification of a nucleic acid molecule in a cell to introduce ADRB1 and/or ADRB2 (or a precursor, variant, or fragment thereof) or another molecule that increases ADRB1 and/or ADRB2 activity. These modifications can include, but are not limited to, site-specific insertions, and replacements of nucleotides (a “knock-in”), such that an increase in ADRB1 and/or ADRB2 activity is produced. These modifications can be made anywhere within the genome, for example, in genomic elements, including, among others, coding sequences, regulatory elements, and non-coding DNA sequences. However, in some examples, an insertion is made into a safe harbor locus (see Pavani and Amendola, Front Genome Ed., https://doi.org/10.3389/fgeed.2020.609650, 20 January 2021).

Any number of such insertions can be made, in any order or combination. Such methods may be used to modify expression of a gene, such as to increase expression of ADRB1 and/or ADRB2. These modifications include a “knock-in” of a nucleic acid molecule encoding ADRB1 and/or ADRB2. Techniques for making such modifications by genome editing include, for example, use of CRISPR-Cas systems, zinc-finger nucleases (ZFNs), and transcription activator-like effector nucleases (TALENs), among others. These modifications can be used to introduce (knock-in) a nucleic acid molecule encoding ADRB1 and/or ADRB2 (or a precursor, variant, or fragment thereof) into a safe harbor locus in a genome. Cas9 mediated integration of genes is disclosed, for example, in Li et al., G3 10(2):467-473. doi: 10.1534/g3.119.400810 (2020) and Jacinto et al., J Cell Mol Med. 24:3766-3778, DOI: 10.1111/jcmm.14916 (2020).

A typical set of CRISPR system is composed of two components, a CRISPR-associated nuclease 9 (Cas9) and one or more guide RNAs (gRNAs), each of which contains a CRISPR RNA (crRNA) and a trans-activating CRISPR RNA (tracrRNA). Simple gene disruptions can be generated by cleavage of the target site, followed by alteration of nucleic acids, such as a deletion, and repair by the non-homologous-end-joining pathway (NHEJ). Target recognition by crRNAs occurs through complementary base pairing with target DNA, which directs cleavage of foreign sequences by means of Cas proteins. In some examples, DNA recognition by guide RNA and consequent cleavage by the endonuclease requires complementary base-pairing with a protospacer adjacent motif (PAM) (e.g., 5′-NGG-3′) and with a protospacer region in the target. (Jinek et. al., Science. 337:816-821, 2012). The PAM motif recognized by a Cas9 varies for different Cas9 proteins. Any Cas9 protein can be used in the systems and methods disclosed herein, including mutant Cas9 proteins (such as those having an R691A, D10A, H840A, or combination of such substitutions). In other examples of the systems and methods disclosed herein, a promoter, is operably linked to the nucleic acid encoding Cas9.

As noted above, the Cas9 RNA guide system includes a mature crRNA that is base-paired to trans-activating crRNA (tracrRNA), forming a two-RNA structure that directs Cas9 to the locus of a desired double-stranded (ds) break in target DNA. In some examples base-paired tracrRNA:crRNA combination is engineered as a single RNA chimera to produce a guide sequence (e.g., gRNA) which preserves the ability to direct sequence-specific Cas9 dsDNA cleavage. In some examples, the Cas9-guide sequence complex results in cleavage of one or both strands at a target sequence within a safe harbor locus, which allows a knock in. Thus, the Cas9 endonuclease and the gRNA molecules are used sequence-specific target recognition, cleavage, and genome editing of the safe harbor locus. In one example, the cleavage site is at a specific nucleotide, such as, but not limited to the 16, 17, or 18th nucleotide (nt) of a 20-nt target. In one non-limiting example, the cleavage site is at the 17th nucleotide of a 20-nt target sequence. The cleavage can be a double stranded cleavage.

In some examples, the gRNA molecule is selected so that the target genomic targets bear a protospacer adjacent motif (PAM). In some examples, DNA recognition by guide RNA and consequent cleavage by the endonuclease requires the presence of a protospacer adjacent motif (PAM) (e.g., 5′-NGG-3′) in immediately after the target. The PAM is present in the targeted nucleic acid sequence but not in the crRNA that is produced to target it. In some examples, the proto-spacer adjacent motif (PAM) corresponds to 2 to 5 nucleotides starting immediately or in the vicinity of the proto-spacer at the leader distal end. The PAM motif also can be NNAGAA, NAG, NGGNG, AWG, CC, CC, CCN, TCN, or TTC.

In some examples, cleavage occurs at a site about 3 base-pairs upstream from the PAM. In some examples, the Cas9 nuclease cleaves a double stranded nucleic acid sequence.

In some examples, the guide sequence is selected to reduce the degree of secondary structure within the sequence. Secondary structure may be determined by any suitable polynucleotide folding algorithm. Some programs are based on calculating the minimal Gibbs free energy. An example of one such algorithm is mFold (Zuker and Stiegler, Nucleic Acids Res. 9 (1981), 133-148). Another example folding algorithm is the online webserver RNAfold, which uses the centroid structure prediction algorithm (see e.g., Gruber et al., 2008, Cell 106(1): 23-24; and Can and Church, 2009, Nature Biotechnology 27(12): 1151-62). Guide sequences can be designed using the MIT CRISPR design tool found at crispr.mit.edu, Harvard and University of Bergen CHOPCHOP web tool found at chopchop.cbu.uib.no, or the E-CRISP tool found at e-crisp.org/E-CRISP. Additional tools for designing tracrRNA and guide sequences are described in Naito et al., Bioinformatics. 2014 Nov. 20, and Ma et al. BioMed Research International, Volume 2013 (2013), Article ID 270805. The crRNA can be 18-48 nucleotides in length. The crRNA can be 18, 19, 20, 21, 22, 23, 24 or 25 nucleotides in length. In one example, the crRNA is 20 nucleotides in length.

The system can introduce double stranded DNA breaks, such that the target, e.g., the safe harbor locus, is cleaved by Cas9. This allows insertion of a nucleic acid sequence encoding ADRB1 and/or ADRB2 (or a precursor, variant, or fragment thereof). In some examples, more than one DNA break can be introduced by using more than one gRNA. For example, two gRNAs can be utilized, such that two breaks are achieved. When two or more gRNAs are used to position two or more cleavage events, in a target nucleic acid, it is contemplated that in an example the two or more cleavage events may be made by the same or different Cas9 proteins. For example, when two gRNAs are used to position two double strand breaks, a single Cas9 nuclease may be used to create both double strand breaks.

In some examples, the disclosed methods include the use of one or more vectors comprising: a) a promoter operably linked to a nucleotide sequence encoding a Type II Cas9 nuclease, b) a promoter, such as a U6 promoter, operably linked to one or more nucleotide sequences encoding one or more CRISPR-Cas guide RNAs that hybridize with a safe harbor locus in a target cell, such as a human PBMC; and c) a nucleic acid molecule encoding ADRB1 and/or ADRB2 (or a precursor, variant, or fragment thereof). These components can be located on same or different vectors, whereby the one or more guide RNAs target the noncoding region, such that the Cas9 protein cleaves the DNA and the nucleic acid molecule encoding ADRB1 and/or ADRB2 (or a precursor, variant, or fragment thereof) is introduced. In specific non-limiting examples, the one or more vectors are viral vectors such as lentiviral vectors. In other non-limiting examples, the viral vectors are adenovirus vectors, AAV vectors, or retroviral vectors. Without limitation, Cas9 and gRNAs can be delivered to the using AAV, a lentivirus, piggybac, an episomal constructs, or injected as purified nanoparticles constituted by pure Cas9 protein and pure guides RNAs (see, for example, Steyer et al., Drug Discov Today Technol 28: 3-12, 2018).

### V. Modified PBMCs

Modified PBMCs With Reduced Expression of Adrb1 and/or Adrb2

Provided herein are modified PBMCs with reduced expression of Adrb1 and/or Adrb2, reduced activity of ADRB1 and/or ADRB2, or any combination thereof. In some examples, modified PBMCs have reduced expression of Adrb1 and/or reduced activity of ADRB1. In some examples, modified PBMCs have reduced expression of Adrb2 (and/or reduced activity of ADRB2). In some examples, modified PBMCs have reduced expression of Adrb1 (and/or reduced activity of ADRB1) and reduced expression of Adrb2 (and/or reduced activity of ADRB2).

In some examples, expression of Adrb1 and/or Adrb2 is reduced by at least 10%, at least 20%, at least 30%, at least 40%, at least 50%, at least 60%, at least 70%, at least 80%, at least 90%, at least 95%, at least 98%, or at least 100% relative to a suitable control (e.g., a PBMC prior to modification). In some examples, activity of ADRB1 and/or ADRB2 is reduced by at least 10%, at least 20%, at least 30%, at least 40%, at least 50%, at least 60%, at least 70%, at least 80%, at least 90%, at least 95%, at least 98%, or 100% relative to a suitable control (e.g., a PBMC prior to modification). Reducing activity includes reducing any measurable biological function of ADRB1 and/or ADRB2, for example, reduced signal transduction when exposed to their respective ligands. In some examples, the modified PBMC with reduced expression of Adrb1 and/or Adrb2, reduced activity of ADRB1 and/or ADRB2, or any combination thereof, has increased effector activity (e.g., anti-tumor) relative to a suitable control (e.g., unmodified PBMC). In some examples, the modified PBMC is a T cell, and the T cell has increased resistance to T cell exhaustion relative to a suitable control (e.g., unmodified PBMC).

The modified PBMC can further include additional modifications, for example, the PBMC can express or otherwise contain a chimeric antigen receptor (CAR) or engineered T cell receptor (TCR).

In some examples, the modified PBMC is a T cell, for example, a CD4+, a CD8+ or a CD3+ T cell. The T cell can be reactive to a tumor-specific antigen, for example, CD19, CD20, BCMA, MUC1, PSA. CEA, HER1, HER2, TRP-2, EpCAM, GPC3, mesothelin 1 (MSLN), or EGFR. In some examples, the T cell is a tumor-infiltrating lymphocyte (TIL). In some examples, the T cell is a therapeutic T cell, or will be used as a therapeutic T cell, for example, as an ACT therapy. In some examples, the T cell is an exhausted T cell (including terminally exhausted T cells). “Exhausted T cells” are dysfunctional (hyporesponsive) T cells commonly found in cancer environments. T cell exhaustion can be characterized by a progressive loss of effector function (for example, loss of IL-2, TNF-α, and IFN-γ production) and sustained expression of inhibitory receptors such as PD-1, TIM-3, CTLA-4, LAG-3, CD101, TIGIT, and CD160. In some examples, the exhausted T cell is a CD3+ T cell, CD4+ T cell or CD8+ T cell. In some examples, the exhausted T cell is a terminally exhausted T cell (a terminally differentiated T cell that is exhausted). In some examples the exhausted cell is an “exhausted progenitor T cell” (TEX_{Prog}). TEX_{Prog} may have high or persistent expression of any or all of TOX, PD-1, SLAMF6, ID3, CD127. CXCR5 and TCF1 relative to other T cells (PD-1+SLAMPF6+ and/or TCF1+ T cells), in some examples the exhausted cell is an “exhausted effector-like T cell” (TEX_{Eff}). TEX_{Eff} may have high or persistent expression of any or all of PD-1 CX3CR1, TBX21 and TIM3 relative to other T cells (PD-1+ CX3CR1+TBX2I+ and/or TIM3+). “Terminally Exhausted T cells” (TEX_{Term}) may have high or persistent expression of any or all of TIM3, PD-1, CXCR6, CD39, LAYN. EOMES and CD101 relative to other T cells (TIM3+ PD-1+ CD101+ T cells). Expression of the above proteins can be determined by FACs analysis, for example, by FACs analysis of a population of T cells. In some examples the T cell is a tissue resident memory T cell (TRM).

In some aspects, the modified PBMC includes an agent that reduces Adrb1 and/or Adrb2 expression, for example, one or more of the disclosed inhibitory RNA (RNAi) specific for gene or transcript, or one or more guide RNA (gRNAs) specific for Adrb1 and/or Adrb2 gene or transcript (for example in combination with a Cas nuclease or dead Cas nuclease, such as an RNP).

In some examples, the agent that reduces Adrb1 and/or Adrb2 expression is one or more of the disclosed inhibitory RNA (RNAi), for example, a short hairpin RNA (shRNA), short interfering RNA (siRNA), microRNA (miRNA), or an antisense RNA specific to Adrb1 and/or Adrb2. In specific, non-limiting examples, the RNAi is a shRNA specific for Adrb1 and/or Adrb2 gene or transcript, for example, the siRNA is specific to a sequence comprising at least 70%, at least 80%, at least 90%, at least 95%, at least 98%, at least 99%, or 100% sequence identity to SEQ ID NOs: 2, 3, 5, and/or 6. In some examples, the shRNA is specific to a sequence with at least 90% sequence identity to a unique, contiguous portion of SEQ ID NOs: 2, 3, 5, and/or 6.

In some examples, the agent is a disclosed gRNA specific for Adrb1 and/or Adrb2 gene or transcript, for example, the gRNA is specific for a sequence with at 70%, at least 80%, at least 90%, at least 95%, at least 98%, at least 99%, or 100% sequence identity to SEQ ID NOs: 2, 3, 5, and/or 6. For example, the gRNA can be specific to a sequence with at least 90% sequence identity to SEQ ID NOs: 2, 3, 5, and/or 6. In some examples, the gRNA comprises a targeting sequence specific to Adrb1 and/or Adrb gene or transcript, for example, by having a targeting sequence that is at least 70%, at least 80%, at least 90%, at least 95%, at least 98%, at least 99%, or 100% complementary to a unique, contiguous portion of SEQ ID NOs: 2, 3, 5, and/or 6.

In some examples, the gRNA specific for Adrb1 and/or Adrb2 gene or transcript includes a contiguous sequence at least 70%, at least 80%, at least 90%, at least 95%, at least 98%, at least 99%, or 100% sequence identity to SEQ ID NOs: 7, 8, 9, 10, 11, 12, 13, 14, 15, 16, 17, 18, 19, 20, 21, 22, 23, 24, 25, 26, and/or 28. In some examples, the gRNA specific for Adrb1 and/or Adrb2 gene or transcript includes SEQ ID NOs: 7, 8, 9, 10, 11, 12, 13, 14, 15, 16, 17, 18, 19, 20, 21, 22, 23, 24, 25, 26, and/or 28. In some examples, the modified PBMC includes a RNP complex that includes the disclosed gRNA and a Cas nuclease, such as Cas3, dCas3, Cas9, dCas9, Cas12, dCas12, Cas13a, dCas13a, Cas13b, dCas13b, Cas13d, or dCas13d.

In some examples, the modified PBMC includes a heterologous nucleic acid molecule encoding one or more of the disclosed nucleic acids encoding the RNAi (e.g., shRNA, siRNA, antisense RNA) or gRNA. RNAi or gRNA may be encoded as DNA (for example, encoded on a DNA vector), but expressed as RNA. In some examples, the heterologous nucleic acid molecule encodes the disclosed gRNA specific for Adrb1 and/or Adrb2 and a Cas nuclease (or a dead Cas nuclease). In specific examples, the Cas nuclease is a Cas9 nuclease. In other examples, the Cas nuclease is a Cas13d nuclease, or a Cas12 nuclease.

In some examples, the modified PBMC includes the disclosed vector encoding the RNAi or gRNA. Thus, in some examples, the modified PBMC expresses the RNAi or gRNA. If gRNA is used, a Cas nuclease (e.g., Cas3, dCas3, Cas9, dCas9, Cas12, dCas12, Cas13a, dCas13a, Cas13b, dCas13b, Cas13d, or dCas13d) can also be encoded on the same or different vector, for example, to co-express a Cas nuclease (or dead Cas nuclease) and one or more gRNA specific for Adrb1 and/or Adrb2 in the modified PBMC. In some examples, the gRNA includes a spacer sequence and DR sequence (such as DR-spacer-DR-spacer) and the Cas nuclease is Cas13d, and Adrb1 and/or Adrb2 RNA is edited. In some examples, the gRNA includes a crRNA and tracrRNA (expressed either as two separate molecules, or as one fusion molecule, such as a sgRNA) and the Cas nuclease is Cas9. In some examples, the vector includes a cassette including two or more gRNA specific for Adrb1 and/or Adrb2, wherein the two or more gRNAs have the same or different targeting sequences (e.g., may target two different regions of Adrb1 and/or Adrb2). In some examples the vector includes a single gRNA specific for both Adrb1 and Adrb2. In some examples the vector includes a gRNA specific for Adrb1. In some examples the vector includes a gRNA specific for Adrb2. In some examples the vector includes multiple gRNA specific for any combination of Adrb1 and/or Adrb2 (e.g., a vector with a first gRNA specific for Adrb1 and a second gRNA specific for Adrb2).

Nucleic acids or vectors can be transiently or stably introduced into a PBMC (e.g., T cell). In a specific, non-limiting example, the vector is stably introduced into the modified PBMC, thereby resulting in stable expression of the RNAi or gRNA in the modified PBMC. In some examples, the nucleic acid encoding the RNAi or gRNA is operably linked to a cell specific promoter (e.g., a T cell specific promoter such as Lek, Cd4 (silencer minus) T cell receptor (Tcra)) in the vector. Expression of the RNAi or gRNA can be constitutive or inducible. Exemplary promoters include NFAT, EF1a, PGK, U6, or H1. In one example, gRNA is expressed from a U6 or H1 promoter. In one example, a Cas nuclease (or dead Cas nuclease) is expressed from a CMV promoter.

In some aspects, the modified PBMC includes a non-naturally occurring genetic modification that reduces an amount of functional ADRB1 and/or ADRB2. Reducing functional ADRB1 and/or ADRB2 includes genetic modifications that decrease Adrb1 and/or Adrb2 expression (e.g., decreasing transcription or translation of Adrb1 and/or Adrb2 gene or transcript) in the modified PBMC. In some examples, the genetic modification is a non-naturally occurring genetic modification of a Adrb1 and/or Adrb2 gene. In other examples, the genetic modification is a non-naturally occurring genetic modification of a regulatory element of Adrb1 and/or Adrb2 (e.g., a promoter, response element, enhancer, transcription factor, or other regulator that affects expression of Adrb1 and/or Adrb2). The regulatory element can be cis-acting or trans-acting.

In some examples, the non-naturally occurring genetic modification is a modification that reduces an amount of functional ADRB1 and/or ADRB2 in the modified PBMC. For example, the genetic modification can result in the production of dysfunctional ADRB1 and/or ADRB2. In some examples, the genetic modification results in the production of unstable ADRB1 and/or ADRB2, such that the accumulation of functional ADRB1 and/or ADRB2 is reduced. In some examples the genetic modification can prevent signal transduction via ADRB1 and/or ADRB2. In some examples the genetic modification can prevent ADRB1 and/or ADRB2 from reaching the cell surface. The genetic modification can be any non-naturally occurring modification that results in a decreased amount of ADRB1 and/or ADRB2. Non-limiting examples of genetic modifications include a point mutation, partial deletion, full deletion, or insertion.

Methods of Generating Modified PBMCs with Reduced Adrb1 and/or Adrb2

Also provided herein are methods of generating the disclosed modified PBMCs by introducing the non-naturally occurring genetic modification into a PBMC, thereby generating the modified PBMC with reduced expression of Adrb1 and/or Adrb2 and/or reduced activity of ADRB1 and/or ADRB2. In some aspects, the PBMC is obtained from a subject before introducing the non-naturally occurring genetic modification. PBMCs can be harvested or isolated, for example, from a blood sample, such as a venous blood sample, from the subject. Several techniques for isolating PBMCs can be used, for example, density centrifugation (the Ficoll approach), isolation by cell preparation tubes (CPTs), or isolation by SepMate™ tubes. In some examples, aphersis or leukapheresis is used to harvest PBMCs. Erythrocyte contamination can be evaluated, for example, by microscopic analysis of the sample. Flow cytometry techniques (e.g., FACS) can be used to assess the composition of the isolated PBMC populations, for example, to identify monocytes (e.g., CD14), T cells (e.g., CD3, CD8, CD4), B cells (e.g., CD20), or NK cells (e.g., CD56). FACS techniques can also be used to enrich or deplete a particular cell type from a PBMC (for example, enrich or deplete CD14, CD3, CD8, CD4, CD28, CD20, CD56, or combinations thereof).

In some aspects, the PBMC is harvested or isolated from a solid tissue sample, for example from a tumor. Tumor samples can be surgically resected, enzymatically digested, and PBMCs can subsequently be isolated, for example via the methods of Donia et al., Characterization and Comparison of ‘Standard’ and ‘Young’ Tumour-Infiltrating Lymphocytes for Adoptive Cell Therapy at a Danish Translational Research Institution, Scand. J. Immuno. (2011) 75:157-67, incorporated by reference herein.

In some examples, T cells are isolated from a PBMC sample, or the PBMC sample is enriched for T cells, for example, isolated or enriched for CD3⁺ or CD8⁺ T cells. In some examples, a sample is enriched by negative selection, for example, by selecting and removing unwanted cell types from a sample (e.g., cell types other than T cells, and/or naïve or memory T cells). In some examples, FACS (Fluorescence-activated cell sorting) is used to enrich for a particular PBMC, for example, to enrich for T cells (e.g., CD3, CD4 or CD8 positive T cells). FACS/Flow cytometry can also be used to assess whether exhausted T cells (for example cells expressing PD-1. TOX, SLAMF6, TCF1, CX3CR1, TIM-3, LAG-3, CD39, CD38, TIGIT. TBET, EOMES, CXCR6, and/or CD101) are present in a PBMC sample. FACS/Flow cytometry can be used to assess whether TEX_{Prog}, TEX_{Eff} and/or TEX_{Term} are present in a sample. FACS can be used to sort a PBMC sample to enrich for exhausted T cells (including one of more of TEX_{Prog}, TEX_{Eff} and/or TEX_{Term}), or conversely remove exhausted T cells (including one or more of TEX_{Prog}, a TEX_{Eff} and/or TEX_{Term}). Antigen responsiveness of the PBMCs can be assessed, for example, by measuring release of cytokines, e.g., IFNγ, IL-2, TNFα, and Granzyme B.

In some examples, the PBMCs are obtained from a subject to be treated, such as a subject having cancer or one having a chronic viral infection. In other examples, the PBMCs are obtained from a donor subject, such as a subject who does not have cancer. In some examples, exhausted T cells are obtained from a tumor biopsy or sample (e.g., tumor infiltrating lymphocytes).

In some examples, the agent, non-naturally occurring genetic modification, or inhibitor is introduced into a PBMC ex vivo. In such examples, such methods can further include selecting modified PBMCs having reduced expression of Adrb1 and/or Adrb2, reduced activity of ADRB1 and/or ADRB2 (such as purifying or isolating such cells away from cells not having reduced expression of Adrb1 and/or Adrb2, and not having reduced activity of ADRB1 and/or ADRB2). Such methods can also further include selecting modified PBMCs that are T cells, for example, T cells that are CD3+ or CD8+. Exemplary selection methods include using flow cytometry, panning or magnetic separation. The disclosed methods in some examples further include introducing the selected modified PBMCs having reduced expression of Adrb1 and/or Adrb2, reduced activity of ADRB1 and/or ADRB2, or both, into a subject, such as a subject with a cancer to be treated with the selected modified PBMCs having reduced expression of Adrb1 and/or Adrb2, reduced activity of ADRB1 and/or ADRB2, or any combination the aforementioned.

In some examples, the agent, non-naturally occurring genetic modification, or inhibitor is administered to the subject, and the agent, non-naturally occurring genetic modification, or inhibitor is introduced into a PBMC (e.g., T cells, CD8+ TRM, tumor infiltrating lymphocytes, CAR T cells, or exhausted T cells (including terminally exhausted T cells)) in vivo.

In some aspects, the method of generating the modified PBMC further includes selecting a PBMC or cell type (e.g., T cells, CD8+ TRM, tumor infiltrating lymphocytes, CAR T cells, or exhausted T cells (including terminally exhausted T cells)), for example, from a sample (e.g., tumor biopsy, blood, population of T cells) before introducing the inhibitor, agent, or non-naturally occurring genetic modification. In some examples, the selected PBMC is reactive to a tumor-specific antigen, for examples, one or more of: CD19, CD20, BCMA, MUC1, PSA, CEA, HER1, HER2, TRP-2. EpCAM, GPC3, mesothelin 1 (MSLN), or EGFR. In some examples the selected PBMC is a T cell. In some examples, the T cell is CD8+ or CD3+. In some examples, the T cell is an adoptive cell transfer (ACT) therapy T cell, for example, the selected exhausted T cell can include a chimeric antigen receptor (CAR) or an engineered T cell receptor (TCR) specific for a tumor antigen. In further examples, the selected PBMC is a tumor-infiltrating lymphocyte (TIL). In some examples the PBMC is an exhausted T cell (for example a cell expressing PD-1, SLAMF6, TCF1, CX3CR1, TIM-3, TOX, TIGIT, CD39. LAG-3. CXCR6, EOMES and/or CD101). In some examples the PBMC is a TEX_{Prog}, a TEX_{Eff} and/or a TEX_{Term}.

In some examples, the agent (RNAi or gRNA) is introduced, for example, by contacting a PBMC with the agent, thereby generating the modified PBMC. In other examples, the agent is introduced by transfecting or transforming a PBMC with the disclosed nucleic acid molecule encoding the inhibitor or agent or the vector encoding a disclosed nucleic acid molecule, thereby generating the modified PBMC. Methods of transforming or transfecting a host cell are described herein, and can include: chemical methods (e.g., calcium-phosphate transfection), physical methods (e.g., electroporation, microinjection, particle bombardment), fusion (e.g., liposomes), nucleofection, receptor-mediated endocytosis (e.g., DNA-protein complexes, viral envelope/capsid-DNA complexes) and by biological infection by viruses, such as recombinant viruses. In the case of infection by retroviruses, the infecting retrovirus particles are absorbed by the target cells, resulting in reverse transcription of the retroviral RNA genome and integration of the resulting provirus into the cellular DNA. In some examples, a ribonucleoprotein (RNP) complex including the gRNA and a Cas nuclease or dead nuclease (e.g., Cas3, Cas9, Cas12, or Cas13d) is directly introduced into the PBMC. Methods of introducing a RNP complex into a host cell have been described. For example, the PBMC can be nucleofected with the RNP. In some examples, the PBMC is transfected with the RNP by electroporation (see e.g., Seki and Rutz, (2018) J Exp Med. 215(3): 985-997). In some examples, lipid-containing oligoaminoamides (lipo-OAAs) are used to as a carrier for intracellular delivery of the RNP complex (see e.g., Kuhn et al. (2020) Bioconjugate Chem. 31(3):729-742).

In specific, non-limiting examples, the introduced agent is shRNA, and the shRNA is introduced into the PBMC through infection with a viral vector encoding the shRNA. Introduction by a viral vector allows for stable integration of shRNA and long-term knockdown of the targeted gene. In another specific, non-limiting example, the introduced agent is siRNA, and siRNA is introduced cytosolically into a host cell capable of transfection.

In some examples, the non-naturally occurring genetic modification is introduced into the PBMC. The genetic modification can be any non-naturally occurring modification that results in decreased expression of Adrb1 and/or Adrb2 or reduced activity of ADRB1 and/or ADRB2. Non-limiting examples of genetic modifications include a point mutation, partial deletion, full deletion, or insertion. In some examples, the genetic modification is induced by a targeted genome editing technique, such as CRISPR/Cas, zinc finger nuclease, or TALEN modification of a Adrb1 and/or Adrb2 gene. Methods of genome editing have been previously described, for example, in Nemudryi et al (2014) Acta Naturae; 6(3): 19-40, herein incorporated by reference in its entirety. In some examples, the genetic modification reduces Adrb1 and/or Adrb2 expression, for example, by at least 20%, at least 30%, at least 40%, at least 50%, at least 60%, at least 70%, at least 80%, at least 90%, at least 95%, at least 98%, or 100%. In some examples, the genetic modification reduces ADRB1 and/or ADRB2 activity, for example, by at least 20%, at least 30%, at least 40%, at least 50%, at least 60%, at least 70%, at least 80%, at least 90%, at least 95%, at least 98%, or 100%.

In some examples the agent that reduces Adrb1 expression or a non-naturally occurring genetic modification that reduces an amount of functional ADRB1 comprises a zinc finger nuclease (ZFN) or transcription activator-like effector nuclease (TALEN) specific for Adrb1. In further examples the agent that reduces Adrb2 expression or a non-naturally occurring genetic modification that reduces an amount of functional ADRB2 comprises a zinc finger nuclease (ZFN) or transcription activator-like effector nuclease (TALEN) specific for Adrb2.

In some examples, the modified PBMC is incubated with at least one cytokine selected from the group consisting of interleukin 2 (IL-2), interleukin 7 (IL-7), interleukin 15 (IL-15), TGF-$, and retinoic acid TGF-3, and retinoic acid.

In some examples, introducing the non-naturally occurring genetic modification reduces activity of ADRB1 and/or ADRB2 in the modified PBMC by at least 10%, at least 20%, at least 30%, at least 40%, at least 50%, at least 60%, at least 70%, at least 80%, at least 90%, or at least 100% relative to a suitable control (e.g., an unmodified PBMC). In some examples, introducing the non-naturally occurring genetic modification protein levels of ADRB1 and/or ADRB2 by at least 10%, at least 20%, at least 30%, at least 40%, at least 50%, at least 60%, at least 70%, at least 80%, at least 90%, at least 100% or more relative to a suitable control. In some examples, introducing the non-naturally occurring genetic modification reduces activity of ADRB1 and/or ADRB2 by at least 10%, at least 20%, at least 30%, at least 40%, at least 50%, at least 60%, at least 70%, at least 80%, at least 90%, at least 100% or more relative to a suitable control. Reducing activity includes reducing any measurable biological function of ADRB1 and/or ADRB2, for example, reducing the interaction between ADRB1 and/or ADRB2 and the genome.

In some examples, decreasing expression of Adrb1 and/or Adrb2 or activity of ADRB1 and/or ADRB2 in a PBMC increases effector function, reduces exhaustion, increases resistance to exhaustion, or combinations thereof. In some examples, the PBMC is a T cell, and decreasing expression of Adrb1 and/or Adrb2 or activity of ADRB1 and/or ADRB2 in the PBMC increases effector function of the T cell, reduces exhaustion of the T cell, or causes the T cell to express at least one of Itgae. Itga1, Runx3. Cxcr3, Prdm1, Notch2, Tcf7, Cxcr5, I17r, 1d3, or Cd69 and/or causes reduced expression of S1pr1, Klf2, Klf3, Pdcd1, Tox, Entpd1, Cxcr6, Eomes, Tbx21, Tigit, Cd38, Lag3, Cx3cr1, Cd101, or Haver2 by the T cell. In some examples decreasing expression of Adrb1 and/or Adrb2 or activity of ADRB1 and/or ADRB2 in a PBMC causes cells to express CD69 and/or CD103.

In further examples, the PBMC is a T cell and decreasing expression of Adrb1 and/or Adrb2 or activity of ADRB1 and/or ADRB2 in the PBMC increases resistance to T cell exhaustion. In some examples, the disclosed modified PBMCs, such as modified T cells, do not become exhausted (e.g., do not express PD-1, TOX, SLAMF6, TCF1, CX3CR1, TIM-3, LAG-3, CD39, CD38, TIGIT, TBET, EOMES, CXCR6, and/or CD101). In some examples, the disclosed modified PBMCs, such as modified T cells, become exhausted at a slower rate, for example the number of days to progress to an exhausted cell (e.g., expression of PD-1, TOX, SLAMF6, TCF1, CX3CR1, TIM-3, LAG-3, CD39, CD38, TIGIT, TBET, EOMES, CXCR6, and/or CD101) is increased by at least 10%, at least 25%, at least 50%, at least 75%, at least 90%, at least 95%, or at least 99%, for example relative to a PBMC/T cell with native ADRB1 and/or ADRB2 expression/activity. In some examples, the disclosed modified PBMCs, such as modified T cells, results in a population of modified PBMCs, such as modified T cells, with fewer exhausted cells (e.g., PD-1, TOX, SLAMF6, TCF1, CX3CR1, TIM-3, LAG-3, CD39, CD38, TIGIT, TBET, EOMES. CXCR6, and/or CD101 expressing cells), such as a reduction of at least 10%, at least 25%, at least 50%, at least 75%, at least 90%, at least 95%, or at least 99%, for example relative to a PBMC/T cell with native ADRB1 and/or ADRB2 expression/activity. In some examples, the disclosed modified PBMCs, such as antigen presenting cells, result in a separate population of PBMCs, such as unmodified T cells with fewer exhausted cells (e.g., PD-1, TOX, SLAMF6, TCF1, CX3CR1, TIM-3, LAG-3, CD39, CD38, TIGIT, TBET, EOMES, CXCR6, and/or CD101 expressing cells).

Modified PBMCs with Increased Expression of Adrb1 and/or Adrb2

Also provided herein are modified peripheral blood mononuclear cells (PBMCs) with increased expression of Adrb1 and/or Adrb2. In some examples, modified PBMCs have increased expression of Adrb1 and/or increased activity of ADRB1. In some examples, modified PBMCs have increased expression of Adrb2 and/or increased activity of ADRB2. In some examples, modified PBMCs have increased expression of Adrb1 (and/or increased activity of ADRB1) and increased expression of Adrb2 (and/or increased activity of ADRB2).

In some examples, expression of Adrb1 and/or Adrb2 is increased by at least 10%, at least 20%, at least 30%, at least 40%, at least 50%, at least 60%, at least 70%, at least 80%, at least 90%, at least 95%, at least 98%, or at least 100% relative to a suitable control (e.g., a PBMC prior to modification). In some examples, activity of ADRB1 and/or ADRB2 is increased by at least 10%, at least 20%, at least 30%, at least 40%, at least 50%, at least 60%, at least 70%, at least 80%, at least 90%, at least 95%, at least 98%, or 100% relative to a suitable control (e.g., a PBMC prior to modification). In some examples, the modified PBMC is a T cell, and the T cell has increased tolerance relative to a suitable control (e.g., unmodified PBMC). In some examples the modified PBMC is an antigen presenting cell, and the antigen presenting cell expresses immune inhibitory surface proteins relative to a suitable control (e.g., unmodified PBMC). The modified PBMC can further include additional modifications, for example, the PBMC can express or otherwise contain a chimeric antigen receptor (CAR) or engineered T cell receptor (TCR).

In some examples, the modified PBMC is a T cell, for example, a CD4+, a CD8+ or a CD3+ T cell. The T cell can be reactive to an autoantigen, for example a citrullinated antigen, a nucleosome antigen, nuclear antigen (ANA), dsDNA, cytoplasmic antigen (ANCA), endomysium, smooth muscle, TTG_A, TTG_G, Ro, La, CCP3, Smith, RNP. Gliadin_A, Gliadin_G, Scl70, centromer, Jo1, Mi-2, MPO, PR3, Jp1, IgG-Fc, C1q, myosin, actin, myclin basic protein MBP, thyroglobulin, thyroid peroxidase or Ro52/Ro60. In some examples, the T cell is a therapeutic T cell, or will be used as a therapeutic T cell.

In some aspects, the modified PBMC includes an agent that increases Adrb1 and/or Adrb2 expression, for example an expression vector encoding Adrb1 and/or Adrb2, optionally operably linked to a promoter: or for example a heterologous nucleic acid encoding Adrb1 and/or Adrb2 optionally operably linked to a promoter. In some examples the agent that increases Adrb1 and/or Adrb2 expression includes a gRNA and a Cas nuclease. In some examples the agent that increases Adrb1 and/or Adrb2 includes a contiguous sequence at least 70%, at least 80%, at least 90%, at least 95%, at least 98%, at least 99%, or 100% sequence identity to SEQ ID NOs: 2, 3, 5, and/or 6. In some examples the agent that increases Adrb1 and/or Adrb2 includes SEQ ID NOs: 2, 3, 5, and/or 6. In some examples, the modified PBMC includes a RNP complex that includes the disclosed gRNA and a Cas nuclease, such as Cas3, dCas3. Cas9, dCas9, Cas12, dCas12, Cas13a, dCas13a, Cas13b, dCas13b, Cas13d, or dCas13d.

Nucleic acids or vectors can be transiently or stably introduced into a PBMC (e.g., T cell). In a specific, non-limiting example, the vector is stably introduced into the modified PBMC, thereby resulting in stable expression of the agent that increases Adrb1 and/or Adrb2. In some examples, the nucleic acid encoding the agent that increases Adrb1 and/or Adrb2 is operably linked to a cell specific promoter (e.g., a T cell specific promoter such as Lck, Cd4 (silencer minus) T cell receptor (Tcra)), or an antigen presenting cell specific promoter) in the vector. Expression of the agent that increases Adrb1 and/or Adrb2 can be constitutive or inducible. Exemplary promoters include NFAT, EF1a, PGK, U6, or H1. In one example, agent that increases Adrb1 and/or Adrb2 is expressed from a U6 or H1 promoter. In one example, a Cas nuclease (or dead Cas nuclease) is expressed from a CMV promoter.

In some aspects, the modified PBMC includes a non-naturally occurring genetic modification that increases an amount of functional ADRB1 and/or ADRB2. Increasing functional ADRB1 and/or ADRB2 includes genetic modifications that increase Adrb1 and/or Adrb2 expression (e.g., increasing transcription or translation of Adrb1 and/or Adrb2 gene or transcript) in the modified PBMC. In some examples, the genetic modification is a non-naturally occurring genetic modification of a Adrb1 and/or Adrb2 gene. In other examples, the genetic modification is a non-naturally occurring genetic modification of a regulatory element of Adrb1 and/or Adrb2 (e.g., a promoter, response element, enhancer, transcription factor, or other regulator that affects expression of Adrb1 and/or Adrb2). The regulatory element can be cis-acting or trans-acting.

In some examples, the non-naturally occurring genetic modification is a modification that increases an amount of functional ADRB1 and/or ADRB2 in the modified PBMC. In one example the genetic modification results in the production of ADRB1 and/or ADRB2 with additional signaling domains. In one example the genetic modification results in the production of increased amounts of ADRB1 and/or ADRB2. The genetic modification can be any non-naturally occurring modification that results in an increased amount of ADRB1 and/or ADRB2. Non-limiting examples of genetic modifications include a point mutation, partial deletion, full deletion, or insertion.

Methods of Generating Modified PBMCs with Increased Adrb1 and/or Adrb2

Also provided herein are methods of generating the disclosed modified PBMCs by introducing the non-naturally occurring genetic modification into a PBMC, thereby generating the modified PBMC with increased expression of Adrb1 and/or Adrb2 and/or increased activity of ADRB1 and/or ADRB2. In some aspects, the PBMC is obtained from a subject before introducing the non-naturally occurring genetic modification. PBMCs can be harvested or isolated, for example, from a blood sample, such as a venous blood sample, from the subject. Several techniques for isolating PBMCs can be used, for example, density centrifugation (the Ficoll approach), isolation by cell preparation tubes (CPTs), or isolation by SepMate™ tubes. In some examples, aphersis or leukapheresis is used to harvest PBMCs. Erythrocyte contamination can be evaluated, for example, by microscopic analysis of the sample. Flow cytometry techniques (e.g., FACS) can be used to assess the composition of the isolated PBMC populations, for example, to identify monocytes (e.g., CD14), T cells (e.g., CD3, CD8, CD4), B cells (e.g., CD20), or NK cells (e.g., CD56). FACS techniques can also be used to enrich or deplete a particular cell type from a PBMC (for example, enrich or deplete CD14, CD3, CD8, CD4, CD28, CD20, CD56, or combinations thereof).

In some aspects, the PBMC is harvested or isolated from a solid tissue sample, for example from a tumor. Tumor samples can be surgically resected, enzymatically digested, and PBMCs can subsequently be isolated, for example via the methods of Donia et al., Characterization and Comparison of ‘Standard’ and ‘Young’ Tumour-Infiltrating Lymphocytes for Adoptive Cell Therapy at a Danish Translational Research Institution, Scand. J. Immuno. (2011) 75:157-67, incorporated by reference herein. In some aspects the PBMC is harvested or isolated from a solid tissue sample taken from an organ affected by autoimmune disease.

In some examples, T cells are isolated from a PBMC sample, or the PBMC sample is enriched for T cells, for example, isolated or enriched for CD3⁺ or CD8⁺ T cells. In some examples, a sample is enriched by negative selection, for example, by selecting and removing unwanted cell types from a sample (e.g., cell types other than T cells, and/or naïve or memory T cells). In some examples, FACS is used to enrich for a particular PBMC, for example, to enrich for T cells (e.g., CD3 or CD8 positive T cells). FACS/Flow cytometry can also be used to assess whether immunologically tolerant cells (for example cells expressing EGR2) are present in a PBMC sample. FACS can be used to sort a PBMC sample to enrich for immunologically tolerant cells, or conversely to remove immunologically hyperresponsive cells. Antigen responsiveness of the PBMCs can be assessed, for example, by measuring production of cytokines, e.g., IFNγ, IL-2 and TNFα.

In some examples, the PBMCs are obtained from a subject to be treated, such as a subject having an autoimmune disease. In other examples, the PBMCs are obtained from a donor subject, such as a subject who does not have an autoimmune disease. In some examples, the PBMCs are obtained from a biopsy or sample of an organ affected by autoimmune disease.

In some examples, the agent or non-naturally occurring genetic modification is introduced into a PBMC ex vivo. In such examples, such methods can further include selecting modified PBMCs having increased expression of Adrb1 and/or Adrb2, increased activity of ADRB1 and/or ADRB2 (such as purifying or isolating such cells away from cells not having increased expression of Adrb1 and/or Adrb2, and not having increased activity of ADRB1 and/or ADRB2). Such methods can also further include selecting modified PBMCs that are T cells, for example, T cells that are CD3+, CD4+, CD25+, or CD8+. Such methods can include selecting modified PBMCs that are Tregs, dendritic cells, natural killer cells, B cells, and/or monocyte/macrophages. Exemplary selection methods include using flow cytometry, panning or magnetic separation. The disclosed methods in some examples further include introducing the selected modified PBMCs having increased expression of Adrb1 and/or Adrb2, increased activity of ADRB1 and/or ADRB2, or both, into a subject, such as a subject with a autoimmunity to be treated with the selected modified PBMCs having increased expression of Adrb1 and/or Adrb2, increased activity of ADRB1 and/or ADRB2, or any combination the aforementioned. In some examples, the agent or non-naturally occurring genetic modification is administered to the subject, and the agent, non-naturally occurring genetic modification introduced into a PBMC (e.g., T cells, antigen presenting cells) in vivo.

In some aspects, the method of generating the modified PBMC further includes selecting a PBMC or cell type (e.g., T cells, antigen presenting cells), for example, from a sample (e.g., tissue biopsy, blood, population of T cells) before introducing the agent or non-naturally occurring genetic modification. In some examples, the selected PBMC is reactive to a autoantigen, for examples, one or more of: citrullinated antigens, nucleosome antigens, and/or Ro52/Ro60. In some examples the selected PBMC is a T cell. In some examples, the T cell is CD8+, CD4+, CD25+, or CD3+.

In some examples, the agent is introduced, for example, by contacting a PBMC with the agent, thereby generating the modified PBMC. In other examples, the agent is introduced by transfecting or transforming a PBMC with the disclosed nucleic acid molecule encoding the inhibitor or agent or the vector encoding a disclosed nucleic acid molecule, thereby generating the modified PBMC. Methods of transforming or transfecting a host cell are described herein, and can include: chemical methods (e.g., calcium-phosphate transfection), physical methods (e.g., electroporation, microinjection, particle bombardment), fusion (e.g., liposomes), nucleofection, receptor-mediated endocytosis (e.g., DNA-protein complexes, viral envelope/capsid-DNA complexes) and by biological infection by viruses, such as recombinant viruses. In the case of infection by retroviruses, the infecting retrovirus particles are absorbed by the target cells, resulting in reverse transcription of the retroviral RNA genome and integration of the resulting provirus into the cellular DNA. In some examples, a ribonucleoprotein (RNP) complex including the gRNA and a Cas nuclease or dead nuclease (e.g., Cas3, Cas9, Cas12, or Cas13d) is directly introduced into the PBMC. Methods of introducing a RNP complex into a host cell have been described. For example, the PBMC can be nucleofected with the RNP. In some examples, the PBMC is transfected with the RNP by electroporation (see e.g., Seki and Rutz, (2018) J Exp Med. 215(3): 985-997). In some examples, lipid-containing oligoaminoamides (lipo-OAAs) are used to as a carrier for intracellular delivery of the RNP complex (see e.g., Kuhn et al. (2020) Bioconjugate Chem. 31(3):729-742).

In some examples, the non-naturally occurring genetic modification is introduced into the PBMC. The genetic modification can be any non-naturally occurring modification that results in increased expression of Adrb1 and/or Adrb2 or increased activity of ADRB1 and/or ADRB2. Non-limiting examples of genetic modifications include a point mutation, partial deletion, full deletion, or insertion. In some examples, the genetic modification is induced by a targeted genome editing technique, such as CRISPR/Cas, zinc finger nuclease, or TALEN modification of a Adrb1 and/or Adrb2 gene. Methods of genome editing have been previously described, for example, in Nemudryi et al. (2014) Acta Naturae; 6(3): 19-40, herein incorporated by reference in its entirety.

In some aspects, increasing expression of Adrb1 or activity of ADRB1 in a PBMC decreases effector function or increases T cell exhaustion or terminal differentiation and decreases memory-formation. In some aspects, increasing expression of Adrb2 or activity of ADRB2 in a PBMC decreases terminal differentiation and/or promotes memory-formation. In some aspects, the PBMC is a T cell, and increasing expression of Adrb1 or activity of ADRB1 in the PBMC causes the T cell to express at least one of KLRG1, PD-1, CXCR6, CD39, TIM-3, CX3CR1, CD101, CREM. In some examples, the PBMC is a T cell, and increasing expression of Adrb2 or activity of ADRB2 in the PBMC causes the T cell to express at least one of TCF1, SLAMF6, CD127.

### VI. Exemplary ADRB1 and ADRB2 Inhibitory Agents

In one example, the disclosed methods of treatment include administration to a subject of one or more agents that inhibit ADRB1 signaling, ADRB2 signaling, or both (herein ADRB1/ADRB2 antagonists). In one example, such ADRB1/ADRB2 antagonists include one or more beta-blockers, such as one or more of bisoprolol, metoprolol, propranolol, bucindolol, oxprenolol, carteolol, pindolol, oxprenolol, penbutolol, betaxolol, celiprolol, acebutolol, labetalol, carvedilol, pronethalol, sotalol, nebivolol, esmolol, butaxamine, alprenolol, bupranolol, nadolol, and timolol. In some examples the agent that inhibits ADRB1 is CGP 20712A. In some examples, the agent that inhibits ADRB2 is ICI 118551. In one example, such ADRB1/ADRB2 antagonists include one or more agents that agonize a beta-receptor, such as one or more of salbutamol, terbutaline, and salmeterol (and others used in asthma therapy). In some examples, use of the one or more ADRB1/ADRB2 antagonists is used in combination with one or more ICB agents (e.g., anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, and anti-CD27). For example, the one or more ADRB1/ADRB2 antagonists can be administered before, after or concurrently with one or more ICB agents (e.g., anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, and anti-CD27). For example, the one or more agents that agonize a beta-receptor, can be administered before, after or concurrently with one or more ICB agents (e.g., anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, and anti-CD27).

Therapeutically effective doses of Adrb1/Adrb2 antagonists (such as beta-blockers or beta-receptor agonists), in some examples, are from about 0.01 ug to about 1 g per day, such as 0.1 ug to 500 mg/day, 1 mg to 1 g/day, or 10 mg to 1 g/day. In one example, a beta-blocker is administered orally or ophthalmically at a dose of 1 to 1000 mg per day (such as 1 mg. 2.5 mg. 5 mg. 10 mg, 25 mg, 30 mg, 50 mg, 100 mg, 200 mg, 500 mg, or 1000 mg/day such as 2.5-5 mg/day, 20-40 mg/day, 50-450 mg/day, 80-160 mg/day, 50-200 mg/day, 400-800 mg/day, 200-1000 mg/day. Such doses may be administered as divided doses (e.g., given as 2, 3, 4, 6 or 8 separate doses). In one example, a beta-receptor agonist is administered at a dose of 50 ug to 1000 mg per day (such as 50 ug. 100 ug, 1 mg, 4 mg, 5 mg, 10 mg, 12 mg, 15 mg, 16 mg, 25 mg, 30 mg, 50 mg, 100 mg, 200 mg, 500 mg, or 1000 mg/day such as 4-16 mg/day, 4-50 mg/day, 10-50 mg/day, 50-450 mg/day, 80-160 mg/day, 50-200 mg/day, 400-800 mg/day, 200-1000 mg/day), such as via inhalation or orally. Such doses may be administered as divided doses (e.g., given as 2, 3, 4, 6 or 8 separate doses).

### VII Pharmaceutical Compositions

Also disclosed herein are pharmaceutical compositions useful for treating cancer, increasing response to immunotherapy, treating a viral infection (such as a chronic infection), and/or treating an autoimmune disease. In some examples, the pharmaceutical composition includes (1) one or more of: the disclosed RNAi specific to Adrb1 and/or Adrb2, one or more gRNAs specific for Adrb1 and/or Adrb2, the nucleic acid or vector encoding the RNAi or gRNA, the inhibitor (e.g., the agent that inhibits ADRB1 and/or ADRB2 signaling), one or more ICB agents, or the modified PBMC; and (2) a pharmaceutically acceptable carrier. In some examples the pharmaceutical composition includes (1) one or more of: the disclosed agent that increases Adrb1 and/or Adrb2, one or more gRNAs, or the modified PBMC and (2) a pharmaceutically acceptable carrier. In some examples the pharmaceutical composition includes (1) one or more of: an agent that inhibits ADRB1 and/or ADRB2 signaling and/or a therapeutically effective amount of an ICB agent and (2) a pharmaceutically acceptable carrier. In specific, non-limiting examples, the pharmaceutical composition includes a modified PBMC and a pharmaceutically acceptable carrier, such as water or saline.

In some examples, the pharmaceutical composition includes (1) one or more of: the RNAi specific to Adrb1 and/or Adrb2, the gRNAs specific for Adrb1 and/or Adrb2, the nucleic acid or vector encoding the RNAi or gRNA, the Adrb1 and/or Adrb2 inhibitor, or the modified PBMC; (2) a cancer immunotherapy; and (3) a pharmaceutically acceptable carrier. In some examples, the cancer immunotherapy is an ACT therapy (e.g., CAR-T, TCR, TIL), a monoclonal antibody (e.g., anti-PD-1, anti-EGFR, anti-CTLA4), a T cell agonist antibody, or an oncolytic virus. In one example, the cancer immunotherapy includes one or more ICB agents. In a specific, non-limiting example, the pharmaceutical composition includes the modified PBMC, an antibody cancer immunotherapy, and a pharmaceutically acceptable carrier. In another, non-limiting example, the pharmaceutical composition includes: one or more of the RNAi specific to Adrb1 and/or Adrb2, the gRNAs specific for Adrb1 and/or Adrb2, the nucleic acid or vector encoding the RNAi or gRNA; an ACT immunotherapy (e.g., CAR-T, TCR, TIL); and a pharmaceutically acceptable carrier.

In one example, the pharmaceutical composition includes: one or more of the RNAi specific to Adrb1, the gRNAs specific for Adrb1, the nucleic acid or vector encoding the RNAi or gRNA: one or more ICB agents (e.g., anti-PD1, anti-PD-L1, anti-CTLA4); and a pharmaceutically acceptable carrier. In one example, the pharmaceutical composition includes: one or more of the RNAi specific to Adrb2, the gRNAs specific for Adrb2, the nucleic acid or vector encoding the RNAi or gRNA: one or more ICB agents (e.g., anti-PD1, anti-PD-L1, anti-CTLA4); and a pharmaceutically acceptable carrier.

In further examples, the pharmaceutical composition includes (1) one or more of: the RNAi specific to Adrb1 and/or Adrb2, the gRNAs specific for Adrb1 and/or Adrb2, the nucleic acid or vector encoding the RNAi or gRNA, the Adrb1 and/or Adrb2 inhibitor, or the modified PBMC; (2) an antiviral agent; and (3) a pharmaceutically acceptable carrier. In some examples, the antiviral agent is aciclovir, ganciclovir, zidovudine, interferon alpha, or another direct acting antiviral agents.

In some examples, the pharmaceutical composition includes (1) one or more of: an expression vector encoding Adrb1 and/or Adrb2, optionally operably linked to a promoter, a heterologous nucleic acid encoding Adrb1 and/or Adrb2 optionally operably linked to a promoter, a gRNA and a Cas nuclease, one or more immunosuppressive agents and (2) a pharmaceutically acceptable carrier. In some examples the immunosuppressive agent is one or more of corticosteroids, cyclosporine, tacrolimus, azathioprine, methotrexate, anti-TNF antibodies, daclizumab and basiliximab.

In some examples the pharmaceutical composition includes (1) one of more of: an agent that inhibits ADRB1 signaling, an agent that inhibits ADRB2 signaling, an ICB agent and (2) a pharmaceutically acceptable carrier. In some examples the ICB agent includes anti-PD-1, anti-PD-L1, anti-CTLA-4, anti-LAG3 anti-GITR, anti-4-1BB, anti-CD40, anti-CD40L, and anti-OX40, anti-TIGIT, anti-VISTA, anti-CD73, anti-CD39, anti-HVEM, anti-BTLA, anti-CD27, or any combination thereof. In some examples the agent that inhibits ADRB1 signaling or the agent that inhibits ADRB2 signaling are one or more of: atenolol, bisoprolol, metoprolol, propranolol, bucindolol, oxprenolol, carteolol, pindolol, oxprenolol, penbutolol, betaxolol, celiprolol, acebutolol, labetalol, carvedilol, pronethalol, sotalol, nebivolol, esmolol, butaxamine, alprenolol, bupranolol, nadolol, timolol. In some examples the agent that inhibits ADRB1 signaling is CGP 20712A. In some examples the agent that inhibits ADRB2 signaling is ICI 118551.

A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington's Pharmaceutical Sciences, 23rd Edition, Academic Press, Elsevier, (2020)). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Methods for preparing administrable compositions include those provided in Remington's Pharmaceutical Sciences, 23rd Edition, Academic Press, Elsevier, (2020). In some examples, the pharmaceutical composition is formulated for intravenous administration.

### VIII. Methods of Treating Cancer, Viruses, and Autoimmunity

Also disclosed herein are methods of treating cancer, a tumor, and/or a viral infection (such as a chronic viral infection) in a subject by administering an effective amount of a disclosed composition (such as the RNAi specific to Adrb1 and/or Adrb2, the gRNA specific to Adrb1 and/or Adrb2 and a Cas nuclease or dead Cas nuclease (which may be administered as an RNP complex), a nucleic acid or vector encoding the RNAi or gRNA (wherein in some examples the vector also expresses and a Cas nuclease or Cas dead nuclease), the modified PBMC, the agents that inhibit ADRB1 and/or ADRB2 or the pharmaceutical composition disclosed herein (hereinafter collectively referred to as “composition”)), to the subject, thereby treating the cancer, tumor, or virus. In a specific, non-limiting example, the administered composition is an effective amount of the modified PBMCs disclosed herein. In some examples, PBMCs are removed from the subject and modified as disclosed herein ex vivo, then the modified cells are introduced into the subject. In some examples, PBMCs are modified in vivo, for example by introducing a therapeutic molecule provided herein (e.g., RNAi specific to Adrb1 and/or Adrb2, gRNA specific for Adrb1 and/or Adrb2) into the subject. In some examples, the administered composition includes one or more antiviral agents, such as aciclovir, ganciclovir, zidovudine, interferon alpha, and/or direct acting antiviral agents. In some examples, the administered composition includes one or more ICB agents.

Also disclosed herein are methods of increasing a response to immunotherapy (for example by at least 20%, at least 50%, at least 75%, at least 90%, at least 100%, or at least 200%, for example as compared to the immunotherapy alone) in a subject by administering an effective amount of the disclosed composition, thereby increasing a response to immunotherapy. In a specific, non-limiting example, the method is a method of increasing a response to immunotherapy in a subject and the composition is the disclosed vector encoding the RNAi or gRNA.

Further disclosed herein are methods of treating an autoimmune disease or disorder in a subject by administering an effective amount of a disclosed composition (such as one or more of: an agent that inhibits ADRB1 signaling, an agent that inhibits ADRB2 signaling, and an ICB agent) to the subject, thereby treating the autoimmune disease or disorder. In a specific non-limiting example, the administered composition is an amount of a beta-blocker effective to inhibit ADRB1 signaling on PBMCs localized in a tumor and a therapeutically effective amount of an ICB agent, such as PD-1. In some examples, multiple ICB agents are administered in tandem.

In some examples, the subject has a tumor or cancer. In some examples, the subject has a solid tumor or cancer, such as breast carcinomas (e.g. lobular and duct carcinomas, such as a triple negative breast cancer), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors. Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma (such as an HPV-positive HNSCC), and lymphatic tumors (including B-cell and T-cell malignant lymphoma).

In some examples, the subject has a liquid tumor or cancer, such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), a lymphoma (such as Hodgkin's lymphoma or non-Hodgkin's lymphoma), or a myeloma.

In further-non limiting examples, the subject has an acute or chronic leukemia, Hodgkin or Non-Hodgkin lymphoma, myeloma, gastric cancer, esophageal cancer, colorectal cancer, hepatocellular carcinoma or other liver cancer, cholangiocellular carcinoma, melanoma, cervical cancer, uterine cancer, lung cancer, ovarian cancer, bladder cancer, urothelial cancer, breast cancer, pancreatic cancer, renal cell carcinoma, prostate cancer, testicular cancer, glioblastoma, nephroblastoma, neuroblastoma, neuroendocrine cancer, pheochromocytoma or adrenal carcinoma, sarcoma, thyroid cancer, laryngeal cancer or head and neck cancer.

In some examples the subject has a viral infection, such as a chronic viral infection, such as an infection caused by: adenovirus (Ad), a herpes simplex virus (HSV, type 1 and 2), a hepatitis B virus (HBV), a hepatitis C virus (HCV), a hepatitis D virus (HDV), a hepatitis E virus (HEV), a vesicular stomatitis virus (VSV), a human immunodeficiency virus (HIV), an influenza virus, a varicella zoster virus (VZV), a human papillomavirus (HPV), an Epstein-Barr virus (EBV), a cytomegalovirus (CMV), a human herpesvirus (HHV-6, HHV-7), a human T-cell leukemia virus (HTLV-1, HTLV-2), JC virus, BK virus, an enterovirus, a parvovirus, a paramyxovirus (e.g. measles), a togavirus, SARS-CoV, SARS-CoV2, or a flavivirus.

In some examples the subject has an autoimmune disease or disorder such as rheumatoid arthritis, systemic lupus erythematosus, type 1 and type 2 diabetes, multiple sclerosis, acute disseminated encephalomyelitis, Sjögren's syndrome, Graves' disease, myasthenia gravis, ulcerative colitis. Hashimoto's thyroiditis, celiac disease, Crohn's disease, arthritis, inflammatory bowel disease, psoriasis, autoimmune hepatitis, autoimmune pancreatitis, autoimmune encephalitis, or scleroderma, autoimmune hemolytic anemia, autoimmune thrombocytopenia, autoimmune neutropenia, atopic dermatitis, alopecia, ankylosing spondylitis, Addison's disease, alopecia areata, anti-phospholipid antibody syndrome, Goodpasure's Syndrome. Grave's disease, Guillain-Barre syndrome, IgA Nephropathy, pemphigoid, pemphigus, polyglandular autoimmune syndrome type 2, psoriatic arthritis, Takayasu's arteriosus, or undifferentiated connective tissue disease (UCTD).

In some examples, the subject is receiving, has received, or will receive immunotherapy, for example, one or more ICB agent targeting PD-1, PD-L1, CTLA-4, LAG3 GITR, 4-1BB, CD40, CD40L, and OX40, TIGIT, VISTA, CD73, CD39, HVEM, BTLA, CD27, CDK4, CDK6, or any combination of two or more of thereof. Exemplary checkpoint inhibitors include ipilimumab, nivolumab, pembrolizumab, atezolizumab, avelumab, durvalumab, cemiplimab, palbociclib, ribociclib, abemaciclib, pidilizumab, cosibelimab, envafolimab, BMS-936559, BMS935559, MEDI-4736, MPDL-3280A, MEDI-4737, and tremelimumab. In some examples, the effective amount of the composition is an amount that increases a response of the subject to an immunotherapy (e.g., a checkpoint inhibitor or ACT); for example, an amount that when administered with the immunotherapy, is more effective at treating cancer or a tumor relative to administration of the immunotherapy (or composition) alone. In some examples, the effective amount is an amount that is synergistic when administered with an immunotherapy, for example, an amount that synergistically prevents, treats, reduces, and/or ameliorates one or more sign or symptom of cancer.

In some examples the subject is receiving, has received, or will receive an immunosuppressive agent. Specific, non-limiting examples of immunosuppressive agents are corticosteroids, cyclosporine A, FK506, and anti-CD4. In additional examples, the agent is a biological response modifier, such as KINERET® (anakinra), ENBREL® (etanercept), or REMICADE® (infliximab), a disease-modifying antirheumatic drug (DMARD), such as ARAVA® (leflunomide). Agents of use to treat inflammation include non-steroidal anti-inflammatory drugs (NSAIDs), specifically a Cyclo-Oxygenase-2 (COX-2) inhibitor, such as CELEBREX® (celecoxib) and VIOXX® (rofecoxib), or another product, such as HYALGAN® (hyaluronan) and SYNVISC® (hylan G-F20).

In some examples, the effective amount of the composition is an amount sufficient to prevent, treat, reduce, and/or ameliorate one or more signs or symptoms of cancer in the subject. For example, an amount sufficient to reduce tumor size or tumor load in the subject by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount sufficient to inhibit or slow metastasis in the subject. For example, by decreasing tumor spread in the subject by at least 10%, at least 20%, at least 30%, at least 40%, at least 50%, at least 60%, at least 70%, at least 80%, at least 90%, at least 95%, or 100% as compared to a baseline measurement for the same subject, or a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 400%, or more. In other examples, the effective amount is an amount sufficient to reduce tumor density in the subject, for example, by at least 10%, at least 20%, at least 30%, at least 40%, at least 50%, at least 60%, at least 70%, at least 80%, at least 90%, or 100% as compared to a baseline measurement for the same subject or other suitable control. Non-limiting examples of suitable controls include untreated subjects or subjects not receiving the composition (e.g., subjects receiving other agents or alternative therapies). In further examples, the effective amount is an amount sufficient to target and eliminate tumor cells, for example, eliminate at least 10%, at least 20%, at least 30%, at least 40%, at least 50%, at least 60%, at least 70%, at least 80%, at least 85%, at least 90%, at least 95%, at least 98%, or even 100%, relative to a suitable control.

In some examples, the effective amount of the composition is an amount sufficient to prevent, treat, reduce, and/or ameliorate one or more signs or symptoms of viral infection in the subject, for example, an amount sufficient to reduce viral load by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount sufficient to inhibit or slow viral replication in the subject for example, an amount sufficient to inhibit or slow viral replication by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 400%, or more. In some one specific non-limiting example, an effective amount is an amount sufficient to increase T cell counts in an HIV infected subject by at least 10%, at least 20%, at least 30%, at least 40%, at least 50%, at least 60%, at least 70%, at least 80%, at least 90%, at least 95%, or 100%, as compared to a baseline measurement for the same subject, or a suitable control.

In some examples, the effective amount of the composition is an amount sufficient to prevent, treat, reduce, and/or ameliorate one or more signs or symptoms of autoimmunity in the subject. For example, an amount sufficient to reduce signs or symptoms of autoimmunity in the subject by at least 10%, at least 20%, at least 30%, at least 40%, at least 50%, at least 60%, at least 70%, at least 80%, at least 90%, at least 95%, or 100%, as compared to a baseline measurement for the same subject, or a suitable control. In some examples, the effective amount is an amount sufficient to reduce the proportion of activated immune cells in the subject. For example, an amount sufficient to reduce the proportion of activated immune cells by at least 10%, at least 20%, at least 30%, at least 40%, at least 50%, at least 60%, at least 70%, at least 80%, at least 90%, at least 95%, or 100% as compared to a baseline measurement for the same subject, or a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 400%, or more. Non-limiting examples of suitable controls include untreated subjects or subjects not receiving the composition (e.g., subjects receiving other agents or alternative therapies).

In some examples, the method reduces expression of Adrb1 and/or Adrb2 or activity of ADRB1 and/or ADRB2 in a target tissue or cell in the subject, for example, in a PBMC. T cell, or exhausted T cell (including terminally exhausted T cells)). In some examples, expression of Adrb1 and/or Adrb2 or activity of ADRB1 and/or ADRB2 is decreased by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method reduces protein levels of ADRB1 and/or ADRB2 (or functional ADRB1 and/or ADRB2), for example, by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method reduces expression of Adrb1 and/or Adrb2 or accumulation of mRNA transcripts by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment).

In some examples, the method increases expression of Adrb1 and/or Adrb2 or activity of ADRB1 and/or ADRB2 in a target tissue or cell in the subject, for example, in a PBMC, T cell, or antigen presenting cell). In some examples, expression of Adrb1 and/or Adrb2 or activity of ADRB1 and/or ADRB2 is increased by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method increases protein levels of ADRB1 and/or ADRB2 (or functional ADRB1 and/or ADRB2), for example, by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment). In some examples, the method increases expression of Adrb1 and/or Adrb2 or accumulation of mRNA transcripts by at least 10%, at least 20%, at least 30%, at least 40%, at least 50%, at least 60%, at least 70%, at least 80%, at least 90%, at least 95%, or 100% relative to a suitable control (e.g., an untreated subject or a baseline reading of the same subject prior to treatment).

In some examples, decreasing expression of Adrb1 and/or Adrb2 or activity of ADRB1 and/or ADRB2 increases T cell effector function or decreases T cell exhaustion. In some examples, decreasing expression of Adrb1 and/or Adrb2 or activity of ADRB1 and/or ADRB2 reduces (including prevents or inhibits) T cell exhaustion or increases resistance to (including prevents or inhibits) T cell exhaustion. In some examples, increasing T cell response or reducing T cell exhaustion in a subject increases response to an immunotherapy in the subject. In some examples increasing expression of Adrb1 and/or Adrb2 or activity of ADRB1 and/or ADRB2 increases immune tolerance.

In a specific, non-limiting example, the method includes administering to the subject the modified PBMC and a pharmaceutically acceptable carrier. When the disclosed PBMC is administered, the composition includes about 10⁴ to 10¹² of the modified PBMCs (for example, about 10⁴-10⁸ cells, about 10⁶-10⁸ cells, about 10⁶-10¹² cells, about 10⁸-10¹² cells, or about 10⁹-10¹⁰ cell). For example, the composition may be prepared such that about 10⁴ to 10¹⁰ modified PBMCs (e.g., about 10⁴, 10⁵, 10⁶, 10⁷, 10⁸, 10⁹, or 10¹⁰ cells/kg) are administered to a subject. In some examples, about 10¹⁰ cells/kg are administered to the subject. In specific examples, the composition includes at least 10⁴, 10⁵, 10⁶, 10⁷, 10⁸, 10⁹, or 10¹⁰ modified PBMCs. In a specific, non-limiting example, about 10⁸-10¹⁰ modified PBMCs are administered to the subject. An appropriate dose can be determined by a treating clinician based on factors such as the subject, the cancer being treated, treatment history, tumor load and type, clinical stage and grade of the disease, viral load, overall health of the subject, and other factors.

In some examples, non-modified lymphocytes are depleted in the subject prior to administering the disclosed composition. In some examples, the subject is also administered one or more cytokine(s) (such as IL-2, IL-7, IL-15, IL-21, and/or IL-12), for example, to support survival and/or growth of the disclosed modified PBMCs and/or an additional ACT therapy administered in combination, in the subject. In a specific, non-limiting example, at least one of IL-2, IL-7, and IL-15 is also administered to the subject. The cytokine(s) are administered before, after, or substantially simultaneously with the composition. In specific examples, at least one cytokine (e.g., IL-2, IL-7, and/or IL-15) is administered simultaneously, for example, with the composition. In some examples, the modified PBMC is reactive to a tumor-specific antigen in the subject having cancer. In some examples, the antigen is one or more of: CD19, CD20, BCMA, MUC1, PSA, CEA, HER1, HER2, TRP-2, EpCAM, GPC3, mesothelin 1 (MSLN), or EGFR.

Administration of any of the disclosed compositions can be local or systemic. Exemplary routes of administration include, but are not limited to, oral, injection (such as subcutaneous, intramuscular, intradermal, intraperitoneal, intravenous), sublingual, rectal, transdermal (for example, topical), intranasal, vaginal, and inhalation routes. In some examples, the agent is injected or infused into a tumor, or close to a tumor (local administration), or administered to the peritoneal cavity. Appropriate routes of administration can be determined by a treating clinician based on factors such as the subject, the condition being treated, and other factors.

Multiple doses of the composition can be administered to a subject. For example, the compositions can be administered daily, every other day, twice per week, weekly, every other week, every three weeks, monthly, or less frequently. A treating clinician can select an administration schedule based on the subject, the condition being treated, the previous treatment history, and other factors.

In some examples, the subject having cancer receives additional treatment, such as one or more of surgery, radiation, chemotherapy, biologic therapy, immunotherapy, or other therapeutic. Exemplary chemotherapeutic agents include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of chemotherapy drugs that can be used include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol.

In some examples, the subject treated is administered an additional therapeutic, such as a monoclonal antibody cancer immunotherapy (e.g., anti-CTLA-4, anti-PD1, or anti-PDL1), a T cell agonist antibody, an oncolytic virus, an adoptive cell transfer (ACT) therapy, or any combination of two or more thereof. The administration of an additional therapeutic may be before, after, or substantially simultaneously with the administration of the disclosed composition. In some examples, the additional therapeutic is a cell cycle or checkpoint inhibitor. In some examples, the checkpoint inhibitor targets PD-1, PD-L1, CTLA-4, CDK4, and/or CDK6. Exemplary inhibitors include ipilimumab, nivolumab, pembrolizumab, atezolizumab, avelumab, durvalumab, cemiplimab, palbociclib, ribociclib, and abemaciclib.

In some examples, the subject treated is also administered an ACT therapy, for example, a chimeric antigen receptor (CAR)-expressing T cell, engineered TCR T cell, or a tumor-infiltrating lymphocyte (TIL). In some examples, the subject is administered an effective amount of the composition and the ACT therapy, and an effective amount of the composition is an amount that increases effectiveness of the ACT (e.g., increases elimination of cancerous cells relative to ACT therapy alone).

The additional therapeutic may be administered substantially simultaneously with the disclosed composition. In some examples, the additional therapeutic is administered prior to administering the composition, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the additional therapeutic can be administered to a subject, for example, administered twice daily, once daily, every other day, twice per week, weekly, every other week, every three weeks, monthly, or less frequently. A treating clinician can select an administration schedule based on the subject, the condition being treated, the previous treatment history, tumor load and type, clinical stage and grade of the disease and overall health of the subject, and other factors.

### IX. Kits

Also provided are compositions and kits that can be used with the disclosed methods. In some examples, the composition or kit includes one or more of: the RNAi specific to Adrb1 and/or Adrb2, the gRNA specific to Adrb1 and/or Adrb2, a nucleic acid or vector encoding the RNAi or gRNA, and the modified PBMC, the agent that inhibits ADRB1 and/or ADRB2 signaling, the ICB agent, the agent that increases Adrb1 and/or Adrb2 expression, an immunosuppressive agent, a pharmaceutically acceptable carrier, and optionally instructions for use. In some examples, the kit includes one or more gRNA specific for Adrb1 and/or Adrb2 and a Cas nuclease or Cas dead nuclease (which may be an RNP complex). In a specific, non-limiting example, the kit includes a vector encoding one or more gRNA specific for Adrb1 and/or Adrb2, which can further encode a Cas nuclease or Cas dead nuclease. In further examples, the kit includes the disclosed modified PBMCs. In some examples the kit includes an expression vector encoding Adrb1 and/or Adrb2, optionally operably linked to a promoter, and/or a heterologous nucleic acid encoding Adrb1 and/or Adrb2 optionally operably linked to a promoter. In some examples the kit includes an agent that inhibits ADRB1 and/or ADRB2 signaling.

In some examples, the kit includes ICB agents, optionally in a separate container. In some examples, the ICB agents target PD-1, PD-L1, CTLA-4, LAG3 GITR, 4-IBB, CD40, CD40L, and OX40, TIGIT, VISTA, CD73, CD39, HVEM, BTLA, CD27, CDK4, and/or CDK6. In some examples the kit includes other anti-tumor agents, such as a chemotherapeutic agent, optionally in a separate container. In some examples, the kit includes anti-viral agents, optionally in a separate container. In some aspects, the anti-viral agents could include small molecules that inhibit virus replication, and/or antibodies which neutralize a virus. In some examples the kit includes immunosuppressive agents, optionally in a separate container.

The kit can include additional reagents, such as one or more of anti-CD3, anti-CD28, IL-2, and IL-15. In some examples, the reagents are present in separate containers. In one example, anti-CD3 and anti-CD28 are in the same container, and may be present, for example, on a bead. In some examples, the kit further includes one or more of a transfection reagent, culture medium, antibiotic, cytokines (e.g., IL-2, IL-15, and IL-7), optionally wherein such reagents are present in separate containers. In some examples the kit or composition includes media in which the PBMCs can be cultured or expanded ex vivo, such as AIM V® media.

### EXAMPLES

The following examples are provided to illustrate particular features of certain aspects of the disclosure, but the scope of the claims should not be limited to those features exemplified.

### Example 1

### Materials and Methods

This example illustrates the materials and methods used by the following examples.

### Mice

C57BL/6J mice were purchased from Jackson Laboratories. P14⁺ mice have been previously described⁵⁰. Adrb1^{fl/fl} Granzyme B^{Cre+} P14⁺ Thy1.1 mice were generated by crossing Adrb1^{fl/fl} mice to Granzyme B^{Cre+} P14⁺ Thy1.1 mice. Both male and female mice between 6-12 weeks of age were used. In all experiments age and sex-matched mice were randomly assigned to experimental groups. All experiments involved control samples and the respective treatment conditions. Blinding was performed for abdominal ultrasound measurements. Sample size is indicated in the figure legends. No statistical methods were used to predetermine sample sizes. Animals were housed in specific-pathogen-free facilities with a 6 am to 6 pm light/6 pm to 6 am dark cycle, room temperatures from 68-72 degrees Fahrenheit and a humidity range of 30-70%. The maximal tumor size permitted of 2 cm³ for subcutaneous tumors or experimental endpoints for orthotopic PDAC tumors including lethargy and greater than 10% loss of body weight were not exceeded in any experiments.

For betablocker experiments mice were treated with atenolol or propranolol (Sigma-Aldrich) in drinking water (0.5 mg/ml). CGP 20712 dihydrochloride (Tocris) was administered in drinking water at a dose of 10 μg/ml. ICI 118551 hydrochloride (EMD Millipore) was administered at 0.2 μg/g i.p. daily. Treatment was started 2 days before infection or tumor implantation and continued throughout the experiment. For virus experiments, LCMV-clone 13 infected mice were treated with 200 μg anti-PD-L1 (10F.9G2, Leinco) antibody or isotype control IgG2B (MPC-11, Leinco) every 3 days between day 23 and 36 p.i. For PDAC and YUMMER experiments, mice were treated with 100 μg anti-PD-1 (RMP1-14, Leinco) and 100 μg anti-CTLA-4 (9H10, Leinco) or isotype control antibodies IgG2A (C1.18.4, Leinco) and IgG2B (MPC-11, Leinco) twice per week. For CD8⁺ depletion assays mice were treated with anti-mouse CD8a (2.43, Leinco) 200 μg i.p. for 3 subsequent days and then once per week.

### Human Blood Samples

Blood samples from non-viremic HIV infected donors and uninfected donors were processed and frozen on the day of the venipuncture. After thawing, cells were rested at 37° C. overnight before staining for flow cytometric analysis.

### Adoptive Transfer

P14⁺ CD8⁺ T cells congenic for Ly5 or Thy1 were adoptively transferred into recipient mice by retroorbital injection at the cell numbers indicated in the respective figure legend. Recipient and donor mice were sex and age matched or female P14⁺ cells were transferred into male recipient mice. Littermate P14⁺ cells of the same sex were transferred for all mixed transfers. Mixed adoptive transfers were performed with a 1:1 ratio, if transduced cells were used for adoptive transfers 1:1 mix was performed for GFP⁺ or ametrine⁺ cells respectively. If naïve cells were transferred, mice were infected with LCMV-clone 13 at 2×10⁶ pfu by retroorbital injection on the next day, if activated cells were transferred, mice were infected on the day of the adoptive transfer.

### Tumor Cell Lines

All tumor cell lines were used for implantation when in exponential growth phase. YUMMER1.7 murine melanoma cell line⁵¹, MC38 colon adenocarcinoma cell line and 6419c5 murine pancreatic ductal adenocarcinoma cell line⁵² were maintained in DMEM/F12 (YUMMER1.7, MC38) or DMEM (6419c5) (Thermo Fisher Scientific) medium supplemented with 10% fetal bovine serum (Omega Scientific), 1% penicillin/streptomycin (Gibco), MEM Non-Essential Amino Acids (Gibco) and 2 mM L-Glutamine (Gibco). 6419c5 PDAC cell line and MC38 colon adenocarcinoma cell line were purchased from Kerafast.

### Tumor Implantation

6419c5 cells were dissociated from culture using 0.25% trypsin (Gibco), washed, and resuspended in fresh culture medium. 5×10³ cells were orthotopically implanted into the pancreas as a mixture of 10 μL growth factor reduced Matrigel (Corning 356231), 2 μL 0.4% Trypan Blue, and 8 μL of cell suspension for a total volume of 20 μL. Mice were anesthetized under continuous isoflurane and given 5 mg/kg Buprenorphine slow release. The left abdominal side was shaved to expose the incision site, which was sterilized using povidone-iodine followed by an alcohol wipe. A small incision in the abdominal skin was made using scissors to expose the underlying muscle. A secondary small incision was made in the muscle layer using sterile scissors, and the pancreas was gently exposed from the abdominal cavity using sterile ring forceps. PDAC cell mixture was injected using a 0.3cc insulin syringe with a 31 G needle (BD) into the tail of the pancreas. The pancreas was returned into the abdominal cavity and the incision site was closed using polysorb sutures (Covidien UL213) for the inner muscle layer and 9 mm steel skin clips for the outer skin. All surgical tools were sterilized between animals using 70% isopropyl alcohol and a heated bead sterilizer. Animals were monitored for 3 days post-surgery for recovery, and skin clips were removed 7-10 days after surgery.

YUMMER1.7 and MC38 cells were dissociated from culture using 0.25% trypsin (Gibco), washed, and resuspended in PBS. Mice were anesthetized under continuous isoflurane, and one flank of the mouse was shaved to expose the injection site. The skin was gently lifted using forceps, and 5×10⁵ cells were subcutaneously injected in a volume of 100 μL using an insulin syringe with a 29-gauge needle (BD). YUMMER1.7 tumors were measured twice weekly beginning at 7 days post-injection using a digital caliper.

### Ultrasound

For growth monitoring of PDAC, mice were subjected to abdominal ultrasound twice per week starting on d7 until harvest on d21 post implantation using a Vevo 3100 ultrasound machine (Visualsonics).

### Tissue Processing, Flow Cytometry and Cell Sorting

Single-cell suspensions of splenocytes were obtained by mechanical disaggregation of spleens through a 70 μm cell strainer (VWR) followed by red blood cell lysis with ACK lysis buffer (KD Medical).

PDAC tumors were dissected out at d21 after tumor implantation and subsequently minced with razor blades in a cell culture dish before being digested with dissociation buffer (10× dissociation buffer: 40 ml RPMI/DMEM (Gibco) with 1% Pen/Strep (Gibco)+1 mM NaPyr (Gibco)+25 mM HEPES (Lonza)+400 mg Collagenase IV (Sigma)+400 mg Soybean Trypsin Inhibitor (Thermo Scientific)+50 mg Dispase II (Sigma)+20 mg DNAse (Sigma)) for 30 min at 37° C. Samples were then strained through a 70 μM cell strainer, spun down at 420 rcf and 4° C. for 4 min and resuspended in RPMI with 10% fetal bovine serum before plating for staining. YUMMER and MC38 tumors were processed similarly to PDAC tumors but no Soybean Trypsin Inhibitor was added.

For flow staining, cells were incubated with viability dye (Ghost Dye™ Red 780, Tonbo) in PBS for 5 minutes at RT before incubation with the indicated surface antibodies for 30 min on ice in PBS supplemented with 2% FBS. The FoxP3 transcription factor staining kit (eBioscience) was used for intracellular and intranuclear staining. For cAMP staining cells were fixed with Fixation Buffer (Biolegend) prior to incubation with anti-cAMP antibody (ab134901, Abeam) for 24 hours at 4° C. and subsequent secondary staining with Anti-rabbit IgG (H+L), F(ab′)2 Fragment. For intracellular cytokine staining in tumor samples cells were stimulated with ionomycin (Iono, Cell Signaling; final concentration 1 μg/ml) and phorbol 12-myristate-13-acetate (PMA, Sigma; final concentration 50 ng/ml) for 5 hours in the presence of brefeldin A (GolgiPlug, BD Biosciences; 0.5 μL/ml) and monensin (GolgiStop, BD Biosciences; 0.325 μL/ml) for 4 hours at 37° C. 2% paraformaldehyde was used to fix the cells after staining. For P14⁺ stimulation P14⁺ cells were stimulated with 0.1 μg/ml gp33 (GenScript) for 6 h in the presence of brefeldin A (GolgiPlug, BD Biosciences; 0.5 μL/ml) and monensin (GolgiStop, BD Biosciences; 0.325 μL/ml) for 5 hours at 37° C. Gp33 tetramers were obtained from the NIH Tetramer Core Facility. Data acquisition was performed on a LSR II (BD Biosciences) and analysis was performed using Flowjo software (TreeStar). All sorting was performed on BD Biosciences Aria or BD Biosciences Influx cell sorter (100-micron nozzle settings, with sample and collection cooling set to 4° C.). For Calcium flux measurements, Adrb1 overexpressing P14⁺ cells were mixed at 1:1 ratio with control P14⁺ cells using P14⁺ mice with distinct congenics. Indo-1 (Thermo Scientific) staining was performed as per manufacturer's instructions followed by surface staining. After staining, cells were resuspended in PBS supplemented with 2 mM Calcium and 2% fetal bovine serum and incubated with 10 μM noradrenaline or adrenaline for 15 minutes. After recording of baseline, cells were stimulated with anti-CD3 (2 μg/ml, 145-2C11, BD)+goat anti-hamster IgG (6 μg/ml, H+L, Thermo Fisher Scientific) or gp33 (0.2 μg/ml. GenScript) and subsequently ionomycin (2 μg/ml, Cell Signaling) as positive control.

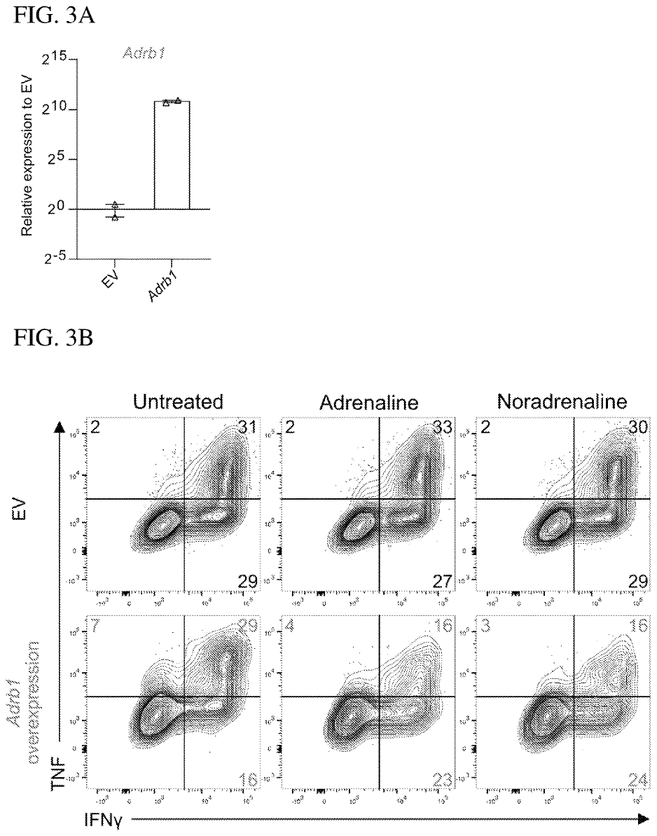

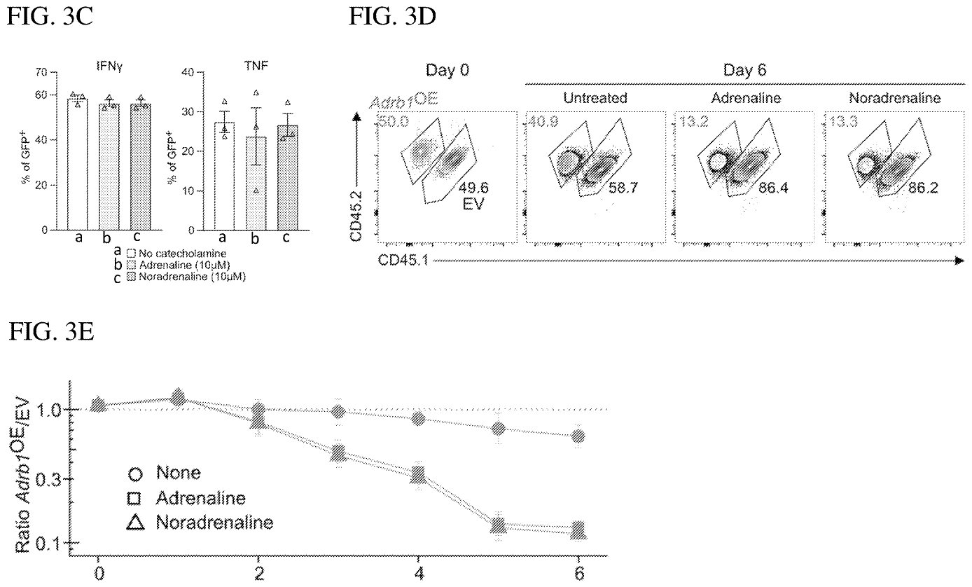

For the gating strategies for flow cytometry experiments, please refer to FIG. 3.

### Generation of Retrovirus and T Cell Transduction

One day before transfection, Plat-E cells were plated in a 10 cm tissue culture plate. Subsequently, each plate was transfected using 5 μg pCL-Eco and 10 μg of the respective plasmid using TransIT-LT1 (Mirus). 2 and 3 days after transfection retroviral supernatant was collected. Splenic CD8⁺ T cells were isolated and enriched using negative magnetic bead selection. 2×10⁶ P14⁺ cells were plated in a 6 well tissue culture plate coated with goat anti-hamster IgG (H+L, Thermo Fisher Scientific), anti-CD3 (145-2C11, BD), and anti-CD28 (37.51, BD). T cell culture media was replaced with retroviral supernatant supplemented with 50 μM 2-mercaptoethanol and 8 μg/mL polybrene (Millipore) 18 hours after cell plating. CD8⁺ T cells were spinfected for 60 min at 800×g at 37° C. and then incubated for 3 hours at 37° C. before retroviral supernatant was replaced by T cell culture media. All GFP⁺ or ametrine⁺ cells as assessed by flow cytometry 24 h after transduction were considered transduced.

For overexpression, the coding DNA sequence of Adrb1 was cloned into MIGR1 vector (Addgene #27490). Empty MIGR1 was used as control overexpression (EV). For knockdown of Crem, the hairpin sequence (TGCTGTTGACAGTGAGCGACAGACTCAGAAGTAATTGATATAGTG-AAGCCACAGATGTATATCAATTACTTCTGAGTCTGCTGCCTACTGCCTCGGA, SEQ ID NO: 27) was cloned into the pLMPd-Amt vector. A hairpin sequence targeting Cd19 was used as a control.

### Cell Culture

Plat-E cells were maintained in DMEM+D-glucose supplemented with 10% fetal bovine serum, 100 U/mL Penicillin, 100 μg/mL Streptomycin, 292 μg/mL L-glutamine, 10 mM HEPES, and 55 μM 2-Mercaptoethanol. Negatively bead-selected CD8⁺ T cells were cultured in RPMI+L-glutamine supplemented with 10% fetal bovine serum, 1% penicillin/streptomycin and 55 μM 2-Mercaptoethanol (Sigma).

For assessment of proliferation, transduced P14⁺ cells with distinct congenics were mixed at 1:1 ratio of GFP⁺ Adrb1 overexpressing cells to MIGR1 overexpressing cells prior to labeling with CellTrace™ violet (Thermo Fisher) according to manufacturer's instructions and cultured with 10 ng/ml IL-2 (PeproTech). Adrenaline and noradrenaline (Sigma) were added at final concentration of 10 μM every day. Catecholamines were solved in 0.5M HCl and 0.5M HCl was added in all control conditions without catecholamine addition. CTV staining was assessed by flow cytometry. For assessment of proliferation based on ratio changes in culture, IL-2 was added every two days at 10 ng/ml final concentration. To assess influence of ADRB1 on T cell function, Adrb1 overexpressing P14⁺ cells were stimulated with gp33 in the presence or absence of adrenaline or noradrenaline (10 μM) for 6 hours before intracellular cytokine staining.

### Catecholamine ELISA

Noradrenaline high sensitive ELISA (Eagle Biosciences) was performed on mouse serum and as per manufacturer's instructions. Mice subjected to acute cold and restraint stress were used as positive control.

### CRISPR/Cas9 RNP

Adrb1 knockout was performed in naïve P14⁺ cells using murine Adrb1 guide from Synthego TGGCCATCGCCAAGACCCCG, SEQ ID NO: 28, a Lonza 4D Nucleofector™ and a protocol modified from Nussing et al⁵³. After RNP, cells were activated with 0.1 μg/ml gp33 (GenScript) and cultured with 10 ng/ml IL-2 (PeproTech) for 3 days in vitro before staining for CREM and analysis by flow cytometry.

qPCR

RNA was extracted following spleen homogenization using Trizol RNA extraction according to the manufacturer's protocol (Invitrogen). cDNA was synthesized using superscript IV transcriptase. For Adrb1 knockout validation in Adrb1^{fl/fl} Granzyme B^{Cre+} mice the following primers were used: fwd: CTCATCGTGGTGGGTAACGTG, SEQ ID NO: 29; rev: ACACACAGCACATCTACCGAA, SEQ ID NO: 30. The same primers were used for Adrb1 overexpression validation. For Crem knockdown validation the following primers were used: fwd: GCAAATGTGGCAGGAAAAAGT, SEQ ID NO: 31; rev: TGATCCAGCTACAGAAACCTGA, SEQ ID NO: 32. Hprt was used as housekeeping gene.

### Viral Titers

LCMV Fluorescence Focus Unit titration was performed seeding Vero cells at a density of 30 k cells/1001 in a 96 well flat bottom plate in DMEM+10% FBS+2% HEPES+1% Pen/Strep. On the next day, tissues were homogenized on ice, spun down at 1000 g for 5 min at 4° C. and supernatants or serum were diluted in 10-fold steps. Diluted samples were added to Vero cells and incubated at 37° C., 5% CO₂ for ~20 h. Subsequently, inocula were aspirated and wells were incubated with 4% PFA for 30 min at RT before washing with PBS. VL-4 antibody (BioXCell) was conjugated using the Invitrogen AF488 conjugation kit and added to the wells in dilution buffer containing 3% BSA and 0.3% Triton (Thermo Fisher Scientific) in PBS). Cells were incubated at 4° C. overnight before washing with PBS and counting foci under the microscope. FFU were calculated per this formula: FFU/ml=Number of plaques/(dilution*volume of diluted virus added to the plate).

scRNA Seq

For scRNA Seq of PDAC tumors 4 tumors each per treatment condition were pooled and stained for sorting. The following cell populations were sorted into RPMI supplemented with 20% fetal bovine serum: Thy1.2⁺ CD45.2⁺ (T cells), Thy1.2⁻ CD45.2⁺ (Non T cell leukocytes), YFP⁺ CD45.2⁻ (tumor cells). Sorted cells were spun down at 420 rcf at 4° C. for 4 minutes, resuspended in PBS+2% fetal bovine serum and mixed at 8 k Thy1.2⁺ CD45.2⁺, 10 k Thy1.2⁻ CD45.2⁺, 2 k YFP⁺ CD45.2⁻ for a total of 20 k cells per sample.

Samples were then loaded onto Chromium Chip G (10× Genomics) and partitioned into Gel Bead-in-Emulsions using a chromium controller (10× Genomics). Single cell RNA libraries were generated following the manufacturer's instructions of the Chromium Next GEM Single Cell 3′ Reagent Kit v3.1 (10× Genomics) followed by sequencing on a NovaSeq6000 at the NGS Core of the Salk Institute. Cellranger count was used to align reads to the mm10 genome and YFP. The resulting counts matrix was then processed using the R package scran⁵⁴ and outlying cells identified with quickPerCellQC (stats, percent_subsets=“subsets_Mito_percent”) were discarded. Data was log normalized using quickCluster, computeSumFactors and logNormCounts. The top 10% most variable genes were calculated using modelGeneVarByPoisson and getTopHVGs and then used in the PCA calculation using denoisePCA. The top 50 principal components were used to calculate a UMAP dimensional reduction using the runUMAP function (metric=“cosine”, min_dist=0.3 and n_neighbors=30). Leiden clustering was performed with igraph's cluster_leiden (with resolution=0.05) after running buildSNNGraph (with k=25). Cell types were annotated using SingleR⁵⁵ and the ImmGenData labels as a reference using default parameters. For further analysis the dataset was filtered on T cells according to the SingleR labels and variable genes, PCA and UMAP were calculated with the same settings described above. Clustering of T cells was performed using cluster_leiden with the resolution set to 1.8. For visualization, data imputation was performed using MAGIC⁵⁶ using the exact solver. To assign biological relevance to the T cell clusters, a heatmap of the median log normalized expression (using aggregateAcrossCells) of selected markers was generated and visualized using ComplexHeatmap⁵⁷. Differential genes for the clusters were identified by performing pairwise comparison of one cluster against all other clusters using the findMarkers function (test.type=“binom”, lfc=0.1, direction=“up”). Gene signatures for T cell exhaustion and T_{R}n cells were obtained from Wherry et al and Milner et al respectively^{45,58} in addition to the MSigDB hallmark gene sets. Gene signature scores were calculated using AUCell⁵⁹ (aucMaxRank set to 5%). To determine the effects of the treatment conditions, differential genes within the CD8⁺ T cells were obtained using findMarkers(test.type=“wilcox”, FDR<0.05) compared to the IgG treatment control. The annotation using CD8⁺ T cell exhaustion subsets was performed using PRJNA497086 as a reference for SingleR.

Analysis of Public RNA Seq and scRNA Seq Datasets

For the analysis of the Miller et al dataset, the raw data was downloaded from the SRA archive and aligned against the mm10 reference genome as described here: combine-lab.github.io/alevin-tutorial/2020/alevin-velocity /. After importing the count data into R, quality control, normalization and dimensional reduction was performed as described above. Gene expression was imputed using MAGIC and visualized on a UMAP. Annotation of the different exhaustion subsets was performed using canonical exhaustion markers (Tcf7, Cx3cr1, Cd101, Havcr2, Tox, Pded1, Tbx21, Slamf6, Entpd1).

To analyze human PDAC data, processed data was downloaded from GEO (GSE155698) and imported into R. Quality control, normalization, variance modelling and dimensional reduction were performed as described above. Clustering was performed using cluster_louvain with default settings. Cells were annotated using SingleR with BlueprintEncodeData as a reference dataset and subsequently subsetted to T cells. The upregulated genes in CD8⁺ T cells identified in our dataset between betablocker treated and IgG treated mice were defined as a gene signature and translated into human genes using BioMart. Scores were calculated using UCell⁶⁰ and patients were stratified according to their co-medication. CD8⁺ T cells were further divided into ADRB1^{high} and ADRB1^{low} cells based on an expression cutoff of 0.2 in MAGIC imputed ADRB1 expression. Plots with other exhaustion markers were generated using MAGIC imputed expression values. Statistics were calculated using a mixed model (R package lme4) based on normalized expression data with the following formula: expression ~ADRB1_high_or_low+(1|Sample). Human colorectal cancer data (GSE200997) was analyzed accordingly.

To analyze the human HIV data, count data was downloaded from GEO (GSE157829) and imported into R. Quality control was performed as described above. Normalization and variance modelling was done with scater's functions quickCluster (with the sample as a blocking factor), followed by computeSumFactors, logNormCounts, modelGeneVarByPoisson and getTopHVGs. Dimensional reduction was done as described above and the cells were annotated with SingleR and the MonacoImmuneData as a reference and subsequently filtered on T cell subsets. For visualization, MAGIC imputation was performed using the exact solver. UMAP dimensional reduction is colored by MAGIC imputed gene expression and the color scale is limited to the 95^{th} expression percentile.

To analyze previously published RNA Seq data (PRJNA497086), raw data was downloaded from the SRA archive, aligned against the mm10 genome using Salmon and imported into R. Expression data was normalized using the normTransform function from DESeq2 and a heatmap showing the mean normalized expression value was generated using ComplexHeatmap.

### Immunofluorescence Microscopy

Spleens were dissected 7, 14 or 31 days post LCMV-clone 13 infection and fixed in 1% paraformaldehyde (Santa Cruz Biotechnology) for 24 hours in 4° C. The fixed spleens were rinsed in PBS (Gibco), embedded in 4% low-melting agarose (Sigma Aldrich), then sectioned into 50 μm sections using a vibratome. The tissues were kept free-floating at 4° C. in Blocking Buffer (1% BSA (Sigma Aldrich) and 0.05% NaN3 (MP Biomedicals) in PBS). Prior to staining with antibodies of interest, the sections were additionally blocked in Fc antibody (Biolegend) to prevent non-specific binding for 24 hours at 4° C. then washed twice in Staining Buffer (2% BSA, 0.01% NaN3, and 0.5% Tween 20 (Thermo Fisher Scientific) in PBS). Primary antibodies were diluted in Staining Buffer and added to free floating sections for 48-72 hours at 4° C. Tissues were washed twice in Staining Buffer then secondary antibodies were diluted in Staining Buffer and added to free floating sections for 24 hours at 4° C. The tissues were washed twice in Staining Buffer, mounted onto Polysine Slides (Epredia) using prolong antifade mounting reagent (Invitrogen) then covered with No. 25 Glass Coverslips (Epredia).

Images were collected on a Zeiss LSM 880 Rear Port Laser Scanning Confocal Microscope with Airyscan FAST module using either 10× or 20× air objectives. To visualize proximity of WT P14⁺ and Adrb1 cKO P14⁺ cells to tyrosine hydroxylase (TH) expressing nerves, images from day 31 p.i. spleens were collected using a 10× objective with 0.7 zoom through a ~40 μm Z stack. To quantify the proximity of these cells, the images were analyzed in Imaris software version 9.9.0. Images were first flattened to a Maximum Z Projection then subjected to deconvolution and background subtraction. In Imaris, WT P14⁺, Adrb1 cKO P14⁺, and TH⁺ cells were separately defined as surfaces. To separate TH positive nerves from other TH expressing cells, a “sphericity filter” was applied to exclude rounded cells which do not exhibit nerve morphology. This was further refined by manually excluding TH cells with non-nerve morphology. Then the “Shortest Distance” function in Imaris was applied to quantify the shortest distance from WT P14⁺ and Adrb1 cKO P14⁺ to TH nerves across each image. This process was conducted in 6 total regions of interest across spleens from 2 different mice. To visualize proximity of WT P14⁺ CD101⁺ and WT P14⁺ CD101⁻ cells to TH, images from day 14 p.i. spleens were collected using a 20× objective across a 3×3 tiled scan area. In Imaris, WT P14 and CD101 signals were separately defined as “spots” and TH nerves were separately defined as surfaces. Subsequently, a new “spot” surface was defined as the spots where distance between P14 signal and CD101 signal is less than 1 μm to identify colocalized P14⁺ CD101⁺ spots. The “Shortest Distance” function in Imaris was applied to quantify the shortest distance from WT P14⁺ CD101⁺ and WT P14⁺ CD101⁻ cells to TH across each image. This process was conducted in 4 total regions of interest across spleens from 2 different mice.

### IBEX

Superfrost Plus microscopy slides (VWR, Cat #: 48311-703) were coated with 10 μl chrome alum gelatin to increase adhesion for IBEX. For a detailed IBEX protocol, see Radtke et al PNAS 2020, and Radtke et al Nature Protocols 2022^{61,62}. In brief, coated slides were dried in an oven at 60° C. for 60 min. OCT-embedded tumors were then sectioned on a cryostat in 40 μm thick sections onto the coated slides, and dried in an oven at 37° C. for 60 min. Sections were blocked and permeabilized using 0.3% Triton X-100 with Fc block for 1 h at room temperature (RT), and washed in PBS. Tissue sections were next incubated with IBEX cycle 1 antibodies diluted in PBS for 3 h at RT. After washing 3× in PBS (5 min per wash) at RT, the secondary antibody for TH staining was added for 1 h at RT. After repeating the washes, samples were stained with Hoechst and mounted in Fluoromount-G (SouthernBiotech, Cat #: 0100-01), which was allowed to cure for a minimum of 1 h at RT. All imaging was performed using No. 1.5 cover glass (VWR, Cat #: 48393-241) on a Leica SP8 upright confocal microscope. After cycle 1 imaging was completed, the cover glass was lifted via soaking in PBS for at least 1 h. fluorophores were bleached with lithium borohydride, and re-stained with cycle 2 antibodies by repeating the protocol above.

### Human NSCLC Tissue Microarrays and Multiplexed Immunofluorescence Staining

Samples included retrospectively collected formalin-fixed, paraffin-embedded (FFPE) tumor samples from a cohort of 164 stage I-IV NSCLCs represented in tissue microarray (TMA) format. The TMAs were constructed as previously reported⁶³ by selecting areas containing viable tumor cells and stromal elements on Hematoxylin & Eosin-stained preparations (as assessed by a pathologist) and without enriching for specific tumor regions, tissue structures or immune-related features. Multiplexed fluorescence staining in human FFPE tumor tissue sections was conducted using a previously reported protocol based on the use of isotype specific antibodies⁶³. Briefly, consecutive 4-μm thick TMA sections containing the primary NSCLCs were deparaffinized and rehydrated, antigen retrieval was performed with EDTA solution (96° C. pH 8.0 for 1 h). Endogenous peroxidases were blocked using a 1% hydrogen peroxidase in methanol solution (RT, 30 min). Non-specific antigens were blocked using 0.3% bovine serum albumin in TBST. Each section was stained using a different and previously standardized multiplexed immunofluorescence panel. The first panel mapped exhausted T cells and included the markers DAPI, CD8, LAG3. PD-1 and TIM3; the second panel analyzed TH expression and included the markers DAPI, TH and cytokeratin (CK). Primary antibodies for CD8 were incubated for 1 h at RT for the first panel. For the localized TH panel, primary TH antibody was incubated overnight at 4° C. and a conjugated Pancytokeratin antibody was then incubated at RT for 1 h. Secondary antibodies were subsequently incubated for 1 h at RT. Biotin beading (PerkinElmer) was done before labeling with a streptavidin-ligated Cy7 fluorophore (Invitrogen). Residual HRP activity between incubations with secondary antibodies was eliminated by incubation with a solution containing benzoic hydrazide (0.136 mg) and hydrogen peroxide (50 μl). DAPI (BD systems) was used to stain nuclei. Two control/index TMAs were stained at the same time to ensure staining reproducibility. Stained slides were scanned in a Vectra Polaris microscope (PerkinElmer), the images obtained were analyzed using the automated image analysis software, InForm (V.2.4.8, PerkinElmer). For each sample, a tissue and cell segmentation algorithm were created using 10 representative spots to train the software to define background, tumor and stromal compartments based on the marker staining. Then, the nuclear DAPI staining was used to identify and segment individual cells. Next, the software was trained to recognize cell subtypes based on expression or absence of the markers TH, CD8, LAG3, PD-1, TIM3 and CK for tumor epithelial cells and absence of this marker for stromal cells. The algorithm was applied to all images of each individual case. The images were visually reviewed to ascertain reproducibility and those cells with suboptimal phenotyping were excluded.

### Western Blot

For assessment of PLCγ1 phosphorylation, murine CD8⁺ T cells were transduced with either empty vector or Adrb1 overexpression retrovirus and sorted on GFP⁺ cells the day after transduction. Cells were expanded in the presence of IL-2. Before TCR stimulation, cells were starved in serum-free RPMI. Cells were preincubated with 10 μM noradrenaline if indicated, followed by TCR stimulation with 0.1 μg/ml gp33 for 5 minutes and lysed on ice for 30 min immediately after TCR stimulation (Lysis buffer: 50 mM Tris/HCL pH 7.6, 150 mM NaCl, 5 mM MgCl₂, 0.1% NP40, supplemented fresh with 1× cOmplete™, EDTA-free Protease Inhibitor Cocktail (Sigma 11873580001), 1 mM DTT, 1 mM Benzamidine, Phosphatase Inhibitor Cocktail 2 and 3 (Sigma P5726 and P0044)). Lysates were spun down and supernatant stored at −80° C. Protein concentration was determined using a BCA assay and 15-20 μg protein were used for Western Blot. Western Blot was performed using the NuPAGE electrophoresis system. Western Blots were stained with Phospho-PLCγ1 (Tyr783) (D6M9S) Rabbit mAb #14008 (Cell Signaling Technology) and developed using Amersham™ ECL™ Western Blotting Detection Reagents (Sigma GERPN2134). Quantification was performed using ImageJ.

### Immunohistochemistry

Tumor tissues were fixed in 10% neutral buffered formalin (LabChem LC146702) for 18-24 hours. Fixed tissues were rinsed twice with 1×PBS (Gibco) to remove residual formalin, followed by sequential dehydration in 50% (4 hours), 75% (overnight), 95% EtOH (overnight) at 4° C. on an orbital shaker. Dehydrated samples were stored in 100% EtOH and submitted for paraffin embedding, sectioning, and immunohistochemistry (IHC). For IHC, tissue was baked at 60° C. before clearing and rehydrating through successive alcohols (3× Xylene, 2×100% EtOH, 2×95% EtOH, 2×70% EtOH, diH2O) followed by antigen retrieval in Antigen Unmasking Solution (Citrate Based, pH6) (Vector, H-3300) at 95° C. Staining was performed on Intellipath Automated IHC Stainer (Biocare): Peroxidase block Bloxall (Vector, SP-6000), Protein block Blotto (Thermo, PI37530), anti-Ki67 primary antibody (Rabbit, GeneTex, 16667, 1:50), anti-rabbit HRP Polymer (Cell IDX, 2RH-100), DAB (brown) Chromogen (VWR, 95041-478), Mayer's Hematoxylin (Sigma, 51275-500 ml) before washing, dehydrating, clearing, and mounting with xylene based mountant. Slides were scanned and data analyzed using QuPath version 0.4.3.

### Antibodies Used

Ghost Dye Red 780 Tonbo Biosciences #13-0865 Dilution 1:1000; TCF1 PacificBlue (C63D9) Cell Signaling #9066S Dilution 1:200; CX3CR1 BV510 (SA011F11) Biolegend #149025 Dilution 1:200; Tim3 BV605 (RMT3-23) Biolegend #119721 Dilution 1:200; PD-1 BV785 (29F.1A12) Biolegend #135225 Dilution 1:200; Thy1.1 A488 (OX-7) Biolegend #202506 Dilution 1:400; Thy1.2 PerCP Cy5.5 (30-H12) Biolegend #105338 Dilution 1:400; CD101 PeCy7 (Moushil01) eBioscience #25-1011-80 Dilution 1:100; Slamf6 A647 (13G3) BD #561547 Dilution 1:200; CD8a BV711 (53-6.7) Biolegend #100747 Dilution 1:200; CD127 BV421 (A7R34) Biolegend #135024 Dilution 1:50; CD44 A700 (IM7) Biolegend #103026 Dilution 1:200; CX3CR1 A488 (SA011F11) Biolegend #149022 Dilution 1:200; KLRG1 PeCy7 (2F1) Biolegend #138416 Dilution 1:200; TOX e660 (TXRX10) Thermo Fisher Scientific #50-6502-82 Dilution 1:100; CXCR6 PE (SA051D1) Biolegend #151104 Dilution 1:200: CD39 Pe-Cy7 (24DMS1) eBioscience #25-0391-82 Dilution 1:200: PD-1 BV605 (29F.1A12) Biolegend #135219 Dilution 1:200; Tim3 BV421 (RMT3-23) Biolegend #119723 Dilution 1:200: CD45 BV711 (30-F11) Biolegend #103147 Dilution 1:200; IFNg BV421 (XMG1.2) Biolegend #505829 Dilution 1:200: TNF PE (MP6-XT22) Biolegend #506306 Dilution 1:100; Granzyme B PeCy7 (QA16A02) Biolegend #372214 Dilution 1:200; Thy1.2 BV421 (30-H12) Biolegend #105341 Dilution 1:400: Thy1.1 BV421 (OX-7) Biolegend #202529 Dilution 1:400; Ly5.1 BV421 (A20) Biolegend #110731 Dilution 1:200; Ly5.2 PerCP (104) Biolegend #109828 Dilution 1:200; Tim3 PeCy7 (RMT3-23) Biolegend #119716 Dilution 1:200; KLRG1 FITC (2F1/KLRG1) Biolegend #138410 Dilution 1:200; CD45 PerCP Cy5.5 (30-F11) Biolegend #103132 Dilution 1:200; Tyrosine hydroxylase unconjugated (polyclonal) EMD Millipore #AB152 Dilution 1:1000; CD8a PeCy7 (53-6.7) Biolegend #100722 Dilution 1:200; gp33 BV421 NIH Tetramer Facility Dilution 1:100; gp33 A488 NIH Tetramer Facility Dilution 1:100; gp33 A647 NIH Tetramer Facility Dilution 1:100; GFP A488 (polyclonal) Invitrogen #A-21311 Dilution 1:1000; CREM FITC (polyclonal) Biorbyt #orb7831 Dilution 1:100; cAMP unconjugated (EP8471) Abcam #ab134901 Dilution 1:100; ADRB1 PE (polyclonal) Bioss #BS-0498R-PE Dilution 1:100; Anti-rabbit IgG (H+L) F(ab′)2 Fragment A647 Cell Signaling #4414S Dilution 1:1000; CD101 PE (Moushil01) Invitrogen #12-1011-82 Dilution 1:50; Ly51 PE (A20) Biolegend #110708 Dilution 1:50: B220 BV421 (RA3-6B2) BD #562922 Dilution 1:50; F4/80 A647 (BM8) Biolegend #123121 Dilution 1:50: Goat anti-Rabbit IgG (H+L) A647 (polyclonal) Invitrogen #A-21245 Dilution 1:200; Donkey anti-Sheep IgG (H+L) A647 (polyclonal) Invitrogen #A-21448 Dilution 1:200; Tyrosine hydroxylase unconjugated (polyclonal) Invitrogen #PA1-4679 Dilution 1:1000: TruStain FcX™ (anti-mouse CD16/32) Biolegend #101320 Dilution 1:500; huCD8 V500 (SKI) BD Biosciences #561617 Dilution 1:200; huCD3 A700 (UCHTI) Biolegend #300424 Dilution 1:200; huPD-1 BV421 (EH12.2H7) Biolegend #329920 Dilution 1:200; huCD39 PeCy7 (A1) Thermofisher #25-0399-42 Dilution 1:200; huEOMES APCe780 (WD1928) Thermofisher #47-4877-42 Dilution 1:200; huTBET PerCP Cy5.5 (4B10) Biolegend #644805 Dilution 1:200; LIVE/DEAD™ Fixable Red Dead Cell Stain Kit for 488 nm excitation Thermofisher #L23102 Dilution 1:1000; CD4 eF570 (RM4-5) invitrogen #41-0042-82 Dilution 1:50; Tyrosine hydroxylase unconjugated (polyclonal) Novus biologicals #NB300-110 Dilution 1:50; Donkey anti-sheep AF594 invitrogen #A11016 Dilution 1:500; CD3 AF647 (17A2) BioLegend #100209 Dilution 1:50; B3 tubulin AF488 (TUJ1) BioLegend #801203 Dilution 1:50; CD31 AF594 (MEC13.3) BioLegend #102520 Dilution 1:50; CD39 AF647 (Duha59) BioLegend #143808 Dilution 1:50; CD45 AF532 (30-F11) invitrogen #58-0451-82 Dilution 1:50; PD-1 AF647 (RMP1-30) BioLegend #109118 Dilution 1:50; Phospho-PLCγ1 (Tyr783) (D6M9S Rabbit mAb) Cell Signaling Technology #14008 Dilution 1:4000; beta-Actin (8H10D10 Mouse mAb) Cell Signaling Technology #3700 Dilution 1:10000; Ki67 (SP6) GeneTex #GTX16667 Dilution 1:50; CD8 unconjugated (C8/144B) DAKO #M7103 Dilution 1:250; Anti mouse IgG1 HRP (M1-14D12) ThermoFisher #18-4015-82 Dilution 1:100; PD-1 unconjugated (EH33) CST #43248 Dilution 1:200; Anti mouse IgG2a HRP (polyclonal) Abeam #ab97245 Dilution 1:200; LAG3 unconjugated (D2G40) CST #15372 Dilution 1:500; anti-Rabbit IgG Poly-HRP Leica #PV6119 Dilution N/A; TIM3 unconjugated (D5D5R) CST #45208 Dilution 1:500; TH unconjugated (E2L6M) CST #58844 Dilution 1:500; Pan cytokeratin A488 (AE1/AE3) ThermoFisher #53-9003-82 Dilution 1:100; anti-PD-1 (RMP1-14) Leinco #P362 Dose 100 μg; anti-CTLA-4 (9H10) Leinco #C1614 Dose 100 μg: IgG2A (C1.18.4) Leinco #I-118 Dose 100 μg; IgG2B (MPC-11) Leinco #I-119 Dose 100 μg; anti-mouse CD8a (2.43) Leinco #C380 Dose 200 μg.

### Statistics

FlowJo v10 (FlowJo LLC, USA) was used for analysis of cytometric data. Statistical analysis was performed using GraphPad version 9 (Prism Software Inc., USA) and R version 4.2.0. Statistical tests used are indicated in the figure legends. A p value<0.05 was considered significant. If not specified otherwise, **** indicates a p value<0.0001, ***<0.001, **<0.01. *<0.05. Unless otherwise specified, mean±SEM is indicated in scatter plots. Boxplots show median. The lower and upper hinges correspond to the first and third quartiles. The upper whisker extends from the hinge to the largest value no further than 1.5*IQR from the hinge. Two-sided statistical tests were used. Original data plots are depicted using FlowJo as 2% contour plot with outlier setting.

### Data Availability

The following published datasets were used in addition to data generated: PRJNA497086, GSE122713, GSE157829, GSE155698, GSE200997. The mouse reference genome mm10 and the human reference genome GRCh38 were used for RNA-Seq and scRNA-Seq analysis.

Code Availability The R scripts that support the findings disclosed herein are available from the github/aglobig.

### Example 2

### Exhausted T Cells Upregulate ADRB1

This example illustrates that exhausted CD8+ T cells upregulate the adrenergic receptor ADRB1 and localize near sympathetic nerves.

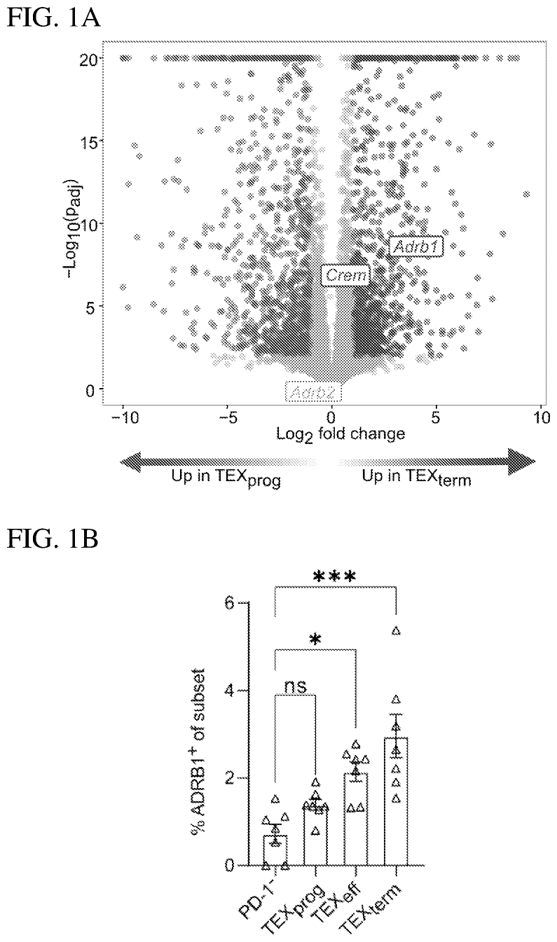

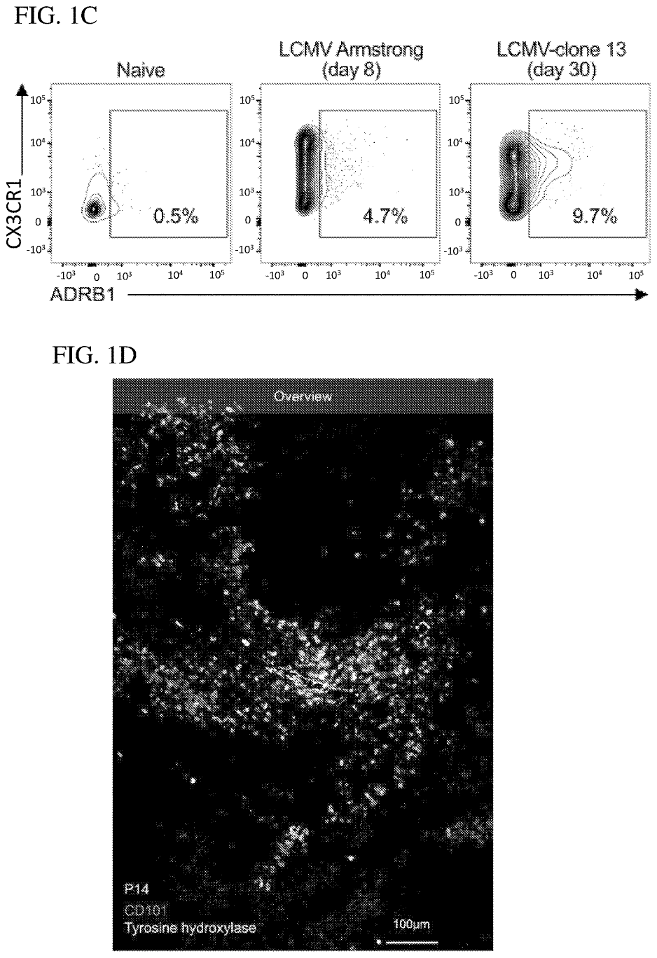

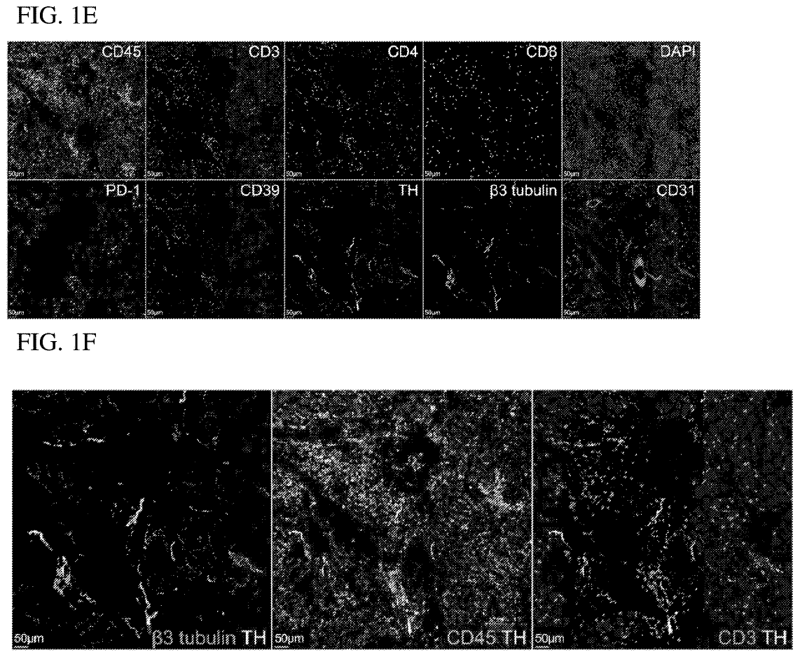

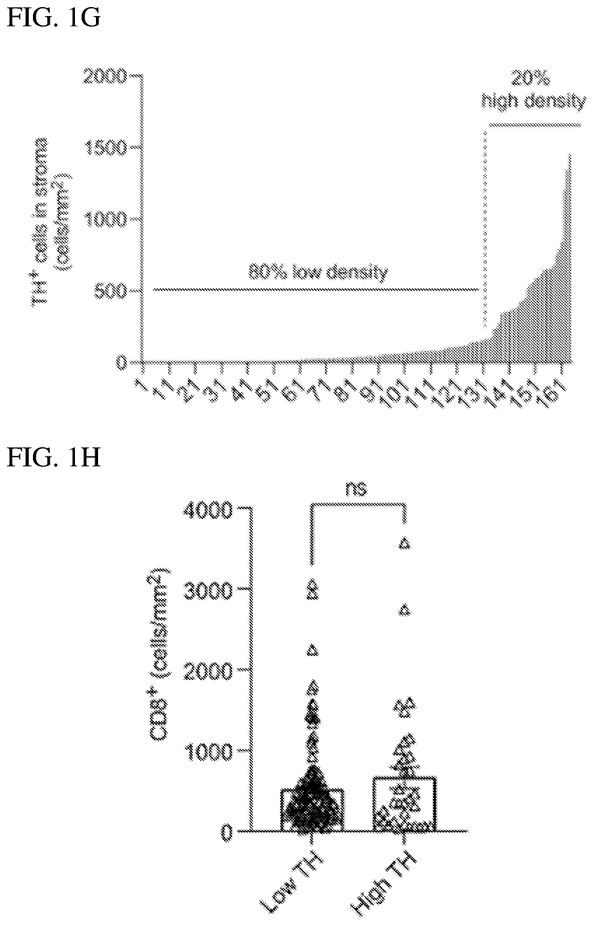

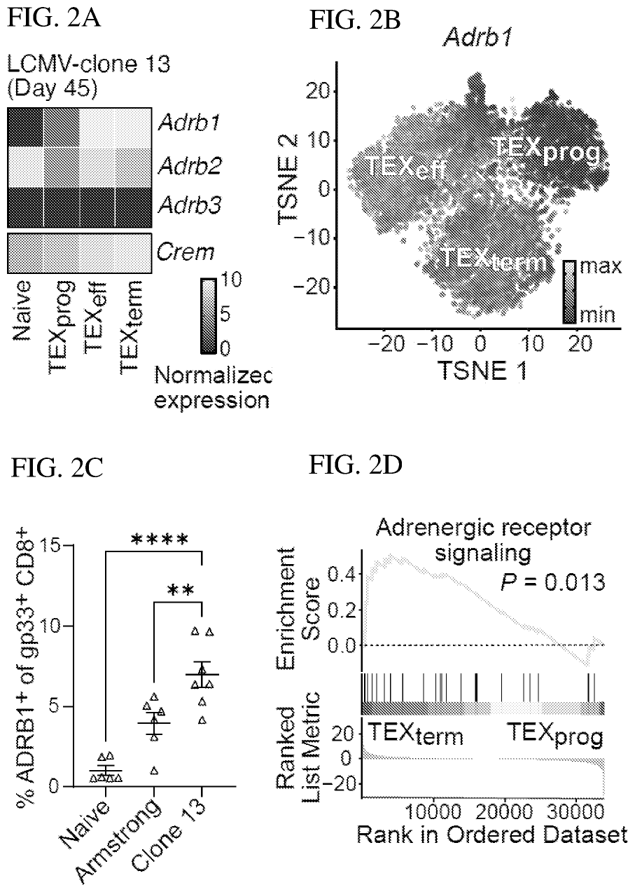

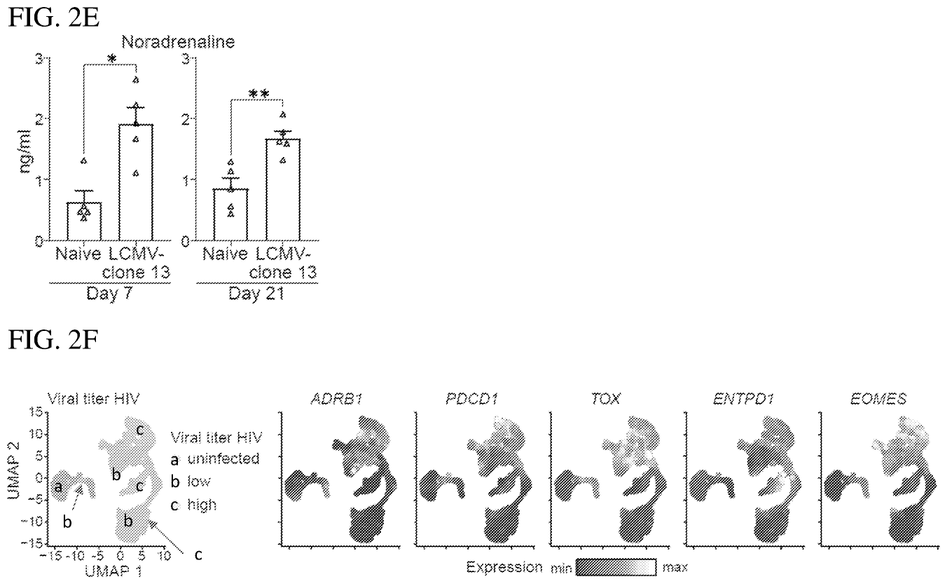

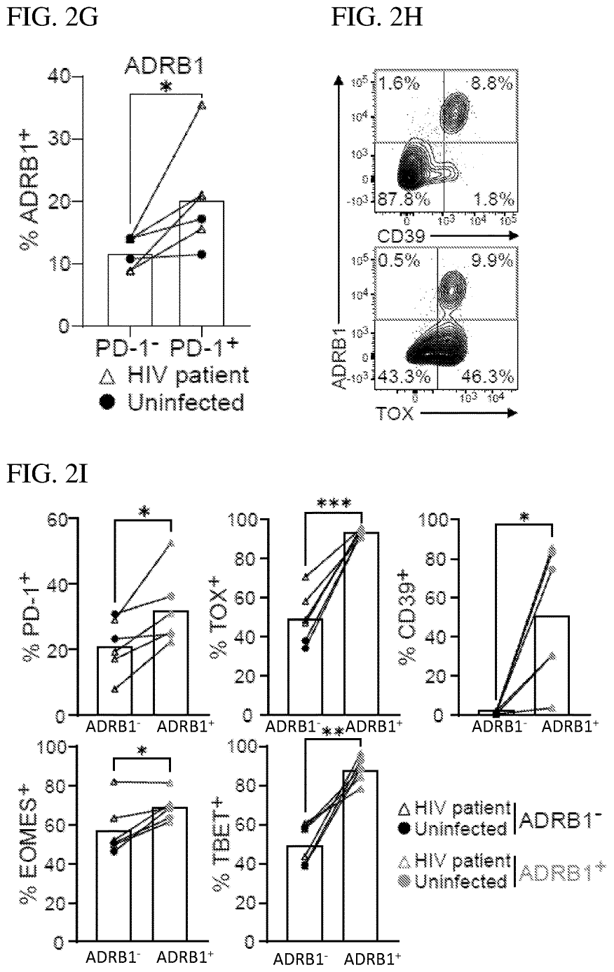

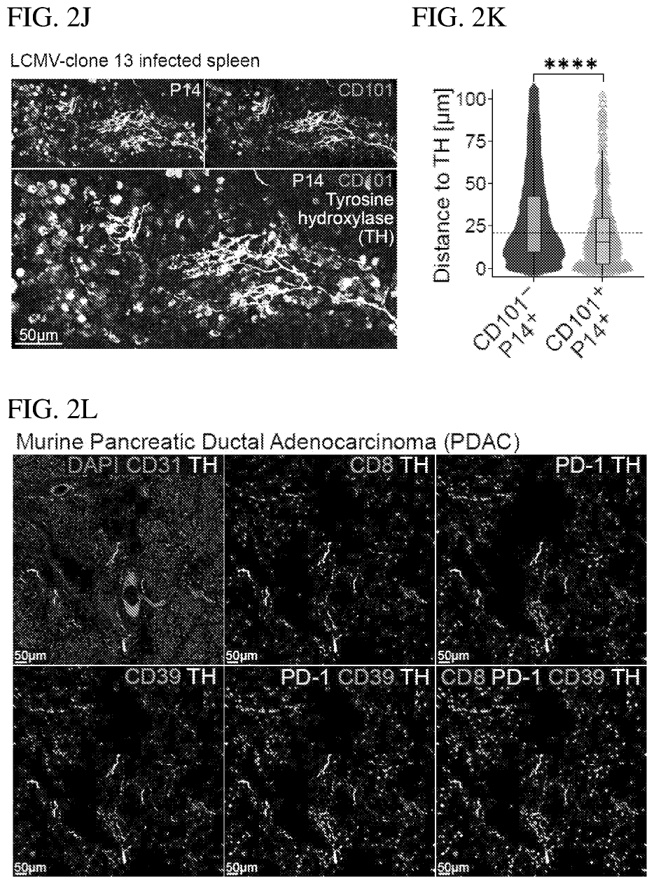

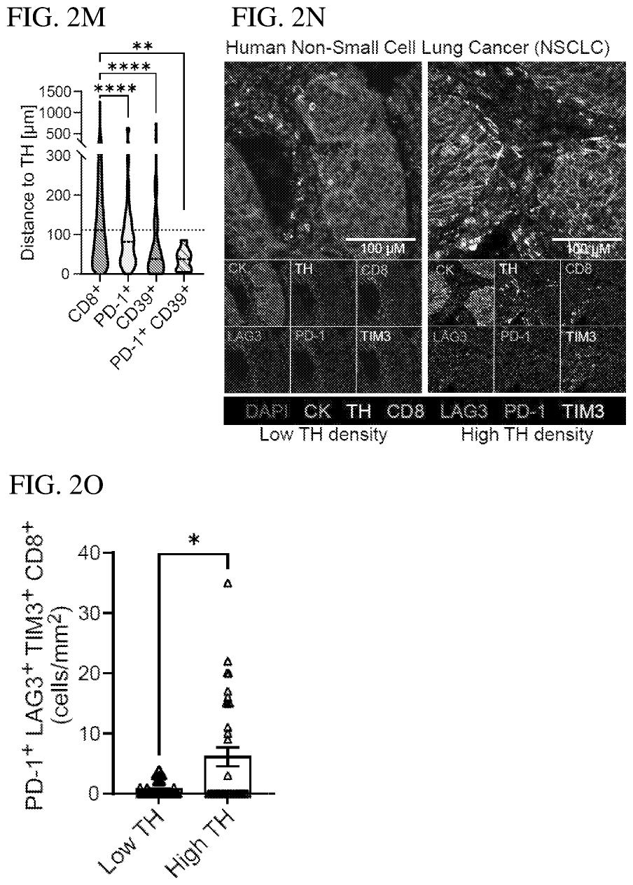

Novel physiological mechanisms regulating the differentiation from progenitor to exhausted states were identified by querying genes upregulated in each state in T cell receptor (TCR) transgenic P14⁺ cells that recognize the gp33-41 epitope of lymphocytic choriomeningitis virus (LCMV) during chronic murine LCMV-clone 13 infection (FIG. 1A). Among the genes selectively upregulated in terminally exhausted P14⁺ CD8⁺ T cells was the β-adrenergic receptor Adrb1, a C-protein coupled receptor that responds to the endogenous stress hormones adrenaline and noradrenaline. The expression of Adrb1 was specifically increased in the more differentiated TEX_{eff} and exhausted TEXₜₑᵣₘ CD8⁺ T cell subsets compared to TEX_{prog} and naïve cells during chronic infection (FIGS. 1A, 1B. 2A, 2B). The other β-adrenergic receptors Adrb2 and Adrb3 were not similarly increased in exhausted CD8+ T cells. Adrb2 was most highly expressed in naïve CD8+ T cells and Adrb3 had no expression in the examined T cell subsets (FIG. 2A). Adrb1 became the predominantly expressed adrenergic receptor on TEX cells during the transition of TEX_{prog} to TEXₜₑᵣₘ or TEX_{eff}, indicating a specific role for Adrb1 in T cell exhaustion. Using MHC-I tetramers, it was confirmed that endogenous virus-specific CD8⁺ T cells in mice with chronic LCMV infection expressed higher levels of ADRB1 compared to activated CD8⁺ T cells in acute infection or to naïve CD8⁺ T cells (FIGS. 1C, 2C). Pathway analysis of exhausted CD8⁺ T cells revealed an overall enrichment of the adrenergic receptor signaling pathway in TEXₜₑᵣₘ compared to TEX_{prog} (FIG. 2D). Consistent with this, mice infected with LCMV-clone 13 also exhibited significantly higher systemic levels of the stress hormone noradrenaline (FIG. 2E).

In humans, higher expression of ADRB1 was also observed by CD8⁺ T cells from HIV patients with high viral titers, and ADRB1 expression colocalized with exhaustion markers (PDCD1, TOX, ENTPD1 (encoding CD39), and EOMES) in UMAP dimensional reduction (FIG. 2F). Human peripheral blood CD8⁺ T cells from both HIV⁺ and HIV donors were next examined and while the sample numbers were too small to distinguish between the two groups it was clear that human PD-1⁺ CD8⁺ T cells expressed higher levels of ADRB1 than their PD-1-counterparts (FIG. 2G). Closer examination revealed that ADRB1-expressing human CD8⁺ T cells expressed higher levels of the exhaustion markers PD-1, TOX, CD39, EOMES and TBET corresponding to cells with an activation/exhaustion phenotype (FIGS. 2H, 2I)^{45,24}. Moreover, ADRB1 signals via activation of adenylate cyclase thereby increasing intracellular cAMP levels. Of note, the expression pattern of the transcription factor cAMP-responsive element modulator (Crem) mirrored the expression of Adrb1 in exhausted T cell subsets in chronic viral infection (FIGS. 1A, 2A). CREM is a transcription factor downstream of adrenergic receptor signaling in the cAMP pathway²⁵ and is a regulator of CD4⁺ T cell exhaustion²⁶.

The expression of ADRB1 on TEX cells raised the question of where CD8⁺ T cells may encounter catecholamines in tissues. The association of exhausted CD8⁺ T cells with sympathetic nerves was examined in three settings: (1) First, chronic LCMV infected spleens were imaged and P14⁺ CD8⁺ T cells were found expressing CD101, a distinguishing trait of TEXₜₑᵣₘ cells, located closer to tyrosine hydroxylase-expressing (TH⁺) sympathetic nerves than their CD101 counterparts (FIGS. 1D, 2J, 2K) (2) Similarly, in a model of murine pancreatic cancer PD-1⁺ CD39⁺ CD8⁺ T cells were located closer to TH⁺ sympathetic nerves than the total CD8⁺ tumor infiltrating lymphocytes (TILs) (FIGS. 1E, 1F, 2L, 2M). (3) 164 human non-small cell lung cancer (NSCLC) samples were examined and substantially elevated TH^{high} staining was found in ~20% of the samples (FIGS. 1G, 2N). Comparison of the TH^{high} vs. TH^{low}-density NSCLC samples revealed that although there was no difference in the total density of CD8⁺ TILs between the two groups (FIG. 1H), the TH^{high} tumors were significantly enriched with PD-1⁺ LAG3⁺ TIM3⁺ CD8⁺ TILs (FIGS. 2N, 2O). These findings reveal that TEX-like cells have close range interactions with sympathetic nerves in both chronically virally infected and malignant tissues, supporting the idea that TEX cells can respond to physiological stress signals in tissues.

### Example 3

### Adrb1 Activity Promotes Exhaustion

This example illustrates that Adrb1 activity induces T cell exhaustion.

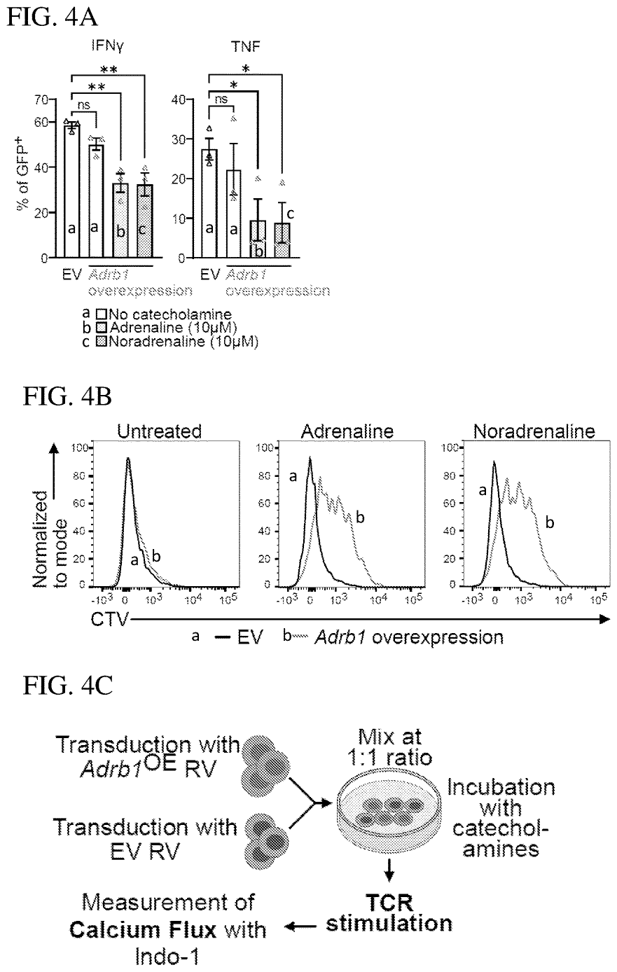

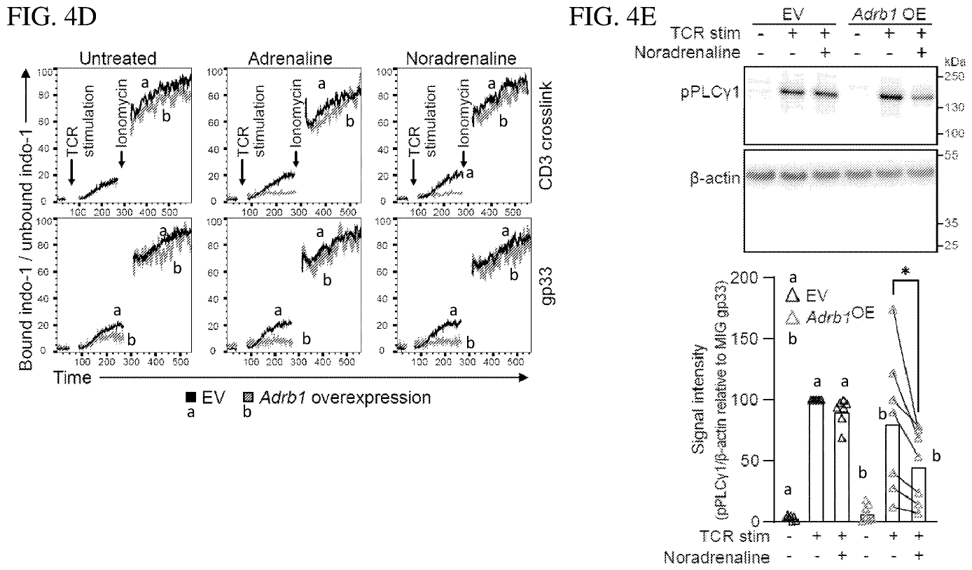

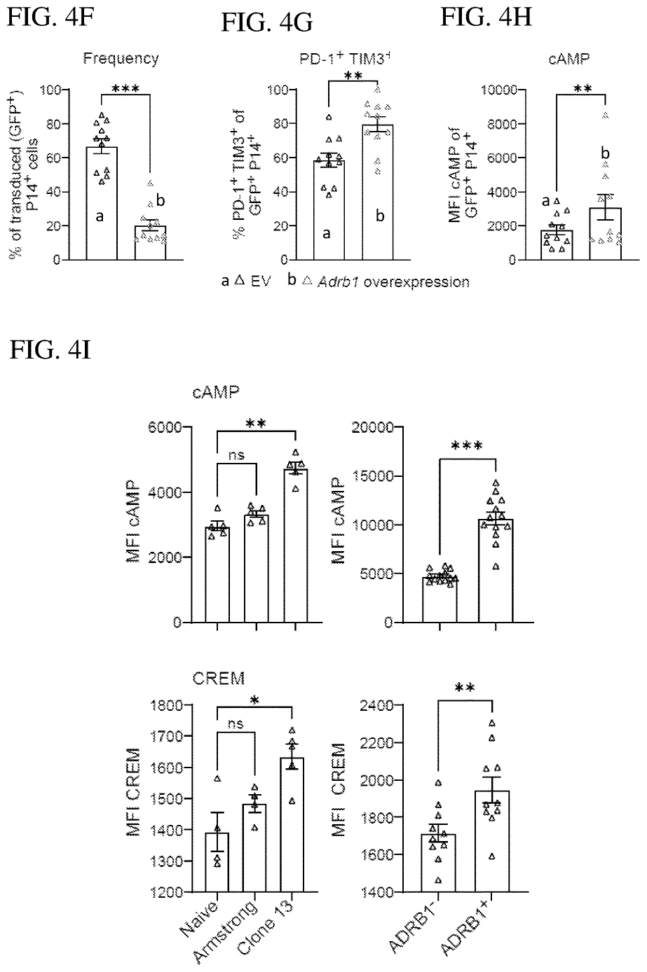

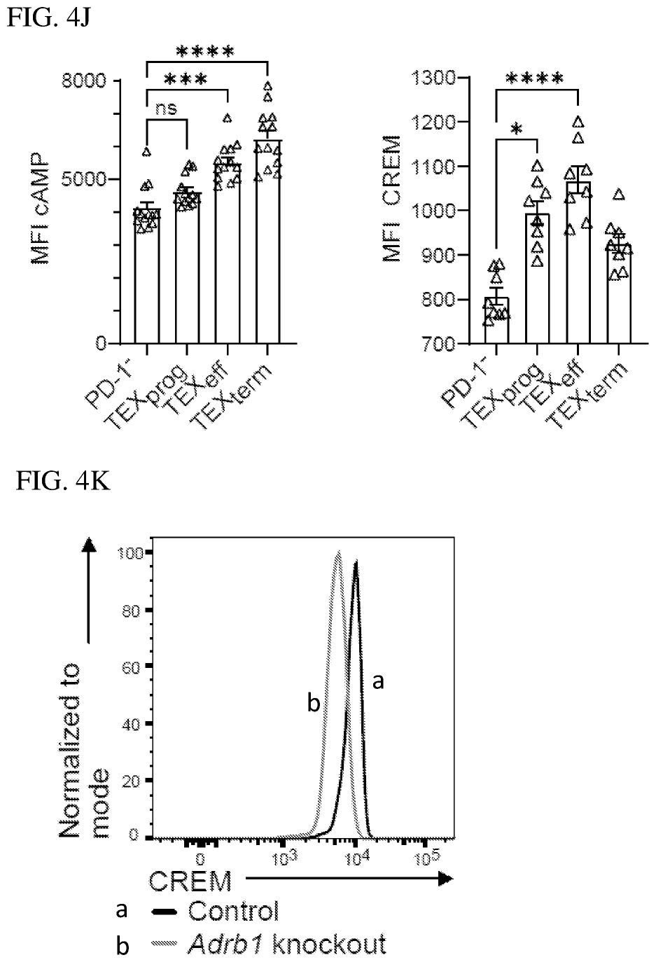

As CD8⁺ T cells become exhausted, their TCR signaling capacity declines due to increased expression of inhibitory receptors and other negative feedback pathways²⁷⁻²⁹. To further elucidate the effects of high Adrb1 expression on CD8⁺ T cells, Adrb1 was overexpressed (OE) using retrovirus (RV) in CD8⁺ T cells and it was found that this did not kill activated CD8⁺ T cells, but rather impaired proliferation and cytokine production upon antigen-specific TCR stimulation (FIGS. 3A-3E, 4A, 4B)—two characteristic features of T cell exhaustion⁴. It was hypothesized that the reduced cytokine production by Adrb1 overexpressing CD8⁺ T cells upon antigen stimulation might be due to impaired TCR signaling. Indeed, in the presence of catecholamines, Adrb1 OE CD8⁺ T cells were unable to flux calcium as well as empty-vector (EV) control cells when the TCR was activated by anti-CD3 or virus-specific peptides, in accord with the reduced phosphorylation of PLCγ1 in the Adrb1 OE cells (FIGS. 4C-4E). Next, to investigate the effects of Adrb1 OE on the differentiation of CD8⁺ T cells in vivo, Adrb1 OE and control EV TCR-transgenic P14⁺ cells were transferred into recipient mice that were infected with LCMV-clone 13. At d7 post infection (p.i.), Adrb1 OE P14⁺ cells were present at reduced frequency compared to control EV cells, consistent with impaired proliferation of Adrb1 OE cells in vivo (FIG. 4F). Adrb1 OE also augmented the frequency of PD-1⁺ TIM3⁺ P14⁺ cells and amounts of cAMP relative to control EV cells (FIGS. 4G, 4H), supporting a role for Adrb1 in driving TEX differentiation. Next, virus-specific CD8⁺ T cells were analyzed in chronically infected mice and it was found that they contain higher amounts of cAMP than those in acutely infected mice or naïve CD8⁺ T cells, which was more pronounced in ADRB1⁺ CD8⁺ T cells (FIG. 41). CREM expression mirrored the increase of cAMP in ADRB1⁺ CD8⁺ T cells (FIG. 41) and correspondingly, both CAMP and CREM levels were increased in exhausted CD8⁺ T cell subsets (FIG. 4J). Lastly, knocking out Adrb1 in in vitro activated P14⁺ cells decreased CREM expression (FIG. 4K). Taken together, these results reveal an ADRB1-CAMP-CREM signaling axis in CD8⁺ T cells and indicate that elevated expression of Adrb1 suppresses CD8⁺ TCR activity and promotes exhaustion via cAMP-CREM signaling.

### Example 4

### Adrb1 Knockout Prevents Terminal Exhaustion

This example illustrates that Adrb1 knockout prevents terminal exhaustion of antigen-specific CD8+ T cells in chronic viral infection and synergizes with ICB.

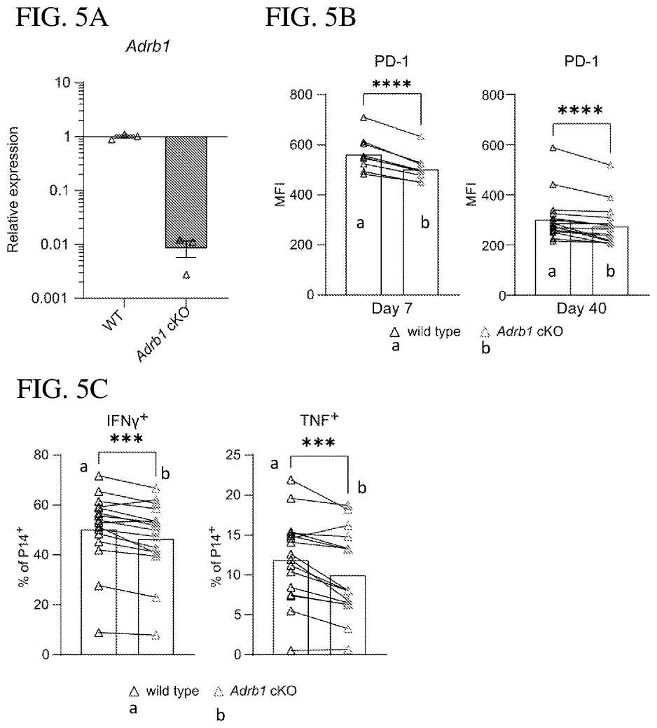

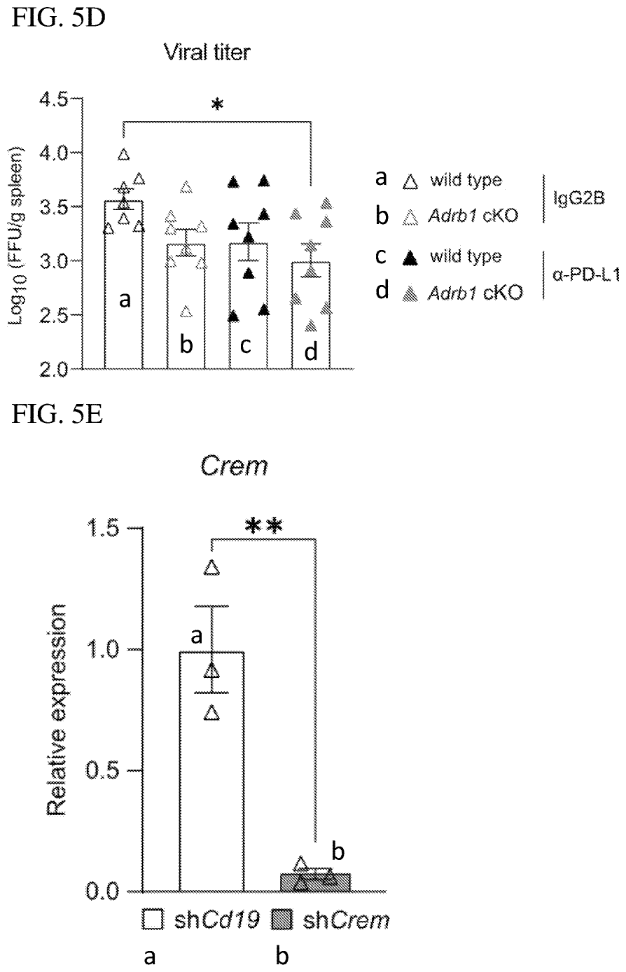

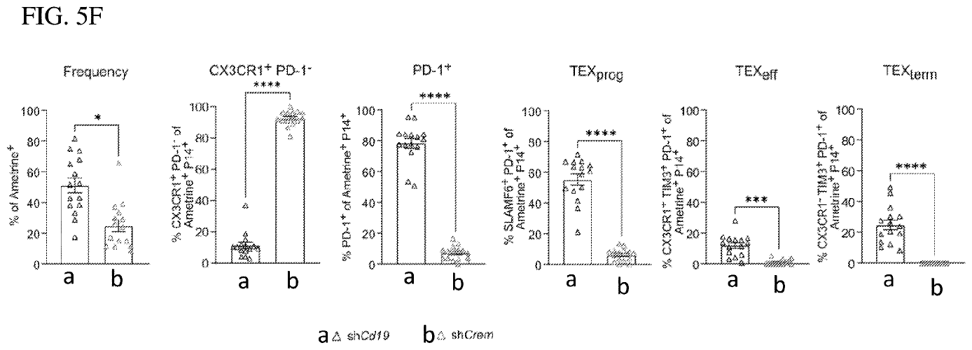

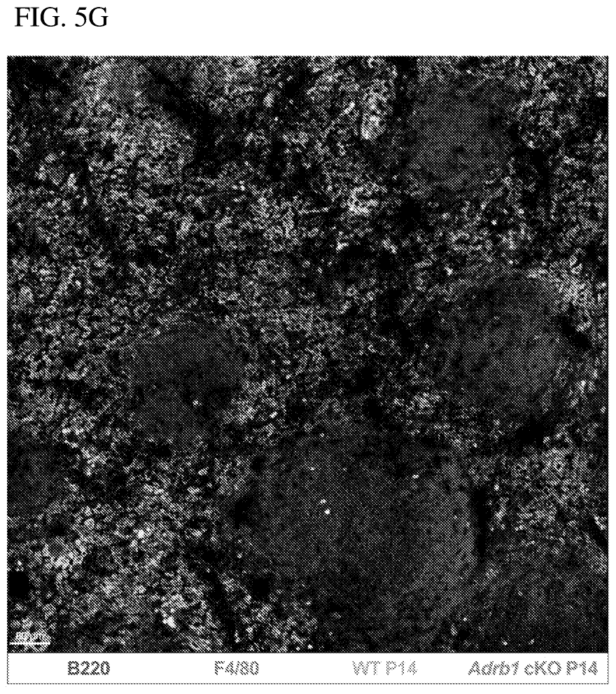

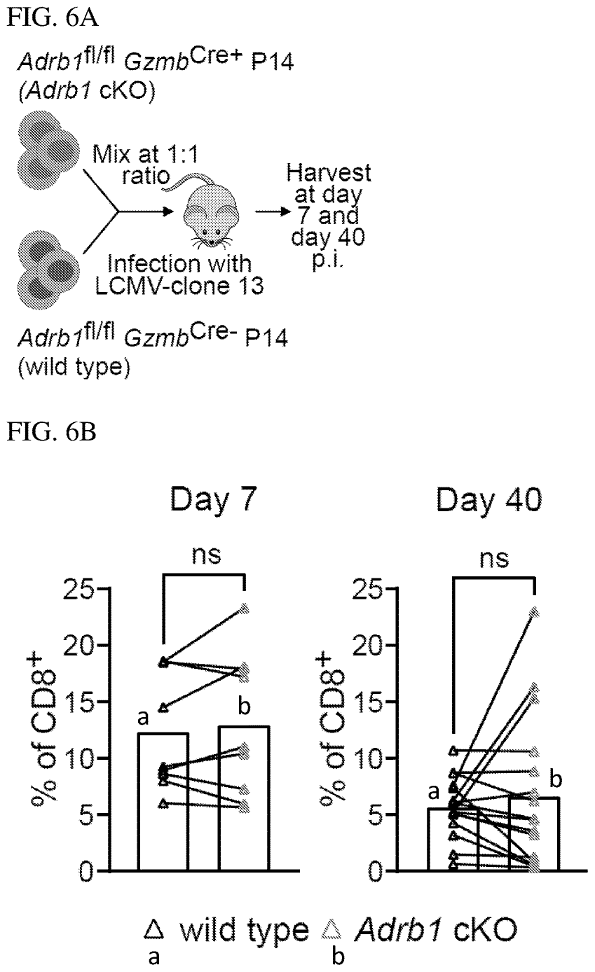

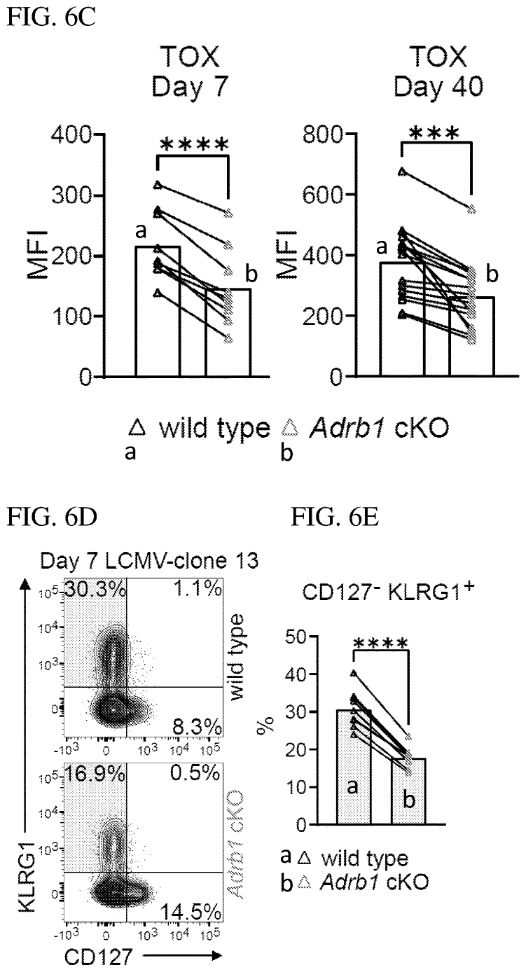

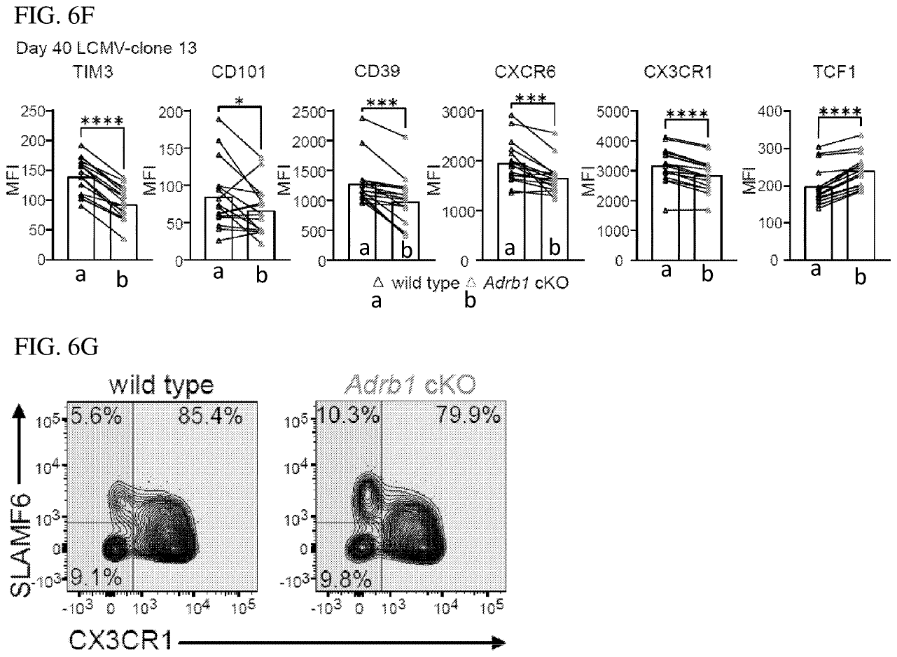

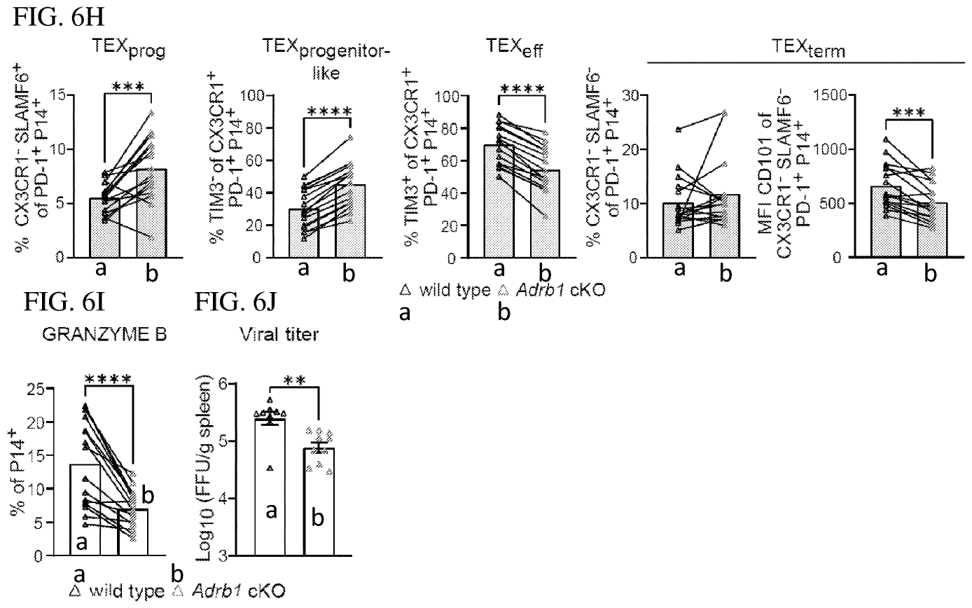

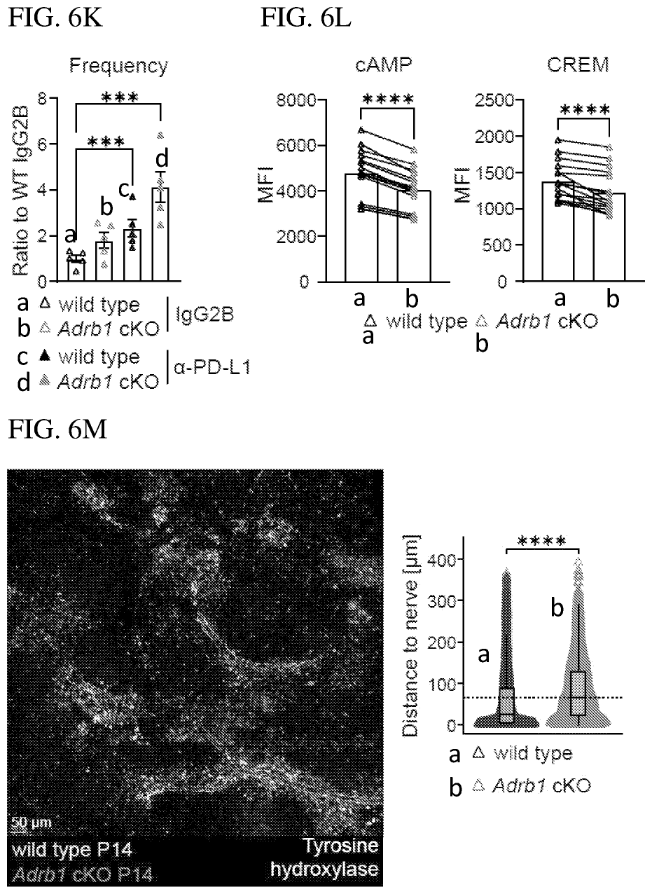

To examine the role of Adrb1 in antigen-specific CD8⁺ T cell differentiation during chronic viral infection more closely, Adrb11/fl mice were crossed to TCR transgenic P14⁺ Granzyme B (GzmB)^{Cre+} mice, hereafter referred to as Adrb1 cKO (conditional knockout) P14⁺ cells (FIG. 5A). Littermate controls were P14⁺ cells that contain Adrb1^{fl/fl}, but not GzmB^{Cre}, hereafter referred to as wild type (WT) P14⁺ cells. P14⁺ CD8⁺ T cells lacking Adrb1 and the littermate WT control cells were co-transferred at a 1:1 ratio into WT B6 recipient mice that were subsequently infected with LCMV-clone 13 and examined at d7 and d40 p.i. (FIG. 6A). The overall frequency, and hence, numbers of Adrb1 cKO cells were similar to that of WT controls at d7 and d40 p.i. (FIG. 6B). While the expression of PD-1 was comparable between Adrb1 cKO and WT cells (FIG. 5B), the expression of TOX was substantially reduced in Adrb1 cKO cells compared to WT control cells (FIG. 6C). At day 7 p.i., Adrb1 cKO cells differentiated less into CD127-KLRG1⁺ terminal effector-like cells^{31,32} (FIGS. 6D, 6E). As the infection progressed, Adrb1 cKO cells showed fewer features of terminally exhausted CD8⁺ T cells including lower expression of TIM3, CD101, CD39 and CXCR6 and in turn expressed more TCF1, a transcription factor characteristic of progenitor cells (FIG. 6F). Closer inspection at d40 p.i. revealed that compared to WT cells, the virus-specific Adrb1 ckO P14⁺ cell population contained higher frequencies of progenitor- or stem-like cells that could be divided into two subsets of CX3CR1-SLAMF6⁺ TEX_{prog} cells and CX3CR1⁺ TIM3⁻ TEX_{progenitor-like} cells as defined previously^{11,14}, and fewer terminally differentiated CX3CR1⁺ TIM3⁺ TEX_{eff} and CD101-expressing CX3CR1⁻ SLAMF6⁻ TEXₜₑᵣₘ cells (FIGS. 6G, 6H) 10.14. Thus, Adrb1 promotes the differentiation of terminally exhausted CD8⁺ T cells during chronic viral infection, and cells lacking this receptor were more capable of maintaining less differentiated, stem-like states (FIGS. 6F-6H). Consistent with the loss of the more highly cytotoxic TEX_{eff} subset and the gain of the TEX_{prog}-like subset^{10,11,15} reduced Granzyme B production and marginally reduced IFNγ and TNF production upon antigen-specific stimulation in Adrb1 cKO P14⁺ cells were observed (FIGS. 5C, 6I). Despite this, the Adrb1 cKO cells displayed better viral control than the WT cells at d30 p.i, with LCMV-clone 13, likely due to the increased presence of TEX_{prog} cells that sustain the anti-viral T cell responses (FIG. 6J)^{6,8,12}.

To determine if there was synergy between genetic ablation of Adrb1 and ICB, LCMV-clone 13 infected mice containing Adrb1 cKO and WT P14⁺ cells were treated with anti-PD-L1 or an isotype control antibody from days 23-36 p.i. Relative to the WT cells or isotype control treated mice, the Adrb1 cKO P14⁺ donor cells displayed larger responses to ICB based on increased frequency of cells (FIG. 6K), and trended to having lower viral titers albeit it was not significantly different than animals containing WT cells treated with ICB (FIG. 5D). This result indicates that Adrb1 cKO cells had more robust responses to ICB than WT cells.

Additionally, in agreement with ADRB1 signaling regulating cAMP and CREM levels in virus-specific CD8⁺ T cells (FIGS. 4H-4K), the Adrb1 cKO cells had lower intracellular cAMP levels and CREM expression than the WT controls (FIG. 6L). While overexpression of Crem was lethal to adoptively transferred P14⁺ cells in LCMV-clone 13 infection in vivo, Crem knockdown using shRNA led to a lower frequency of P14⁺ cells, yet prevented the cells from acquiring traits of CD8⁺ T cell exhaustion including increased PD-1 and TIM3-expression, instead causing the cells to acquire a CX3CR1⁺ PD-1⁻ terminal effector CD8⁺ T cell phenotype³³ (FIGS. 5E, 5F). These findings thus extend the role of CREM for exhaustion differentiation to CD8⁺ T cells.

Lastly, because exhausted CD8⁺ T cells were observed to localize closer to TH⁺ nerves, it was asked whether Adrb1 deletion influences the location of the T cells in the LCMV-clone 13 infected spleen at d31 p.i. Microscopy revealed that whereas WT P14⁺ CD8⁺ T cells largely localized adjacent to TH⁺ sympathetic nerves in the red pulp, Adrb1 cKO P14⁺ cells did not surround the nerves and mostly occupied niches in the red pulp that contain F4/80⁺ macrophages (FIGS. 5G, 6M). Thus, Adrb1 determines the peri-neural location of CD8⁺ TEX cells around sympathetic nerves in the chronically infected spleen. Taken together, these results demonstrate that deletion of Adrb1 on virus-specific CD8⁺ T cells promotes the maintenance of TEX_{prog} states, impairs migration towards sympathetic nerves and synergizes with ICB therapy.

### Example 5

### ADRB1-Blockade Curbs TEXₜₑᵣₘ Formation

This example illustrates that pharmacological blockade of ADRB1 in chronic infection curbs TEXₜₑᵣₘ differentiation of virus-specific CD8⁺ T cells.

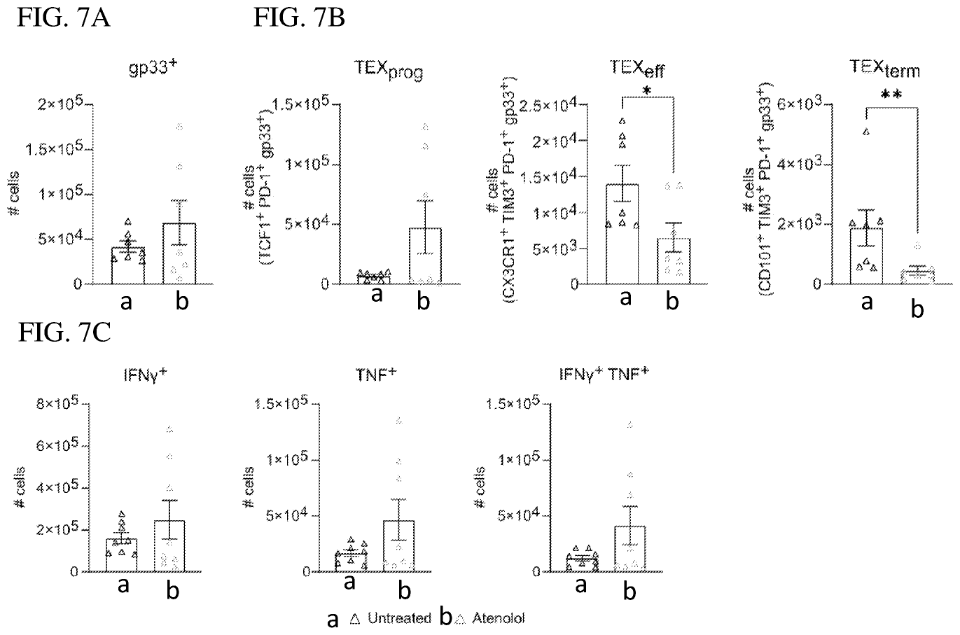

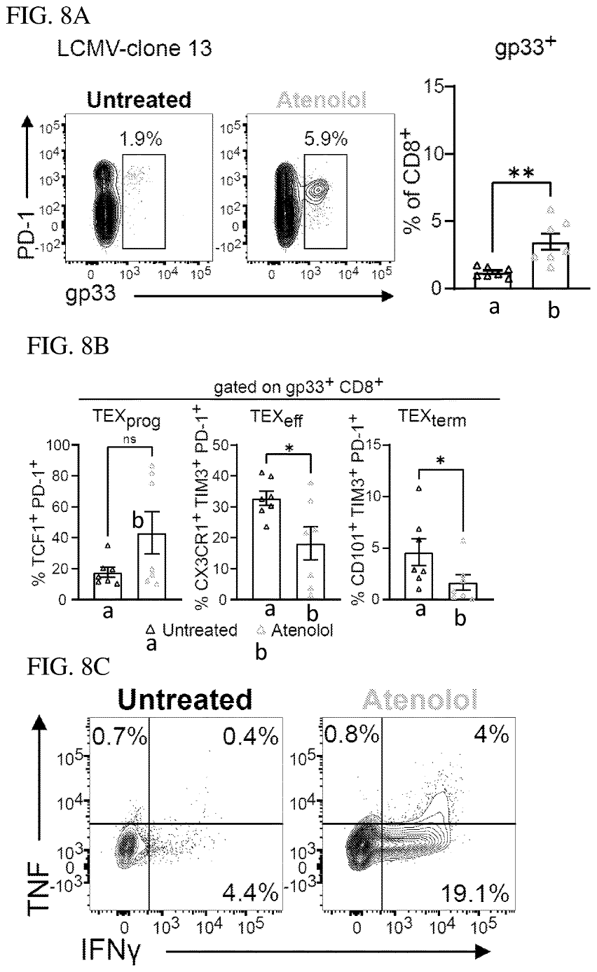

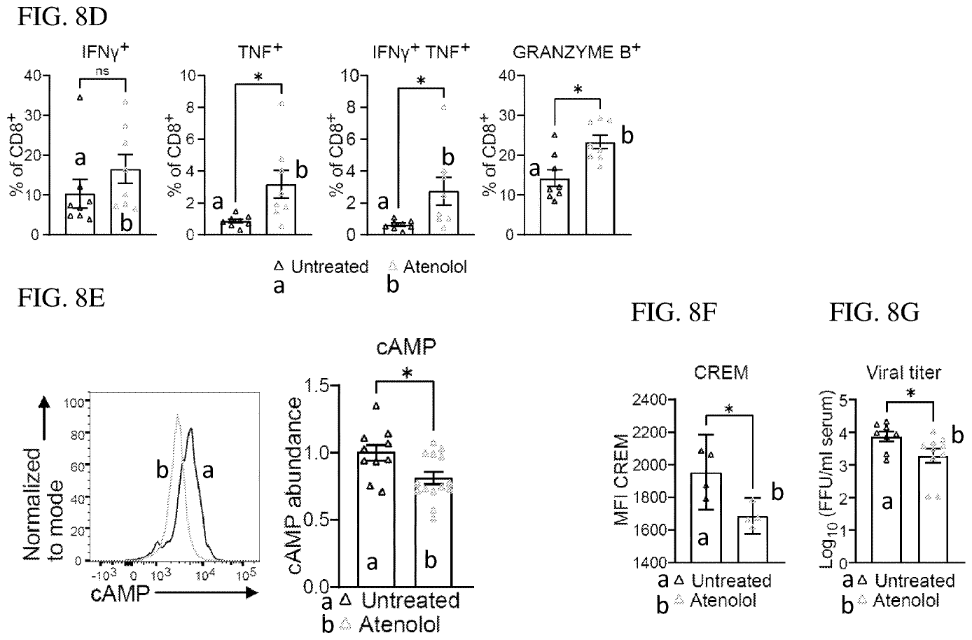

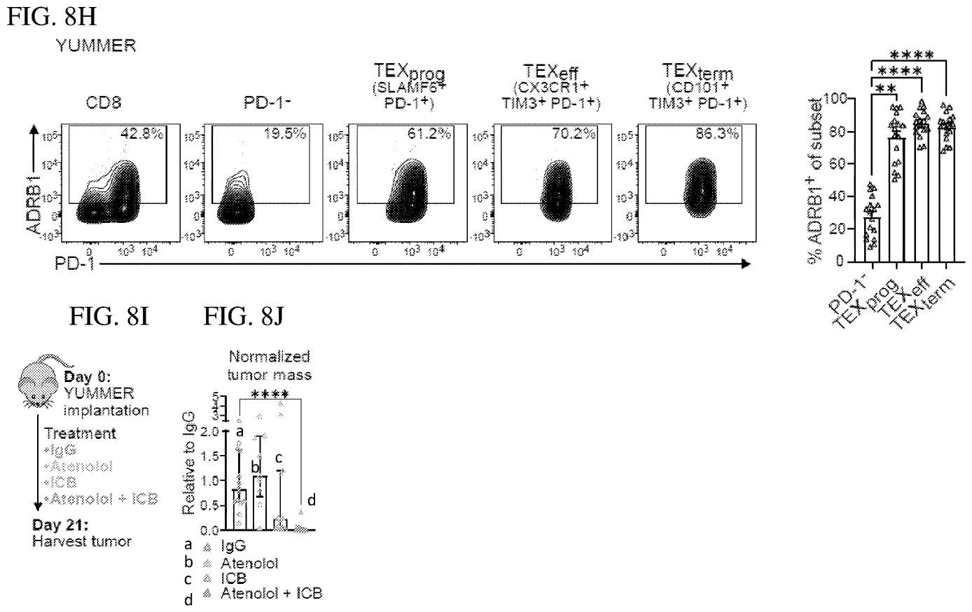

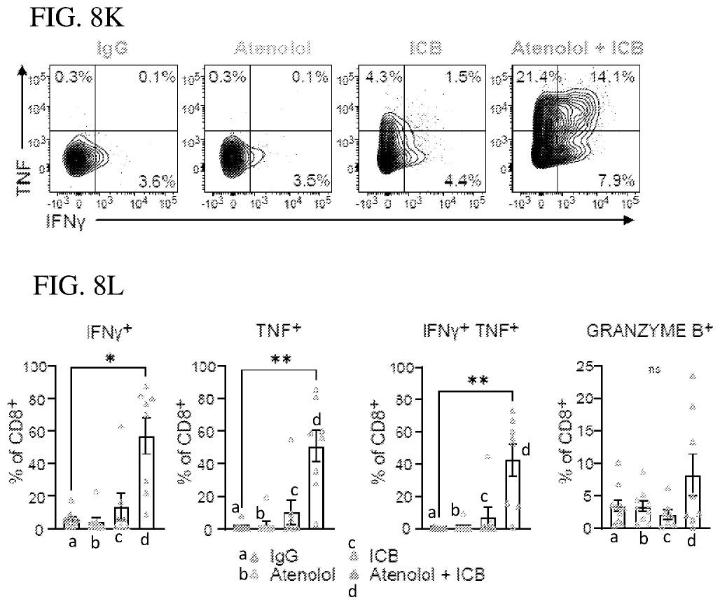

Beta-blockers are a widely prescribed class of drugs mainly used for the treatment of cardiac diseases. Atenolol is a commonly used beta-blockers and is clinically considered ADRB1-selective³⁴. Given the findings disclosed herein using the Adrb1 knockout model, it was examined whether pharmacologically blocking the ADRB1 receptor in chronically infected mice would exert a similar influence on T cell exhaustion as Adrb1 deletion (FIGS. 8A-8F). Atenolol treatment induced greater expansion of virus-specific CD8⁺ T cells that displayed features of TBX_{prog} at the expense of developing into TEX_{eff} and TEXₜₑᵣₘ (FIGS. 7A, 7B, 8A, 8B). The atenolol treated virus-specific CD8⁺ T cells also generated higher frequencies of IFNγ-, TNF- and GZMB-producing cells (FIGS. 7C, 8C, 8D). Consistent with the genetic deletion of Adrb1, atenolol treatment lowered the amounts of cAMP and CREM in virus-specific CD8⁺ T cells (FIGS. 8E, 8F) and resulted in lower viral titers (FIG. 8G). Taken together these findings show that pharmacological blockade of ADRB1 prevents functional exhaustion of antigen-specific CD8⁺ T cells and highlight a novel adjuvant therapy for patients with chronic infections.

### Example 6

### ADRB1-Blockade Rejuvenates TILs

This example illustrates that pharmacological blockade of ADRB1 prevents functional exhaustion in tumor infiltrating T cells.

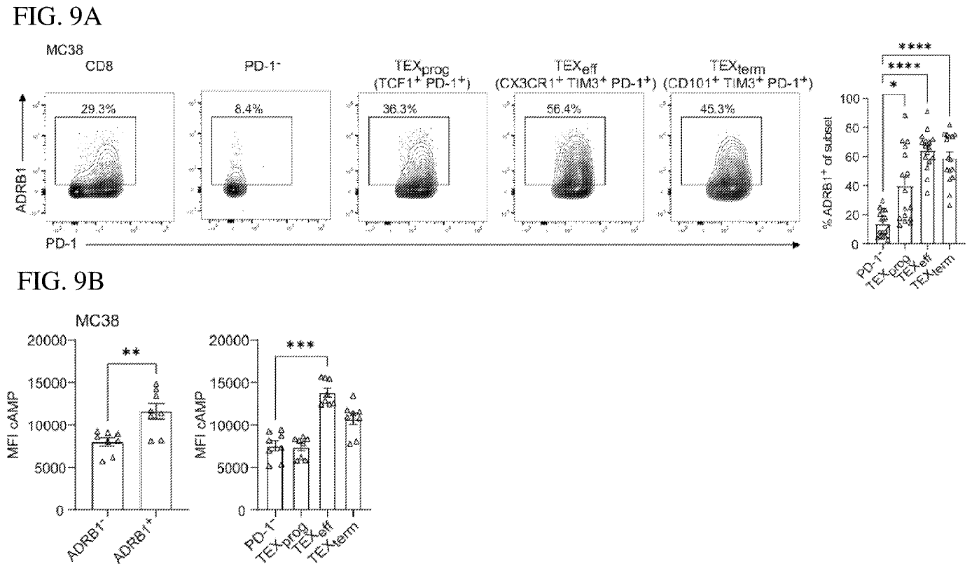

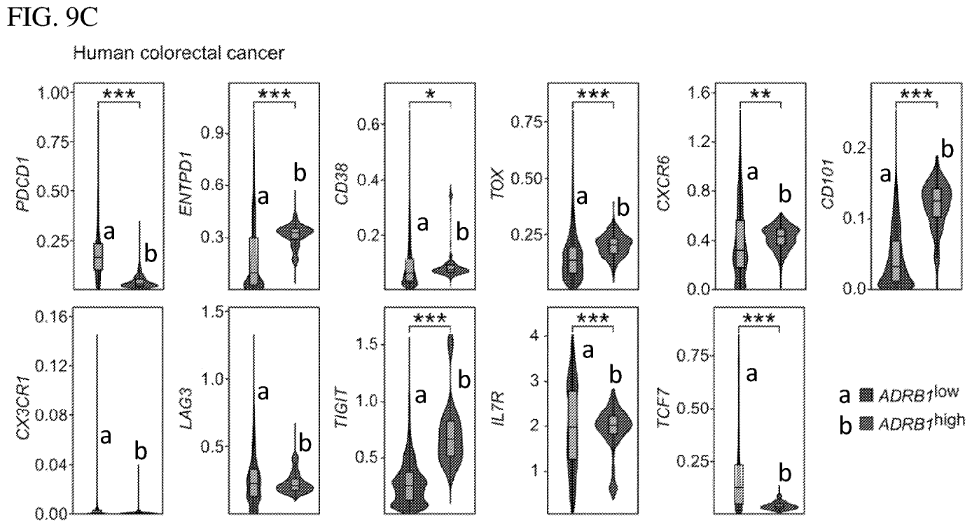

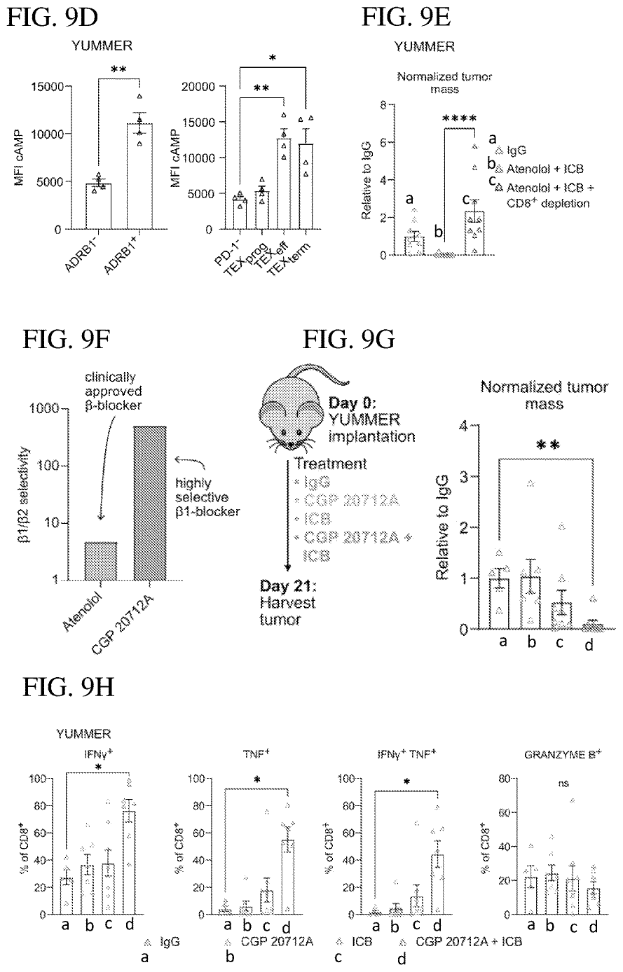

T cell exhaustion limits effective T cell responses in cancer¹. In murine models of colon cancer (MC38) and ICB-responsive melanoma (YUMMER1.7), exhausted TILs express high levels of ADRB1 and elevated intracellular cAMP (FIGS. 8H, 9A-D), similar to what is observed in mice infected with LCMV-clone 13. Furthermore, ADRB1-expressing CD8⁺ TILs in human colorectal cancer express higher levels of multiple markers of terminally exhausted cells such as ENTPD1, TOX, CXCR6, CD101 and lower levels of markers of less exhausted cells such as TCF7 (FIG. 9C).

Given the high expression of ADRB1 by TILs, the effects of pharmacological blockade of ADRB1 using atenolol alone or in combination with ICB (anti-PD-1⁺ anti-CTLA-4) on the T cell response against YUMMER1.7 melanomas were assessed (FIG. 8I). Compared to untreated tumors or those treated with atenolol alone, tumors in mice treated with the combination therapy were significantly smaller (FIG. 8J), and this effect was dependent on CD8⁺ T cells (FIG. 9E). While the effects of the combination therapy on differentiation of exhausted T cells did not extend beyond those of the checkpoint therapy alone, the cytokine production by CD8⁺ T cells was dramatically increased in mice treated with the combination therapy (FIGS. 8K, 8L). Despite being clinically used as β1-selective, atenolol can also block the β2-adrenergic receptor to a lower degree³⁵. Therefore, the combination therapy was repeated in melanoma-bearing mice with the β1-antagonist CGP 20712A, that has an ADRB1 vs. ADRB2 selectivity ratio of 501.2³⁵ (FIGS. 9F, 9G). Again, the tumors in mice treated with the combination therapy of ADRB1 inhibition and ICB were significantly smaller (FIG. 9G) and cytokine production by CD8⁺ T cells was significantly increased (FIG. 9H). Thus, blocking ADRB1 synergizes profoundly with immunotherapy to improve anti-tumor TIL responses in an ICB-responsive model of melanoma. In combination with the results relating to chronic viral infection disclosed herein, this indicates that ADRB1 expression is a conserved, and targetable, feature of T cell exhaustion across different etiologies, tumor types and species.

### Example 7

### Beta-Blockade and ICB Synergize in PDAC

This example illustrates that pharmacological blockade of adrenergic receptors enables effective checkpoint therapy in pancreatic adenocarcinoma.

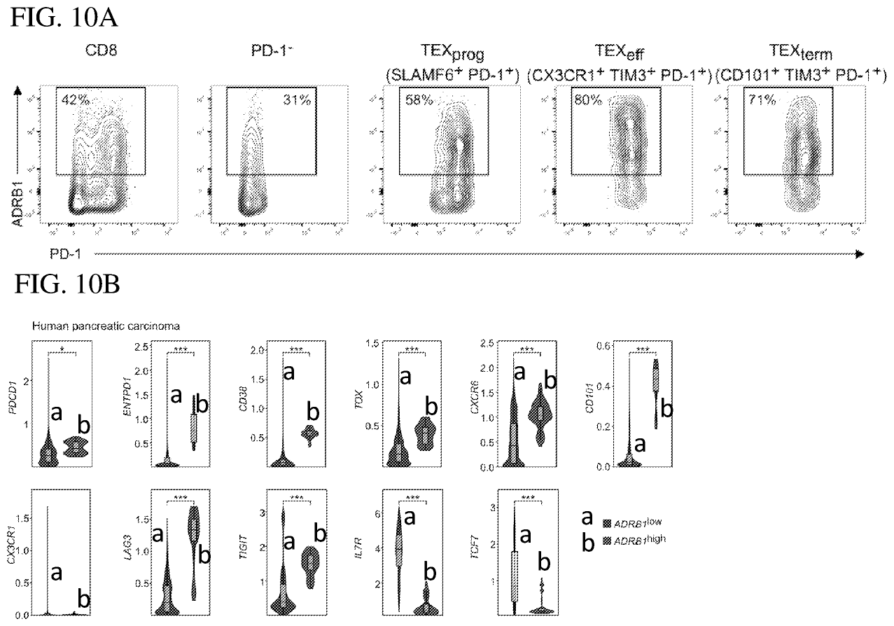

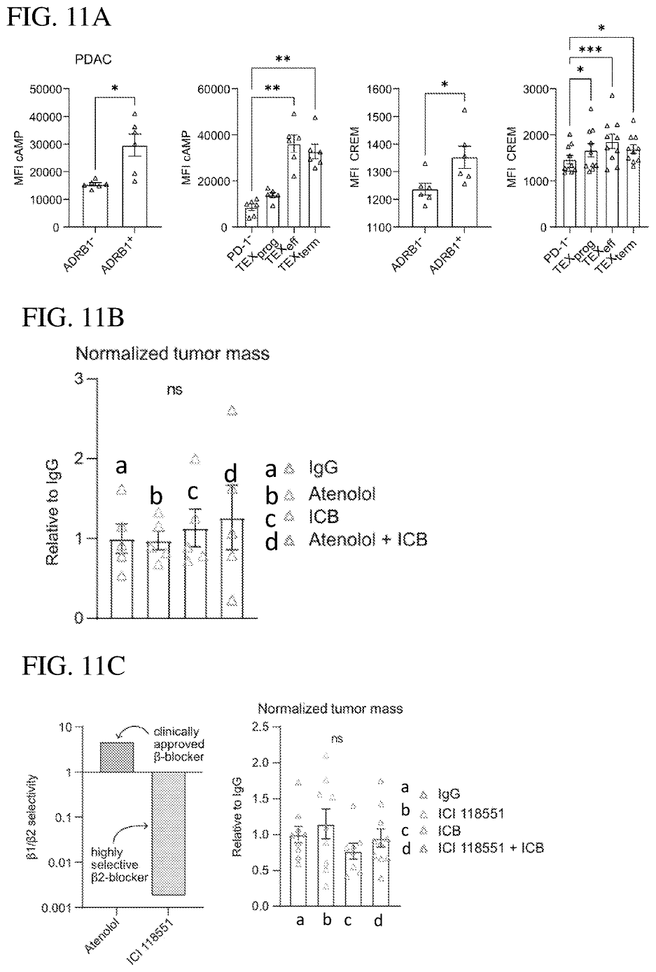

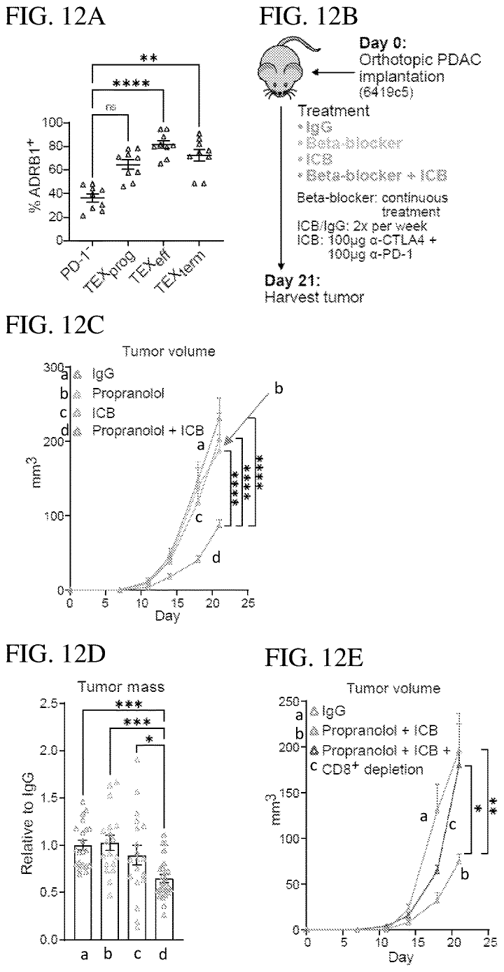

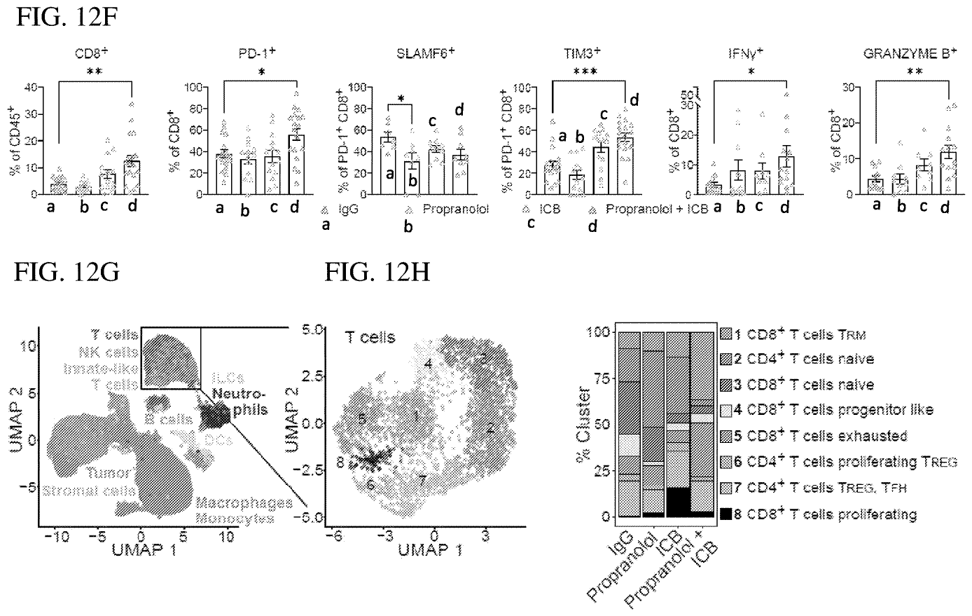

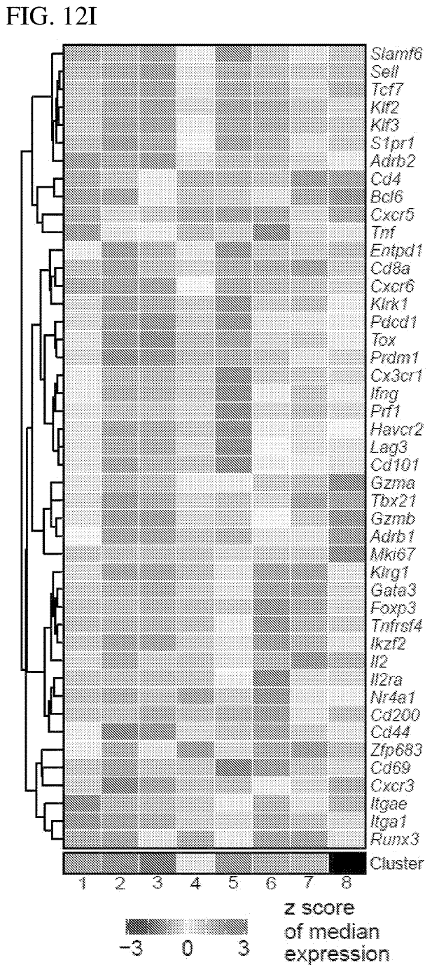

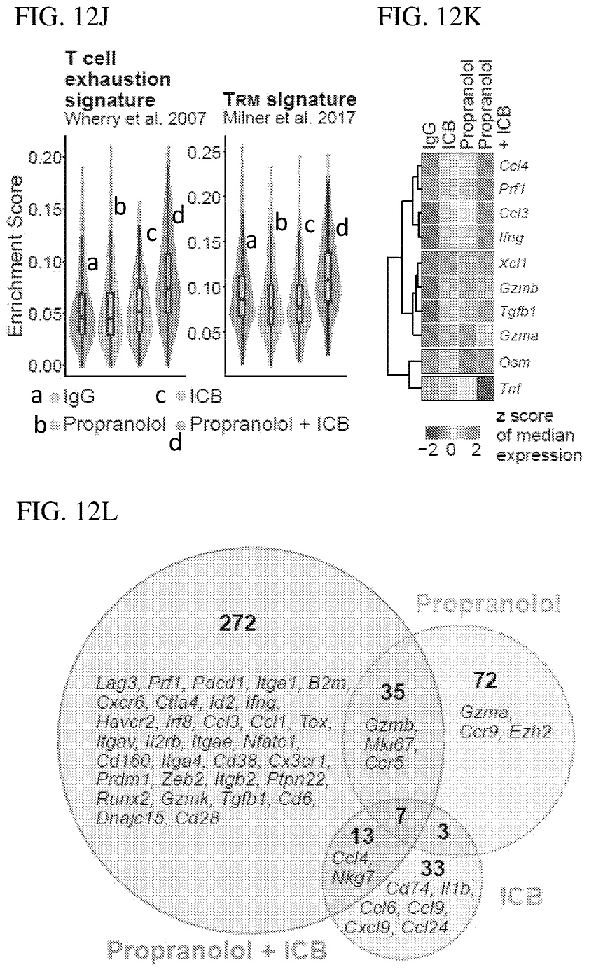

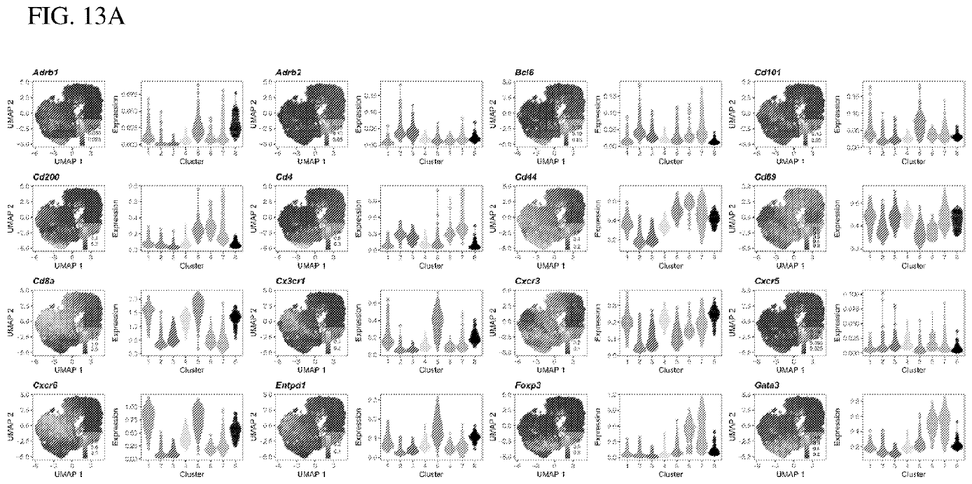

It was assessed whether beta-blocker treatment improves therapeutic responses in a tumor type insensitive to ICB alone. Pancreatic ductal adenocarcinoma (PDAC) is the most common form of pancreatic cancer and has a very poor prognosis with a 5-year survival rate of 9% due to late manifestation of symptoms and lack of effective therapies, including immunotherapy^{17,36}. Notably, the pancreas is highly innervated by adrenergic sympathetic nerves that are involved in its development and regulating endocrine function³⁷. Therefore, the influence of pharmacological blockade of adrenergic receptors on the T cell response in an immunologically ‘cold’ orthotopic murine model of PDAC was explored. As seen in the other tumor models, ADRB1 was highly expressed on TILs in murine orthotopic PDAC, especially on PD-1⁺ exhausted T cells (FIGS. 10A, 12A) and ADRB1-expressing CD8⁺ TILs in human pancreatic cancer expressed significantly higher levels of multiple exhaustion markers (e.g., PDCD1, ENTPD1, CD38, TOX, CXCR6, CD101, LAG3, TIGIT) and lower levels of markers found in less differentiated cells (e.g., IL7R, TCF7) (FIG. 10B). Exhausted CD8⁺ T cells in PDAC also displayed higher cAMP and CREM levels that were even more pronounced in ADRB1 positive cells (FIG. 11A). Despite this, blockade of ADRB1 alone with atenolol or in combination with ICB failed to suppress tumor growth (FIGS. 11B, 12B).

Selective blockade of ADRB2 using the highly selective B2-antagonist ICI 11855135 alone or in combination with ICB also failed to suppress tumor growth in the ‘cold’ PDAC model (FIG. 11C). Therefore, attention was focused on pan-ADRB1 and ADRB2 beta-blockade using the non-selective beta-blocker propranolol that blocks both ADRB1 and ADRB2 as monotherapy or in combination with ICB (FIG. 12B). This showed that tumor growth was significantly reduced using combination treatment with propranolol based on both abdominal ultrasound measurements and tumor mass (FIGS. 12C, 12D), an effect dependent on CD8⁺ T cells (FIG. 12E). While there was no response to ICB alone, the combination of propranolol and ICB led to an increase in CD8⁺ T cells in the tumors as well as higher expression of the activation/exhaustion markers PD-1 and TIM3 (FIG. 12F). Notably, increased production of IFNγ and GZMB were observed (FIG. 12F).

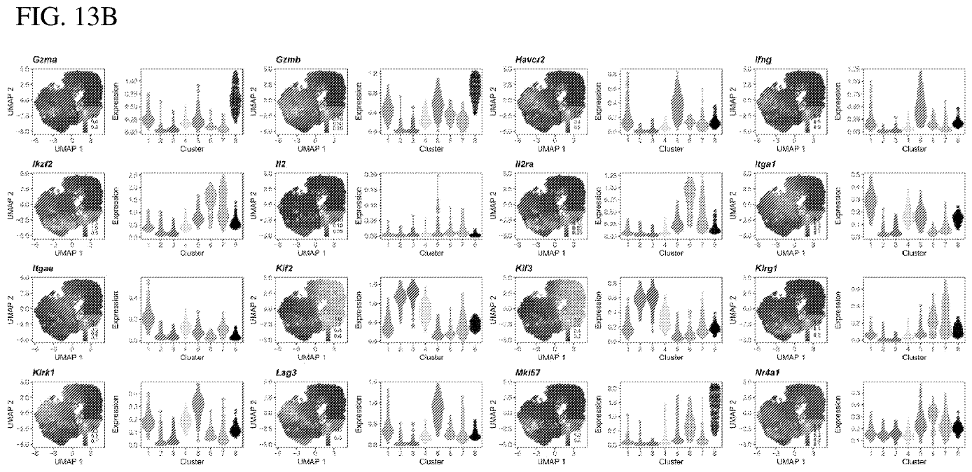

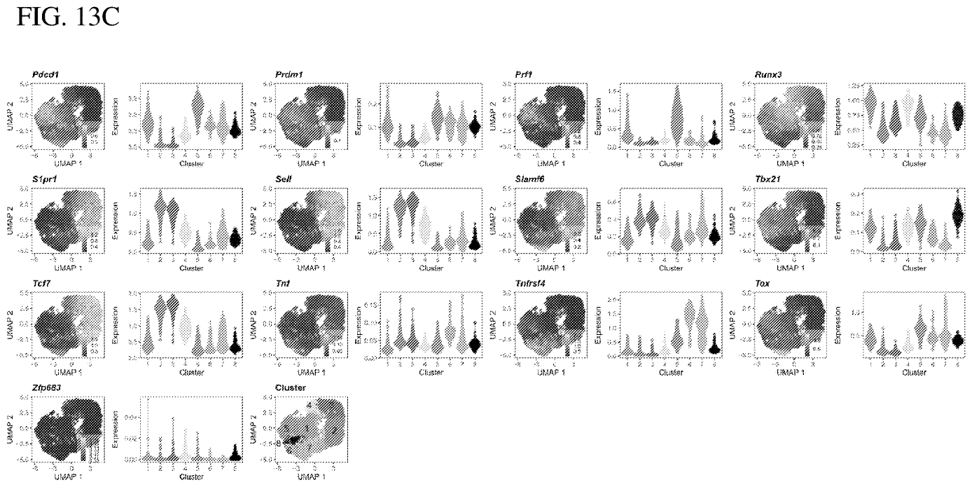

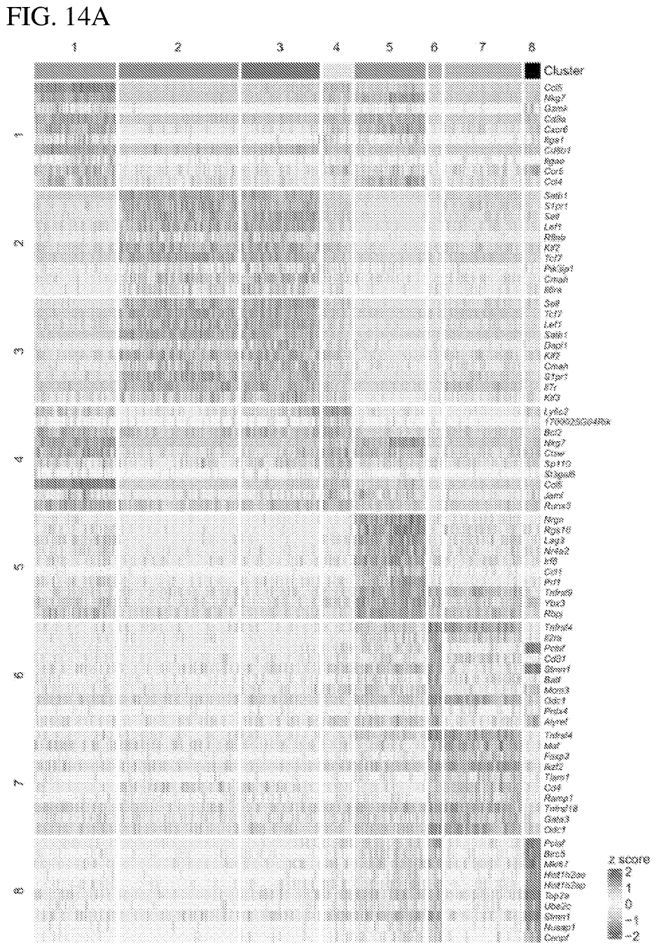

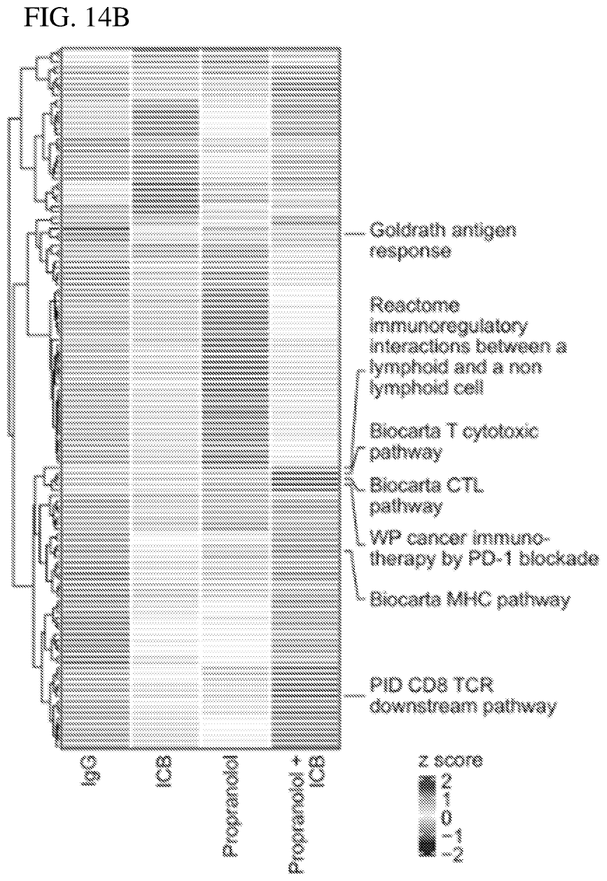

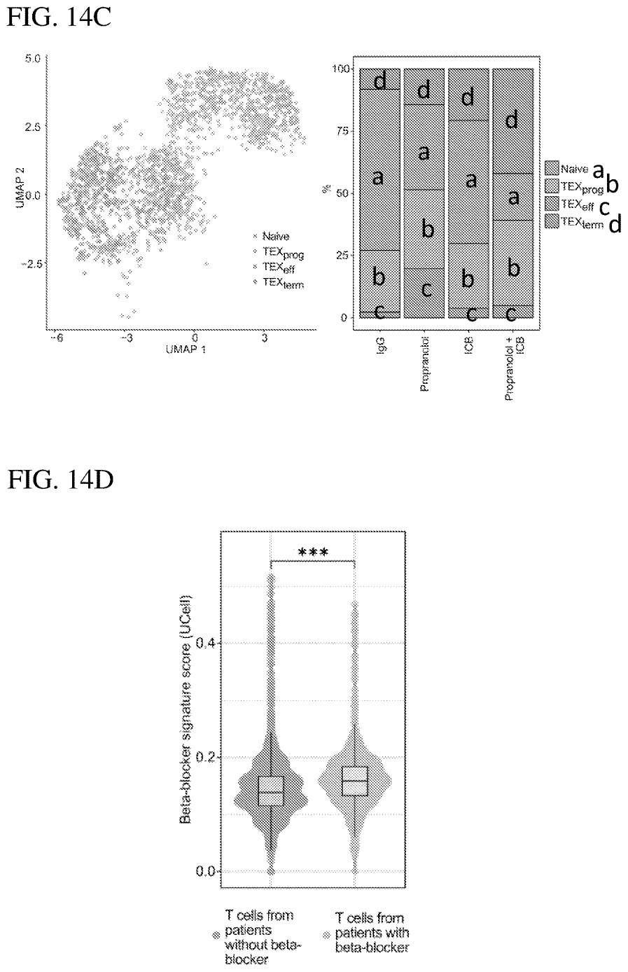

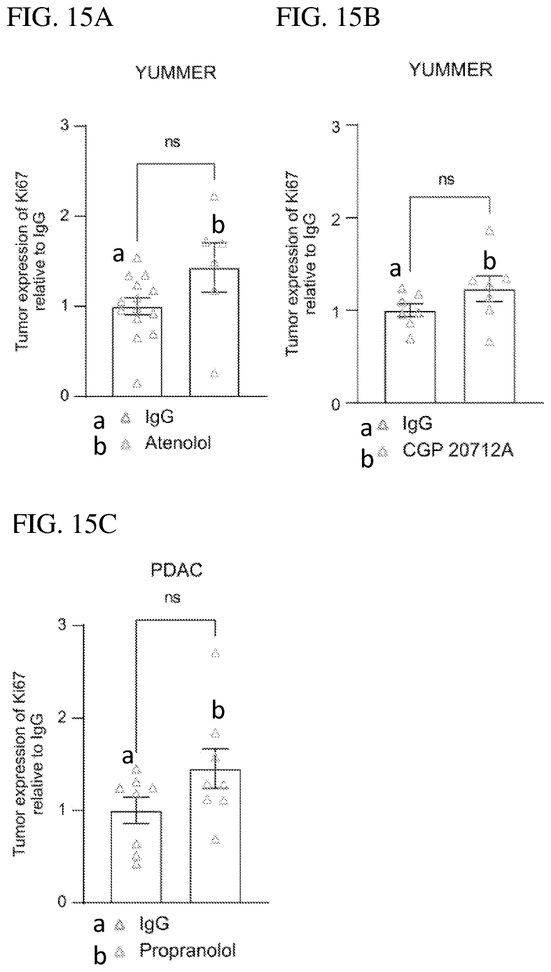

To further dissect how beta-blocker treatment rendered PDAC tumors sensitive to ICB, scRNA-seq was performed to identify 8 distinct T cell clusters. Cluster 1 and 5 in particular were enriched under the combination treatment (FIGS. 12G, 12H, 13A-C). CD8⁺ T cells in cluster 5 expressed high levels of multiple exhaustion markers (Tox, Cd101, Lag3, Haver2, Cx3cr1, Pded1, Cxcr6, Entpd1; FIGS. 12I, 13A-13C, 14A). CD8⁺ T cells in cluster 1 expressed multiple markers characteristic of tissue resident memory (T_{RM}) cells such as Itgae, Itga1, Runx3, Cxcr3, Cd69, low S1pr1, low Klf2/3 (FIG. 12I, 13A-13C, 14A), which have been correlated with a positive prognosis and response to immune checkpoint therapy⁴⁴⁻⁴⁶. Signatures for T cell response, exhaustion and TRM were enriched in the combination treatment condition (FIG. 12J, 14B, 14C). CD8⁺ T cells from animals receiving the combination treatment displayed enhanced cytokine and chemokine production such as increased Gzma, Tgfb1, Gzmb, Xcl1, Ccl3, Ccl4, Ifng, Prf1 (FIG. 12K). Analysis of differentially upregulated genes revealed synergistic effects of propranolol and ICB therapy in driving an enhanced T cell response with 272 upregulated genes compared to the isotype control (FIG. 12L). The identified beta-blocker signature (FIG. 12L) was also found to be enriched in human PDAC patients on beta-blocker therapy (FIG. 14D), confirming a translational human relevance of the altered CD8⁺ T cell transcriptome identified. While adrenergic receptor blockade and ICB synergized to reprogram the anti-tumor T cell response, tumor cell proliferation was not directly affected by the beta-blocker treatment (FIG. 15).

### REFERENCES

1 Amezquita, R. A. & Kaech, S. M. Immunology: The chronicles of T-cell exhaustion. Nature 543, 190-191, doi:10.1038/nature21508 (2017).
2 Xie, H. et al. Association of increased circulating catecholamine and glucocorticoid levels with risk of psychological problems in oral neoplasm patients. PloS one 9, e99179, doi:10.1371/journal.pone.0099179 (2014).
3 Bastos, D. B. et al. Circulating catecholamines are associated with biobehavioral factors and anxiety symptoms in head and neck cancer patients. PloS one 13, e0202515, doi:10.1371/journal.pone.0202515 (2018).
4 Wherry, E. J. & Kurachi, M. Molecular and cellular insights into T cell exhaustion. Nat Rev Immunol 15, 486-499, doi:10.1038/nri3862 (2015).
5 Alfei, F. et al. TOX reinforces the phenotype and longevity of exhausted T cells in chronic viral infection. Nature 571, 265-269, doi:10.1038/s41586-019-1326-9 (2019).
6 Khan. O. et al. TOX transcriptionally and epigenetically programs CD8(+) T cell exhaustion.

Nature 571, 211-218, doi:10.1038/s41586-019-1325-x (2019).
7 Paley, M. A. et al. Progenitor and terminal subsets of CD8+ T cells cooperate to contain chronic viral infection. Science 338, 1220-1225, doi:10.1126/science.1229620 (2012).
8 Im, S. J. et al. Defining CD8⁺ T cells that provide the proliferative burst after PD-1 therapy. Nature 537, 417-421, doi:10.1038/nature19330 (2016).
9 Miller, B. C. et al. Subsets of exhausted CD8(+) T cells differentially mediate tumor control and respond to checkpoint blockade. Nature immunology 20, 326-336, doi:10.1038/s41590-019-0312-6 (2019).
10 Hudson, W. H. et al. Proliferating Transitory T Cells with an Effector-like Transcriptional Signature Emerge from PD-1(+) Stem-like CD8(+) T Cells during Chronic Infection. Immunity 51, 1043-1058.e1044, doi:10.1016/j.immuni.2019.11.002 (2019).
11 Raju, S. et al. Identification of a T-bet(hi) Quiescent Exhausted CD8 T Cell Subpopulation That Can Differentiate into TIM3(+)CX3CR1 (+) Effectors and Memory-like Cells. Journal of immunology (Baltimore, Md.: 1950) 206, 2924-2936, doi:10.4049/jimmunol.2001348 (2021).
12 Zehn, D., Thimme, R., Lugli. E., de Almeida, G. P. & Oxenius, A. ‘Stem-like’ precursors are the fount to sustain persistent CD8(+) T cell responses. Nature immunology 23, 836-847, doi:10.1038/s41590-022-01219-w (2022).
13 Chung, H. K., McDonald, B. & Kaech, S. M. The architectural design of CD8+ T cell responses in acute and chronic infection: Parallel structures with divergent fates. The Journal of experimental medicine 218, doi:10.1084/jem.20201730 (2021).
14 Chen, Y. et al. BATF regulates progenitor to cytolytic effector CD8(+) T cell transition during chronic viral infection. Nature immunology 22, 996-1007, doi:10.1038/s41590-021-00965-7 (2021).
15 Kasmani, M. Y. et al. Clonal lineage tracing reveals mechanisms skewing CD8⁺ T cell fate decisions in chronic infection. The Journal of experimental medicine 220, doi:10.1084/jem.20220679 (2023).
16 Ribas, A. & Wolchok, J. D. Cancer immunotherapy using checkpoint blockade. Science 359, 1350-1355, doi:10.1126/science.aar4060 (2018).
17 Schmiechen, Z. C. & Stromnes, I. M. Mechanisms Governing Immunotherapy Resistance in Pancreatic Ductal Adenocarcinoma. Frontiers in immunology 11, 613815, doi:10.3389/fimmu.2020.613815 (2020).
18 Mueller, S. N. Neural control of immune cell trafficking. The Journal of experimental medicine 219, doi:10.1084/jem.20211604 (2022).
19 Devi, S. et al. Adrenergic regulation of the vasculature impairs leukocyte interstitial migration and suppresses immune responses. Immunity 54, 1219-1230.e1217, doi:10.1016/j.immuni.2021.03.025 (2021).
20 Grebe, K. M. et al. Sympathetic nervous system control of anti-influenza CD8+ T cell responses. Proc Natl Acad Sci USA 106, 5300-5305, doi:10.1073/pnas.0808851106 (2009).
21 Sanders, V. M. et al. Differential expression of the beta2-adrenergic receptor by Th1 and Th2 clones: implications for cytokine production and B cell help. Journal of immunology (Baltimore, Md.: 1950) 158, 4200-4210 (1997).
22 Daher, C. et al. Blockade of β-Adrenergic Receptors Improves CD8(+) T-cell Priming and Cancer Vaccine Efficacy. Cancer Immunol Res 7, 1849-1863, doi:10.1158/2326-6066.Cir-18-0833 (2019).
23 Felten, S. Y. & Olschowka, J. Noradrenergic sympathetic innervation of the spleen: II. Tyrosine hydroxylase (TH)-positive nerve terminals form synapticlike contacts on lymphocytes in the splenic white pulp. J Neurosci Res 18, 37-48, doi:10.1002/jnr.490180108 (1987).
24 Giles, J. R. et al. Human epigenetic and transcriptional T cell differentiation atlas for identifying functional T cell-specific enhancers. Immunity 55, 557-574.e557, doi:10.1016/j.immuni.2022.02.004 (2022).
25 Sassone-Corsi, P. The cyclic AMP pathway. Cold Spring Harb Perspect Biol 4, doi:10.1101/cshperspect.a011148 (2012).
26 Maine, C. J., Teijaro, J. R., Marquardt, K. & Sherman. L. A. PTPN22 contributes to exhaustion of T lymphocytes during chronic viral infection. Proc Natl Acad Sci USA 113, E7231-e7239, doi:10.1073/pnas.1603738113 (2016).
27 Sandu, I., Cerletti, D., Claassen, M. & Oxenius, A. Exhausted CD8(+) T cells exhibit low and strongly inhibited TCR signaling during chronic LCMV infection. Nature communications 11, 4454, doi:10.1038/s41467-020-18256-4 (2020).
28 Staron, M. M. et al. The transcription factor FoxO1 sustains expression of the inhibitory receptor PD-1 and survival of antiviral CD8(+) T cells during chronic infection. Immunity 41, 802-814, doi:10.1016/j.immuni.2014.10.013 (2014).
29 Riley, J. L. PD-1 signaling in primary T cells. Immunol Rev 229, 114-125, doi:10.1111/j.1600-065X.2009.00767.x (2009).
30 Aandahl, E. M. et al. Protein kinase A type I antagonist restores immune responses of T cells from HIV-infected patients. Faseb j 12, 855-862. doi:10.1096/fasebj.12.10.855 (1998).
31 Joshi, N. S. et al. Inflammation directs memory precursor and short-lived effector CD8(+) T cell fates via the graded expression of T-bet transcription factor. Immunity 27, 281-295, doi:10.1016/j.immuni.2007.07.010 (2007).
32 Sarkar, S. et al. Functional and genomic profiling of effector CD8 T cell subsets with distinct memory fates. The Journal of experimental medicine 205, 625-640, doi:10.1084/jem.20071641 (2008).
33 Gerlach, C. et al. The Chemokine Receptor CX3CR1 Defines Three Antigen-Experienced CD8 T Cell Subsets with Distinct Roles in Immune Surveillance and Homeostasis. Immunity 45, 1270-1284, doi:10.1016/j.immuni.2016.10.018 (2016).
34 Gorre, F. & Vandekerckhove, H. Beta-blockers: focus on mechanism of action. Which beta-blocker, when and why? Acta Cardiol 65, 565-570, doi:10.1080/ac.65.5.2056244 (2010).
35 Baker, J. G. The selectivity of beta-adrenoceptor antagonists at the human beta1, beta2 and beta3 adrenoceptors. Br J Pharmacol 144, 317-322, doi:10.1038/sj.bjp.0706048 (2005).
36 Rawla, P., Sunkara, T. & Gaduputi. V. Epidemiology of Pancreatic Cancer: Global Trends, Etiology and Risk Factors. World J Oncol 10, 10-27, doi:10.14740/wjon1166 (2019).
37 Woods, S. C. & Porte, D., Jr. Neural control of the endocrine pancreas. Physiol Rev 54, 596-619, doi:10.1152/physrev.1974.54.3.596 (1974).
38 Bayne, L. J. et al. Tumor-derived granulocyte-macrophage colony-stimulating factor regulates myeloid inflammation and T cell immunity in pancreatic cancer. Cancer Cell 21, 822-835, doi:10.1016/j.ccr.2012.04.025 (2012).
39 Stromnes, I. M. et al. Targeted depletion of an MDSC subset unmasks pancreatic ductal adenocarcinoma to adaptive immunity. Gut 63, 1769-1781, doi:10.1136/gutjnl-2013-306271 (2014).
40 Mohammadpour, H. et al. 32 adrenergic receptor-mediated signaling regulates the immunosuppressive potential of myeloid-derived suppressor cells. The Journal of clinical investigation 129, 5537-5552, doi:10.1172/jci129502 (2019).
41 Kokolus, K. M. et al. Beta blocker use correlates with better overall survival in metastatic melanoma patients and improves the efficacy of immunotherapies in mice. Oncoimmunology 7, e1405205, doi:10.1080/2162402x.2017.1405205 (2018).
42 Gandhi, S. et al. Phase I Clinical Trial of Combination Propranolol and Pembrolizumab in Locally Advanced and Metastatic Melanoma: Safety, Tolerability, and Preliminary Evidence of Antitumor Activity. Clin Cancer Res 27, 87-95, doi:10.1158/1078-0432.Ccr-20-2381 (2021).
43 Fjæstad, K. Y. et al. Blockade of beta-adrenergic receptors reduces cancer growth and enhances the response to anti-CTLA4 therapy by modulating the tumor microenvironment. Oncogene 41, 1364-1375, doi:10.1038/s41388-021-02170-0 (2022).
44 Ganesan, A. P. et al. Tissue-resident memory features are linked to the magnitude of cytotoxic T cell responses in human lung cancer. Nature immunology 18, 940-950. doi:10.1038/ni.3775 (2017).
45 Milner, J. J. et al. Runx3 programs CD8(+) T cell residency in non-lymphoid tissues and tumours. Nature 552, 253-257, doi:10.1038/nature24993 (2017).
46 Banchereau, R. et al. Intratumoral CD103+ CD8+ T cells predict response to PD-L1 blockade. J Immunother Cancer 9, doi:10.1136/jitc-2020-002231 (2021).
47 Kennedy, O. J. et al. Prognostic and predictive value of β-blockers in the EORTC 1325/KEYNOTE-054 phase III trial of pembrolizumab versus placebo in resected high-risk stage III melanoma. Eur J Cancer 165, 97-112, doi:10.1016/j.ejca.2022.01.017 (2022).
48 Udumyan, R. et al. Beta-Blocker Drug Use and Survival among Patients with Pancreatic Adenocarcinoma. Cancer Res 77, 3700-3707, doi:10.1158/0008-5472.Can-17-0108 (2017).
49 Beg, M. S. et al. Impact of Concurrent Medication Use on Pancreatic Cancer Survival-SEER-Medicare Analysis. Am J Clin Oncol 41, 766-771, doi:10.1097/coc.0000000000000359 (2018).
50 Pircher. H., Bürki. K., Lang, R., Hengartner, H. & Zinkernagel, R. M. Tolerance induction in double specific T-cell receptor transgenic mice varies with antigen. Nature 342, 559-561, doi:10.1038/342559a0 (1989).
51 Wang, J. et al. UV-induced somatic mutations elicit a functional T cell response in the YUMMER1.7 mouse melanoma model. Pigment Cell Melanoma Res 30, 428-435, doi:10.1111/pcmr.12591 (2017).
52 Li, J. et al. Tumor Cell-Intrinsic Factors Underlie Heterogeneity of Immune Cell Infiltration and Response to Immunotherapy. Immunity 49, 178-193.e177, doi:10.1016/j.immuni.2018.06.006 (2018).
53 Nüssing, S. et al. Efficient CRISPR/Cas9 Gene Editing in Uncultured Naive Mouse T Cells for In Vivo Studies. Journal of immunology (Baltimore, Md.: 1950) 204, 2308-2315, doi:10.4049/jimmunol.1901396 (2020).
54 Lun, A. T., McCarthy, D. J. & Marioni. J. C. A step-by-step workflow for low-level analysis of single-cell RNA-seq data with Bioconductor. F1000Res 5, 2122, doi:10.12688/f1000research.9501.2 (2016).
55 Aran, D. et al. Reference-based analysis of lung single-cell sequencing reveals a transitional profibrotic macrophage. Nature immunology 20, 163-172, doi:10.1038/s41590-018-0276-y (2019).
56 van Dijk, D. et al. Recovering Gene Interactions from Single-Cell Data Using Data Diffusion. Cell 174, 716-729.e727, doi:10.1016/j.cell.2018.05.061 (2018).
57 Gu, Z., Eils, R. & Schlesner, M. Complex heatmaps reveal patterns and correlations in multidimensional genomic data. Bioinformatics 32, 2847-2849, doi:10.1093/bioinformatics/btw313 (2016).
58 Wherry, E. J. et al. Molecular signature of CD8+ T cell exhaustion during chronic viral infection. Immunity 27, 670-684, doi:10.1016/j.immuni.2007.09.006 (2007).
59 Aibar, S. et al. SCENIC: single-cell regulatory network inference and clustering. Nat Methods 14, 1083-1086, doi:10.1038/nmeth.4463 (2017).
60 Andreatta, M. & Carmona, S. J. UCell: Robust and scalable single-cell gene signature scoring. Comput Struct Biotechnol J 19, 3796-3798, doi:10.1016/j.csbj.2021.06.043 (2021).
61 Radtke, A. J. et al. IBEX: an iterative immunolabeling and chemical bleaching method for high-content imaging of diverse tissues. Nat Protoc 17, 378-401, doi:10.1038/s41596-021-00644-9 (2022).
62 Radtke, A. J. et al. IBEX: A versatile multiplex optical imaging approach for deep phenotyping and spatial analysis of cells in complex tissues. Proc Natl Acad Sci USA 117, 33455-33465, doi:10.1073/pnas.2018488117 (2020).
63 Schalper, K. A. et al. Objective measurement and clinical significance of TILs in non-small cell lung cancer. J Natl Cancer Inst 107, doi:10.1093/jnci/dju435 (2015).

It will be apparent that the precise details of the methods or compositions described may be varied or modified without departing from the spirit of the described aspects of the disclosure. We claim all such modifications and variations that fall within the scope and spirit of the claims below.

1. A modified peripheral blood mononuclear cell (PBMC), comprising:
a) an agent that reduces Adrb1 expression or a non-naturally occurring genetic modification that reduces an amount of functional ADRB1; and/or
b) an agent that reduces Adrb2 expression or a non-naturally occurring genetic modification that reduces an amount of functional ADRB2.

2. The modified PBMC of claim 1, comprising:
a) the agent that reduces Adrb1 expression or the non-naturally occurring genetic modification that reduces the amount of functional ADRB1; and
b) the agent that reduces Adrb2 expression or the non-naturally occurring genetic modification that reduces the amount of functional ADRB2.

3. The modified PBMC of claim 1, wherein:
a) the agent that reduces Adrb1 expression comprises an inhibitory RNA (RNAi) specific for Adrb1 or a guide RNA (gRNA) specific for Adrb1; or
b) the agent that reduces Adrb2 expression comprises an RNAi specific for Adrb2 or a gRNA specific for Adrb2.

4. The modified PBMC of claim 3, wherein:
a) the RNAi specific for Adrb1 is a short hairpin RNA (shRNA) molecule, short interfering RNA (siRNA) molecule, or antisense RNA molecule; or
b) the RNAi specific for Adrb2 is a shRNA molecule, siRNA molecule, or antisense RNA molecule.

5. The modified PBMC of claim 3, wherein the agent that reduces Adrb1 expression or Adrb2 expression comprises a heterologous nucleic acid molecule; and
a) wherein the heterologous nucleic acid molecule encodes:
i) the RNAi specific for Adrb1 gene or transcript, wherein the RNAi specific for Adrb1 comprises at least 90% complementarity to a portion of the Adrb1 gene or transcript;
ii) the gRNA specific for Adrb1 gene or transcript, wherein the gRNA specific for Adrb1 comprises at least 90% sequence identity to a portion of the Adrb1 gene or transcript; or
iii) the gRNA specific for Adrb1 gene or transcript, wherein the gRNA specific for Adrb1 comprises at least 90% sequence identity to a portion of the Adrb1 gene or transcript and a Cas nuclease; or
b) wherein the heterologous nucleic acid molecule encodes:
i) the RNAi specific for Adrb2 gene or transcript, wherein the RNAi specific for Adrb2 comprises at least 90% complementarity to a portion of the Adrb2 gene or transcript;
ii) the gRNA specific for Adrb2 gene or transcript, wherein the gRNA specific for Adrb2 comprises at least 90% sequence identity to a portion of the Adrb2 gene or transcript; or
iii) the gRNA specific for Adrb2 gene or transcript, wherein the gRNA specific for Adrb2 comprises at least 90% sequence identity to a portion of the Adrb2 gene or transcript and a Cas nuclease.

6. The modified PBMC of claim 3, wherein the agent that reduces Adrb1 expression or Adrb2 expression comprises a heterologous nucleic acid molecule encoding:
a) the gRNA specific for Adrb1, comprising SEQ ID NOs: 7, 8, 9, 10, 11, 12, 13, 14, 15, or 16; or
b) the gRNA specific for Adrb2, comprising SEQ ID NO: 17, 18, 19, 20, 21, 22, 23, 24, 25 or 26.

7. The modified PBMC of claim 5, wherein the modified PBMC comprises an expression vector encoding the heterologous nucleic acid.

8. The modified PBMC of claim 5, comprising the gRNA specific for the Adrb1 or Adrb2 gene or transcript and the Cas nuclease, wherein the Cas nuclease is a Cas3, dCas3, Cas9, dCas9, Cas12, dCas12, Cas13a, dCas13a, Cas13b, dCas13b, Cas13d, or dCas13d nuclease.

9. The modified PBMC of claim 1, comprising the genetic modification that reduces ADRB1 or ADRB2, wherein:
a) the genetic modification that reduces ADRB1 is a point mutation, a partial deletion, full deletion, or insertion of Adrb1 that reduces expression of Adrb1 and/or reduces activity of ADRB1; or
b) the genetic modification that reduces ADRB2 is a point mutation, a partial deletion, full deletion, or insertion of Adrb2 that reduces expression of Adrb2 and/or reduces activity of ADRB2.

10. The modified PBMC of claim 1, wherein the modified PBMC is a T cell, or an antigen presenting cell.

11. The modified PBMC of claim 10,
wherein the T cell is:
a CD8+ T cell or a CD4+ T cell;
a therapeutic T cell;
an exhausted T cell, a tissue resident memory T cell (TRM), a chimeric antigen receptor (CAR) T cell, an engineered T cell receptor (TCR) T cell, or a tumor-infiltrating lymphocyte (TIL); and/or
reactive to a tumor-specific antigen or a viral antigen; or
wherein the antigen presenting cell is a monocyte/macrophage, dendritic cell, NK cell, or B cell.

12.-14. (canceled)

15. The modified PBMC of claim 11, wherein the tumor-specific antigen is one or more of CD19, CD20, BCMA, MUC1, PSA, CEA, HER1, HER2, TRP-2, EpCAM, GPC3, mesothelin 1 (MSLN), and EGFR.

16.-17. (canceled)

18. A method of generating the modified PBMC of claim 1, comprising:
a) introducing the agent that reduces Adrb1 expression or non-naturally occurring genetic modification that reduces functional ADRB1 into a PBMC, thereby generating the modified PBMC with reduced expression of Adrb1, reduced activity of ADRB1, or both; or
b) introducing the agent that reduces Adrb2 expression or non-naturally occurring genetic modification that reduces functional ADRB2 into a PBMC, thereby generating the modified PBMC with reduced expression of Adrb2, reduced activity of ADRB2, or both.

19. The method of claim 18,
wherein the PBMC is a T cell, a monocyte/macrophage, a dendritic cell, NK cell, or a B cell;
wherein the modified PBMC is reactive to a tumor-specific antigen or a viral antigen; and/or
wherein reduced expression of Adrb1, reduced activity of ADRB1, reduced expression of Adrb2, or reduced activity of ADRB2, increases effector function of the T cell, reduces exhaustion of the T cell, causes the T cell to express Itgae, Itga1, Runx3, Cxcr3, Prdm1, Notch2, Tcf7, Cxcr5, I17r, Id3, or Cd69 and/or causes reduced expression of S1pr1, Klf2, Klf3, Pdcd1, Tox, Entpd1, Cxcr6, Eomes, Tbx21, Tigit, Cd38, Lag3, Cx3cr1, Cd101, Havcr2 by the T cell.

20. The method of claim 18, wherein the method further comprises incubating the modified PBMC with interleukin 2, (IL-2), interleukin 7 (IL-7), interleukin 15 (IL-15), TGF-beta, retinoic acid or a combination thereof.

21. (canceled)

22. The method of claim 19, wherein the tumor-specific antigen is one or more of CD19, CD20, BCMA, MUC1, PSA, CEA, HER1, HER2, TRP-2, EpCAM, GPC3, mesothelin 1 (MSLN), and EGFR.

23.-24. (canceled)

25. The method of claim 18, further comprising:
a) selecting the modified PBMC with reduced expression of Adrb1, reduced activity of ADRB1, or both; or
b) selecting the modified PBMC with reduced expression of Adrb2, reduced activity of ADRB2, or both; and
optionally introducing the selected modified PBMC into a subject.

26. The method of claim 25, wherein the selecting step comprises use of flow cytometry, panning, or magnetic separation.

27. The method of claim 25, wherein the subject has cancer or a viral infection.

28. (canceled)

29. A pharmaceutical composition comprising: the modified PBMC of claim 1, and a pharmaceutically acceptable carrier, optionally wherein the composition is in an intravenous formulation, and optionally further comprising one or more immune checkpoint blockade (ICB) agents, one or more additional anti-cancer agents, one or more antiviral agents, or combinations thereof.

30. A method for treating cancer or a tumor in a subject, comprising:
administering a therapeutically effective amount of the modified PBMC of claim 1, a to the subject having cancer or the tumor, thereby treating the cancer or the tumor.

31.-44. (canceled)

45. A method for treating a viral infection in a subject, comprising:
administering a therapeutically effective amount of the modified PBMC of claim 1, to the subject having the viral infection, thereby treating the viral infection.

46-49. (canceled)

50. A modified peripheral blood mononuclear cell (PBMC) with increased expression of Adrb1, Adrb2, or both, wherein the modified PBMC comprises:
a) an agent that increases Adrb1 expression or a non-naturally occurring genetic modification that increases an amount of functional ADRB1; and/or
b) an agent that increases Adrb2 expression or a non-naturally occurring genetic modification that increases an amount of functional ADRB2.

51.-65. (canceled)

66. A method of preventing or treating cancer in a subject, comprising administering to a subject with cancer:
a) an agent that inhibits ADRB1 signaling administered in an amount effective to inhibit ADRB1 signaling on PBMCs and/or an agent that inhibits ADRB2 signaling administered in an amount effective to inhibit ADRB2 signaling on the PBMCs; and
b) a therapeutically effective amount of immune checkpoint blockade (ICB) agent.

67.-84. (canceled)
